SEQUENCE LISTING (277 sequences)

SEQ ID NO: 1 - protein, 246 aa, Artificial Sequence
  synthetic construct; heavy chain Adalimumab
  misc (226)..(229): amino acids 226 to 229 can be present or absent
  misc (230)..(236): amino acids 230 to 236 can be present or absent
  misc (237)..(246): amino acids 237 to 246 can be present or absent
EVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFL

SEQ ID NO: 2 - protein, 214 aa, Artificial Sequence
  synthetic construct; light chain Adalimumab
DIQMTQSPSSLSASVGDRVTITCRASQGIRNYLAWYQQKPGKAPKLLIYAASTLQSGVPSRFSGSGSGTDFTLTISSLQPEDVATYYCQRYNRAPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 245 aa, Artificial Sequence
  synthetic construct; heavy chain Infliximab
  misc (224)..(228): amino acids 224 to 228 can be present or absent
  misc (229)..(234): amino acids 229 to 234 can be present or absent
  misc (234)..(245): amino acids 235 to 245 can be present or absent
EVKLEESGGGLVQPGGSMKLSCVASGFIFSNHWMNWVRQSPEKGLEWVAEIRSKSINSATHYAESVKGRFTISRDDSKSAVYLQMTDLRTEDTGVYYCSRNYYGSTYDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFL

SEQ ID NO: 4 - protein, 214 aa, Artificial Sequence
  synthetic construct; light chain Infliximab
DILLTQSPAILSVSPGERVSFSCRASQFVGSSIHWYQQRTNGSPRLLIKYASESMSGIPSRFSGSGSGTDFTLSINTVESEDIADYYCQQSHSWPFTFGSGTNLEVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 5 - protein, 254 aa, Artificial Sequence
  synthetic construct; heavy chain Golimumab
  misc (234)..(237): amino acids 234 to 237 can be present or absent
  misc (238)..(243): amino acids 238 to 243 can be present or absent
  misc (244)..(254): amino acids 238 to 243 can be present or absent
  misc (244)..(254): amino acids 244 to 254 can be present or absent
SKLQVQLVESGGGVVQPGRSLRLSCAASGFIFSSYAMHWVRQAPGNGLEWVAFMSYDGSNKKYADSVKGRFTISRDNSKNTLYLQMNSLRAEDTAVYYCARDRGIAAGGNYYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFL

SEQ ID NO: 6 - protein, 218 aa, Artificial Sequence
  synthetic construct; light chain Golimumab
AGSEIVLTQSPATLSLSPGERATLSCRASQSVYSYLAWYQQKPGQAPRLLIYDASNRATGIPARFSGSGSGTDFTLTISSLEPEDFAVYYCQQRSNWPPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 7 - protein, 235 aa, Artificial Sequence
  synthetic construct; heavy chain Satralizumab
  misc (225)..(230): amino acids 225 to 230 can be present or absent
  misc (231)..(235): amino acids 231 to 235 can be present or absent
QVQLQESGPGLVKPSETLSLTCAVSGHSISHDHAWSWVRQPPGEGLEWIGFISYSGITNYNPSLQGRVTISRDNSKNTLYLQMNSLRAEDTAVYYCARSLARTTAMDYWGEGTLVTVSSASTKGPSVFPLAPSSKSTSGGTAALGCLVKDYFPEPVTVSWNSGALTSGVHTFPAVLQSSGLYSLSSVVTVPSSNFGTQTYTCNVDHKPSNTKVDKTVERKSCVECPPCPAPPVAG

SEQ ID NO: 8 - protein, 214 aa, Artificial Sequence
  synthetic construct; light chain Satralizumab
DIQMTQSPSSLSASVGDSVTITCQASTDISSHLNWYQQKPGKAPELLIYYGSHLLSGVPSRFSGSGSGTDFTFTISSLEAEDAATYYCGQGNRLPYTFGQGTKVEIERTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 9 - protein, 241 aa, Artificial Sequence
  synthetic construct; heavy chain Sarilumab
  misc (221)..(224): amino acids 221 to 224 can be present or absent
  misc (225)..(230): amino acids 225 to 230 can be present or absent
  misc (231)..(241): amino acids 231 to 241 can be present or absent
EVQLVESGGGLVQPGRSLRLSCAASRFTFDDYAMHWVRQAPGKGLEWVSGISWNSGRIGYADSVKGRFTISRDNAENSLFLQMNGLRAEDTALYYCAKGRDSFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFL

SEQ ID NO: 10 - protein, 214 aa, Artificial Sequence
  sythetic construct; light chain Sarilumab
DIQMTQSPSSVSASVGDRVTITCRASQGISSWLAWYQQKPGKAPKLLIYGASSLESGVPSRFSGSGSGTDFTLTISSLQPEDFASYYCQQANSFPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 244 aa, Artificial Sequence
  synthetic construct; heavy chain Siltuximab
  misc (224)..(227): amino acids 224 to 227 can be present or absent
  misc (228)..(233): amino acids 228 to 233 can be present or absent
  misc (234)..(244): amino acids 234 to 244 can be present or absent
EVQLVESGGKLLKPGGSLKLSCAASGFTFSSFAMSWFRQSPEKRLEWVAEISSGGSYTYYPDTVTGRFTISRDNAKNTLYLEMSSLRSEDTAMYYCARGLWGYYALDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFL

SEQ ID NO: 12 - protein, 213 aa, Artificial Sequence
  synthetic construct; light chain Siltuximab
QIVLIQSPAIMSASPGEKVTMTCSASSSVSYMYWYQQKPGSSPRLLIYDTSNLASGVPVRFSGSGSGTSYSLTISRMEAEDAATYYCQQWSGYPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 13 - protein, 245 aa, Artificial Sequence
  synthetic construct; heavy chain Clazakizumab
  misc (225)..(228): amino acids 225 to 228 can be present or absent
  misc (229)..(234): amino acids 229 to 234 can be present or absent
  misc (235)..(245): amino acids 235 to 245 can be present or absent
EVQLVESGGGLVQPGGSLRLSCAASGFSLSNYYVTWVRQAPGKGLEWVGIIYGSDETAYATSAIGRFTISRDNSKNTLYLQMNSLRAEDTAVYYCARDDSSDWDAKFNLWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFL

SEQ ID NO: 14 - protein, 217 aa, Artificial Sequence
  synthetic construct; light chain Clazakizumab
AIQMTQSPSSLSASVGDRVTITCQASQSINNELSWYQQKPGKAPKLLIYRASTLASGVPSRFSGSGSGTDFTLTISSLQPDDFATYYCQQGYSLRNIDNAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 15 - protein, 244 aa, Artificial Sequence
  synthetic construct; heavy chain Sirukumab
  misc (224)..(227): amino acids 224 to 227 can be present or absent
  misc (228)..(233): amino acids 228 to 233 can be present or absent
  misc (234)..(244): amino acids 234 to 244 can be present or absent
EVQLVESGGGLVQPGGSLRLSCAASGFTFSPFAMSWVRQAPGKGLEWVAKISPGGSWTYYSDTVTGRFTISRDNAKNSLYLQMNSLRAEDTAVYYCARQLWGYYALDIWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFL

SEQ ID NO: 16 - protein, 213 aa, Artificial Sequence
  synthetic construct; light chain Sirukumab
EIVLTQSPATLSLSPGERATLSCSASISVSYMYWYQQKPGQAPRLLIYDMSNLASGIPARFSGSGSGTDFTLTISSLEPEDFAVYYCMQWSGYPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 17 - protein, 242 aa, Artificial Sequence
  synthetic construct; heavy chain Olokizumab
  mis (223)..(231): amino acids 223 to 231 can be present or absent
  mis (232)..(242): amino acids 232 to 242 can be present or absent
EVQLVESGGGLVQPGGSLRLSCAASGFNFNDYFMNWVRQAPGKGLEWVAQMRNKNYQYGTYYAESLEGRFTISRDDSKNSLYLQMNSLKTEDTAVYYCARESYYGFTSYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFL

SEQ ID NO: 18 - protein, 214 aa, Artificial Sequence
  synthetic construct; light chain Olokizumab
DIQMTQSPSSLSASVGDRVTITCQASQDIGISLSWYQQKPGKAPKLLIYNANNLADGVPSRFSGSGSGTDFTLTISSLQPEDFATYYCLQHNSAPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 19 - protein, 247 aa, Artificial Sequence
  synthetic construct; heavy chain Gerilimzumab
  misc (227)..(230): amino acids 227 to 230 can be present or absent
  misc (231)..(236): amino acids 231 to 236 can be present or absent
  misc (237)..(247): amino acids 237 to 247 can be present or absent
EVQLQESGPGLVKPSQTLSLTCTVSGGSITSRYYAWSWIRQPPGKGLEWIGVIDYDGDTYYSPSLKSRVSISWDTSKNQFSLKLSSVTPADTAVYYCARDPDVVTGFHYDYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFL

SEQ ID NO: 20 - protein, 216 aa, Artificial Sequence
  synthetic construct; light chain Gerilimzumab
QSALTQPPSVSGTPGQSVTISCAGANNDIGTYAYVSWYQQLPGTAPKLMIYKVTTRASGIPDRFSGSKSGNTASLTISGLQAEDEADYYCASYRNFNNAVFGTGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 21 - protein, 244 aa, Artificial Sequence
  synthetic construct; heavy chain Tocilizumab
  misc (224)..(227): amino acids 224 to 227 can be present or absent
  misc (228)..(233): amino acids 228 to 233 can be present or absent
  misc (234)..(244): amino acids 234 to 244 can be present or absent
QVQLQESGPGLVRPSQTLSLTCTVSGYSITSDHAWSWVRQPPGRGLEWIGYISYSGITTYNPSLKSRVTMLRDTSKNQFSLRLSSVTAADTAVYYCARSLARTTAMDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFL

SEQ ID NO: 22 - protein, 214 aa, Artificial Sequence [sequence omitted]

SEQ ID NO: 23 - protein, 451 aa, Artificial Sequence
  synthetic construct; Adalimumab Heavy Chain
EVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 24 - protein, 730 aa, Artificial Sequence
  synthetic construct; Adalimumab Vectorized IgG- fully encoded protein
MYRMQLLLLIALSLALVTNSEVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKRKRRGSGEGRGSLLTCGDVEENPGPMYRMQLLLLIALSLALVTNSDIQMTQSPSSLSASVGDRVTITCRASQGIRNYLAWYQQKPGKAPKLLIYAASTLQSGVPSRFSGSGSGTDFTLTISSLQPEDVATYYCQRYNRAPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 25 - protein, 507 aa, Artificial Sequence
  synthetic construct; Adalimumab Vectorized Fab- fully encoded protein
MYRMQLLLLIALSLALVTNSEVQLVESGGGLVQPGRSLRLSCAASGFTFDDYAMHWVRQAPGKGLEWVSAITWNSGHIDYADSVEGRFTISRDNAKNSLYLQMNSLRAEDTAVYYCAKVSYLSTASSLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHRKRRGSGEGRGSLLTCGDVEENPGPMYRMQLLLLIALSLALVTNSDIQMTQSPSSLSASVGDRVTITCRASQGIRNYLAWYQQKPGKAPKLLIYAASTLQSGVPSRFSGSGSGTDFTLTISSLQPEDVATYYCQRYNRAPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 26 - protein, 738 aa, Artificial Sequence
  synthetic construct; antibody fragment
  misc (676)..(687): nucleotides 676 to 687 can be present or absent
  misc (688)..(705): nucleotides 688 to 705 can be present or absent
  misc (706)..(738): nucleotides706 to 738 can be present or absent
GAGGTGCAGCTGGTGGAGAGCGGCGGCGGCCTGGTGCAGCCCGGCAGGAGCCTGAGGCTGAGCTGCGCCGCCAGCGGCTTCACCTTCGACGACTACGCCATGCACTGGGTGAGGCAGGCCCCCGGCAAGGGCCTGGAGTGGGTGAGCGCCATCACCTGGAACAGCGGCCACATCGACTACGCCGACAGCGTGGAGGGCAGGTTCACCATCAGCAGGGACAACGCCAAGAACAGCCTGTACCTGCAGATGAACAGCCTGAGGGCCGAGGACACCGCCGTGTACTACTGCGCCAAGGTGAGCTACCTGAGCACCGCCAGCAGCCTGGACTACTGGGGCCAGGGCACCCTGGTGACCGTGAGCAGCGCCAGCACCAAGGGCCCCAGCGTGTTCCCCCTGGCCCCCAGCAGCAAGAGCACCAGCGGCGGCACCGCCGCCCTGGGCTGCCTGGTGAAGGACTACTTCCCCGAGCCCGTGACCGTGAGCTGGAACAGCGGCGCCCTGACCAGCGGCGTGCACACCTTCCCCGCCGTGCTGCAGAGCAGCGGCCTGTACAGCCTGAGCAGCGTGGTGACCGTGCCCAGCAGCAGCCTGGGCACCCAGACCTACATCTGCAACGTGAACCACAAGCCCAGCAACACCAAGGTGGACAAGAAGGTGGAGCCCAAGAGCTGCGACAAGACCCACACCTGCCCCCCCTGCCCCGCCCCCGAGCTGCTGGGCGGCCCCAGCGTGTTCCTG

SEQ ID NO: 27 - DNA, 642 bp, Artificial Sequence
  synthetic construct; antibody
gacatccagatgacccagagccccagcagcctgagcgccagcgtgggcgacagggtgaccatcacctgcagggccagccagggcatcaggaactacctggcctggtaccagcagaagcccggcaaggcccccaagctgctgatctacgccgccagcaccctgcagagcggcgtgcccagcaggttcagcggcagcggcagcggcaccgacttcaccctgaccatcagcagcctgcagcccgaggacgtggccacctactactgccagaggtacaacagggccccctacaccttcggccagggcaccaaggtggagatcaagaggaccgtggccgcccccagcgtgttcatcttcccccccagcgacgagcagctgaagagcggcaccgccagcgtggtgtgcctgctgaacaacttctaccccagggaggccaaggtgcagtggaaggtggacaacgccctgcagagcggcaacagccaggagagcgtgaccgagcaggacagcaaggacagcacctacagcctgagcagcaccctgaccctgagcaaggccgactacgagaagcacaaggtgtacgcctgcgaggtgacccaccagggcctgagcagccccgtgaccaagagcttcaacaggggcgagtgc

SEQ ID NO: 28 - DNA, 734 bp, Artificial Sequence
  synthetic construct; antibody fragment
  misc (673)..(684): nucleotides 673 to 684 can be present or absent
  misc (685)..(702): nucleotides 685 to 702 can be present or absent
  misc (703)..(734): nucleotides 703 to 734 can be present or absent
gaggtgaagctggaggagagcggcggcggcctggtgcagcccggcggcagcatgaagctgagctgcgtggccagcggcttcatcttcagcaaccactggatgaactgggtgaggcagagccccgagaagggcctggagtgggtggccgagatcaggagcaagagcatcaacagcgccacccactacgccgagagcgtgaagggcaggttcaccatcagcagggacgacagcaagagcgccgtgtacctgcagatgaccgacctgaggaccgaggacaccggcgtgtactactgcagcaggaactactacggcagcacctacgactactggggccagggcaccaccctgaccgtgagcagcgccagcaccaagggccccagcgtgttccccctggcccccagcagcaagagcaccagcggcggcaccgccgccctgggctgcctggtgaaggactacttccccgagcccgtgaccgtgagctggaacagcggcgccctgaccagcggcgtgcacaccttccccgccgtgctgcagagcagcggcctgtacagcctgagcagcgtggtgaccgtgcccagcagcagcctgggcacccagacctacatctgcaacgtgaaccacaagcccagcaacaccaaggtggacaagaaggtggagcccaagagctgcgacaagacccacacctgccccccctgccccgcccccgagctgctgggcggccccagcggttcctg

SEQ ID NO: 29 - DNA, 642 bp, Artificial Sequence
  synthetic construct; antibody fragment
gacatcctgctgacccagagccccgccatcctgagcgtgagccccggcgagagggtgagcttcagctgcagggccagccagttcgtgggcagcagcatccactggtaccagcagaggaccaacggcagccccaggctgctgatcaagtacgccagcgagagcatgagcggcatccccagcaggttcagcggcagcggcagcggcaccgacttcaccctgagcatcaacaccgtggagagcgaggacatcgccgactactactgccagcagagccacagctggcccttcaccttcggcagcggcaccaacctggaggtgaagaggaccgtggccgcccccagcgtgttcatcttcccccccagcgacgagcagctgaagagcggcaccgccagcgtggtgtgcctgctgaacaacttctaccccagggaggccaaggtgcagtggaaggtggacaacgccctgcagagcggcaacagccaggagagcgtgaccgagcaggacagcaaggacagcacctacagcctgagcagcaccctgaccctgagcaaggccgactacgagaagcacaaggtgtacgcctgcgaggtgacccaccagggcctgagcagccccgtgaccaagagcttcaacaggggcgagtgc

SEQ ID NO: 30 - DNA, 720 bp, Artificial Sequence
  synthetic construct; antibody fragment
  misc (659)..(669): amino acids 659 to 669 can be present or absent
  misc (670)..(687): amino acids 670 to 687 can be present or absent
  misc (688)..(720): amino acids 688 to 720 can be present or absent
agcaagctgcaggtgcagctggtggagagcggcggcggcgtggtgcagcccggcaggagcctgaggctgagctgcgccgccagcggcttcatcttcagcagctacgccatgcactgggtgaggcaggcccccggcaacggcctggagtgggtggccttcatgagctacgacggcagcaacaagaagtacgccgacagcgtgaagggcaggttcaccatcagcagggacaacagcaagaacaccctgtacctgcagatgaacagcctgagggccgaggacaccgccgtgtactactgcgccagggacaggggcatcgccgccggcggcaactactactactacggcatggacgtgtggggccagggcaccaccgtgaccgtgagcagcgccagcaccaagggccccagcgtgttccccctggcccccagcagcaagagcaccagcggcggcaccgccgccctgggctgcctggtgaaggactacttccccgagcccgtgaccgtgagctggaacagcggcgccctgaccagcggcgtgcacaccttccccgccgtgctgcagagcagcggcctgtacagcctgagcagcgtggtgaccgtgcccagcagcagcctgggcacccagacctacatctgcaacgtgaaccacaagcccagcaagacccacacctgccccccctgccccgcccccgagctgctgggcggccccagcgtgttcctg

SEQ ID NO: 31 - DNA, 654 bp, Artificial Sequence
  synthetic construct; antibody fragment
gccggcagcgagatcgtgctgacccagagccccgccaccctgagcctgagccccggcgagagggccaccctgagctgcagggccagccagagcgtgtacagctacctggcctggtaccagcagaagcccggccaggcccccaggctgctgatctacgacgccagcaacagggccaccggcatccccgccaggttcagcggcagcggcagcggcaccgacttcaccctgaccatcagcagcctggagcccgaggacttcgccgtgtactactgccagcagaggagcaactggccccccttcaccttcggccccggcaccaaggtggacatcaagaggaccgtggccgcccccagcgtgttcatcttcccccccagcgacgagcagctgaagagcggcaccgccagcgtggtgtgcctgctgaacaacttctaccccagggaggccaaggtgcagtggaaggtggacaacgccctgcagagcggcaacagccaggagagcgtgaccgagcaggacagcaaggacagcacctacagcctgagcagcaccctgaccctgagcaaggccgactacgagaagcacaaggtgtacgcctgcgaggtgacccaccagggcctgagcagccccgtgaccaagagcttcaacaggggcgagtgc

SEQ ID NO: 32 - DNA, 705 bp, Artificial Sequence
  synthetic construct; antibody fragment
  misc (673)..(690): amino acids 673 to 690 can be present or absent
  misc (691)..(705): amino acids 691 to 705 can be present or absent
caggtgcagctgcaggagagcggccccggcctggtgaagcccagcgagaccctgagcctgacctgcgccgtgagcggccacagcatcagccacgaccacgcctggagctgggtgaggcagccccccggcgagggcctggagtggatcggcttcatcagctacagcggcatcaccaactacaaccccagcctgcagggcagggtgaccatcagcagggacaacagcaagaacaccctgtacctgcagatgaacagcctgagggccgaggacaccgccgtgtactactgcgccaggagcctggccaggaccaccgccatggactactggggcgagggcaccctggtgaccgtgagcagcgccagcaccaagggccccagcgtgttccccctggcccccagcagcaagagcaccagcggcggcaccgccgccctgggctgcctggtgaaggactacttccccgagcccgtgaccgtgagctggaacagcggcgccctgaccagcggcgtgcacaccttccccgccgtgctgcagagcagcggcctgtacagcctgagcagcgtggtgaccgtgcccagcagcaacttcggcacccagacctacacctgcaacgtggaccacaagcccagcaacaccaaggtggacaagaccgtggagaggaagagctgcgtggagtgccccccctgccccgccccccccgtggccggc

SEQ ID NO: 33 - DNA, 642 bp, Artificial Sequence
  synthetic construct; antibody fragment
gacatccagatgacccagagccccagcagcctgagcgccagcgtgggcgacagcgtgaccatcacctgccaggccagcaccgacatcagcagccacctgaactggtaccagcagaagcccggcaaggcccccgagctgctgatctactacggcagccacctgctgagcggcgtgcccagcaggttcagcggcagcggcagcggcaccgacttcaccttcaccatcagcagcctggaggccgaggacgccgccacctactactgcggccagggcaacaggctgccctacaccttcggccagggcaccaaggtggagatcgagaggaccgtggccgcccccagcgtgttcatcttcccccccagcgacgagcagctgaagagcggcaccgccagcgtggtgtgcctgctgaacaacttctaccccagggaggccaaggtgcagtggaaggtggacaacgccctgcagagcggcaacagccaggagagcgtgaccgagcaggacagcaaggacagcacctacagcctgagcagcaccctgaccctgagcaaggccgactacgagaagcacaaggtgtacgcctgcgaggtgacccaccagggcctgagcagccccgtgaccaagagcttcaacaggggcgagtgc

SEQ ID NO: 34 - DNA, 723 bp, Artificial Sequence
  synthetic construct; antibody fragment
  misc (661)..(672): nucleotids 661 to 672 can be present or absent
  misc (673)..(690): nucleotids 673 to 690 can be present or absent
  misc (691)..(723): nucleotids 691 to 723 can be present or absent
gaggtgcagctggtggagagcggcggcggcctggtgcagcccggcaggagcctgaggctgagctgcgccgccagcaggttcaccttcgacgactacgccatgcactgggtgaggcaggcccccggcaagggcctggagtgggtgagcggcatcagctggaacagcggcaggatcggctacgccgacagcgtgaagggcaggttcaccatcagcagggacaacgccgagaacagcctgttcctgcagatgaacggcctgagggccgaggacaccgccctgtactactgcgccaagggcagggacagcttcgacatctggggccagggcaccatggtgaccgtgagcagcgccagcaccaagggccccagcgtgttccccctggcccccagcagcaagagcaccagcggcggcaccgccgccctgggctgcctggtgaaggactacttccccgagcccgtgaccgtgagctggaacagcggcgccctgaccagcggcgtgcacaccttccccgccgtgctgcagagcagcggcctgtacagcctgagcagcgtggtgaccgtgcccagcagcagcctgggcacccagacctacatctgcaacgtgaaccacaagcccagcaacaccaaggtggacaagaaggtggagcccaagagctgcgacaagacccacacctgccccccctgccccgcccccgagctgctgggcggccccagcgtgttcctg

SEQ ID NO: 35 - DNA, 642 bp, Artificial Sequence
  synthetic construct; antibody fragment
gacatccagatgacccagagccccagcagcgtgagcgccagcgtgggcgacagggtgaccatcacctgcagggccagccagggcatcagcagctggctggcctggtaccagcagaagcccggcaaggcccccaagctgctgatctacggcgccagcagcctggagagcggcgtgcccagcaggttcagcggcagcggcagcggcaccgacttcaccctgaccatcagcagcctgcagcccgaggacttcgccagctactactgccagcaggccaacagcttcccctacaccttcggccagggcaccaagctggagatcaagaggaccgtggccgcccccagcgtgttcatcttcccccccagcgacgagcagctgaagagcggcaccgccagcgtggtgtgcctgctgaacaacttctaccccagggaggccaaggtgcagtggaaggtggacaacgccctgcagagcggcaacagccaggagagcgtgaccgagcaggacagcaaggacagcacctacagcctgagcagcaccctgaccctgagcaaggccgactacgagaagcacaaggtgtacgcctgcgaggtgacccaccagggcctgagcagccccgtgaccaagagcttcaacaggggcgagtgc

SEQ ID NO: 36 - DNA, 732 bp, Artificial Sequence
  synthetic construct; antibody fragment
  misc (670)..(681): nucleotides 670 to 681 can be present or absent
  misc (682)..(699): nucleotides 682 to 699 can be present or absent
  misc (700)..(732): nucleotides 700 to 732 can be present or absent
gaagtgcagctggtggaaagcggcggcaaactgctgaaaccgggcggcagcctgaaactgagctgcgcggcgagcggctttacctttagcagctttgcgatgagctggtttcgccagagcccggaaaaacgcctggaatgggtggcggaaattagcagcggcggcagctatacctattatccggataccgtgaccggccgctttaccattagccgcgataacgcgaaaaacaccctgtatctggaaatgagcagcctgcgcagcgaagataccgcgatgtattattgcgcgcgcggcctgtggggctattatgcgctggattattggggccagggcaccagcgtgaccgtgagcagcgcgagcaccaaaggcccgagcgtgtttccgctggcgccgagcagcaaaagcaccagcggcggcaccgcggcgctgggctgcctggtgaaagattattttccggaaccggtgaccgtgagctggaacagcggcgcgctgaccagcggcgtgcatacctttccggcggtgctgcagagcagcggcctgtatagcctgagcagcgtggtgaccgtgccgagcagcagcctgggcacccagacctatatttgcaacgtgaaccataaaccgagcaacaccaaagtggataaaaaagtggaaccgaaaagctgcgataagacccacacctgccccccctgccccgcccccgagctgctgggcggccccagcgtgttcctg

SEQ ID NO: 37 - DNA, 639 bp, Artificial Sequence
  synthetic construct; antibody fragment
cagattgtgctgattcagagcccggcgattatgagcgcgagcccgggcgaaaaagtgaccatgacctgcagcgcgagcagcagcgtgagctatatgtattggtatcagcagaaaccgggcagcagcccgcgcctgctgatttatgataccagcaacctggcgagcggcgtgccggtgcgctttagcggcagcggcagcggcaccagctatagcctgaccattagccgcatggaagcggaagatgcggcgacctattattgccagcagtggagcggctatccgtatacctttggcggcggcaccaaactggaaattaaacgcaccgtggcggcgccgagcgtgtttatttttccgccgagcgatgaacagctgaaaagcggcaccgcgagcgtggtgtgcctgctgaacaacttttatccgcgcgaagcgaaagtgcagtggaaagtggataacgcgctgcagagcggcaacagccaggaaagcgtgaccgaacaggatagcaaagatagcacctatagcctgagcagcaccctgaccctgagcaaagcggattatgaaaaacataaagtgtatgcgtgcgaagtgacccatcagggcctgagcagcccggtgaccaaaagctttaaccgcggcgaatgc

SEQ ID NO: 38 - DNA, 735 bp, Artificial Sequence
  synthetic construct; antibody fragment
  misc (673)..(684): nucleotides 673 to 684 can be present or absent
  misc (685)..(702): nucleotides 685 to 702 can be present or absent
  misc (703)..(735): nucleotides 703 to 735 can be present or absent
gaagtgcagctggtggaaagcggcggcggcctggtgcagccgggcggcagcctgcgcctgagctgcgcggcgagcggctttagcctgagcaactattatgtgacctgggtgcgccaggcgccgggcaaaggcctggaatgggtgggcattatttatggcagcgatgaaaccgcgtatgcgaccagcgcgattggccgctttaccattagccgcgataacagcaaaaacaccctgtatctgcagatgaacagcctgcgcgcggaagataccgcggtgtattattgcgcgcgcgatgatagcagcgattgggatgcgaaatttaacctgtggggccagggcaccctggtgaccgtgagcagcgcgagcaccaaaggcccgagcgtgtttccgctggcgccgagcagcaaaagcaccagcggcggcaccgcggcgctgggctgcctggtgaaagattattttccggaaccggtgaccgtgagctggaacagcggcgcgctgaccagcggcgtgcatacctttccggcggtgctgcagagcagcggcctgtatagcctgagcagcgtggtgaccgtgccgagcagcagcctgggcacccagacctatatttgcaacgtgaaccataaaccgagcaacaccaaagtggataaacgcgtggaaccgaaaagctgcgataagacccacacctgccccccctgccccgcccccgagctgctgggcggccccagcgtgttcctg

SEQ ID NO: 39 - DNA, 732 bp, Artificial Sequence
  synthetic construct; antibody fragment
  misc (670)..(681): nucleotides 670 to 681 can be present or absent
  misc (682)..(699): nucleotides 682 to 699 can be present or absent
  misc (700)..(732): nucleotides 700 to 732 can be present or absent
gcgattcagatgacccagagcccgagcagcctgagcgcgagcgtgggcgatcgcgtgaccattacctgccaggcgagccagagcattaacaacgaactgagctggtatcagcagaaaccgggcaaagcgccgaaactgctgatttatcgcgcgagcaccctggcgagcggcgtgccgagccgctttagcggcagcggcagcggcaccgattttaccctgaccattagcagcctgcagccggatgattttgcgacctattattgccagcagggctatagcctgcgcaacattgataacgcgtttggcggcggcaccaaagtggaaattaaacgcaccgtggcggcgccgagcgtgtttatttttccgccgagcgatgaacagctgaaaagcggcaccgcgagcgtggtgtgcctgctgaacaacttttatccgcgcgaagcgaaagtgcagtggaaagtggataacgcgctgcagagcggcaacagccaggaaagcgtgaccgaacaggatagcaaagatagcacctatagcctgagcagcaccctgaccctgagcaaagcggattatgaaaaacataaagtgtatgcgtgcgaagtgacccatcagggcctgagcagcccggtgaccaaaagctttaaccgcggcgaatgcgaaccgaaaagctgcgataagacccacacctgccccccctgccccgcccccgagctgctgggcggccccagcgtgttcctg

SEQ ID NO: 40 - DNA, 732 bp, Artificial Sequence
  synthetic construct; antibody fragment
  misc (670)..(681): nucleotides 670 to 681 can be present or absent
  misc (682)..(699): nucleotides 682 to 699 can be present or absent
  misc (700)..(732): nucleotides 700 to 732 can be present or absent
gaagtgcagctggtggaaagcggcggcggcctggtgcagccgggcggcagcctgcgcctgagctgcgcggcgagcggctttacctttagcccgtttgcgatgagctgggtgcgccaggcgccgggcaaaggcctggaatgggtggcgaaaattagcccgggcggcagctggacctattatagcgataccgtgaccggccgctttaccattagccgcgataacgcgaaaaacagcctgtatctgcagatgaacagcctgcgcgcggaagataccgcggtgtattattgcgcgcgccagctgtggggctattatgcgctggatatttggggccagggcaccaccgtgaccgtgagcagcgcgagcaccaaaggcccgagcgtgtttccgctggcgccgagcagcaaaagcaccagcggcggcaccgcggcgctgggctgcctggtgaaagattattttccggaaccggtgaccgtgagctggaacagcggcgcgctgaccagcggcgtgcatacctttccggcggtgctgcagagcagcggcctgtatagcctgagcagcgtggtgaccgtgccgagcagcagcctgggcacccagacctatatttgcaacgtgaaccataaaccgagcaacaccaaagtggataaaaaagtggaaccgaaaagctgcgataagacccacacctgccccccctgccccgcccccgagctgctgggcggccccagcgtgttcctg

SEQ ID NO: 41 - DNA, 639 bp, Artificial Sequence
  synthetic construct; antibody fragment
gaaattgtgctgacccagagcccggcgaccctgagcctgagcccgggcgaacgcgcgaccctgagctgcagcgcgagcattagcgtgagctatatgtattggtatcagcagaaaccgggccaggcgccgcgcctgctgatttatgatatgagcaacctggcgagcggcattccggcgcgctttagcggcagcggcagcggcaccgattttaccctgaccattagcagcctggaaccggaagattttgcggtgtattattgcatgcagtggagcggctatccgtatacctttggcggcggcaccaaagtggaaattaaacgcaccgtggcggcgccgagcgtgtttatttttccgccgagcgatgaacagctgaaaagcggcaccgcgagcgtggtgtgcctgctgaacaacttttatccgcgcgaagcgaaagtgcagtggaaagtggataacgcgctgcagagcggcaacagccaggaaagcgtgaccgaacaggatagcaaagatagcacctatagcctgagcagcaccctgaccctgagcaaagcggattatgaaaaacataaagtgtatgcgtgcgaagtgacccatcagggcctgagcagcccggtgaccaaaagctttaaccgcggcgaatgc

SEQ ID NO: 42 - DNA, 726 bp, Artificial Sequence
  synthetic construct; antibody fragment
  misc (667)..(693): nucleotides 667 to 693 can be present or absent
  misc (694)..(726): nucleotides 694 to 726 can be present or absent
gaagtgcagctggtggaaagcggcggcggcctggtgcagccgggcggcagcctgcgcctgagctgcgcggcgagcggctttaactttaacgattattttatgaactgggtgcgccaggcgccgggcaaaggcctggaatgggtggcgcagatgcgcaacaaaaactatcagtatggcacctattatgcggaaagcctggaaggccgctttaccattagccgcgatgatagcaaaaacagcctgtatctgcagatgaacagcctgaaaaccgaagataccgcggtgtattattgcgcgcgcgaaagctattatggctttaccagctattggggccagggcaccctggtgaccgtgagcagcgcgagcaccaaaggcccgagcgtgtttccgctggcgccgtgcagccgcagcaccagcgaaagcaccgcggcgctgggctgcctggtgaaagattattttccggaaccggtgaccgtgagctggaacagcggcgcgctgaccagcggcgtgcatacctttccggcggtgctgcagagcagcggcctgtatagcctgagcagcgtggtgaccgtgccgagcagcagcctgggcaccaaaacctatacctgcaacgtggatcataaaccgagcaacaccaaagtggataaacgcgtggagagcaagtacggccccccctgccccccctgccccgcccccgagttcctgggcggccccagcgtgttcctg

SEQ ID NO: 43 - DNA, 642 bp, Artificial Sequence
  synthetic construct; antibody fragment
gatattcagatgacccagagcccgagcagcctgagcgcgagcgtgggcgatcgcgtgaccattacctgccaggcgagccaggatattggcattagcctgagctggtatcagcagaaaccgggcaaagcgccgaaactgctgatttataacgcgaacaacctggcggatggcgtgccgagccgctttagcggcagcggcagcggcaccgattttaccctgaccattagcagcctgcagccggaagattttgcgacctattattgcctgcagcataacagcgcgccgtatacctttggccagggcaccaaactggaaattaaacgcaccgtggcggcgccgagcgtgtttatttttccgccgagcgatgaacagctgaaaagcggcaccgcgagcgtggtgtgcctgctgaacaacttttatccgcgcgaagcgaaagtgcagtggaaagtggataacgcgctgcagagcggcaacagccaggaaagcgtgaccgaacaggatagcaaagatagcacctatagcctgagcagcaccctgaccctgagcaaagcggattatgaaaaacataaagtgtatgcgtgcgaagtgacccatcagggcctgagcagcccggtgaccaaaagctttaaccgcggcgaatgc

SEQ ID NO: 44 - DNA, 741 bp, Artificial Sequence
  synthetic construct; antibody fragment
  misc (679)..(690): nucleotides 679 to 690 can be present or absent
  misc (691)..(708): nucleotides 691 to 708 can be present or absent
  misc (709)..(741): nucleotides 709 to 741 can be present or absent
gaagtgcagctgcaggaaagcggcccgggcctggtgaaaccgagccagaccctgagcctgacctgcaccgtgagcggcggcagcattaccagccgctattatgcgtggagctggattcgccagccgccgggcaaaggcctggaatggattggcgtgattgattatgatggcgatacctattatagcccgagcctgaaaagccgcgtgagcattagctgggataccagcaaaaaccagtttagcctgaaactgagcagcgtgaccccggcggataccgcggtgtattattgcgcgcgcgatccggatgtggtgaccggctttcattatgattattggggccagggcaccatggtgaccgtgagcagcgcgagcaccaaaggcccgagcgtgtttccgctggcgccgagcagcaaaagcaccagcggcggcaccgcggcgctgggctgcctggtgaaagattattttccggaaccggtgaccgtgagctggaacagcggcgcgctgaccagcggcgtgcatacctttccggcggtgctgcagagcagcggcctgtatagcctgagcagcgtggtgaccgtgccgagcagcagcctgggcacccagacctatatttgcaacgtgaaccataaaccgagcaacaccaaagtggataaaaaagtggaaccgaaaagctgcgataagacccacacctgccccccctgccccgcccccgagctgctgggcggccccagcgtgttcctg

SEQ ID NO: 45 - DNA, 648 bp, Artificial Sequence
  synthetic construct; antibody fragment
cagagcgcgctgacccagccgccgagcgtgagcggcaccccgggccagagcgtgaccattagctgcgcgggcgcgaacaacgatattggcacctatgcgtatgtgagctggtatcagcagctgccgggcaccgcgccgaaactgatgatttataaagtgaccacccgcgcgagcggcattccggatcgctttagcggcagcaaaagcggcaacaccgcgagcctgaccattagcggcctgcaggcggaagatgaagcggattattattgcgcgagctatcgcaactttaacaacgcggtgtttggcaccggcaccaaactgaccgtgctgggccagccgaaagcggcgccgagcgtgaccctgtttccgccgagcagcgaagaactgcaggcgaacaaagcgaccctggtgtgcctgattagcgatttttatccgggcgcggtgaccgtggcgtggaaagcggatagcagcccggtgaaagcgggcgtggaaaccaccaccccgagcaaacagagcaacaacaaatatgcggcgagcagctatctgagcctgaccccggaacagtggaaaagccatcgcagctatagctgccaggtgacccatgaaggcagcaccgtggaaaaaaccgtggcgccgaccgaatgcagc

SEQ ID NO: 46 - DNA, 4513 bp, Artificial Sequence
  synthetic construct; antibody fragment
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgccaccatgtacagaatgcagctgctgctgctcattgccctgtctctggccctggtcaccaattctgaagtgcagctggtggaaagtggtggtggactggtgcagcctggcagaagcctgagactgtcttgtgctgcctctggcttcacctttgatgactatgccatgcactgggtcagacaggcccctggcaaaggactggaatgggtgtcagccatcacctggaactctggccacattgactatgctgactctgtggaaggcagattcaccatcagcagagacaatgccaagaacagcctgtacctgcagatgaactccctgagagctgaggacacagcagtgtactactgtgccaaggtgtcctacctgagcacagccagcagcctggattattggggccagggcacactggttacagtgtcctctgccagcacaaagggcccctctgtttttccactggctcccagcagcaagagcaccagtggtggaacagctgccctgggctgtctggtcaaggattacttccctgagcctgtgacagtgtcttggaactcaggggctctgacctctggggtgcacacatttccagctgtgctgcagtcctctggcctgtactctctgtcctctgtggtcacagtgcctagctctagcctgggcacccagacctacatctgcaatgtgaaccacaagcctagcaacaccaaggtggacaagaaggtggaacccaagagctgtgacaagacccacacctgtcctccatgtcctgctccagaactgcttggaggcccttctgtgttcctgtttcctccaaagcctaaggacaccctgatgatcagcagaacccctgaagtgacctgtgtggtggttgatgtgtcccatgaggacccagaagtgaagttcaattggtatgtggatggggttgaagtgcacaatgctaagaccaagcctagagaggaacagtacaacagcacctacagagtggtgtctgtgctgacagtgctgcatcaggactggctgaatggcaaagagtacaagtgcaaagtgtccaacaaggccctgcctgctcctattgagaaaaccatctccaaggccaagggccagccaagagaaccccaggtttacacactgccacctagcagagatgagctgaccaagaaccaggtgtccctgacctgcctggttaagggcttctacccctctgacattgctgtggaatgggagagcaatggccagcctgagaacaactacaagacaacccctcctgtgctggactctgatggctcattcttcctgtacagcaagctgactgtggacaagtccagatggcagcaggggaatgtgttcagctgctctgtgatgcatgaggccctgcacaaccactacacccagaaaagtctgagtctgagccctggcaagagaaagagaagaggctctggagaaggcagaggctccctgctgacatgtggggatgttgaagagaatcctgggcctatgtataggatgcaactgctcctcctgattgctctgagcctggctcttgtgaccaactctgacatccagatgacacagagccctagcagcctgtctgcttctgtgggagacagagtgaccatcacatgcagagccagccagggaatcagaaactacctggcctggtatcagcaaaagcctggcaaggcccctaagctgctgatctatgcagccagcacactgcagtcaggggtgccaagcagattttcaggctctggctctggcacagacttcaccctgaccatttctagcctgcagcctgaggatgtggccacctactactgccagagatacaacagagccccatacacctttggacagggcacaaaggtggaaatcaagagaacagtggctgccccatctgtgttcatcttcccaccatctgatgaacagctgaagtctggcactgcctctgttgtgtgcctgctgaacaacttctaccctagagaagccaaggtgcagtggaaggttgacaatgccctgcagtctggcaatagccaagaatctgtgacagagcaggactccaaggattccacctacagcctgagcagcaccctgacactgagcaaggctgactatgagaagcacaaagtgtatgcctgtgaagtgacacaccagggactgagcagcccagtgaccaagagcttcaacaggggagagtgctgataactcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 47 - DNA, 4092 bp, Artificial Sequence
  synthetic construct; antibody fragment
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgccaccatgtacagaatgcagctgctgctgctcattgccctgtctctggccctggtcaccaattctgaagtgcagctggtggaaagtggtggtggactggtgcagcctggcagaagcctgagactgtcttgtgctgcctctggcttcacctttgatgactatgccatgcactgggtcagacaggcccctggcaaaggactggaatgggtgtcagccatcacctggaactctggccacattgactatgctgactctgtggaaggcagattcaccatcagcagagacaatgccaagaacagcctgtacctgcagatgaactccctgagagctgaggacacagcagtgtactactgtgccaaggtgtcctacctgagcacagccagcagcctggattattggggccagggcacactggttacagtgtcctctgccagcacaaagggcccctctgtttttccactggctcccagcagcaagagcaccagtggtggaacagctgccctgggctgtctggtcaaggattacttccctgagcctgtgacagtgtcttggaactcaggggctctgacctctggggtgcacacatttccagctgtgctgcagtcctctggcctgtactctctgtcctctgtggtcacagtgcctagctctagcctgggcacccagacctacatctgcaatgtgaaccacaagcctagcaacaccaaggtggacaagaaggtggaacccaagagctgtgacaagacccacacctgtcctccatgtcctgctccagaactgcttggaggcccttctgtgttcctgtttcctccaaagcctaaggacaccctgatgatcagcagaacccctgaagtgacctgtgtggtggttgatgtgtcccatgaggacccagaagtgaagttcaattggtatgtggatggggttgaagtgcacaatgctaagaccaagcctagagaggaacagtacaacagcacctacagagtggtgtctgtgctgacagtgctgcatcaggactggctgaatggcaaagagtacaagtgcaaagtgtccaacaaggccctgcctgctcctattgagaaaaccatctccaaggccaagggccagccaagagaaccccaggtttacacactgccacctagcagagatgagctgaccaagaaccaggtgtccctgacctgcctggttaagggcttctacccctctgacattgctgtggaatgggagagcaatggccagcctgagaacaactacaagacaacccctcctgtgctggactctgatggctcattcttcctgtacagcaagctgactgtggacaagtccagatggcagcaggggaatgtgttcagctgctctgtgatgcatgaggccctgcacaaccactacacccagaaaagtctgagtctgagccctggcaagagaaagagaagaggctctggagaaggcagaggctccctgctgacatgtggggatgttgaagagaatcctgggcctatgtataggatgcaactgctcctcctgattgctctgagcctggctcttgtgaccaactctgacatccagatgacacagagccctagcagcctgtctgcttctgtgggagacagagtgaccatcacatgcagagccagccagggaatcagaaactacctggcctggtatcagcaaaagcctggcaaggcccctaagctgctgatctatgcagccagcacactgcagtcaggggtgccaagcagattttcaggctctggctctggcacagacttcaccctgaccatttctagcctgcagcctgaggatgtggccacctactactgccagagatacaacagagccccatacacctttggacagggcacaaaggtggaaatcaagagaacagtggctgccccatctgtgttcatcttcccaccatctgatgaacagctgaagtctggcactgcctctgttgtgtgcctgctgaacaacttctaccctagagaagccaaggtgcagtggaaggttgacaatgccctgcagtctggcaatagccaagaatctgtgacagagcaggactccaaggattccacctacagcctgagcagcaccctgacactgagcaaggctgactatgagaagcacaaagtgtatgcctgtgaagtgacacaccagggactgagcagcccagtgaccaagagcttcaacaggggagagtgctgataactcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcg

SEQ ID NO: 48 - DNA, 2193 bp, Artificial Sequence
  synthetic construct; antibody fragment
atgtacagaatgcagctgctgctgctcattgccctgtctctggccctggtcaccaattctgaagtgcagctggtggaaagtggtggtggactggtgcagcctggcagaagcctgagactgtcttgtgctgcctctggcttcacctttgatgactatgccatgcactgggtcagacaggcccctggcaaaggactggaatgggtgtcagccatcacctggaactctggccacattgactatgctgactctgtggaaggcagattcaccatcagcagagacaatgccaagaacagcctgtacctgcagatgaactccctgagagctgaggacacagcagtgtactactgtgccaaggtgtcctacctgagcacagccagcagcctggattattggggccagggcacactggttacagtgtcctctgccagcacaaagggcccctctgtttttccactggctcccagcagcaagagcaccagtggtggaacagctgccctgggctgtctggtcaaggattacttccctgagcctgtgacagtgtcttggaactcaggggctctgacctctggggtgcacacatttccagctgtgctgcagtcctctggcctgtactctctgtcctctgtggtcacagtgcctagctctagcctgggcacccagacctacatctgcaatgtgaaccacaagcctagcaacaccaaggtggacaagaaggtggaacccaagagctgtgacaagacccacacctgtcctccatgtcctgctccagaactgcttggaggcccttctgtgttcctgtttcctccaaagcctaaggacaccctgatgatcagcagaacccctgaagtgacctgtgtggtggttgatgtgtcccatgaggacccagaagtgaagttcaattggtatgtggatggggttgaagtgcacaatgctaagaccaagcctagagaggaacagtacaacagcacctacagagtggtgtctgtgctgacagtgctgcatcaggactggctgaatggcaaagagtacaagtgcaaagtgtccaacaaggccctgcctgctcctattgagaaaaccatctccaaggccaagggccagccaagagaaccccaggtttacacactgccacctagcagagatgagctgaccaagaaccaggtgtccctgacctgcctggttaagggcttctacccctctgacattgctgtggaatgggagagcaatggccagcctgagaacaactacaagacaacccctcctgtgctggactctgatggctcattcttcctgtacagcaagctgactgtggacaagtccagatggcagcaggggaatgtgttcagctgctctgtgatgcatgaggccctgcacaaccactacacccagaaaagtctgagtctgagccctggcaagagaaagagaagaggctctggagaaggcagaggctccctgctgacatgtggggatgttgaagagaatcctgggcctatgtataggatgcaactgctcctcctgattgctctgagcctggctcttgtgaccaactctgacatccagatgacacagagccctagcagcctgtctgcttctgtgggagacagagtgaccatcacatgcagagccagccagggaatcagaaactacctggcctggtatcagcaaaagcctggcaaggcccctaagctgctgatctatgcagccagcacactgcagtcaggggtgccaagcagattttcaggctctggctctggcacagacttcaccctgaccatttctagcctgcagcctgaggatgtggccacctactactgccagagatacaacagagccccatacacctttggacagggcacaaaggtggaaatcaagagaacagtggctgccccatctgtgttcatcttcccaccatctgatgaacagctgaagtctggcactgcctctgttgtgtgcctgctgaacaacttctaccctagagaagccaaggtgcagtggaaggttgacaatgccctgcagtctggcaatagccaagaatctgtgacagagcaggactccaaggattccacctacagcctgagcagcaccctgacactgagcaaggctgactatgagaagcacaaagtgtatgcctgtgaagtgacacaccagggactgagcagcccagtgaccaagagcttcaacaggggagagtgctga

SEQ ID NO: 49 - DNA, 3844 bp, Artificial Sequence
  synthetic construct; antibody fragment
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgccaccatgtacagaatgcagctgctgctgctcattgccctgtctctggccctggtcaccaattctgaagtgcagctggtggaaagtggtggtggactggtgcagcctggcagaagcctgagactgtcttgtgctgcctctggcttcacctttgatgactatgccatgcactgggtcagacaggcccctggcaaaggactggaatgggtgtcagccatcacctggaactctggccacattgactatgctgactctgtggaaggcagattcaccatcagcagagacaatgccaagaacagcctgtacctgcagatgaactccctgagagctgaggacacagcagtgtactactgtgccaaggtgtcctacctgagcacagccagcagcctggattattggggccagggcacactggttacagtgtcctctgccagcacaaagggcccctctgtttttccactggctcccagcagcaagagcaccagtggtggaacagctgccctgggctgtctggtcaaggattacttccctgagcctgtgacagtgtcttggaactcaggggctctgacctctggggtgcacacatttccagctgtgctgcagtcctctggcctgtactctctgtcctctgtggtcacagtgcctagctctagcctgggcacccagacctacatctgcaatgtgaaccacaagcctagcaacaccaaggtggacaagaaggtggaacccaagagctgtgacaagacccacagaaagagaagaggctctggagaaggcagaggctccctgctgacatgtggggatgttgaagagaatcctgggcctatgtataggatgcaactgctcctcctgattgctctgagcctggctcttgtgaccaactctgacatccagatgacacagagccctagcagcctgtctgcttctgtgggagacagagtgaccatcacatgcagagccagccagggcatcagaaactacctggcctggtatcagcagaagccaggcaaggcccctaagctgctgatctatgcagccagcacactgcagagtggggtgccaagcagattttcaggctctggctctggcacagacttcaccctgaccatttctagcctgcagcctgaggatgtggccacctactactgccagagatacaacagagccccatacacctttggacagggcacaaaggtggaaatcaagagaacagttgcagcaccctcagttttcatcttccccccctcagatgaacagctgaagtctggcactgcctctgttgtgtgcctgctgaacaacttctaccccagagaagccaaggtgcagtggaaggttgacaatgccctgcagtcaggcaacagccaagaatctgtgactgaacaggattccaaggatagcacctacagcctgagcagcaccctgacactgagcaaggctgactatgagaagcacaaagtgtatgcctgtgaagtgacccaccagggactgagcagcccagtgaccaagagcttcaacaggggagagtgctgataactcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 50 - DNA, 3423 bp, Artificial Sequence
  synthetic construct; antibody fragment
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgccaccatgtacagaatgcagctgctgctgctcattgccctgtctctggccctggtcaccaattctgaagtgcagctggtggaaagtggtggtggactggtgcagcctggcagaagcctgagactgtcttgtgctgcctctggcttcacctttgatgactatgccatgcactgggtcagacaggcccctggcaaaggactggaatgggtgtcagccatcacctggaactctggccacattgactatgctgactctgtggaaggcagattcaccatcagcagagacaatgccaagaacagcctgtacctgcagatgaactccctgagagctgaggacacagcagtgtactactgtgccaaggtgtcctacctgagcacagccagcagcctggattattggggccagggcacactggttacagtgtcctctgccagcacaaagggcccctctgtttttccactggctcccagcagcaagagcaccagtggtggaacagctgccctgggctgtctggtcaaggattacttccctgagcctgtgacagtgtcttggaactcaggggctctgacctctggggtgcacacatttccagctgtgctgcagtcctctggcctgtactctctgtcctctgtggtcacagtgcctagctctagcctgggcacccagacctacatctgcaatgtgaaccacaagcctagcaacaccaaggtggacaagaaggtggaacccaagagctgtgacaagacccacagaaagagaagaggctctggagaaggcagaggctccctgctgacatgtggggatgttgaagagaatcctgggcctatgtataggatgcaactgctcctcctgattgctctgagcctggctcttgtgaccaactctgacatccagatgacacagagccctagcagcctgtctgcttctgtgggagacagagtgaccatcacatgcagagccagccagggcatcagaaactacctggcctggtatcagcagaagccaggcaaggcccctaagctgctgatctatgcagccagcacactgcagagtggggtgccaagcagattttcaggctctggctctggcacagacttcaccctgaccatttctagcctgcagcctgaggatgtggccacctactactgccagagatacaacagagccccatacacctttggacagggcacaaaggtggaaatcaagagaacagttgcagcaccctcagttttcatcttccccccctcagatgaacagctgaagtctggcactgcctctgttgtgtgcctgctgaacaacttctaccccagagaagccaaggtgcagtggaaggttgacaatgccctgcagtcaggcaacagccaagaatctgtgactgaacaggattccaaggatagcacctacagcctgagcagcaccctgacactgagcaaggctgactatgagaagcacaaagtgtatgcctgtgaagtgacccaccagggactgagcagcccagtgaccaagagcttcaacaggggagagtgctgataactcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcg

SEQ ID NO: 51 - DNA, 1524 bp, Artificial Sequence
  synthetic construct; antibody fragment
atgtacagaatgcagctgctgctgctcattgccctgtctctggccctggtcaccaattctgaagtgcagctggtggaaagtggtggtggactggtgcagcctggcagaagcctgagactgtcttgtgctgcctctggcttcacctttgatgactatgccatgcactgggtcagacaggcccctggcaaaggactggaatgggtgtcagccatcacctggaactctggccacattgactatgctgactctgtggaaggcagattcaccatcagcagagacaatgccaagaacagcctgtacctgcagatgaactccctgagagctgaggacacagcagtgtactactgtgccaaggtgtcctacctgagcacagccagcagcctggattattggggccagggcacactggttacagtgtcctctgccagcacaaagggcccctctgtttttccactggctcccagcagcaagagcaccagtggtggaacagctgccctgggctgtctggtcaaggattacttccctgagcctgtgacagtgtcttggaactcaggggctctgacctctggggtgcacacatttccagctgtgctgcagtcctctggcctgtactctctgtcctctgtggtcacagtgcctagctctagcctgggcacccagacctacatctgcaatgtgaaccacaagcctagcaacaccaaggtggacaagaaggtggaacccaagagctgtgacaagacccacagaaagagaagaggctctggagaaggcagaggctccctgctgacatgtggggatgttgaagagaatcctgggcctatgtataggatgcaactgctcctcctgattgctctgagcctggctcttgtgaccaactctgacatccagatgacacagagccctagcagcctgtctgcttctgtgggagacagagtgaccatcacatgcagagccagccagggcatcagaaactacctggcctggtatcagcagaagccaggcaaggcccctaagctgctgatctatgcagccagcacactgcagagtggggtgccaagcagattttcaggctctggctctggcacagacttcaccctgaccatttctagcctgcagcctgaggatgtggccacctactactgccagagatacaacagagccccatacacctttggacagggcacaaaggtggaaatcaagagaacagttgcagcaccctcagttttcatcttccccccctcagatgaacagctgaagtctggcactgcctctgttgtgtgcctgctgaacaacttctaccccagagaagccaaggtgcagtggaaggttgacaatgccctgcagtcaggcaacagccaagaatctgtgactgaacaggattccaaggatagcacctacagcctgagcagcaccctgacactgagcaaggctgactatgagaagcacaaagtgtatgcctgtgaagtgacccaccagggactgagcagcccagtgaccaagagcttcaacaggggagagtgctga

SEQ ID NO: 52 - DNA, 3159 bp, Artificial Sequence
  synthetic construct; antibody fragment
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagagggccccagaagcctggtggttgtttgtccttctcaggggaaaagtgaggcggccccttggaggaaggggccgggcagaatgatctaatcggattccaagcagctcaggggattgtctttttctagcaccttcttgccactcctaagcgtcctccgtgaccccggctgggatttagcctggtgctgtgtcagccccgggctcccaggggcttcccagtggtccccaggaaccctcgacagggccagggcgtctctctcgtccagcaagggcagggacgggccacaggccaagggccaggtgagtatctcagggatccagacatggggatatgggaggtgcctctgatcccagggctcactgtgggtctctctgttcacaggttgaattcgccaccatgtacagaatgcagctgctgctgctcattgccctgtctctggccctggtcaccaattctgaagtgcagctggtggaaagtggtggtggactggtgcagcctggcagaagcctgagactgtcttgtgctgcctctggcttcacctttgatgactatgccatgcactgggtcagacaggcccctggcaaaggactggaatgggtgtcagccatcacctggaactctggccacattgactatgctgactctgtggaaggcagattcaccatcagcagagacaatgccaagaacagcctgtacctgcagatgaactccctgagagctgaggacacagcagtgtactactgtgccaaggtgtcctacctgagcacagccagcagcctggattattggggccagggcacactggttacagtgtcctctgccagcacaaagggcccctctgtttttccactggctcccagcagcaagagcaccagtggtggaacagctgccctgggctgtctggtcaaggattacttccctgagcctgtgacagtgtcttggaactcaggggctctgacctctggggtgcacacatttccagctgtgctgcagtcctctggcctgtactctctgtcctctgtggtcacagtgcctagctctagcctgggcacccagacctacatctgcaatgtgaaccacaagcctagcaacaccaaggtggacaagaaggtggaacccaagagctgtgacaagacccacacctgtcctccatgtcctgctccagaactgcttggaggcccttctgtgttcctgtttcctccaaagcctaaggacaccctgatgatcagcagaacccctgaagtgacctgtgtggtggttgatgtgtcccatgaggacccagaagtgaagttcaattggtatgtggatggggttgaagtgcacaatgctaagaccaagcctagagaggaacagtacaacagcacctacagagtggtgtctgtgctgacagtgctgcatcaggactggctgaatggcaaagagtacaagtgcaaagtgtccaacaaggccctgcctgctcctattgagaaaaccatctccaaggccaagggccagccaagagaaccccaggtttacacactgccacctagcagagatgagctgaccaagaaccaggtgtccctgacctgcctggttaagggcttctacccctctgacattgctgtggaatgggagagcaatggccagcctgagaacaactacaagacaacccctcctgtgctggactctgatggctcattcttcctgtacagcaagctgactgtggacaagtccagatggcagcaggggaatgtgttcagctgctctgtgatgcatgaggccctgcacaaccactacacccagaaaagtctgagtctgagccctggcaagagaaagagaagaggctctggagaaggcagaggctccctgctgacatgtggggatgttgaagagaatcctgggcctatgtataggatgcaactgctcctcctgattgctctgagcctggctcttgtgaccaactctgacatccagatgacacagagccctagcagcctgtctgcttctgtgggagacagagtgaccatcacatgcagagccagccagggaatcagaaactacctggcctggtatcagcaaaagcctggcaaggcccctaagctgctgatctatgcagccagcacactgcagtcaggggtgccaagcagattttcaggctctggctctggcacagacttcaccctgaccatttctagcctgcagcctgaggatgtggccacctactactgccagagatacaacagagccccatacacctttggacagggcacaaaggtggaaatcaagagaacagtggctgccccatctgtgttcatcttcccaccatctgatgaacagctgaagtctggcactgcctctgttgtgtgcctgctgaacaacttctaccctagagaagccaaggtgcagtggaaggttgacaatgccctgcagtctggcaatagccaagaatctgtgacagagcaggactccaaggattccacctacagcctgagcagcaccctgacactgagcaaggctgactatgagaagcacaaagtgtatgcctgtgaagtgacacaccagggactgagcagcccagtgaccaagagcttcaacaggggagagtgctgataactcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 53 - DNA, 2751 bp, Artificial Sequence
  synthetic construct; antibody fragment
gggccccagaagcctggtggttgtttgtccttctcaggggaaaagtgaggcggccccttggaggaaggggccgggcagaatgatctaatcggattccaagcagctcaggggattgtctttttctagcaccttcttgccactcctaagcgtcctccgtgaccccggctgggatttagcctggtgctgtgtcagccccgggctcccaggggcttcccagtggtccccaggaaccctcgacagggccagggcgtctctctcgtccagcaagggcagggacgggccacaggccaagggccaggtgagtatctcagggatccagacatggggatatgggaggtgcctctgatcccagggctcactgtgggtctctctgttcacaggttgaattcgccaccatgtacagaatgcagctgctgctgctcattgccctgtctctggccctggtcaccaattctgaagtgcagctggtggaaagtggtggtggactggtgcagcctggcagaagcctgagactgtcttgtgctgcctctggcttcacctttgatgactatgccatgcactgggtcagacaggcccctggcaaaggactggaatgggtgtcagccatcacctggaactctggccacattgactatgctgactctgtggaaggcagattcaccatcagcagagacaatgccaagaacagcctgtacctgcagatgaactccctgagagctgaggacacagcagtgtactactgtgccaaggtgtcctacctgagcacagccagcagcctggattattggggccagggcacactggttacagtgtcctctgccagcacaaagggcccctctgtttttccactggctcccagcagcaagagcaccagtggtggaacagctgccctgggctgtctggtcaaggattacttccctgagcctgtgacagtgtcttggaactcaggggctctgacctctggggtgcacacatttccagctgtgctgcagtcctctggcctgtactctctgtcctctgtggtcacagtgcctagctctagcctgggcacccagacctacatctgcaatgtgaaccacaagcctagcaacaccaaggtggacaagaaggtggaacccaagagctgtgacaagacccacacctgtcctccatgtcctgctccagaactgcttggaggcccttctgtgttcctgtttcctccaaagcctaaggacaccctgatgatcagcagaacccctgaagtgacctgtgtggtggttgatgtgtcccatgaggacccagaagtgaagttcaattggtatgtggatggggttgaagtgcacaatgctaagaccaagcctagagaggaacagtacaacagcacctacagagtggtgtctgtgctgacagtgctgcatcaggactggctgaatggcaaagagtacaagtgcaaagtgtccaacaaggccctgcctgctcctattgagaaaaccatctccaaggccaagggccagccaagagaaccccaggtttacacactgccacctagcagagatgagctgaccaagaaccaggtgtccctgacctgcctggttaagggcttctacccctctgacattgctgtggaatgggagagcaatggccagcctgagaacaactacaagacaacccctcctgtgctggactctgatggctcattcttcctgtacagcaagctgactgtggacaagtccagatggcagcaggggaatgtgttcagctgctctgtgatgcatgaggccctgcacaaccactacacccagaaaagtctgagtctgagccctggcaagagaaagagaagaggctctggagaaggcagaggctccctgctgacatgtggggatgttgaagagaatcctgggcctatgtataggatgcaactgctcctcctgattgctctgagcctggctcttgtgaccaactctgacatccagatgacacagagccctagcagcctgtctgcttctgtgggagacagagtgaccatcacatgcagagccagccagggaatcagaaactacctggcctggtatcagcaaaagcctggcaaggcccctaagctgctgatctatgcagccagcacactgcagtcaggggtgccaagcagattttcaggctctggctctggcacagacttcaccctgaccatttctagcctgcagcctgaggatgtggccacctactactgccagagatacaacagagccccatacacctttggacagggcacaaaggtggaaatcaagagaacagtggctgccccatctgtgttcatcttcccaccatctgatgaacagctgaagtctggcactgcctctgttgtgtgcctgctgaacaacttctaccctagagaagccaaggtgcagtggaaggttgacaatgccctgcagtctggcaatagccaagaatctgtgacagagcaggactccaaggattccacctacagcctgagcagcaccctgacactgagcaaggctgactatgagaagcacaaagtgtatgcctgtgaagtgacacaccagggactgagcagcccagtgaccaagagcttcaacaggggagagtgctgataactcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcg

SEQ ID NO: 54 - DNA, 1353 bp, Artificial Sequence
  synthetic construct; antibody fragment
gaagtgcagctggtggaaagtggtggtggactggtgcagcctggcagaagcctgagactgtcttgtgctgcctctggcttcacctttgatgactatgccatgcactgggtcagacaggcccctggcaaaggactggaatgggtgtcagccatcacctggaactctggccacattgactatgctgactctgtggaaggcagattcaccatcagcagagacaatgccaagaacagcctgtacctgcagatgaactccctgagagctgaggacacagcagtgtactactgtgccaaggtgtcctacctgagcacagccagcagcctggattattggggccagggcacactggttacagtgtcctctgccagcacaaagggcccctctgtttttccactggctcccagcagcaagagcaccagtggtggaacagctgccctgggctgtctggtcaaggattacttccctgagcctgtgacagtgtcttggaactcaggggctctgacctctggggtgcacacatttccagctgtgctgcagtcctctggcctgtactctctgtcctctgtggtcacagtgcctagctctagcctgggcacccagacctacatctgcaatgtgaaccacaagcctagcaacaccaaggtggacaagaaggtggaacccaagagctgtgacaagacccacacctgtcctccatgtcctgctccagaactgcttggaggcccttctgtgttcctgtttcctccaaagcctaaggacaccctgatgatcagcagaacccctgaagtgacctgtgtggtggttgatgtgtcccatgaggacccagaagtgaagttcaattggtatgtggatggggttgaagtgcacaatgctaagaccaagcctagagaggaacagtacaacagcacctacagagtggtgtctgtgctgacagtgctgcatcaggactggctgaatggcaaagagtacaagtgcaaagtgtccaacaaggccctgcctgctcctattgagaaaaccatctccaaggccaagggccagccaagagaaccccaggtttacacactgccacctagcagagatgagctgaccaagaaccaggtgtccctgacctgcctggttaagggcttctacccctctgacattgctgtggaatgggagagcaatggccagcctgagaacaactacaagacaacccctcctgtgctggactctgatggctcattcttcctgtacagcaagctgactgtggacaagtccagatggcagcaggggaatgtgttcagctgctctgtgatgcatgaggccctgcacaaccactacacccagaaaagtctgagtctgagccctggcaag

SEQ ID NO: 55 - DNA, 363 bp, Artificial Sequence
  synthetic construct; antibody fragment
gaagtgcagctggtggaaagtggtggtggactggtgcagcctggcagaagcctgagactgtcttgtgctgcctctggcttcacctttgatgactatgccatgcactgggtcagacaggcccctggcaaaggactggaatgggtgtcagccatcacctggaactctggccacattgactatgctgactctgtggaaggcagattcaccatcagcagagacaatgccaagaacagcctgtacctgcagatgaactccctgagagctgaggacacagcagtgtactactgtgccaaggtgtcctacctgagcacagccagcagcctggattattggggccagggcacactggttacagtgtcctct

SEQ ID NO: 56 - DNA, 294 bp, Artificial Sequence
  synthetic construct; antibody fragment
gccagcacaaagggcccctctgtttttccactggctcccagcagcaagagcaccagtggtggaacagctgccctgggctgtctggtcaaggattacttccctgagcctgtgacagtgtcttggaactcaggggctctgacctctggggtgcacacatttccagctgtgctgcagtcctctggcctgtactctctgtcctctgtggtcacagtgcctagctctagcctgggcacccagacctacatctgcaatgtgaaccacaagcctagcaacaccaaggtggacaagaaggtg

SEQ ID NO: 57 - DNA, 651 bp, Artificial Sequence
  synthetic construct; antibody fragment
gctccagaactgcttggaggcccttctgtgttcctgtttcctccaaagcctaaggacaccctgatgatcagcagaacccctgaagtgacctgtgtggtggttgatgtgtcccatgaggacccagaagtgaagttcaattggtatgtggatggggttgaagtgcacaatgctaagaccaagcctagagaggaacagtacaacagcacctacagagtggtgtctgtgctgacagtgctgcatcaggactggctgaatggcaaagagtacaagtgcaaagtgtccaacaaggccctgcctgctcctattgagaaaaccatctccaaggccaagggccagccaagagaaccccaggtttacacactgccacctagcagagatgagctgaccaagaaccaggtgtccctgacctgcctggttaagggcttctacccctctgacattgctgtggaatgggagagcaatggccagcctgagaacaactacaagacaacccctcctgtgctggactctgatggctcattcttcctgtacagcaagctgactgtggacaagtccagatggcagcaggggaatgtgttcagctgctctgtgatgcatgaggccctgcacaaccactacacccagaaaagtctgagtctgagccctggcaag

SEQ ID NO: 58 - DNA, 651 bp, Artificial Sequence; identical to SEQ ID NO: 57
  synthetic construct; antibody fragment

SEQ ID NO: 59 - DNA, 321 bp, Artificial Sequence
  synthetic construct; antibody fragment
gacatccagatgacacagagccctagcagcctgtctgcttctgtgggagacagagtgaccatcacatgcagagccagccagggaatcagaaactacctggcctggtatcagcaaaagcctggcaaggcccctaagctgctgatctatgcagccagcacactgcagtcaggggtgccaagcagattttcaggctctggctctggcacagacttcaccctgaccatttctagcctgcagcctgaggatgtggccacctactactgccagagatacaacagagccccatacacctttggacagggcacaaaggtggaaatcaag

SEQ ID NO: 60 - DNA, 324 bp, Artificial Sequence
  synthetic construct; antibody fragment
agaacagttgcagcaccctcagttttcatcttccccccctcagatgaacagctgaagtctggcactgcctctgttgtgtgcctgctgaacaacttctaccccagagaagccaaggtgcagtggaaggttgacaatgccctgcagtcaggcaacagccaagaatctgtgactgaacaggattccaaggatagcacctacagcctgagcagcaccctgacactgagcaaggctgactatgagaagcacaaagtgtatgcctgtgaagtgacccaccagggactgagcagcccagtgaccaagagcttcaacaggggagagtgctga

SEQ ID NO: 61 - protein, 205 aa, Artificial Sequence
  synthetic construct; antibody fragment
FPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 62 - protein, 205 aa, Artificial Sequence
  synthetic construct; antibody fragment
FPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 63 - protein, 205 aa, Artificial Sequence
  synthetic construct; antibody fragment
FPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 64 - protein, 205 aa, Artificial Sequence; identical to SEQ ID NO: 61
  synthetic construct; antibody fragment

SEQ ID NO: 65 - protein, 205 aa, Artificial Sequence; identical to SEQ ID NO: 61
  synthetic construct; antibody fragment

SEQ ID NO: 66 - protein, 205 aa, Artificial Sequence; identical to SEQ ID NO: 61
  synthetic construct; antibody fragment

SEQ ID NO: 67 - protein, 203 aa, Artificial Sequence
  synthetic construct; antibody fragment
FPPKPKDTLMISRTPEVTCVVVDVSQEDPEVQFNWYVDGVEVHNAKTKPREEQFNSTFRVVSVLTVVHQDWLNGKEYKCKVSNKGLPAPIEKTISKTKGQPREPQVYTLPPSQEEMTKNQVSLTCLVKGFYPSDIAVEWESNGQPENNYKTTPPMLDSDGSFFLYSKLTVDKSRWQEGNVFSCSVMHEALHAHYTQKSLSLSP

SEQ ID NO: 68 - protein, 205 aa, Artificial Sequence; identical to SEQ ID NO: 61
  synthetic construct; antibody fragment

SEQ ID NO: 69 - protein, 205 aa, Artificial Sequence
  synthetic construct; antibody fragment
FPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 70 - protein, 205 aa, Artificial Sequence; identical to SEQ ID NO: 61
  synthetic construct; antibody fragment

SEQ ID NO: 71 - protein, 205 aa, Artificial Sequence; identical to SEQ ID NO: 63
  synthetic construct; antibody fragment

SEQ ID NO: 72 - protein, 204 aa, Artificial Sequence
  synthetic construct; antibody fragment
FPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 73 - protein, 1725 aa, Artificial Sequence
  synthetic construct; promoter
GACATTGATTATTGACTAGTTATTAATAGTAATCAATTACGGGGTCATTAGTTCATAGCCCATATATGGAGTTCCGCGTTACATAACTTACGGTAAATGGCCCGCCTGGCTGACCGCCCAACGACCCCCGCCCATTGACGTCAATAATGACGTATGTTCCCATAGTAACGCCAATAGGGACTTTCCATTGACGTCAATGGGTGGAGTATTTACGGTAAACTGCCCACTTGGCAGTACATCAAGTGTATCATATGCCAAGTACGCCCCCTATTGACGTCAATGACGGTAAATGGCCCGCCTGGCATTATGCCCAGTACATGACCTTATGGGACTTTCCTACTTGGCAGTACATCTACGTATTAGTCATCGCTATTACCATGGTCGAGGTGAGCCCCACGTTCTGCTTCACTCTCCCCATCTCCCCCCCCTCCCCACCCCCAATTTTGTATTTATTTATTTTTTAATTATTTTGTGCAGCGATGGGGGCGGGGGGGGGGGGGGGGCGCGCGCCAGGCGGGGCGGGGCGGGGCGAGGGGCGGGGCGGGGCGAGGCGGAGAGGTGCGGCGGCAGCCAATCAGAGCGGCGCGCTCCGAAAGTTTCCTTTTATGGCGAGGCGGCGGCGGCGGCGGCCCTATAAAAAGCGAAGCGCGCGGCGGGCGGGAGTCGCTGCGCGCTGCCTTCGCCCCGTGCCCCGCTCCGCCGCCGCCTCGCGCCGCCCGCCCCGGCTCTGACTGACCGCGTTACTCCCACAGGTGAGCGGGCGGGACGGCCCTTCTCCTCCGGGCTGTAATTAGCGCTTGGTTTAATGACGGCTTGTTTCTTTTCTGTGGCTGCGTGAAAGCCTTGAGGGGCTCCGGGAGGGCCCTTTGTGCGGGGGGAGCGGCTCGGGGGGTGCGTGCGTGTGTGTGTGCGTGGGGAGCGCCGCGTGCGGCTCCGCGCTGCCCGGCGGCTGTGAGCGCTGCGGGCGCGGCGCGGGGCTTTGTGCGCTCCGCAGTGTGCGCGAGGGGAGCGCGGCCGGGGGCGGTGCCCCGCGGTGCGGGGGGGGCTGCGAGGGGAACAAAGGCTGCGTGCGGGGTGTGTGCGTGGGGGGGTGAGCAGGGGGTGTGGGCGCGTCGGTCGGGCTGCAACCCCCCCTGCACCCCCCTCCCCGAGTTGCTGAGCACGGCCCGGCTTCGGGTGCGGGGCTCCGTACGGGGCGTGGCGCGGGGCTCGCCGTGCCGGGCGGGGGGTGGCGGCAGGTGGGGGTGCCGGGCGGGGCGGGGCCGCCTCGGGCCGGGGAGGGCTCGGGGGAGGGGCGCGGCGGCCCCCGGAGCGCCGGCGGCTGTCGAGGCGCGGCGAGCCGCAGCCATTGCCTTTTATGGTAATCGTGCGAGAGGGCGCAGGGACTTCCTTTGTCCCAAATCTGTGCGGAGCCGAAATCTGGGAGGCGCCGCCGCACCCCCTCTAGCGGGCGCGGGGCGAAGCGGTGCGGCGCCGGCAGGAAGGAAATGGGCGGGGAGGGCCTTCGTGCGTCGCCGCGCCGCCGTCCCCTTCTCCCTCTCCAGCCTCGGGGCTGTCCGCGGGGGGACGGCTGCCTTCGGGGGGGACGGGGCAGGGCGGGGTTCGGCTTCTGGCGTGTGACCGGCGGCTCTAGAGCCTCTGCTAACCATGTTCATGCCTTCTTCTTTTTCCTACAGCTCCTGGGCAACGTGCTGGTTATTGTGCTGTCTCATCATTTTGGCAAAG

SEQ ID NO: 74 - DNA, 1676 bp, Artificial Sequence
  synthetic construct; promoter
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacag

SEQ ID NO: 75 - DNA, 251 bp, Artificial Sequence
  synthetic construct; promoter
atggaggcggtactatgtagatgagaattcaggagcaaactgggaaaagcaactgcttccaaatatttgtgatttttacagtgtagttttggaaaaactcttagcctaccaattcttctaagtgttttaaaatgtgggagccagtacacatgaagttatagagtgttttaatgaggcttaaatatttaccgtaactatgaaatgctacgcatatcatgctgttcaggctccgtggccacgcaactcatact

SEQ ID NO: 76 - DNA, 212 bp, Artificial Sequence
  synthetic construct; promoter
gggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacag

SEQ ID NO: 77 - DNA, 295 bp, Artificial Sequence
  synthetic construct; promoter
gggccccagaagcctggtggttgtttgtccttctcaggggaaaagtgaggcggccccttggaggaaggggccgggcagaatgatctaatcggattccaagcagctcaggggattgtctttttctagcaccttcttgccactcctaagcgtcctccgtgaccccggctgggatttagcctggtgctgtgtcagccccgggctcccaggggcttcccagtggtccccaggaaccctcgacagggccagggcgtctctctcgtccagcaagggcagggacgggccacaggccaagggc

SEQ ID NO: 78 - DNA, 127 bp, Artificial Sequence
  synthetic construct; polyA
gatctttttccctctgccaaaaattatggggacatcatgaagccccttgagcatctgacttctggctaataaaggaaatttattttcattgcaatagtgtgttggaattttttgtgtctctcactcg

SEQ ID NO: 79 - DNA, 133 bp, Artificial Sequence
  synthetic construct; chimeric intron
gtaagtatcaaggttacaagacaggtttaaggagaccaatagaaactgggcttgtcgagacagagaagactcttgcgtttctgataggcacctattggtcttactgacatccactttgcctttctctccacag

SEQ ID NO: 80 - DNA, 82 bp, Artificial Sequence
  synthetic construct; intron
gtgagtatctcagggatccagacatggggatatgggaggtgcctctgatcccagggctcactgtgggtctctctgttcacag

SEQ ID NO: 81 - DNA, 130 bp, Artificial Sequence
  synthetic construct; ITR
ctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 82 - DNA, 106 bp, Artificial Sequence
  synthetic construct; ITR
ctgcgcgctcgctcgctcactgaggccgcccgggcaaagcccgggcgtcgggcgacctttggtcgcccggcctcagtgagcgagcgagcgcgcagagagggagtgg

SEQ ID NO: 83 - DNA, 143 bp, Artificial Sequence
  synthetic construct; ITR
gaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaa

SEQ ID NO: 84 - DNA, 110 bp, Artificial Sequence
  synthetic construct; ITR
ttggccactccctctctgcgcgctcgctcgctcactgaggccgggcgaccaaaggtcgcccgacgcccgggctttgcccgggcggcctcagtgagcgagcgagcgcgcag

SEQ ID NO: 85 - protein, 20 aa, Artificial Sequence
  synthetic construct; signal peptide
MYRMQLLLLIALSLALVTNS

SEQ ID NO: 86 - DNA, 60 bp, Artificial Sequence
  synthetic construct; signal sequence
atgtataggatgcaactgctcctcctgattgctctgagcctggctcttgtgaccaactct

SEQ ID NO: 87 - protein, 26 aa, Artificial Sequence
  synthetic construct; signal peptide
MNFLLSWVHWSLALLLYLHHAKWSQA

SEQ ID NO: 88 - protein, 29 aa, Artificial Sequence
  synthetic construct; signal peptide
MERAAPSRRVPLPLLLLGGLALLAAGVDA

SEQ ID NO: 89 - protein, 19 aa, Artificial Sequence
  synthetic construct; signal peptide
MAPLRPLLILALLAWVALA

SEQ ID NO: 90 - protein, 18 aa, Artificial Sequence
  synthetic construct; signal peptide
MRLLAKIICLMLWAICVA

SEQ ID NO: 91 - protein, 19 aa, Artificial Sequence
  synthetic construct; signal peptide
MRLLAFLSLLALVLQETGT

SEQ ID NO: 92 - protein, 18 aa, Artificial Sequence
  synthetic construct; signal peptide
MKWVTFISLLFLFSSAYS

SEQ ID NO: 93 - protein, 18 aa, Artificial Sequence
  synthetic construct; signal peptide
MAFLWLLSCWALLGTTFG

SEQ ID NO: 94 - protein, 20 aa, Artificial Sequence
  synthetic construct; signal peptide
MYRMQLLSCIALILALVTNS

SEQ ID NO: 95 - protein, 15 aa, Artificial Sequence
  synthetic construct; signal peptide
MNLLLILTFVAAAVA

SEQ ID NO: 96 - protein, 18 aa, Artificial Sequence
  synthetic construct; signal peptide
MKWVTFISLLFLFSSAYS

SEQ ID NO: 97 - protein, 24 aa, Artificial Sequence
  synthetic construct; signal peptide
MPSSVSWGILLLAGLCCLVPVSLA

SEQ ID NO: 98 - protein, 18 aa, Artificial Sequence
  synthetic construct; signal peptide
MKAAVLTLAVLFLTGSQA

SEQ ID NO: 99 - protein, 18 aa, Artificial Sequence
  synthetic construct; signal peptide
MKLLAATVLLLTICSLEG

SEQ ID NO: 100 - protein, 27 aa, Artificial Sequence
  synthetic construct; signal peptide
MDPPRPALLALLALPALLLLLLAGARA

SEQ ID NO: 101 - protein, 28 aa, Artificial Sequence
  synthetic construct; signal peptide
MQRVNMIMAESPGLITICLLGYLLSAEC

SEQ ID NO: 102 - protein, 20 aa, Artificial Sequence
  synthetic construct; signal peptide
MGPLMVLFCLLFLYPGLADS

SEQ ID NO: 103 - protein, 18 aa, Artificial Sequence
  synthetic construct; signal peptide
MWLLVSVILISRISSVGG

SEQ ID NO: 104 - protein, 18 aa, Artificial Sequence
  synthetic construct; signal peptide
MLLLFSVILISWVSTVGG

SEQ ID NO: 105 - protein, 19 aa, Artificial Sequence
  synthetic construct; signal peptide
MFSMRIVCLVLSVVGTAWT

SEQ ID NO: 106 - protein, 30 aa, Artificial Sequence
  synthetic construct; signal peptide
MKRMVSWSFHKLKTMKHLLLLLLCVFLVKS

SEQ ID NO: 107 - protein, 26 aa, Artificial Sequence
  synthetic construct; signal peptide
MSWSLHPRNLILYFYALLFLSSTCVA

SEQ ID NO: 108 - protein, 18 aa, Artificial Sequence
  synthetic construct; signal peptide
MKSLVLLLCLAQLWGCHS

SEQ ID NO: 109 - protein, 23 aa, Artificial Sequence
  synthetic construct; signal peptide
MARVLGAPVALGLWSLCWSLAIA

SEQ ID NO: 110 - protein, 18 aa, Artificial Sequence
  synthetic construct; signal peptide
MKLITILFLCSRLLLSLT

SEQ ID NO: 111 - protein, 20 aa, Artificial Sequence
  synthetic construct; signal peptide
MSLFPSLPLLLLSMVAASYS

SEQ ID NO: 112 - protein, 19 aa, Artificial Sequence
  synthetic construct; signal peptide
MEHKEVVLLLLLFLKSGQG

SEQ ID NO: 113 - protein, 24 aa, Artificial Sequence
  synthetic construct; signal peptide
MAHVRGLQLPGCLALAALCSLVHS

SEQ ID NO: 114 - protein, 29 aa, Artificial Sequence
  synthetic construct; signal peptide
MISRMEKMTMMMKILIMFALGMNYWSCSG

SEQ ID NO: 115 - protein, 32 aa, Artificial Sequence
  synthetic construct; signal peptide
MYSNVIGTVTSGKRKVYLLSLLLIGFWDCVTC

SEQ ID NO: 116 - protein, 19 aa, Artificial Sequence
  synthetic construct; signal peptide
MRLAVGALLVCAVLGLCLA

SEQ ID NO: 117 - protein, 17 aa, Artificial Sequence
  synthetic construct; signal peptide
MRAWIFFLLCLAGRALA

SEQ ID NO: 118 - protein, 22 aa, Artificial Sequence
  synthetic construct; signal peptide
MFSFVDLRLLLLLAATALLTHG

SEQ ID NO: 119 - protein, 19 aa, Artificial Sequence
  synthetic construct; signal peptide
MKLVFLVLLFLGALGLCLA

SEQ ID NO: 120 - protein, 22 aa, Artificial Sequence
  synthetic construct; signal peptide
MGPTSGPSLLLLLLTHLPLALG

SEQ ID NO: 121 - protein, 18 aa, Artificial Sequence
  synthetic construct; signal peptide
MSLSAFTLFLALIGGTSG

SEQ ID NO: 122 - protein, 27 aa, Artificial Sequence
  synthetic construct; signal peptide
MAPHRPAPALLCALSLALCALSLPVRA

SEQ ID NO: 123 - protein, 22 aa, Artificial Sequence
  synthetic construct; signal peptide
MWATLPLLCAGAWLLGVPVCGA

SEQ ID NO: 124 - protein, 19 aa, Artificial Sequence
  synthetic construct; signal peptide
MQALVLLLCIGALLGHSSC

SEQ ID NO: 125 - protein, 23 aa, Artificial Sequence
  synthetic construct; signal peptide
MQMSPALTCLVLGLALVFGEGSA

SEQ ID NO: 126 - protein, 20 aa, Artificial Sequence
  synthetic construct; signal peptide
MQPSSLLPLALCLLAAPASA

SEQ ID NO: 127 - protein, 23 aa, Artificial Sequence
  synthetic construct; signal peptide
MAPFEPLASGILLLLWLIAPSRA

SEQ ID NO: 128 - protein, 3 aa, Artificial Sequence
  synthetic construct; linker
GSG

SEQ ID NO: 129 - protein, 4 aa, Artificial Sequence
  synthetic construct; linker
RKRR

SEQ ID NO: 130 - protein, 4 aa, Artificial Sequence
  synthetic construct; linker
RRRR

SEQ ID NO: 131 - protein, 4 aa, Artificial Sequence
  synthetic construct; linker
RRKR

SEQ ID NO: 132 - protein, 4 aa, Artificial Sequence
  synthetic construct; linker
RKKR

SEQ ID NO: 133 - protein, 18 aa, Artificial Sequence
  synthetic construct; linker
EGRGSLLTCGDVEENPGP

SEQ ID NO: 134 - protein, 21 aa, Artificial Sequence
  synthetic construct; linker
GSGEGRGSLLTCGDVEENPGP

SEQ ID NO: 135 - protein, 19 aa, Artificial Sequence
  synthetic construct; linker
ATNFSLLKQAGDVEENPGP

SEQ ID NO: 136 - protein, 22 aa, Artificial Sequence
  synthetic construct; linker
GSGATNFSLLKQAGDVEENPGP

SEQ ID NO: 137 - protein, 20 aa, Artificial Sequence
  synthetic construct; linker
QCTNYALLKLAGDVESNPGP

SEQ ID NO: 138 - protein, 23 aa, Artificial Sequence
  synthetic construct; linker
GSGQCTNYALLKLAGDVESNPGP

SEQ ID NO: 139 - protein, 24 aa, Artificial Sequence
  synthetic construct; linker
APVKQTLNFDLLKLAGDVESNPGP

SEQ ID NO: 140 - protein, 27 aa, Artificial Sequence
  synthetic construct; linker
GSGAPVKQTLNFDLLKLAGDVESNPGP

SEQ ID NO: 141 - protein, 22 aa, Artificial Sequence
  synthetic construct; linker
RKRREGRGSLLTCGDVEENPGP

SEQ ID NO: 142 - protein, 25 aa, Artificial Sequence
  synthetic construct; linker
RKRRGSGEGRGSLLTCGDVEENPGP

SEQ ID NO: 143 - protein, 28 aa, Artificial Sequence
  synthetic construct; linker
RKRRAPVKQTLNFDLLKLAGDVESNPGP

SEQ ID NO: 144 - protein, 31 aa, Artificial Sequence
  synthetic construct; linker
RKRRGSGAPVKQTLNFDLLKLAGDVESNPGP

SEQ ID NO: 145 - DNA, 75 bp, Artificial Sequence
  synthetic construct; linker
agaaagagaagaggctctggagaaggcagaggctccctgctgacatgtggggatgttgaagagaatcctgggcct

SEQ ID NO: 146 - DNA, 12 bp, Artificial Sequence
  synthetic construct; linker
agaaagagaaga

SEQ ID NO: 147 - DNA, 21 bp, Artificial Sequence
  synthetic construct; linker
agaaagagaagaggctctgga

SEQ ID NO: 148 - DNA, 9 bp, Artificial Sequence
  synthetic construct; linker
ggctctgga

SEQ ID NO: 149 - DNA, 54 bp, Artificial Sequence
  synthetic construct; linker
gaaggcagaggctccctgctgacatgtggggatgttgaagagaatcctgggcct

SEQ ID NO: 150 - protein, 6 aa, Artificial Sequence
  synthetic construct; hinge region
CPPCPA

SEQ ID NO: 151 - protein, 5 aa, Artificial Sequence
  synthetic construct; hinge region
CPPCP

SEQ ID NO: 152 - protein, 5 aa, Artificial Sequence
  synthetic construct; hinge region
CPPCA

SEQ ID NO: 153 - protein, 22 aa, Artificial Sequence
  synthetic construct; hinge region
EPKSCDKTHTCPPCPAPELLGG

SEQ ID NO: 154 - protein, 22 aa, Artificial Sequence
  synthetic construct; hinge region
EPKSCDKTHLCPPCPAPELLGG

SEQ ID NO: 155 - protein, 10 aa, Artificial Sequence
  synthetic construct; hinge region
EPKSCDKTHL

SEQ ID NO: 156 - protein, 10 aa, Artificial Sequence
  synthetic construct; hinge region
EPKSCDKTHT

SEQ ID NO: 157 - protein, 16 aa, Artificial Sequence
  synthetic construct; hinge region
EPKSCDKTHTCPPCPA

SEQ ID NO: 158 - protein, 16 aa, Artificial Sequence
  synthetic construct; hinge region
EPKSCDKTHLCPPCPA

SEQ ID NO: 159 - protein, 27 aa, Artificial Sequence
  synthetic construct; hinge region
EPKSCDKTHTCPPCPAPELLGGPSVFL

SEQ ID NO: 160 - protein, 27 aa, Artificial Sequence
  synthetic construct; hinge region
EPKSCDKTHLCPPCPAPELLGGPSVFL

SEQ ID NO: 161 - protein, 22 aa, Artificial Sequence
  synthetic construct; hinge region
EPKSCDKTHTCPPCPAPEAAGG

SEQ ID NO: 162 - protein, 27 aa, Artificial Sequence
  synthetic construct; hinge region
EPKSCDKTHLCPPCPAPEAAGGPSVFL

SEQ ID NO: 163 - protein, 18 aa, Artificial Sequence
  synthetic construct; hinge region
ERKSCVECPPCPAPPVAG

SEQ ID NO: 164 - protein, 13 aa, Artificial Sequence
  synthetic construct; hinge region
ERKSCVECPPCPA

SEQ ID NO: 165 - protein, 19 aa, Artificial Sequence
  synthetic construct; hinge region
ESKYGPPCPPCPAPEFLGG

SEQ ID NO: 166 - protein, 13 aa, Artificial Sequence
  synthetic construct; hinge region
ESKYGPPCPPCPA

SEQ ID NO: 167 - protein, 13 aa, Artificial Sequence
  synthetic construct; hinge region
ESKYGPPCPSCPA

SEQ ID NO: 168 - protein, 24 aa, Artificial Sequence
  synthetic construct; hinge region
ESKYGPPCPSCPAPEFLGGPSVFL

SEQ ID NO: 169 - protein, 24 aa, Artificial Sequence
  synthetic construct; hinge region
ESKYGPPCPPCPAPEFLGGPSVFL

SEQ ID NO: 170 - protein, 18 aa, Artificial Sequence
  synthetic construct; hinge region
ERKCCVECPPCPAPPVAG

SEQ ID NO: 171 - protein, 13 aa, Artificial Sequence
  synthetic construct; hinge region
ERKCCVECPPCPA

SEQ ID NO: 172 - protein, 22 aa, Artificial Sequence
  synthetic construct; hinge region
EPKSCDKTHTCPPCPAPELAGA

SEQ ID NO: 173 - protein, 27 aa, Artificial Sequence
  synthetic construct; hinge region
EPKSCDKTHTCPPCPAPELAGAPSVFL

SEQ ID NO: 174 - protein, 27 aa, Artificial Sequence
  synthetic construct; hinge region
EPKSCDKTHLCPPCPAPELAGAPSVFL

SEQ ID NO: 175 - protein, 22 aa, Artificial Sequence
  synthetic construct; hinge region
EPKSCDKTHTCPPCPAPEFEGG

SEQ ID NO: 176 - protein, 27 aa, Artificial Sequence
  synthetic construct; hinge region
EPKSCDKTHTCPPCPAPEFEGGPSVFL

SEQ ID NO: 177 - protein, 27 aa, Artificial Sequence
  synthetic construct; hinge region
EPKSCDKTHLCPPCPAPEFEGGPSVFL

SEQ ID NO: 178 - protein, 15 aa, Artificial Sequence
  synthetic construct; signal peptide
MWCIVLFSLLAWVYA

SEQ ID NO: 179 - protein, 17 aa, Artificial Sequence
  synthetic construct; signal peptide
MNPTLILAAFCLGIASA

SEQ ID NO: 180 - protein, 17 aa, Artificial Sequence
  synthetic construct; signal peptide
MWQLWASLCCLLVLANA

SEQ ID NO: 181 - protein, 16 aa, Artificial Sequence
  synthetic construct; signal peptide
MLLILLSVALLAFSSA

SEQ ID NO: 182 - protein, 20 aa, Artificial Sequence
  synthetic construct; signal peptide
MWKRWLALALALVAVAWVRA

SEQ ID NO: 183 - DNA, 732 bp, Artificial Sequence
  synthetic construct; antibody heavy chain
  misc (670)..(681): nucleotides 670-681 can be present or absent
  misc (682)..(699): nucleotides 682-699 can be present or absent
  misc (700)..(732): nucleotides 700-732 can be present or absent
caggtgcagctgcaggaaagcggcccgggcctggtgcgcccgagccagaccctgagcctgacctgcaccgtgagcggctatagcattaccagcgatcatgcgtggagctgggtgcgccagccgccgggccgcggcctggaatggattggctatattagctatagcggcattaccacctataacccgagcctgaaaagccgcgtgaccatgctgcgcgataccagcaaaaaccagtttagcctgcgcctgagcagcgtgaccgcggcggataccgcggtgtattattgcgcgcgcagcctggcgcgcaccaccgcgatggattattggggccagggcagcctggtgaccgtgagcagcgcgagcaccaaaggcccgagcgtgtttccgctggcgccgagcagcaaaagcaccagcggcggcaccgcggcgctgggctgcctggtgaaagattattttccggaaccggtgaccgtgagctggaacagcggcgcgctgaccagcggcgtgcatacctttccggcggtgctgcagagcagcggcctgtatagcctgagcagcgtggtgaccgtgccgagcagcagcctgggcacccagacctatatttgcaacgtgaaccataaaccgagcaacaccaaagtggataaaaaagtggaaccgaaaagctgcgataagacccacacctgccccccctgccccgcccccgagctgctgggcggccccagcgtgttcctg

SEQ ID NO: 184 - DNA, 642 bp, Artificial Sequence
  synthetic construct; antibody light chain
gatattcagatgacccagagcccgagcagcctgagcgcgagcgtgggcgatcgcgtgaccattacctgccgcgcgagccaggatattagcagctatctgaactggtatcagcagaaaccgggcaaagcgccgaaactgctgatttattataccagccgcctgcatagcggcgtgccgagccgctttagcggcagcggcagcggcaccgattttacctttaccattagcagcctgcagccggaagatattgcgacctattattgccagcagggcaacaccctgccgtatacctttggccagggcaccaaagtggaaattaaacgcaccgtggcggcgccgagcgtgtttatttttccgccgagcgatgaacagctgaaaagcggcaccgcgagcgtggtgtgcctgctgaacaacttttatccgcgcgaagcgaaagtgcagtggaaagtggataacgcgctgcagagcggcaacagccaggaaagcgtgaccgaacaggatagcaaagatagcacctatagcctgagcagcaccctgaccctgagcaaagcggattatgaaaaacataaagtgtatgcgtgcgaagtgacccatcagggcctgagcagcccggtgaccaaaagctttaaccgcggcgaatgc

SEQ ID NO: 185 - protein, 205 aa, Artificial Sequence; identical to SEQ ID NO: 61
  synthetic construct; Fc domain

SEQ ID NO: 186 - protein, 736 aa, Artificial Sequence
  synthetic constructs; AAV vector
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEEVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDEDKFFPMSGVMIFGKESAGASNTALDNVMITDEEEIKATNPVATERFGTVAVNFQSSSTDPATGDVHAMGALPGMVWQDRDVYLQGPIWAKIPHTDGHFHPSPLMGGFGLKNPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 187 - protein, 735 aa, Artificial Sequence [sequence omitted]

SEQ ID NO: 188 - protein, 736 aa, Artificial Sequence
  synthetic constructs; AAV vector
MAADGYLPDWLEDNLSEGIREWWALKPGVPQPKANQQHQDNRRGLVLPGYKYLGPGNGLDKGEPVNEADAAALEHDKAYDQQLKAGDNPYLKYNHADAEFQERLQEDTSFGGNLGRAVFQAKKRILEPLGLVEEAAKTAPGKKGAVDQSPQEPDSSSGVGKSGKQPARKRLNFGQTGDSESVPDPQPLGEPPAAPTSLGSNTMASGGGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKKLSFKLFNIQVRGVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTVANNLQSSNTAPTTGTVNHQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPRPIGTRYLTRNL

SEQ ID NO: 189 - protein, 736 aa, Artificial Sequence; identical to SEQ ID NO: 188
  synthetic constructs; AAV vector

SEQ ID NO: 190 - protein, 736 aa, Artificial Sequence
  synthetic constructs; AAV vector
MAADGYLPDWLEDNLSEGIREWWALKPGVPQPKANQQHQDNRRGLVLPGYKYLGPGNGLDKGEPVNEADAAALEHDKAYDQQLKAGDNPYLKYNHADAEFQERLQEDTSFGGNLGRAVFQAKKRILEPLGLVEEAAKTAPGKKRPVDQSPQEPDSSSGVGKSGKQPARKRLNFGQTGDSESVPDPQPLGEPPAAPTSLGSNTMASGGGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKKLSFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTVANNLQSSNTAPTTRTVNDQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPRPIGTRYLTRNL

SEQ ID NO: 191 - protein, 734 aa, Artificial Sequence
  synthetic constructs; AAV vector
MTDGYLPDWLEDNLSEGVREWWALQPGAPKPKANQQHQDNARGLVLPGYKYLGPGNGLDKGEPVNAADAAALEHDKAYDQQLKAGDNPYLKYNHADAEFQQRLQGDTSFGGNLGRAVFQAKKRVLEPLGLVEQAGETAPGKKRPLIESPQQPDSSTGIGKKGKQPAKKKLVFEDETGAGDGPPEGSTSGAMSDDSEMRAAAGGAAVEGGQGADGVGNASGDWHCDSTWSEGHVTTTSTRTWVLPTYNNHLYKRLGESLQSNTYNGFSTPWGYFDFNRFHCHFSPRDWQRLINNNWGMRPKAMRVKIFNIQVKEVTTSNGETTVANNLTSTVQIFADSSYELPYVMDAGQEGSLPPFPNDVFMVPQYGYCGLVTGNTSQQQTDRNAFYCLEYFPSQMLRTGNNFEITYSFEKVPFHSMYAHSQSLDRLMNPLIDQYLWGLQSTTTGTTLNAGTATTNFTKLRPTNFSNFKKNWLPGPSIKQQGFSKTANQNYKIPATGSDSLIKYETHSTLDGRWSALTPGPPMATAGPADSKFSNSQLIFAGPKQNGNTATVPGTLIFTSEEELAATNATDTDMWGNLPGGDQSNSNLPTVDRLTALGAVPGMVWQNRDIYYQGPIWAKIPHTDGHFHPSPLIGGFGLKHPPPQIFIKNTPVPANPATTFSSTPVNSFITQYSTGQVSVQIDWEIQKERSKRWNPEVQFTSNYGQQNSLLWAPDAAGKYTEPRAIGTRYLTHHL

SEQ ID NO: 192 - protein, 734 aa, Artificial Sequence; identical to SEQ ID NO: 191
  synthetic constructs; AAV vector

SEQ ID NO: 193 - protein, 724 aa, Artificial Sequence
  synthetic constructs; AAV vector
MSFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQEKLADDTSFGGNLGKAVFQAKKRVLEPFGLVEEGAKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 194 - protein, 736 aa, Artificial Sequence
  synthetic constructs; AAV vector
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPFGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQSSSTDPATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 195 - protein, 737 aa, Artificial Sequence
  synthetic constructs; AAV vector
MAADGYLPDWLEDNLSEGIREWWDLKPGAPKPKANQQKQDNGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPAKKRPVEPSPQRSPDSSTGIGKKGQQPARKRLNFGQTGDSESVPDPQPLGEPPAAPSSVGSGTVAAGGGAPMADNNEGADGVGNASGNWHCDSTWLGDRVITTSTRTWALPTYNNHLYKQISSETAGSTNDNTYFGYSTPWGYFDFNRFHCHFSPRDWQRLINNNWGFRPKKLRFKLFNIQVKEVTTNDGVTTIANNLTSTIQVFSDSEYQLPYVLGSAHQGCLPPFPADVFMIPQYGYLTLNNGSQSVGRSSFYCLEYFPSQMLRTGNNFEFSYSFEDVPFHSSYAHSQSLDRLMNPLIDQYLYYLARTQSNPGGTAGNRELQFYQGGPSTMAEQAKNWLPGPCFRQQRVSKTLDQNNNSNFAWTGATKYHLNGRNSLVNPGVAMATHKDDEDRFFPSSGVLIFGKTGATNKTTLENVLMTNEEEIRPTNPVATEEYGIVSSNLQAANTAAQTQVVNNQGALPGMVWQNRDVYLQGPIWAKIPHTDGNFHPSPLMGGFGLKHPPPQILIKNTPVPANPPEVFTPAKFASFITQYSTGQVSVEIEWELQKENSKRWNPEIQYTSNFEKQTGVDFAVDSQGVYSEPRPIGTRYLTRNL

SEQ ID NO: 196 - protein, 738 aa, Artificial Sequence
  synthetic constructs; AAV vector
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 197 - protein, 736 aa, Artificial Sequence
  synthetic constructs; AAV vector
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 198 - protein, 736 aa, Artificial Sequence
  synthetic constructs; AAV vector
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSDGAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 199 - protein, 738 aa, Artificial Sequence
  synthetic constructs; AAV vector
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEPSPQRSPDSSTGIGKKGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYQFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFSQAKLASFITQYSTGQVSVEIEWELQKENSKRWNPEIQYTSNYYKSTNVDFAVNTDGTYSEPRPIGTRYLTRNL

SEQ ID NO: 200 - protein, 738 aa, Artificial Sequence
  synthetic constructs; AAV vector
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEPSPQRSPDSSTGIGKTGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYQFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFSQAKLASFITQYSTGQVSVEIEWELQKENSKRWNPEIQYTSNYYKSTNVDFAVNTEGTYSEPRPIGTRYLTRNL

SEQ ID NO: 201 - protein, 738 aa, Artificial Sequence
  synthetic constructs; AAV vector
MAADGYLPDWLEDNLSEGIREWWALKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEAAKTAPGKKRPVEPSPQRSPDSSTGIGKKGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYTFEDVPFHSSYAHSQSLDRLMNPLIDQYLYYLSRTQSTGGTQGTQQLLFSQAGPANMSAQAKNWLPGPCYRQQRVSTTLSQNNNSNFAWTGATKYHLNGRDSLVNPGVAMATHKDDEERFFPSSGVLMFGKQGAGRDNVDYSSVMLTSEEEIKTTNPVATEQYGVVADNLQQTNTGPIVGNVNSQGALPGMVWQNRDVYLQGPIWAKIPHTDGNFHPSPLMGGFGLKHPPPQILIKNTPVPADPPTTFSQAKLASFITQYSTGQVSVEIEWELQKENSKRWNPEIQYTSNYYKSTNVDFAVNTEGTYSEPRPIGTRYLTRNL

SEQ ID NO: 202 - protein, 738 aa, Artificial Sequence
  synthetic constructs; AAV vector
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSSVGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFSFSYTFEDVPFHSSYAHSQSLDRLMNPLIDQYLYYLSRTQSTGGTAGTQQLLFSQAGPSNMSAQARNWLPGPCYRQQRVSTTLSQNNNSNFAWTGATKYHLNGRDSLVNPGVAMATNKDDEDRFFPSSGILMFGKQGAGKDNVDYSNVMLTSEEEIKTTNPVATEQYGVVADNLQRQNTAPIVGAVNSQGALPGMVWQNRDVYLQGPIWAKIPHTDGNFHPSPLMGGFGLKHPPPQILIKNTPVPADPPTAFNQAKLNSFITQYSTGQVSVEIEWELQKENSKRWNPEIQYTSNYYKSTNVDFAVNTEGVYSEPRPIGTRYLTRNL

SEQ ID NO: 203 - protein, 738 aa, Artificial Sequence
  synthetic constructs; AAV vector
MAADGYLPDWLEDNLSEGIREWWDLKPGAPKPKANQQKQDNGRGLVLPGYKYLGPFNGLDKGEPVNAADAAALEHDKAYDQQLQAGDNPYLRYNHADAEFQERLQEDTSFGGNLGRAVFQAKKRVLEPLGLVESPVKTAPGKKRPVEPSPQRSPDSSTGIGKKGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYNFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFTKAKLASFITQYSTGQVSVEIEWELQKENSKRWNPEIQYTSNYYKSTNVDFAVNTEGTYSEPRPIGTRYLTRNL

SEQ ID NO: 204 - protein, 738 aa, Artificial Sequence
  synthetic constructs; AAV vector
MAADGYLPDWLEDNLSEGIREWWDLKPGAPKPKANQQKQDNGRGLVLPGYKYLGPFNGLDKGEPVNAADAAALEHDKAYDQQLQAGDNPYLRYNHADAEFQERLQEDTSFGGNLGRAVFQAKKRVLEPLGLVESPVKTAPGKKRPVEPSPQRSPDSSTGIGKKGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYNFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFNQAKLASFITQYSTGQVSVEIEWELQKENSKRWNPEIQYTSNYYKSTNVDFAVNTEGTYSEPRPIGTRYLTRNL

SEQ ID NO: 205 - protein, 735 aa, Artificial Sequence
  synthetic constructs; AAV vector
MAADGYLPDWLEDTLSEGIRQWWKLKPGPPPPKPAERHQDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGHQPARKRLNFGQTGDADSVPDPQPLGQPPAAPTSLGSTTMATGSGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRPSFYCLEYFPSQMLRTGNNFTFSYTFEDVPFHSSYAHSQSLDRLMNPLIDQYLYYLNRTQSNSGTLQQSRLLFSQAGPTSMSLQAKNWLPGPCYRQQRLSKQANDNNNSNFPWTAATKYHLNGRDSLVNPGPAMASHKDDEEKFFPMHGTLIFGKQGTNANDADLEHVMITDEEEIRTTNPVATEQYGNVSNNLQNSNTGPTTENVNHQGALPGMVWQDRDVYLQGPIWAKIPHTDGHFHPSPLMGGFGLKHPPPQIMIKNTPVPANPPTNFSSAKFASFITQYSTGQVSVEIEWELQKENSKRWNPEIQYTSNYNKSVNVDFTVDTNGVYSEPRPIGTRYLTRNL

SEQ ID NO: 206 - protein, 735 aa, Artificial Sequence
  synthetic constructs; AAV vector
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRILEPLGLVEEPVKTAPGKKRPVEHSPAEPDSSSGTGKAGQQPARKRLNFGQTGDADSVPDPRPLGQPPAAPSGLGTNTMASGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLSFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTMSRLQFSQAGASDIRDQSRNWLPGPCYRQQRVSKTAADNNNSDYSWTGATKYHLNGRDSLVNPGPAMASHKDDEEKYFPQSGVLIFGKQDSGKTNVDIEKVMITDEEEIRTTNPVATEQYGSVSTNLQSGNTQAATSDVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 207 - protein, 735 aa, Artificial Sequence
  synthetic constructs; AAV vector
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRILEPLGLVEEPVKTAPGKKRPVEHSPAEPDSSSGTGKAGQQPARKRLNFGQTGDADSVPDPQPLGQPPAAPSGLGTNTMASGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLSFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTMSRLQFSQAGASDIRDQSRNWLPGPCYRQQRVSKTAADNNNSDYSWTGATKYHLNGRDSLVNPGPAMASHKDDEEKYFPQSGVLIFGKQDSGKTNVDIEKVMITDEEEIRTTNPVATEQYGSVSTNLQSGNTQAATSDVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 208 - protein, 738 aa, Artificial Sequence
  synthetic constructs; AAV vector
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEAAKTAPGKKRPVEPSPQRSPDSSTGIGKKGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYTFEDVPFHSSYAHSQSLDRLMNPLIDQYLYYLSRTQSTGGTQGTQQLLFSQAGPANMSAQAKNWLPGPCYRQQRVSTTLSQNNNSNFAWTGATKYHLNGRDSLVNPGVAMATHKDDEERFFPSSGVLMFGKQGAGRDNVDYSSVMLTSEEEIKTTNPVATEQYGVVADNLQQTNTGPIVGNVNSQGALPGMVWQNRDVYLQGPIWAKIPHTDGNFHPSPLMGGFGLKHPPPQILIKNTPVPADPPTTFSQAKLASFITQYSTGQVSVEIEWELQKENSKRWNPEIQYTSNYYKSTNVDFAVNTEGTYSEPRPIGTRYLTRNL

SEQ ID NO: 209 - protein, 736 aa, Artificial Sequence
  synthetic constructs; AAV vector
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVGEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSGYAHSQSLDRLMNPLIDQYLYYLSTTNTPSGTTTQSRLQFSQAGASDIRDQSRNWLPGPCYRQQRVSKTSADNNNSEYSWTGATKYHLNGRDSLVNPGPAMASHKDNEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 210 - protein, 734 aa, Artificial Sequence
  synthetic constructs; AAV vector
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPAEPDSSSGTGKAGQQPARKRLNFGQTGDADSVPDPQPLRQPPAAPTSLGSTTMATGSGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQTASGTQQSRLLFSQAGPTSMSLQAKNWLPGPCYRQQRLSKQANDNNNSNFPWTGATKYYLNGRDSLVNPGPAMASHKDDEEKFFPMHGTLIFGKEGTNATNAELENVMITDEEEIRTTNPVATEQYGYVSNNLQNSNTAASTETVNHQGALPGMVWQDRDVYLQGPIWAKIPHTDGHFHPSPLMGGFGLKHPPPQIMIKNTPVPANPPTNFSSAKFASFITQYSTGQVSVEIEWELQKENSKRWNPEIQYTSNYNKSVNVDFTVDTNGVYSEPRPIGTRYLTRNL

SEQ ID NO: 211 - DNA, 1212 bp, Artificial Sequence
  synthetic construct; promoter
gtctaacaaaaaagccaaaaacggccagaatttagcggacaatttactagtctaacactgaaaattacatattgacccaaatgattacatttcaaaaggtgcctaaaaaacttcacaaaacacactcgccaaccccgagcgcatagttcaaaaccggagcttcagctacttaagaagataggtacataaaaccgaccaaagaaactgacgcctcacttatccctcccctcaccagaggtccggcgcctgtcgattcaggagagcctaccctaggcccgaaccctgcgtcctgcgacggagaaaagcctaccgcacacctaccggcaggtggccccaccctgcattataagccaacagaacgggtgacgtcacgacacgacgagggcgcgcgctcccaaaggtacgggtgcactgcccaacggcaccgccataactgccgcccccgcaacagacgacaaaccgagttctccagtcagtgacaaacttcacgtcagggtccccagatggtgccccagcccatctcacccgaataagagctttcccgcattagcgaaggcctcaagaccttgggttcttgccgcccaccatgccccccaccttgtttcaacgacctcacagcccgcctcacaagcgtcttccattcaagactcgggaacagccgccattttgctgcgctccccccaacccccagttcagggcaaccttgctcgcggacccagactacagcccttggcggtctctccacacgcttccgtcccaccgagcggcccggcggccacgaaagccccggccagcccagcagcccgctactcaccaagtgacgatcacagcgatccacaaacaagaaccgcgacccaaatcccggctgcgacggaactagctgtgccacacccggcgcgtccttatataatcatcggcgttcaccgccccacggagatccctccgcagaatcgccgagaagggactacttttcctcgcctgttccgctctctggaaagaaaaccagtgccctagagtcacccaagtcccgtcctaaaatgtccttctgctgatactggggttctaaggccgagtcttatgagcagcgggccgctgtcctgagcgtccgggcggaaggatcaggacgctcgctgcgcccttcgtctgacgtggcagcgctcgccgtgaggaggggggcgcccgcgggaggcgccaaaacccggcgcggaggcc

SEQ ID NO: 212 - DNA, 1724 bp, Artificial Sequence
  synthetic construct; promoter
ggaggctgaggggtggggaaagggcatgggtgtttcatgaggacagagcttccgtttcatgcaatgaaaagagtttggagacggatggtggtgactggactatacacttacacacggtagcgatggtacactttgtattatgtatattttaccacgatctttttaaagtgtcaaaggcaaatggccaaatggttccttgtcctatagctgtagcagccatcggctgttagtgacaaagcccctgagtcaagatgacagcagcccccataactcctaatcggctctcccgcgtggagtcatttaggagtagtcgcattagagacaagtccaacatctaatcttccaccctggccagggccccagctggcagcgagggtgggagactccgggcagagcagagggcgctgacattggggcccggcctggcttgggtccctctggcctttccccaggggccctctttccttggggctttcttgggccgccactgctcccgctcctctccccccatcccaccccctcaccccctcgttcttcatatccttctctagtgctccctccactttcatccacccttctgcaagagtgtgggaccacaaatgagttttcacctggcctggggacacacgtgcccccacaggtgctgagtgactttctaggacagtaatctgctttaggctaaaatgggacttgatcttctgttagccctaatcatcaattagcagagccggtgaaggtgcagaacctaccgcctttccaggcctcctcccacctctgccacctccactctccttcctgggatgtgggggctggcacacgtgtggcccagggcattggtgggattgcactgagctgggtcattagcgtaatcctggacaagggcagacagggcgagcggagggccagctccggggctcaggcaaggctgggggcttcccccagacaccccactcctcctctgctggacccccacttcatagggcacttcgtgttctcaaagggcttccaaatagcatggtggccttggatgcccagggaagcctcagagttgcttatctccctctagacagaaggggaatctcggtcaagagggagaggtcgccctgttcaaggccacccagccagctcatggcggtaatgggacaaggctggccagccatcccaccctcagaagggacccggtggggcaggtgatctcagaggaggctcacttctgggtctcacattcttggatccggttccaggcctcggccctaaatagtctccctgggctttcaagagaaccacatgagaaaggaggattcgggctctgagcagtttcaccacccaccccccagtctgcaaatcctgacccgtgggtccacctgccccaaaggcggacgcaggacagtagaagggaacagagaacacataaacacagagagggccacagcggctcccacagtcaccgccaccttcctggcggggatgggtggggcgtctgagtttggttcccagcaaatccctctgagccgcccttgcgggctcgcctcaggagcaggggagcaagaggtgggaggaggaggtctaagtcccaggcccaattaagagatcaggtagtgtagggtttgggagcttttaaggtgaagaggcccgggctgatcccacaggccagtataaagcgccgtgaccctcaggtgatgcgccagggccggctgccgtcggggacagggctttccatagcc

SEQ ID NO: 213 - DNA, 806 bp, Artificial Sequence
  synthetic construct; promoter
agatcttccccacctagccacctggcaaactgctccttctctcaaaggcccaaacatggcctcccagactgcaacccccaggcagtcaggccctgtctccacaacctcacagccaccctggacggaatctgcttcttcccacatttgagtcctcctcagcccctgagctcctctgggcagggctgtttctttccatctttgtattcccaggggcctgcaaataaatgtttaatgaacgaacaagagagtgaattccaattccatgcaacaaggattgggctcctgggccctaggctatgtgtctggcaccagaaacggaagctgcaggttgcagcccctgccctcatggagctcctcctgtcagaggagtgtggggactggatgactccagaggtaacttgtgggggaacgaacaggtaaggggctgtgtgacgagatgagagactgggagaataaaccagaaagtctctagctgtccagaggacatagcacagaggcccatggtccctatttcaaacccaggccaccagactgagctgggaccttgggacagacaagtcatgcagaagttaggggaccttctcctcccttttcctggatggatcctgagtaccttctcctccctgacctcaggcttcctcctagtgtcaccttggcccctcttagaagccaattaggccctcagtttctgcagcggggattaatatgattatgaacacccccaatctcccagatgctgattcagccaggagcttaggagggggaggtcactttataagggtctgggggggtcagaacccagagtc

SEQ ID NO: 214 - DNA, 1003 bp, Artificial Sequence
  synthetic construct; promoter
tggccctggcattcccctatactgggacatagaaccttcacaggaccaagggcctctcctcccattgatgactgactaggccatcctctagctacataggtggtggagcctagagtccctccttgtgtactctttggtggtggttactctgggggtactaggttagttcgtattgttgttcctcctaggggactgcaaaccccttcagctccttgggtcctttctctagttccttctttggggaccctgtgctcagttcaatggatggccaatttccttcttaaatgcccctagcagtaactgttaggtctcaatcccaagacaaatgtctgaggtgcctatttaacagatcaaagcggacctgtcctcaggttaacccagtcactccctgtacctcagtccctacccatcacaattctccagcccatgagcttcgggctgtacttccccaacgggttctcccattttgggtacatggccttttttttttacctttttggttcctttggccttttggcttttggcttccagggcttctggatcccccccaacccctcccatacacatacacatgtgcactcgtgcactcaacccagcacaggataatgttcattcttgacctttccacatacatctggctatgttctctctcttatctacaataaatctcctccactatacttaggagcagttatgttcttcttctttctttcttttttttttttttttcattcagtaacatcatcagaatcccctagctctggcctacctcctcagtaacaatcagctgatccctggccactaatctgtactcactaatctgttttccaaactcttggcccctgagctaattatagcagtgcttcatgccacccaccccaaccctatccttgttctctgactcccactaatctacacattcagaggattgtggatataagaggctgggaggccagcttagcaaccagagctcgaggctgatgcgagcttcatctcttccctc

SEQ ID NO: 215 - DNA, 523 bp, Artificial Sequence
  synthetic construct; promoter
ttttttttttacctttttggttcctttggccttttggcttttggcttccagggcttctggatcccccccaacccctcccatacacatacacatgtgcactcgtgcactcaacccagcacaggataatgttcattcttgacctttccacatacatctggctatgttctctctcttatctacaataaatctcctccactatacttaggagcagttatgttcttcttctttctttcttttttttttttttttcattcagtaacatcatcagaatcccctagctctggcctacctcctcagtaacaatcagctgatccctggccactaatctgtactcactaatctgttttccaaactcttggcccctgagctaattatagcagtgcttcatgccacccaccccaaccctatccttgttctctgactcccactaatctacacattcagaggattgtggatataagaggctgggaggccagcttagcaaccagagctcgaggctgatgcgagcttcatctcttccctc

SEQ ID NO: 216 - DNA, 215 bp, Artificial Sequence
  synthetic construct; promoter
tgatccctggccactaatctgtactcactaatctgttttccaaactcttggcccctgagctaattatagcagtgcttcatgccacccaccccaaccctatccttgttctctgactcccactaatctacacattcagaggattgtggatataagaggctgggaggccagcttagcaaccagagctcgaggctgatgcgagcttcatctcttccctc

SEQ ID NO: 217 - DNA, 198 bp, Artificial Sequence
  synthetic construct; promoter
gggccccagaagcctggtggttgtttgtccttctcaggggaaaagtgaggcggccccttggaggaaggggccgggcagaatgatctaatcggattccaagcagctcaggggattgtctttttctagcaccttcttgccactcctaagcgtcctccgtgaccccggctgggatttagcctggtgctgtgtcagccccgg

SEQ ID NO: 218 - DNA, 142 bp, Artificial Sequence
  synthetic construct; promoter
aaggactcctttgtggaggtcctggcttagggagtcaagtgacggcggctcagcactcacgtgggcagtgccagcctctaagagtgggcaggggcactggccacagagtcccagggagtcccaccagcctagtcgccagacc

SEQ ID NO: 219 - DNA, 109 bp, Artificial Sequence
  synthetic construct; promoter
gtcaagtgacggcggctcagcactcacgtgggcagtgccagcctctaagagtgggcaggggcactggccacagagtcccagggagtcccaccagcctagtcgccagacc

SEQ ID NO: 220 - DNA, 93 bp, Artificial Sequence
  synthetic construct; promoter
ctcagcactcacgtgggcagtgccagcctctaagagtgggcaggggcactggccacagagtcccagggagtcccaccagcctagtcgccagac

SEQ ID NO: 221 - DNA, 1080 bp, Artificial Sequence
  synthetic construct; promoter
ttaataaacatttgggcgattcttacggcctctaaagaccaagaaccactgctgcctagagctctgctctcttcattgaacaatacaagaggagtgtgtaggtagacacccaccacttccaacagcttaggagagcccttgagtatggattgatgtattaaaatttattgaatcacatgctgagattttcaccagctgcccgtggggatctgggcatttattcccatattgcactggctggctggaagccagcagcataaactccagggctgttctgtcaacccccaccagactcaccccgctccaccagccccggcaggcttctccttccatctctctgaagcaacttactgatgggccctgccagccaatcacagccagaataacgtatgatgtcaccagcagccaatcagagctcctcgtcagcatatgcagaattctgtcattttactagggtgatgaaattcccaagcaacaccatccttttcagataagggcactgaggctgagagaggagctgaaacctacccggcgtcaccacacacaggtggcaaggctgggaccagaaaccaggactgttgactgcagcccggtattcattctttccatagcccacagggctgtcaaagaccccagggcctagtcagaggctcctccttcctggagagttcctggcacagaagttgaagctcagcacagccccctaacccccaactctctctgcaaggcctcaggggtcagaacactggtggagcagatcctttagcctctggattttagggccatggtagagggggtgttgccctaaattccagccctggtctcagcccaacaccctccaagaagaaattagaggggccatggccaggctgtgctagccgttgcttctgagcagattacaagaagggaccaagacaaggactcctttgtggaggtcctggcttagggagtcaagtgacggcggctcagcactcacgtgggcagtgccagcctctaagagtgggcaggggcactggccacagagtcccagggagtcccaccagcctagtcgccagaccttctgtgggatcatcggaccca

SEQ ID NO: 222 - DNA, 2280 bp, Artificial Sequence
  synthetic construct; AAV-antibody construct
ctgcgcgctcgctcgctcactgaggccgcccgggcaaagcccgggcgtcgggcgacctttggtcgcccggcctcagtgagcgagcgagcgcgcagagagggagtggaattcacgcgtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagcaggtgagtatctcagggatccagacatggggatatgggaggtgcctctgatcccagggctcactgtgggtctctctgttcacaggttaccggtgccaccatgtacagaatgcagctgctgctgctcattgccctgtctctggccctggtcaccaattctgaagtgcagctggtggaaagtggtggtggactggtgcagcctggcagaagcctgagactgtcttgtgctgcctctggcttcacctttgatgactatgccatgcactgggtcagacaggcccctggcaaaggactggaatgggtgtcagccatcacctggaactctggccacattgactatgctgactctgtggaaggcagattcaccatcagcagagacaatgccaagaacagcctgtacctgcagatgaactccctgagagctgaggacacagcagtgtactactgtgccaaggtgtcctacctgagcacagccagcagcctggattattggggccagggcacactggttacagtgtcctctgccagcacaaagggcccctctgtttttccactggctcccagcagcaagagcaccagtggtggaacagctgccctgggctgtctggtcaaggattacttccctgagcctgtgacagtgtcttggaactcaggggctctgacctctggggtgcacacatttccagctgtgctgcagtcctctggcctgtactctctgtcctctgtggtcacagtgcctagctctagcctgggcacccagacctacatctgcaatgtgaaccacaagcctagcaacaccaaggtggacaagaaggtggaacccaagagctgtgacaagacccacagaaagagaagaggctctggagaaggcagaggctccctgctgacatgtggggatgttgaagagaatcctgggcctatgtataggatgcaactgctcctcctgattgctctgagcctggctcttgtgaccaactctgacatccagatgacacagagccctagcagcctgtctgcttctgtgggagacagagtgaccatcacatgcagagccagccagggcatcagaaactacctggcctggtatcagcagaagccaggcaaggcccctaagctgctgatctatgcagccagcacactgcagagtggggtgccaagcagattttcaggctctggctctggcacagacttcaccctgaccatttctagcctgcagcctgaggatgtggccacctactactgccagagatacaacagagccccatacacctttggacagggcacaaaggtggaaatcaagagaacagttgcagcaccctcagttttcatcttccccccctcagatgaacagctgaagtctggcactgcctctgttgtgtgcctgctgaacaacttctaccccagagaagccaaggtgcagtggaaggttgacaatgccctgcagtcaggcaacagccaagaatctgtgactgaacaggattccaaggatagcacctacagcctgagcagcaccctgacactgagcaaggctgactatgagaagcacaaagtgtatgcctgtgaagtgacccaccagggactgagcagcccagtgaccaagagcttcaacaggggagagtgctgataaaagcttgatctttttccctctgccaaaaattatggggacatcatgaagccccttgagcatctgacttctggctaataaaggaaatttattttcattgcaatagtgtgttggaattttttgtgtctctcactcggctagcgaagcaattctagcaggcatgctggggagagatcgatctgaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaa

SEQ ID NO: 223 - DNA, 1972 bp, Artificial Sequence
  synthetic construct; AAV-antibody construct
gggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagcaggtgagtatctcagggatccagacatggggatatgggaggtgcctctgatcccagggctcactgtgggtctctctgttcacaggttaccggtgccaccatgtacagaatgcagctgctgctgctcattgccctgtctctggccctggtcaccaattctgaagtgcagctggtggaaagtggtggtggactggtgcagcctggcagaagcctgagactgtcttgtgctgcctctggcttcacctttgatgactatgccatgcactgggtcagacaggcccctggcaaaggactggaatgggtgtcagccatcacctggaactctggccacattgactatgctgactctgtggaaggcagattcaccatcagcagagacaatgccaagaacagcctgtacctgcagatgaactccctgagagctgaggacacagcagtgtactactgtgccaaggtgtcctacctgagcacagccagcagcctggattattggggccagggcacactggttacagtgtcctctgccagcacaaagggcccctctgtttttccactggctcccagcagcaagagcaccagtggtggaacagctgccctgggctgtctggtcaaggattacttccctgagcctgtgacagtgtcttggaactcaggggctctgacctctggggtgcacacatttccagctgtgctgcagtcctctggcctgtactctctgtcctctgtggtcacagtgcctagctctagcctgggcacccagacctacatctgcaatgtgaaccacaagcctagcaacaccaaggtggacaagaaggtggaacccaagagctgtgacaagacccacagaaagagaagaggctctggagaaggcagaggctccctgctgacatgtggggatgttgaagagaatcctgggcctatgtataggatgcaactgctcctcctgattgctctgagcctggctcttgtgaccaactctgacatccagatgacacagagccctagcagcctgtctgcttctgtgggagacagagtgaccatcacatgcagagccagccagggcatcagaaactacctggcctggtatcagcagaagccaggcaaggcccctaagctgctgatctatgcagccagcacactgcagagtggggtgccaagcagattttcaggctctggctctggcacagacttcaccctgaccatttctagcctgcagcctgaggatgtggccacctactactgccagagatacaacagagccccatacacctttggacagggcacaaaggtggaaatcaagagaacagttgcagcaccctcagttttcatcttccccccctcagatgaacagctgaagtctggcactgcctctgttgtgtgcctgctgaacaacttctaccccagagaagccaaggtgcagtggaaggttgacaatgccctgcagtcaggcaacagccaagaatctgtgactgaacaggattccaaggatagcacctacagcctgagcagcaccctgacactgagcaaggctgactatgagaagcacaaagtgtatgcctgtgaagtgacccaccagggactgagcagcccagtgaccaagagcttcaacaggggagagtgctgataaaagcttgatctttttccctctgccaaaaattatggggacatcatgaagccccttgagcatctgacttctggctaataaaggaaatttattttcattgcaatagtgtgttggaattttttgtgtctctcactcg

SEQ ID NO: 224 - DNA, 2319 bp, Artificial Sequence
  synthetic construct; AAV-antibody construct
ctgcgcgctcgctcgctcactgaggccgcccgggcaaagcccgggcgtcgggcgacctttggtcgcccggcctcagtgagcgagcgagcgcgcagagagggagtggaattcacgcgtatggaggcggtactatgtagatgagaattcaggagcaaactgggaaaagcaactgcttccaaatatttgtgatttttacagtgtagttttggaaaaactcttagcctaccaattcttctaagtgttttaaaatgtgggagccagtacacatgaagttatagagtgttttaatgaggcttaaatatttaccgtaactatgaaatgctacgcatatcatgctgttcaggctccgtggccacgcaactcatactcaggtgagtatctcagggatccagacatggggatatgggaggtgcctctgatcccagggctcactgtgggtctctctgttcacaggttaccggtgccaccatgtacagaatgcagctgctgctgctcattgccctgtctctggccctggtcaccaattctgaagtgcagctggtggaaagtggtggtggactggtgcagcctggcagaagcctgagactgtcttgtgctgcctctggcttcacctttgatgactatgccatgcactgggtcagacaggcccctggcaaaggactggaatgggtgtcagccatcacctggaactctggccacattgactatgctgactctgtggaaggcagattcaccatcagcagagacaatgccaagaacagcctgtacctgcagatgaactccctgagagctgaggacacagcagtgtactactgtgccaaggtgtcctacctgagcacagccagcagcctggattattggggccagggcacactggttacagtgtcctctgccagcacaaagggcccctctgtttttccactggctcccagcagcaagagcaccagtggtggaacagctgccctgggctgtctggtcaaggattacttccctgagcctgtgacagtgtcttggaactcaggggctctgacctctggggtgcacacatttccagctgtgctgcagtcctctggcctgtactctctgtcctctgtggtcacagtgcctagctctagcctgggcacccagacctacatctgcaatgtgaaccacaagcctagcaacaccaaggtggacaagaaggtggaacccaagagctgtgacaagacccacagaaagagaagaggctctggagaaggcagaggctccctgctgacatgtggggatgttgaagagaatcctgggcctatgtataggatgcaactgctcctcctgattgctctgagcctggctcttgtgaccaactctgacatccagatgacacagagccctagcagcctgtctgcttctgtgggagacagagtgaccatcacatgcagagccagccagggcatcagaaactacctggcctggtatcagcagaagccaggcaaggcccctaagctgctgatctatgcagccagcacactgcagagtggggtgccaagcagattttcaggctctggctctggcacagacttcaccctgaccatttctagcctgcagcctgaggatgtggccacctactactgccagagatacaacagagccccatacacctttggacagggcacaaaggtggaaatcaagagaacagttgcagcaccctcagttttcatcttccccccctcagatgaacagctgaagtctggcactgcctctgttgtgtgcctgctgaacaacttctaccccagagaagccaaggtgcagtggaaggttgacaatgccctgcagtcaggcaacagccaagaatctgtgactgaacaggattccaaggatagcacctacagcctgagcagcaccctgacactgagcaaggctgactatgagaagcacaaagtgtatgcctgtgaagtgacccaccagggactgagcagcccagtgaccaagagcttcaacaggggagagtgctgataaaagcttgatctttttccctctgccaaaaattatggggacatcatgaagccccttgagcatctgacttctggctaataaaggaaatttattttcattgcaatagtgtgttggaattttttgtgtctctcactcggctagcgaagcaattctagcaggcatgctggggagagatcgatctgaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaa

SEQ ID NO: 225 - DNA, 2011 bp, Artificial Sequence
  synthetic construct; AAV-antibody construct
atggaggcggtactatgtagatgagaattcaggagcaaactgggaaaagcaactgcttccaaatatttgtgatttttacagtgtagttttggaaaaactcttagcctaccaattcttctaagtgttttaaaatgtgggagccagtacacatgaagttatagagtgttttaatgaggcttaaatatttaccgtaactatgaaatgctacgcatatcatgctgttcaggctccgtggccacgcaactcatactcaggtgagtatctcagggatccagacatggggatatgggaggtgcctctgatcccagggctcactgtgggtctctctgttcacaggttaccggtgccaccatgtacagaatgcagctgctgctgctcattgccctgtctctggccctggtcaccaattctgaagtgcagctggtggaaagtggtggtggactggtgcagcctggcagaagcctgagactgtcttgtgctgcctctggcttcacctttgatgactatgccatgcactgggtcagacaggcccctggcaaaggactggaatgggtgtcagccatcacctggaactctggccacattgactatgctgactctgtggaaggcagattcaccatcagcagagacaatgccaagaacagcctgtacctgcagatgaactccctgagagctgaggacacagcagtgtactactgtgccaaggtgtcctacctgagcacagccagcagcctggattattggggccagggcacactggttacagtgtcctctgccagcacaaagggcccctctgtttttccactggctcccagcagcaagagcaccagtggtggaacagctgccctgggctgtctggtcaaggattacttccctgagcctgtgacagtgtcttggaactcaggggctctgacctctggggtgcacacatttccagctgtgctgcagtcctctggcctgtactctctgtcctctgtggtcacagtgcctagctctagcctgggcacccagacctacatctgcaatgtgaaccacaagcctagcaacaccaaggtggacaagaaggtggaacccaagagctgtgacaagacccacagaaagagaagaggctctggagaaggcagaggctccctgctgacatgtggggatgttgaagagaatcctgggcctatgtataggatgcaactgctcctcctgattgctctgagcctggctcttgtgaccaactctgacatccagatgacacagagccctagcagcctgtctgcttctgtgggagacagagtgaccatcacatgcagagccagccagggcatcagaaactacctggcctggtatcagcagaagccaggcaaggcccctaagctgctgatctatgcagccagcacactgcagagtggggtgccaagcagattttcaggctctggctctggcacagacttcaccctgaccatttctagcctgcagcctgaggatgtggccacctactactgccagagatacaacagagccccatacacctttggacagggcacaaaggtggaaatcaagagaacagttgcagcaccctcagttttcatcttccccccctcagatgaacagctgaagtctggcactgcctctgttgtgtgcctgctgaacaacttctaccccagagaagccaaggtgcagtggaaggttgacaatgccctgcagtcaggcaacagccaagaatctgtgactgaacaggattccaaggatagcacctacagcctgagcagcaccctgacactgagcaaggctgactatgagaagcacaaagtgtatgcctgtgaagtgacccaccagggactgagcagcccagtgaccaagagcttcaacaggggagagtgctgataaaagcttgatctttttccctctgccaaaaattatggggacatcatgaagccccttgagcatctgacttctggctaataaaggaaatttattttcattgcaatagtgtgttggaattttttgtgtctctcactcg

SEQ ID NO: 226 - protein, 31 aa, Artificial Sequence
  synthetic construct; signal peptide
MLRGPGPGLLLLAVQCLGTAVPSTGASKSKR

SEQ ID NO: 227 - protein, 5 aa, Artificial Sequence
  synthetic construct; HIF1alpha site
RCGTG

SEQ ID NO: 228 - DNA, 969 bp, Artificial Sequence
  synthetic construct; regulatory element
aggttaatttttaaaaagcagtcaaaagtccaagtggcccttggcagcatttactctctctgtttgctctggttaataatctcaggagcacaaacattccagatccaggttaatttttaaaaagcagtcaaaagtccaagtggcccttggcagcatttactctctctgtttgctctggttaataatctcaggagcacaaacattccagatccggcgcgccagggctggaagctacctttgtctagaaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtacccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagt

SEQ ID NO: 229 - DNA, 1050 bp, Artificial Sequence
  synthetic construct; regulatory element
aggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggtctagaaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtacccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagt

SEQ ID NO: 230 - DNA, 1089 bp, Artificial Sequence
  synthetic construct; regulatory element
aggttaatttttaaaaagcagtcaaaagtccaagtggcccttggcagcatttactctctctgtttgctctggttaataatctcaggagcacaaacattccagatccaggttaatttttaaaaagcagtcaaaagtccaagtggcccttggcagcatttactctctctgtttgctctggttaataatctcaggagcacaaacattccagatccggcgcgccagggctggaagctacctttgacatcatttcctctgcgaatgcatgtataatttctacagaacctattagaaaggatcacccagcctctgcttttgtacaactttcccttaaaaaactgccaattccactgctgtttggcccaatagtgagaactttttcctgctgcctcttggtgcttttgcctatggcccctattctgcctgctgaagacactcttgccagcatggacttaaacccctccagctctgacaatcctctttctcttttgttttacatgaagggtctggcagccaaagcaatcactcaaagttcaaaccttatcattttttgctttgttcctcttggccttggttttgtacatcagctttgaaaataccatcccagggttaatgctggggttaatttataactaagagtgctctagttttgcaatacaggacatgctataaaaatggaaagatgttgctttctgagaggatcttgctaccagtggaacagccactaaggattctgcagtgagagcagagggccagctaagtggtactctcccagagactgtctgactcacgccaccccctccaccttggacacaggacgctgtggtttctgagccaggtacagtgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagt

SEQ ID NO: 231 - DNA, 1430 bp, Artificial Sequence
  synthetic construct; regulatory element
aggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggtctagagcccttaagctagcaggttaatttttaaaaagcagtcaaaagtccaagtggcccttggcagcatttactctctctgtttgctctggttaataatctcaggagcacaaacattccagatccaggttaatttttaaaaagcagtcaaaagtccaagtggcccttggcagcatttactctctctgtttgctctggttaataatctcaggagcacaaacattccagatccggcgcgccagggctggaagctacctttgacatcatttcctctgcgaatgcatgtataatttctacagaacctattagaaaggatcacccagcctctgcttttgtacaactttcccttaaaaaactgccaattccactgctgtttggcccaatagtgagaactttttcctgctgcctcttggtgcttttgcctatggcccctattctgcctgctgaagacactcttgccagcatggacttaaacccctccagctctgacaatcctctttctcttttgttttacatgaagggtctggcagccaaagcaatcactcaaagttcaaaccttatcattttttgctttgttcctcttggccttggttttgtacatcagctttgaaaataccatcccagggttaatgctggggttaatttataactaagagtgctctagttttgcaatacaggacatgctataaaaatggaaagatgttgctttctgagaggatcttgctaccagtggaacagccactaaggattctgcagtgagagcagagggccagctaagtggtactctcccagagactgtctgactcacgccaccccctccaccttggacacaggacgctgtggtttctgagccaggtacagtgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagt

SEQ ID NO: 232 - DNA, 1318 bp, Artificial Sequence
  synthetic construct; regulatory element
aggttaatttttaaaaagcagtcaaaagtccaagtggcccttggcagcatttactctctctgtttgctctggttaataatctcaggagcacaaacattccagatccaggttaatttttaaaaagcagtcaaaagtccaagtggcccttggcagcatttactctctctgtttgctctggttaataatctcaggagcacaaacattccagatccggcgcgccagggctggaagctacctttgacatcatttcctctgcgaatgcatgtataatttctacagaacctattagaaaggatcacccagcctctgcttttgtacaactttcccttaaaaaactgccaattccactgctgtttggcccaatagtgagaactttttcctgctgcctcttggtgcttttgcctatggcccctattctgcctgctgaagacactcttgccagcatggacttaaacccctccagctctgacaatcctctttctcttttgttttacatgaagggtctggcagccaaagcaatcactcaaagttcaaaccttatcattttttgctttgttcctcttggccttggttttgtacatcagctttgaaaataccatcccagggttaatgctggggttaatttataactaagagtgctctagttttgcaatacaggacatgctataaaaatggaaagatgttgctttctgagaggatcttgctaccagtggaacagccactaaggattctgcagtgagagcagagggccagctaagtggtactctcccagagactgtctgactcacgccaccccctccaccttggacacaggacgctgtggtttctgagccaggtacagtgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtaaaacaggtaagtccgctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctggcctctactaaccatgttcatgttttctttttttttctacaggtcctgggtgacgaacag

SEQ ID NO: 233 - DNA, 1579 bp, Artificial Sequence
  synthetic construct; regulatory element
aggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggccactacgggtttaggctgcccatgtaaggaggcaaggcctggggacacccgagatgcctggttataattaacccagacatgtggctgccccccccccccccaacacctgctgcctctaaaaataaccctgtccctggtggatcccactacgggtttaggctgcccatgtaaggaggcaaggcctggggacacccgagatgcctggttataattaacccagacatgtggctgccccccccccccccaacacctgctgcctctaaaaataaccctgtccctggtggatcccactacgggtttaggctgcccatgtaaggaggcaaggcctggggacacccgagatgcctggttataattaacccagacatgtggctgccccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagcgtcgagatcttgctaccagtggaacagccactaaggattctgcagtgagagcagagggccagctaagtggtactctcccagagactgtctgactcacgccaccccctccaccttggacacaggacgctgtggtttctgagccaggtacagtgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagt

SEQ ID NO: 234 - DNA, 1080 bp, Artificial Sequence
  synthetic construct; regulatory element
aggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtacccggggatcttgctaccagtctagaggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctcggccggggccgcattcctgggggccgggcggtgctcccgcccgcctcgataaaaggctccggggccggcggcggcccacgagctacccggaggagcgggaggcgccaagcgtgagtatcgatcttgctaccagtggaacagccactaaggattctgcagtgagagcagagggccagctaagtggtactctcccagagactgtctgactcacgccaccccctccaccttggacacaggacgctgtggtttctgagccaggtacagtgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagt

SEQ ID NO: 235 - DNA, 568 bp, Artificial Sequence
  synthetic construct; regulatory element
gaatggtggacacccaaatatggcgacggttcctcacccgtcgccatatttgggtgtccgccctcggccggggccgcattcctgggggccgggcggtgctcccgcccgcctcgataaaaggctccggggccggcggcggcccacgagctacccggaggagcgggaggcgccaagcgatcttgctaccagtggaacagccactaaggattctgcagtgagagcagagggccagctaagtggtactctcccagagactgtctgactcacgccaccccctccaccttggacacaggacgctgtggtttctgagccaggtacagtgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagt

SEQ ID NO: 236 - DNA, 895 bp, Artificial Sequence
  synthetic construct; regulatory element
aggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggtctagagaatggtggacacccaaatatggcgacggttcctcacccgtcgccatatttgggtgtccgccctcggccggggccgcattcctgggggccgggcggtgctcccgcccgcctcgataaaaggctccggggccggcggcggcccacgagctacccggaggagcgggaggcgccaagcgatcttgctaccagtggaacagccactaaggattctgcagtgagagcagagggccagctaagtggtactctcccagagactgtctgactcacgccaccccctccaccttggacacaggacgctgtggtttctgagccaggtacagtgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagt

SEQ ID NO: 237 - DNA, 1291 bp, Artificial Sequence
  synthetic construct; regulatory element
aggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggccactacgggtttaggctgcccatgtaaggaggcaaggcctggggacacccgagatgcctggttataattaacccagacatgtggctgccccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagcgtcgagatcttgctaccagtggaacagccactaaggattctgcagtgagagcagagggccagctaagtggtactctcccagagactgtctgactcacgccaccccctccaccttggacacaggacgctgtggtttctgagccaggtacagtgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagt

SEQ ID NO: 238 - DNA, 1147 bp, Artificial Sequence
  synthetic construct; regulatory element
aggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagcgtcgagatcttgctaccagtggaacagccactaaggattctgcagtgagagcagagggccagctaagtggtactctcccagagactgtctgactcacgccaccccctccaccttggacacaggacgctgtggtttctgagccaggtacagtgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagt

SEQ ID NO: 239 - DNA, 1490 bp, Artificial Sequence
  synthetic construct; regulatory element
aggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggcccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctgaagctttggcccactacgggtttaggctgcccatgtaaggaggcaaggcctggggacacccgagatgcctggttataattaacccagacatgtggctgccccccccccccccaacacctgctgcctctaaaaataaccctgtccctggtggatcccctgcatgcgaagatcttcgaacaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagcgtcgagatcttgctaccagtggaacagccactaaggattctgcagtgagagcagagggccagctaagtggtactctcccagagactgtctgactcacgccaccccctccaccttggacacaggacgctgtggtttctgagccaggtacagtgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagt

SEQ ID NO: 240 - DNA, 723 bp, Artificial Sequence
  synthetic construct; regulatory element
aggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtacccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagt

SEQ ID NO: 241 - DNA, 100 bp, Artificial Sequence
  synthetic construct; regulatory element
aggttaatttttaaaaagcagtcaaaagtccaagtggcccttggcagcatttactctctctgtttgctctggttaataatctcaggagcacaaacattcc

SEQ ID NO: 242 - DNA, 200 bp, Artificial Sequence
  synthetic construct; regulatory element
aggttaatttttaaaaagcagtcaaaagtccaagtggcccttggcagcatttactctctctgtttgctctggttaataatctcaggagcacaaacattccaggttaatttttaaaaagcagtcaaaagtccaagtggcccttggcagcatttactctctctgtttgctctggttaataatctcaggagcacaaacattcc

SEQ ID NO: 243 - DNA, 321 bp, Artificial Sequence
  synthetic construct; regulatory element
aggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggg

SEQ ID NO: 244 - DNA, 554 bp, Artificial Sequence
  synthetic construct; regulatory element
aggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagagggtctagaaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctg

SEQ ID NO: 245 - DNA, 393 bp, Artificial Sequence
  synthetic construct; regulatory element
gatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagt

SEQ ID NO: 246 - DNA, 393 bp, Artificial Sequence
  synthetic construct; regulatory element
gatcttgctaccagtggaacagccactaaggattctgcagtgagagcagagggccagctaagtggtactctcccagagactgtctgactcacgccaccccctccaccttggacacaggacgctgtggtttctgagccaggtacagtgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagt

SEQ ID NO: 247 - DNA, 144 bp, Artificial Sequence
  synthetic construct; regulatory element
ccactacgggtttaggctgcccatgtaaggaggcaaggcctggggacacccgagatgcctggttataattaacccagacatgtggctgccccccccccccccaacacctgctgcctctaaaaataaccctgtccctggtggatc

SEQ ID NO: 248 - DNA, 288 bp, Artificial Sequence
  synthetic construct; regulatory element
ccactacgggtttaggctgcccatgtaaggaggcaaggcctggggacacccgagatgcctggttataattaacccagacatgtggctgccccccccccccccaacacctgctgcctctaaaaataaccctgtccctggtggatcccactacgggtttaggctgcccatgtaaggaggcaaggcctggggacacccgagatgcctggttataattaacccagacatgtggctgccccccccccccccaacacctgctgcctctaaaaataaccctgtccctggtggatc

SEQ ID NO: 249 - DNA, 432 bp, Artificial Sequence
  synthetic construct; regulatory element
ccactacgggtttaggctgcccatgtaaggaggcaaggcctggggacacccgagatgcctggttataattaacccagacatgtggctgccccccccccccccaacacctgctgcctctaaaaataaccctgtccctggtggatcccactacgggtttaggctgcccatgtaaggaggcaaggcctggggacacccgagatgcctggttataattaacccagacatgtggctgccccccccccccccaacacctgctgcctctaaaaataaccctgtccctggtggatcccactacgggtttaggctgcccatgtaaggaggcaaggcctggggacacccgagatgcctggttataattaacccagacatgtggctgccccccccccccccaacacctgctgcctctaaaaataaccctgtccctggtggatc

SEQ ID NO: 250 - DNA, 199 bp, Artificial Sequence
  synthetic construct; regulatory element
cccttcagattaaaaataactgaggtaagggcctgggtaggggaggtggtgtgagacgctcctgtctctcctctatctgcccatcggccctttggggaggaggaatgtgcccaaggactaaaaaaaggccatggagccagaggggcgagggcaacagacctttcatgggcaaaccttggggccctgctgaagctttggc

SEQ ID NO: 251 - protein, 4 aa, Artificial Sequence
  synthetic construct; cleavage site
  misc (2)..(2): Xaa can be any amino acid
RXKR

SEQ ID NO: 252 - protein, 4 aa, Artificial Sequence
  synthetic construct; cleavage site
  misc (2)..(2): Xaa can be any amino acid
RXRR

SEQ ID NO: 253 - protein, 6 aa, Artificial Sequence
  synthetic construct; sulfation site
DTAVYY

SEQ ID NO: 254 - protein, 7 aa, Artificial Sequence
  synthetic construct; sulfation site
EDIATYY

SEQ ID NO: 255 - protein, 7 aa, Artificial Sequence
  synthetic construct; sulfation site
EDEADYY

SEQ ID NO: 256 - protein, 7 aa, Artificial Sequence
  synthetic construct; sulfation site
EDTAVYY

SEQ ID NO: 257 - protein, 19 aa, Artificial Sequence
  synthetic construct; hinge
ESKYGPPCPSCPAPEFLGG

SEQ ID NO: 258 - protein, 7 aa, Artificial Sequence
  synthetic construct; sulfation site
EDFATYY

SEQ ID NO: 259 - protein, 7 aa, Artificial Sequence
  synthetic construct; sulfation site
EDFAVYY

SEQ ID NO: 260 - protein, 7 aa, Artificial Sequence
  synthetic construct; sulfation site
DDFATYY

SEQ ID NO: 261 - protein, 7 aa, Artificial Sequence
  synthetic construct; sulfation site
EDTAMYY

SEQ ID NO: 262 - protein, 7 aa, Artificial Sequence
  synthetic construct; sulfation site
EDAATYY

SEQ ID NO: 263 - protein, 7 aa, Artificial Sequence
  synthetic construct; sulfation site
FTFDDYA

SEQ ID NO: 264 - protein, 7 aa, Artificial Sequence
  synthetic construct; sulfation site
EDTALYY

SEQ ID NO: 265 - protein, 7 aa, Artificial Sequence
  synthetic construct; sulfation site
EDFASYY

SEQ ID NO: 266 - protein, 6 aa, Artificial Sequence
  synthetic construct; sulfation site
YTCNVD

SEQ ID NO: 267 - protein, 6 aa, Artificial Sequence
  synthetic construct; sulfation site
ELLIYY

SEQ ID NO: 268 - protein, 6 aa, Artificial Sequence
  synthetic construct; sulfation site
YYYGMD

SEQ ID NO: 269 - protein, 7 aa, Artificial Sequence
  synthetic construct; sulfation site
EDTGVYY

SEQ ID NO: 270 - protein, 7 aa, Artificial Sequence
  synthetic construct; sulfation site
EDIADYY

SEQ ID NO: 271 - protein, 7 aa, Artificial Sequence
  synthetic construct; sulfation site
EDVATYY

SEQ ID NO: 272 - protein, 97 aa, Artificial Sequence
  synthetic construct; intron
GTAAGTTTAGTCTTTTTGTCTTTTATTTCAGGTCCCGGATCCGGTGGTGGTGCAAATCAAAGAACTGCTCCTCAGTGGATGTTGCCTTTACTTCTAG

SEQ ID NO: 273 - DNA, 663 bp, Artificial Sequence
  synthetic construct; promoter
gacgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgg

SEQ ID NO: 274 - DNA, 762 bp, Artificial Sequence
  synthetic construct; promoter
gacgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcg

SEQ ID NO: 275 - DNA, 935 bp, Artificial Sequence
  synthetic construct; promoter
gtcgacaaattctgtcattttactagggtgatgaaattcccaagcaacaccatccttttcagataagggcactgaggctgagagaggagctgaaacctacccggggtcaccacacacaggtggcaaggctgggaccagaaaccaggactgttgactgcagcccggtattcattctttccatagcccacagggctgtcaaagaccccagggcctagtcagaggctcctccttcctggagagttcctggcacagaagttgaagctcagcacagccccctaacccccaactctctctgcaaggcctcaggggtcagaacactggtggagcagatcctttagcctctggattttagggccatggtagagggggtgttgccctaaattccagccctggtctcagcccaacaccctccaagaagaaattagaggggccatggccaggctgtgctagccgttgcttctgagcagattacaagaagggactaagacaaggactcctttgtggaggtcctggcttagggagtcaagtgacggcggctcagcactcacgtgggcagtgccagcctctaagagtgggcaggggcactggccacagagtcccagggagtcccaccagcctagtcgccagatctagagggccccagaagcctggtggttgtttgtccttctcaggggaaaagtgaggcggccccttggaggaaggggccgggcagaagatctaatcggattccaagcagctcaggggattgtctttttctagcaccttcttgccactcctaagcgtcctccgtgaccccggctgggatttagcctggtgctgtgtcagccccgggctcccaggggcttcccagtggtccccaggaaccctcgacagggccagggcgtctctctcgtccagcaagggcagggacgggccacaggccaagggccttaagc

SEQ ID NO: 276 - DNA, 3133 bp, Artificial Sequence
  synthetic construct; antibody construct
gacgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggacgcgtcaggtgagtatctcagggatccagacatggggatatgggaggtgcctctgatcccagggctcactgtgggtctctctgttcacaggttgaccgagtgaattcgccaccatgtacagaatgcagctgctgctgctcattgccctgtctctggccctggtcaccaattctgaagtgcagctggtggaaagtggtggtggactggtgcagcctggcagaagcctgagactgtcttgtgctgcctctggcttcacctttgatgactatgccatgcactgggtcagacaggcccctggcaaaggactggaatgggtgtcagccatcacctggaactctggccacattgactatgctgactctgtggaaggcagattcaccatcagcagagacaatgccaagaacagcctgtacctgcagatgaactccctgagagctgaggacacagcagtgtactactgtgccaaggtgtcctacctgagcacagccagcagcctggattattggggccagggcacactggttacagtgtcctctgccagcacaaagggcccctctgtttttccactggctcccagcagcaagagcaccagtggtggaacagctgccctgggctgtctggtcaaggattacttccctgagcctgtgacagtgtcttggaactcaggggctctgacctctggggtgcacacatttccagctgtgctgcagtcctctggcctgtactctctgtcctctgtggtcacagtgcctagctctagcctgggcacccagacctacatctgcaatgtgaaccacaagcctagcaacaccaaggtggacaagaaggtggaacccaagagctgtgacaagacccacacctgtcctccatgtcctgctccagaactgcttggaggcccttctgtgttcctgtttcctccaaagcctaaggacaccctgatgatcagcagaacccctgaagtgacctgtgtggtggttgatgtgtcccatgaggacccagaagtgaagttcaattggtatgtggatggggttgaagtgcacaatgctaagaccaagcctagagaggaacagtacaacagcacctacagagtggtgtctgtgctgacagtgctgcatcaggactggctgaatggcaaagagtacaagtgcaaagtgtccaacaaggccctgcctgctcctattgagaaaaccatctccaaggccaagggccagccaagagaaccccaggtttacacactgccacctagcagagatgagctgaccaagaaccaggtgtccctgacctgcctggttaagggcttctacccctctgacattgctgtggaatgggagagcaatggccagcctgagaacaactacaagacaacccctcctgtgctggactctgatggctcattcttcctgtacagcaagctgactgtggacaagtccagatggcagcaggggaatgtgttcagctgctctgtgatgcatgaggccctgcacaaccactacacccagaaaagtctgagtctgagccctggcaagagaaagagaagaggctctggagaaggcagaggctccctgctgacatgtggggatgttgaagagaatcctgggcctatgtataggatgcaactgctcctcctgattgctctgagcctggctcttgtgaccaactctgacatccagatgacacagagccctagcagcctgtctgcttctgtgggagacagagtgaccatcacatgcagagccagccagggaatcagaaactacctggcctggtatcagcaaaagcctggcaaggcccctaagctgctgatctatgcagccagcacactgcagtcaggggtgccaagcagattttcaggctctggctctggcacagacttcaccctgaccatttctagcctgcagcctgaggatgtggccacctactactgccagagatacaacagagccccatacacctttggacagggcacaaaggtggaaatcaagagaacagtggctgccccatctgtgttcatcttcccaccatctgatgaacagctgaagtctggcactgcctctgttgtgtgcctgctgaacaacttctaccctagagaagccaaggtgcagtggaaggttgacaatgccctgcagtctggcaatagccaagaatctgtgacagagcaggactccaaggattccacctacagcctgagcagcaccctgacactgagcaaggctgactatgagaagcacaaagtgtatgcctgtgaagtgacacaccagggactgagcagcccagtgaccaagagcttcaacaggggagagtgctgataactcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcg

SEQ ID NO: 277 - DNA, 3554 bp, Artificial Sequence
  synthetic construct; antibody construct
ctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctaccagggtaatggggatcctctagaactatagctagtcgacgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggacgcgtcaggtgagtatctcagggatccagacatggggatatgggaggtgcctctgatcccagggctcactgtgggtctctctgttcacaggttgaccgagtgaattcgccaccatgtacagaatgcagctgctgctgctcattgccctgtctctggccctggtcaccaattctgaagtgcagctggtggaaagtggtggtggactggtgcagcctggcagaagcctgagactgtcttgtgctgcctctggcttcacctttgatgactatgccatgcactgggtcagacaggcccctggcaaaggactggaatgggtgtcagccatcacctggaactctggccacattgactatgctgactctgtggaaggcagattcaccatcagcagagacaatgccaagaacagcctgtacctgcagatgaactccctgagagctgaggacacagcagtgtactactgtgccaaggtgtcctacctgagcacagccagcagcctggattattggggccagggcacactggttacagtgtcctctgccagcacaaagggcccctctgtttttccactggctcccagcagcaagagcaccagtggtggaacagctgccctgggctgtctggtcaaggattacttccctgagcctgtgacagtgtcttggaactcaggggctctgacctctggggtgcacacatttccagctgtgctgcagtcctctggcctgtactctctgtcctctgtggtcacagtgcctagctctagcctgggcacccagacctacatctgcaatgtgaaccacaagcctagcaacaccaaggtggacaagaaggtggaacccaagagctgtgacaagacccacacctgtcctccatgtcctgctccagaactgcttggaggcccttctgtgttcctgtttcctccaaagcctaaggacaccctgatgatcagcagaacccctgaagtgacctgtgtggtggttgatgtgtcccatgaggacccagaagtgaagttcaattggtatgtggatggggttgaagtgcacaatgctaagaccaagcctagagaggaacagtacaacagcacctacagagtggtgtctgtgctgacagtgctgcatcaggactggctgaatggcaaagagtacaagtgcaaagtgtccaacaaggccctgcctgctcctattgagaaaaccatctccaaggccaagggccagccaagagaaccccaggtttacacactgccacctagcagagatgagctgaccaagaaccaggtgtccctgacctgcctggttaagggcttctacccctctgacattgctgtggaatgggagagcaatggccagcctgagaacaactacaagacaacccctcctgtgctggactctgatggctcattcttcctgtacagcaagctgactgtggacaagtccagatggcagcaggggaatgtgttcagctgctctgtgatgcatgaggccctgcacaaccactacacccagaaaagtctgagtctgagccctggcaagagaaagagaagaggctctggagaaggcagaggctccctgctgacatgtggggatgttgaagagaatcctgggcctatgtataggatgcaactgctcctcctgattgctctgagcctggctcttgtgaccaactctgacatccagatgacacagagccctagcagcctgtctgcttctgtgggagacagagtgaccatcacatgcagagccagccagggaatcagaaactacctggcctggtatcagcaaaagcctggcaaggcccctaagctgctgatctatgcagccagcacactgcagtcaggggtgccaagcagattttcaggctctggctctggcacagacttcaccctgaccatttctagcctgcagcctgaggatgtggccacctactactgccagagatacaacagagccccatacacctttggacagggcacaaaggtggaaatcaagagaacagtggctgccccatctgtgttcatcttcccaccatctgatgaacagctgaagtctggcactgcctctgttgtgtgcctgctgaacaacttctaccctagagaagccaaggtgcagtggaaggttgacaatgccctgcagtctggcaatagccaagaatctgtgacagagcaggactccaaggattccacctacagcctgagcagcaccctgacactgagcaaggctgactatgagaagcacaaagtgtatgcctgtgaagtgacacaccagggactgagcagcccagtgaccaagagcttcaacaggggagagtgctgataactcgaggacggggtgaactacgcctgaggatccgatctttttccctctgccaaaaattatggggacatcatgaagccccttgagcatctgacttctggctaataaaggaaatttattttcattgcaatagtgtgttggaattttttgtgtctctcactcggaagcaattcgttgatctgaatttcgaccacccataatacccattaccctggtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcag

(19) United States
(12) Patent Application Publication
(10) Pub. No.: US 2023/0391864 A1
(43) Pub. Date: Dec. 7, 2023

(54) VECTORIZED ANTI-TNF-ALPHA ANTIBODIES FOR OCULAR INDICATIONS

(71) Applicant: REGENXBIO, INC., Rockville, MD (US)

(72) Inventors: Xu Wang, Falls Church, VA (US); Devin McDougald, Boston, MA (US); Joseph Bruder, Gaithersburg, MD (US); Ye Liu, Clarksville, MD (US); Olivier Danos, Princeton, NJ (US); Wei-Hua Lee, Darnestown, MD (US); Chunping Qiao, Chapel Hill, NC (US); Ewa Budzynski, Deforest, WI (US); Mikayla Higgins, Gaithersburg, MD (US); Mi Shi, Ellicott City, MD (US)

(21) Appl. No.: 18/034,039

(22) PCT Filed: Oct. 28, 2021
(86) PCT No.: PCT/US2021/057084

§ 371 Date: Apr. 26, 2023

Related U.S. Application Data
(60) Provisional application No. 63/106,832, filed on Oct. 28, 2020.

(51) Int. Cl. C07K 16/24; C12N 15/86; A61P 27/02

(52) U.S. Cl. CPC C07K 16/241; C12N 15/86; A61P 27/02; C12N 2750/14143; C12N 2750/14145

(57) ABSTRACT

Compositions and methods are described for the delivery of a fully human post-translationally modified therapeutic monoclonal antibody, or an antigen binding fragment thereof, that binds to TNF-α, IL6 or IL6-R to a human subject for treatment of an ocular indication, particularly non-infectious uveitis. The nucleotide sequence encoding the antibody is delivered in a rAAV vector that targets ocular tissue cells for expression of the transgene.

### 1. INTRODUCTION

Compositions and methods are described for the delivery of a fully human post-translationally modified (HuPTM) therapeutic monoclonal antibody (“mAb”) that binds to tumor necrosis factor alpha (TNFα), interleukin-6 (IL6), or interleukin-6 receptor (IL6R) or the HuPTM antigen-binding fragment of a therapeutic mAb that binds to TNFα, IL6, or IL6R—e.g., a fully human-glycosylated (HuGlγ) Fab of the therapeutic mAb—to a human subject diagnosed with non-infectious uveitis (NIU).

### 2. BACKGROUND OF THE INVENTION

Therapeutic mAbs have been shown to be effective in treating a number of diseases and conditions. However, because these agents are effective for only a short period of time, repeated injections for long durations are often required, thereby creating considerable treatment burden for patients.

Uveitis includes a group of heterogeneous diseases characterized by inflammation of the uveal tract. Uveitis may be generally classified by the etiology of inflammation as infectious or non-infectious (autoimmune disorders), which could be related or not to a systemic disease. In addition, uveitis can be anatomically classified as anterior, intermediate, posterior or panuveitis, and they may have an acute, chronic or recurrent course. The clinical presentation is variable, the symptoms may include blurred vision, photophobia, ocular pain and significant visual impairment (Valenzuela et al., Front Pharmacol. 2020; 11: 655).

Non-infectious uveitis is a serious, sight-threatening intraocular inflammatory condition characterized by inflammation of the uvea (iris, ciliary body, and choroid). Non-infectious uveitis is thought to result from an immune-mediated response to ocular antigens and is a leading cause of irreversible blindness in working-age population in the developed world. The goal of uveitis treatment is to control inflammation, prevent recurrences, and preserve vision, as well as minimize the adverse effects of medications. Currently, the standard of care for non-infectious uveitis includes the administration of corticosteroids as first-line agents, but in some cases a more aggressive therapy is required. This includes synthetic immunosuppressants, such as antimetabolites (methotrexate, mycophenolate mofetil, and azathioprine), calcineurinic inhibitors (cyclosporine, tacrolimus), and alkylating agents (cyclophosphamide, chlorambucil). In those patients who become intolerant or refractory to corticosteroids and conventional immunosuppressive treatment, biologic agents have arisen as an effective therapy in pediatric and adulthood uveitis, based on targeting relevant immunological pathways involved in disease pathogenesis. Current immunomodulatory therapy includes the inhibition of TNFα, achieved with mAb, such as infliximab, adalimumab, golimumab, and certolizumab-pegol, or with TNF receptor fusion protein, etanercept. In this regard, anti-TNF agents (infliximab and adalimumab) have shown the strongest results in terms of favorable outcomes. When conventional immunosuppressive treatments and/or anti-TNF-α therapies fail, other biological agents are recommended. Some of the newest therapies have focused on blocking interleukin actions (e.g. anti-IL6 therapy) (Ming et al, Drug Des Devel Ther. 2018; 12: 2005-2016).

Adalimumab is an entirely humanized monoclonal antibody against TNF-α which is subcutaneously self-administered. It is the most used and studied biologic medication for the treatment of adulthood non-infectious uveitis since its approval in 2016 (Ming et al, Drug Des Devel Ther. 2018; 12: 2005-2016). Infliximab (Remicade®) is a chimeric monoclonal antibody used since 2001. It has 25% murine and 75% humanized domains. Its use is FDA-approved for RA, psoriatic arthritis, IBD, and AS, but not for non-infectious uveitis. It is only intravenously administered, usually in conjunction with methotrexate to prevent the generation of antibodies against the drug. Infliximab is associated with multitude of side effects on systemic administration such as congestive heart failure, reactivation of latent tuberculosis, and increased risk of infections, all of which can be minimized by administering the drug intravitreally. Golimumab (Simponi®) is a fully humanized monoclonal antibody, subcutaneously administered with a dose of 50 mg every 4 weeks. There is little evidence, but its efficacy has been described in patients with non-infectious uveitis refractory to adalimumab or infliximab, and thus golimumab is usually reserved as treatment for this subset of non-responders.

There is a need for more effective treatments that reduce the treatment burden on patients suffering from non-infectious uveitis. Intravitreal medications have become a promising mode of drug administration in uveitis patients as they provide high volume of drug to the target tissues, eliminating the risk of systemic toxicity. Reducing or eliminating the need for periodic ocular administration would reduce patient burden and improve therapy.

### 3. SUMMARY OF THE INVENTION

Therapeutic antibodies delivered by gene therapy have several advantages over injected or infused therapeutic antibodies that dissipate over time resulting in peak and trough levels. Sustained expression of the transgene product antibody, as opposed to injecting an antibody repeatedly, allows for a more consistent level of antibody to be present at the site of action, and is less risky and more convenient for patients, since fewer injections need to be made. Furthermore, antibodies expressed from transgenes are post-translationally modified in a different manner than those that are directly injected because of the different microenvironment present during and after translation. Without being bound by any particular theory, this results in antibodies that have different diffusion, bioactivity, distribution, affinity, pharmacokinetic, and immunogenicity characteristics, such that the antibodies delivered to the site of action are “biobetters” in comparison with directly injected antibodies. Accordingly, provided herein are compositions and methods for anti-TNFα, anti-IL6, or anti-IL6R gene therapy, particularly recombinant AAV gene therapy, designed to target the eye and generate a depot of transgenes for expression of anti-TNFα antibodies, particularly adalimumab, or an antigen binding fragment thereof, or anti-IL6 or anti-IL6R antibodies, that result in a therapeutic or prophylactic serum levels of the antibody within 20 days, 30 days, 40 days, 50 days, 60 days, or 90 days of administration of the rAAV composition.

Compositions and methods are described for the systemic delivery of an anti-TNFα, anti-IL6, or anti-IL6R HuPTM mAb or an anti-TNFα, anti-IL6, or anti-IL6R HuPTM antigen-binding fragment of a therapeutic mAb (for example, a fully human-glycosylated Fab (HuGlyFab) of a therapeutic mAb, to a patient (human subject) diagnosed with non-infectious uveitis or other condition indicated for treatment with the therapeutic anti-TNFα, anti-IL6, or anti-IL6R mAb. Such antigen-binding fragments of therapeutic mAbs include a Fab, F(ab′)₂, or scFv (single-chain variable fragment) (collectively referred to herein as “antigen-binding fragment”). “HuPTM Fab” as used herein may include other antigen binding fragments of a mAb. In an alternative embodiment, full-length mAbs can be used. Delivery may be advantageously accomplished via gene therapy—e.g., by administering a viral vector or other DNA expression construct encoding a therapeutic anti-TNFα, anti-IL6, or anti-IL6R mAb or its antigen-binding fragment (or a hyperglycosylated derivative of either) diagnosed with a condition indicated for treatment with the therapeutic anti-TNFα, anti-IL6, or anti-IL6R mAb—to create a permanent depot in the eye, or in alternative embodiments, liver and/or muscle, of the patient that continuously supplies the HuPTM mAb or antigen-binding fragment of the therapeutic mAb, e.g., a human-glycosylated transgene product, or peptide to one or more ocular tissues where the mAb or antigen-binding fragment thereof or peptide exerts its therapeutic or prophylactic effect.

Provided are gene therapy vectors, particularly rAAV gene therapy vectors, which when administered to a human subject result in expression of an anti-TNFα, anti-IL6, or anti-IL6R antibody to achieve a maximum or steady state serum concentration, for example, 20, 30, 40, 50, 60 or 90 days after administration of the vector encoding the anti-TNFα, anti-IL6, or anti-IL6R antibody. In certain embodiments, the antibody binds to its target, for example, in an antibody binding assay (e.g. enzyme-linked immunosorbent assay (ELISA) binding assay or surface plasmon resonance (SPR)—based real-time kinetics assay), preferably in the picomolar or nanomolar range, and/or exhibits biological activity in an appropriate assay.

The recombinant vector used for delivering the transgene includes non-replicating recombinant adeno-associated virus vectors (“rAAV”). In embodiments, the AAV type has a tropism for retinal cells, for example AAV8 subtype of AAV. However, other viral vectors may be used, including but not limited to lentiviral vectors; vaccinia viral vectors, or non-viral expression vectors referred to as “naked DNA” constructs. Expression of the transgene can be controlled by constitutive or tissue-specific expression control elements, particularly elements that are ocular tissue, liver and/or muscle specific control elements, for example one or more elements of Tables 1 and 1a.

In certain embodiments, the HuPTM mAb or HuPTM antigen-binding fragment encoded by the transgene can include, but is not limited to, a full-length or an antigen-binding fragment of a therapeutic antibody that binds to TNFα, particularly adalimumab, infliximab or golimumab, or therapeutic antibodies that bind to IL6 or IL6R, including satralizumab, sarilumab, siltuximab, clazakizumab, sirukumab, olokizumab, erilimzumab, gerilizumab, or tocilizumab, see, for example FIGS. 1A and 1B.

Gene therapy constructs for the therapeutic antibodies are designed such that both the heavy and light chains are expressed. The coding sequences for the heavy and light chains can be engineered in a single construct in which the heavy and light chains are separated by a cleavable linker or IRES so that separate heavy and light chain polypeptides are expressed. In particular embodiments, the linker is a Furin T2A linker (SEQ ID NOS:143 or 144). In certain embodiments, the coding sequences encode for a Fab or F(ab′)₂ or an scFv. In certain embodiments the full length heavy and light chains of the antibody are expressed. In other embodiments, the constructs express an scFv in which the heavy and light chain variable domains are connected via a flexible, non-cleavable linker. In certain embodiments, the construct expresses, from the N-terminus, NH₂-V_{L}-linker-V_{H}-COOH or NH₂-V_{H}-linker-V_{L}-COOH.

In addition, antibodies expressed from transgenes in vivo are not likely to contain degradation products associated with antibodies produced by recombinant technologies, such as protein aggregation and protein oxidation. Aggregation is an issue associated with protein production and storage due to high protein concentration, surface interaction with manufacturing equipment and containers, and purification with certain buffer systems. These conditions, which promote aggregation, do not exist in transgene expression in gene therapy. Oxidation, such as methionine, tryptophan, and histidine oxidation, is also associated with protein production and storage, and is caused by stressed cell culture conditions, metal and air contact, and impurities in buffers and excipients. The proteins expressed from transgenes in vivo may also oxidize in a stressed condition. However, humans, and many other organisms, are equipped with an antioxidation defense system, which not only reduces the oxidation stress, but sometimes also repairs and/or reverses the oxidation. Thus, proteins produced in vivo are not likely to be in an oxidized form. Both aggregation and oxidation could affect the potency, pharmacokinetics (clearance), and immunogenicity.

The production of HuPTM mAb or HuPTM Fab in ocular tissue cells of the human subject should result in a “biobetter” molecule for the treatment of disease accomplished via gene therapy—e.g., by administering a viral vector or other DNA expression construct encoding a full-length HuPTM mAb or HuPTM Fab of a therapeutic mAb to a patient (human subject) diagnosed with a disease indication for that mAb, to create a permanent depot in the subject that continuously supplies the human-glycosylated, sulfated transgene product produced by the subject's transduced cells. The cDNA construct for the HuPTMmAb or HuPTM Fab should include a signal peptide that ensures proper co- and post-translational processing (glycosylation and protein sulfation) by the transduced human cells.

As an alternative, or an additional treatment to gene therapy, the full-length HuPTM mAb or HuPTM Fab can be produced in human cell lines by recombinant DNA technology, and the glycoprotein can be administered to patients.

Combination therapies involving systemic delivery of the full-length HuPTM anti-anti-TNFα, anti-IL6, or anti-IL6R, mAb or HuPTM anti-anti-TNFα, anti-IL6, or anti-IL6R Fab to the patient accompanied by administration of other available treatments are encompassed by the methods provided herein. The additional treatments may be administered before, concurrently or subsequent to the gene therapy treatment. Such additional treatments can include but are not limited to co-therapy with the therapeutic mAb.

Also provided are methods of manufacturing the viral vectors, particularly the AAV based viral vectors. In specific embodiments, provided are methods of producing recombinant AAVs comprising culturing a host cell containing an artificial genome comprising a cis expression cassette flanked by AAV ITRs, wherein the cis expression cassette comprises a transgene encoding a therapeutic antibody operably linked to expression control elements that will control expression of the transgene in human cells; a trans expression cassette lacking AAV ITRs, wherein the trans expression cassette encodes an AAV rep and capsid protein operably linked to expression control elements that drive expression of the AAV rep and capsid proteins in the host cell in culture and supply the rep and cap proteins in trans; sufficient adenovirus helper functions to permit replication and packaging of the artificial genome by the AAV capsid proteins; and recovering recombinant AAV encapsidating the artificial genome from the cell culture.

The inventors found that intravenous administration of an AAV8-based vector comprising an optimized expression cassette containing a liver-specific promoter and a codon optimized and CpG depleted transgene with a modified furin-T2A processing signal results in dose-dependent and sustained serum antibody concentrations in non-human primates. Accordingly, provided are compositions comprising rAAV vectors which comprise an optimized expression cassette containing a liver-specific promoter and a codon optimized and CpG depleted transgene with a modified furin-T2A processing signal that express a transgene, for example, heavy and light chains of an anti-TNFα (including adalimumab), anti-IL6, or anti-IL6R therapeutic antibody. Methods of administration and manufacture are also provided.

3.1 Illustrative Embodiments

### Compositions of Matter

1. A pharmaceutical composition for treating non-infectious uveitis in a human subject in need thereof, comprising an adeno-associated virus (AAV) vector having:
   (a) a viral capsid that has a tropism for ocular tissue cells; and
   (b) an artificial genome comprising an expression cassette flanked by AAV inverted terminal repeats (ITRs), wherein the expression cassette comprises a transgene encoding a heavy chain and a light chain of a substantially full-length or full-length anti-TNFα mAb, anti-IL6 mAb, or anti-IL6R mAb, or an antigen-binding fragment thereof, operably linked to one or more regulatory sequences that promote expression of the transgene in human ocular tissue cells;
wherein said AAV vector is formulated for subretinal, intravitreal, intranasal, intracameral, suprachoroidal, or systemic administration to said human subject.
2. The pharmaceutical composition of paragraph 1, wherein the viral capsid is at least 95% identical to the amino acid sequence of AAV serotype 1 (AAV1), serotype 2 (AAV2), serotype AAV2.7m8 (AAV2.7m8), serotype 3 (AAV3), serotype 3B (AAV3B), serotype 4 (AAV4), serotype 5 (AAV5), serotype 6 (AAV6), serotype 7 (AAV7), serotype 8 (AAV8), serotype rh8 (AAVrh8), serotype 9 (AAV9), serotype 9e (AAV9e), serotype rhl 0 (AAVrhl 0), serotype rh20 (AAVrh20), serotype rh39 (AAVrh39), serotype hu.37 (AAVhu.37), serotype rh73 (AAVrh73), or serotype rh74 (AAVrh74), serotype hu51 (AAV.hu51), serotype hu21 (AAV.hu21), serotype hu12 (AAV.hu12), or serotype hu26 (AAV.hu26).
3. The pharmaceutical composition of any of paragraphs 1 or 2, wherein the AAV capsid is AAV8, AAV3B, AAV2.7m8, or AAVrh73.
4. The pharmaceutical composition of paragraphs 1 to 3, wherein the ocular tissue cell is a cornea cell, an iris cell, a ciliary body cell, a schlemm's canal cell, a trabecular meshwork cell, a retinal cell, a RPE-choroid tissue cell, or an optic nerve cell.
5. The pharmaceutical composition of paragraphs 1 to 4, wherein the regulatory sequence includes a regulatory sequence from Table 1 or Table 1a.
6. The pharmaceutical composition of paragraph 5, wherein the regulatory sequence is a CAG promoter (SEQ ID NO: 74), human rhodopsin kinase (GRK1) promoter (SEQ ID NOS:77 or 217), a mouse cone arresting (CAR) promoter (SEQ ID NOS:214-216), a human red opsin (RedO) promoter (SEQ ID NO:212), a CB promoter (SEQ ID NO: 273 or 274), or a Best1/GRK tandem promoter (SEQ ID NO: 275).
7. The pharmaceutical composition of any of paragraphs 1 to 6, wherein the transgene comprises a Furin/2A linker between the nucleotide sequences coding for the heavy and light chains of said mAb.
8. The pharmaceutical composition of paragraph 7, wherein said Furin 2A linker is a Furin/T2A linker having the amino acid sequence RKRR(GSG) APVKQTLNFDLLKLAGDVESNPGP (SEQ ID NOS:143 or 144).
9. The pharmaceutical composition of any of paragraphs 1 to 8, wherein the transgene encodes a signal sequence at the N-terminus of the heavy chain and the light chain of said antigen-binding fragment that directs secretion and post translational modification in said human ocular tissue cells.
10. The pharmaceutical composition of paragraph 9, wherein said signal sequence is MYRMQLLLLIALSLALVTNS (SEQ ID NO:85) or a signal sequence from Table 2.
11. The pharmaceutical composition of any of paragraphs 1 to 10, wherein transgene has the structure: signal sequence-Heavy chain-Furin site-2A site-signal sequence-Light chain-PolyA.
12. The pharmaceutical composition of any of paragraphs 1 to 11, wherein the anti-TNFα antibody is adalimumab, infliximab, or golimumab, or an antigen binding fragment thereof
13. The pharmaceutical composition of any of paragraphs 1 to 12, wherein the full-length mAb or the antigen-binding fragment comprises a heavy chain with an amino acid sequence of SEQ ID NO: 1 and optionally an Fc polypeptide with an amino acid sequence of SEQ ID NO: 64 and a light chain with an amino acid sequence of SEQ ID NO: 2; a heavy chain with an amino acid sequence of SEQ ID NO: 3 and optionally an Fc polypeptide with an amino acid sequence of SEQ ID NO: 65 and a light chain with an amino acid sequence of SEQ ID NO: 4; or a heavy chain with an amino acid sequence of SEQ ID NO: 5 and optionally an Fc polypeptide with an amino acid sequence of SEQ ID NO: 65 and a light chain with an amino acid sequence of SEQ ID NO: 6.
14. The pharmaceutical composition of any of paragraphs 1 to 13, wherein the transgene comprises a nucleotide sequence of SEQ ID NO: 26 encoding the heavy chain and a nucleotide sequence of SEQ ID NO: 27 encoding the light chain; a nucleotide sequence of SEQ ID NO: 28 encoding the heavy chain and a nucleotide sequence of SEQ ID NO: 29 encoding the light chain; or a nucleotide sequence of SEQ ID NO: 30 encoding the heavy chain and a nucleotide sequence of SEQ ID NO: 31 encoding the light chain.
15. The pharmaceutical composition of any of paragraphs 1 to 11, wherein the antiIL6 or anti-IL6R antibody is satralizumab, sarilumab, siltuximab, clazakizumab, sirukumab, olokizumab, gerilizumab, or tocilizumab, or an antigen binding fragment thereof.
16. The pharmaceutical composition of any of paragraphs 1 to 11 or 15, wherein the full-length mAb or the antigen-binding fragment comprises a heavy chain with an amino acid sequence of SEQ ID NO: 7 and optionally an Fc polypeptide with an amino acid sequence of SEQ ID NO: 67 and a light chain with an amino acid sequence of SEQ ID NO: 8; a heavy chain with an amino acid sequence of SEQ ID NO: 9 and optionally an Fc polypeptide with an amino acid sequence of SEQ ID NO:185 and a light chain with an amino acid sequence of SEQ ID NO: 10; a heavy chain with an amino acid sequence of SEQ ID NO: 11 and optionally an Fc polypeptide with an amino acid sequence of SEQ ID NO: 68 and a light chain with an amino acid sequence of SEQ ID NO: 12; comprises a heavy chain with an amino acid sequence of SEQ ID NO: 13 and optionally an Fc polypeptide with an amino acid sequence of SEQ ID NO: 69 and a light chain with an amino acid sequence of SEQ ID NO: 14; a heavy chain with an amino acid sequence of SEQ ID NO: 15 and optionally an Fc polypeptide with an amino acid sequence of SEQ ID NO: and a light chain with an amino acid sequence of SEQ ID NO: 16; a heavy chain with an amino acid sequence of SEQ ID NO: 17 and optionally an Fc polypeptide with an amino acid sequence of SEQ ID NO: 71 and a light chain with an amino acid sequence of SEQ ID NO: 18; a heavy chain with an amino acid sequence of SEQ ID NO: 19 and a light chain with an amino acid sequence of SEQ ID NO: 20; or a heavy chain with an amino acid sequence of SEQ ID NO: 21 and optionally an Fc polypeptide with an amino acid sequence of SEQ ID NO: 72 and a light chain with an amino acid sequence of SEQ ID NO: 22.
17. The pharmaceutical composition of any of paragraphs 1, 11 or 15-16, wherein the transgene comprises a nucleotide sequence of SEQ ID NO: 32 encoding the heavy chain and a nucleotide sequence of SEQ ID NO: 33 encoding the light chain; a nucleotide sequence of SEQ ID NO: 34 encoding the heavy chain and a nucleotide sequence of SEQ ID NO: 35 encoding the light chain; a nucleotide sequence of SEQ ID NO: 36 encoding the heavy chain and a nucleotide sequence of SEQ ID NO: 37 encoding the light chain; wherein the transgene comprises a nucleotide sequence of SEQ ID NO: 38 encoding the heavy chain and a nucleotide sequence of SEQ ID NO: 39 encoding the light chain; a nucleotide sequence of SEQ ID NO: 40 encoding the heavy chain and a nucleotide sequence of SEQ ID NO: 41 encoding the light chain; a nucleotide sequence of SEQ ID NO: 42 encoding the heavy chain and a nucleotide sequence of SEQ ID NO: 43 encoding the light chain; wherein the transgene comprises a nucleotide sequence of SEQ ID NO: 44 encoding the heavy chain and a nucleotide sequence of SEQ ID NO: 45 encoding the light chain; or a nucleotide sequence of SEQ ID NO:183 encoding the heavy chain and a nucleotide sequence of SEQ ID NO:184 encoding the light chain.
18. The pharmaceutical composition of paragraphs 1 to 17, wherein the antigen-binding fragment is a Fab, a F(ab′)₂, or an scFv.
19. The pharmaceutical composition of any of paragraphs 1 to 18, wherein the mAb or the antigen-binding fragment thereof is a hyperglycosylated mutant or wherein the Fc polypeptide of the mAb is glycosylated or aglycosylated.
20. The pharmaceutical composition of any of paragraphs 1 to 19, wherein the artificial genome is self complementary.
21. The pharmaceutical composition of any of paragraphs 1 to 20 wherein the artificial genome is the construct EF1ac.Vh4i.Adalimumab.Fab scAAV, mU1a.Vh4i.Adalimumab.Fab scAAV, CAG.Adalimumab.IgG, CAG.Adalimumab.Fab, GRK1.Vh4i.Adalimumab.IgG, CB.VH4i.adalimumab.IgG, CBlong.VH4.adalimumab.IgG or Best1.GRK.VH4.adalimumab.IgG.
22. A composition comprising an adeno-associated virus (AAV) vector having:
   a. a viral AAV capsid, that is optionally at least 95% identical to the amino acid sequence of AAV serotype 1 (AAV1), serotype 2 (AAV2), serotype 3 (AAV3), serotype 3B (AAV3B), serotype 4 (AAV4), serotype 5 (AAV5), serotype 6 (AAV6), serotype 7 (AAV7), serotype 8 (AAV8), serotype rh8 (AAVrh8), serotype 9 (AAV9), serotype 9e (AAV9e), serotype rh10 (AAVrh10), serotype rh20 (AAVrh20), serotype rh39 (AAVrh39), serotype hu.37 (AAVhu.37), serotype rh73 (AAVrh73), or serotype rh74 (AAVrh74), serotype hu51 (AAV.hu51), serotype hu21 (AAV.hu21), serotype hu12 (AAV.hu12), or serotype hu26 (AAV.hu26); and
   b. an artificial genome comprising an expression cassette flanked by AAV inverted terminal repeats (ITRs), wherein the expression cassette comprises a transgene encoding a heavy and a light chain of a substantially full-length or full-length anti-TNFα mAb anti-IL6 antibody or anti-IL6R antibody, or an antigen-binding fragment thereof, operably linked to one or more regulatory sequences that promote expression of the transgene in human ocular tissue cells;
   c. wherein the transgene encodes a signal sequence at the N-terminus of the heavy chain and the light chain of said mAb that directs secretion and post translational modification of said mAb in ocular tissue cells.
23. The composition of paragraph 22, wherein the ocular tissue cell is a cornea cell, an iris cell, a ciliary body cell, a schlemm's canal cell, a trabecular meshwork cell, a retinal cell, a RPE-choroid tissue cell, or an optic nerve cell.
24. The composition of paragraphs 22 or 23, wherein the AAV capsid is AAV2.7m8, AAV8, AAV3B, or AAVrh73.
25. The composition of paragraphs 22 to 24, wherein the anti-TNFα antibody is adalimumab, infliximab, or golimumab, or an antigen binding fragment thereof
26. The pharmaceutical composition of any of paragraphs 22 to 25, wherein the full-length mAb or the antigen-binding fragment comprises a heavy chain with an amino acid sequence of SEQ ID NO: 1 and optionally an Fc polypeptide with an amino acid sequence of SEQ ID NO: 64 and a light chain with an amino acid sequence of SEQ ID NO: 2; a heavy chain with an amino acid sequence of SEQ ID NO: 3 and optionally an Fc polypeptide with an amino acid sequence of SEQ ID NO: 65 and a light chain with an amino acid sequence of SEQ ID NO: 4; or a heavy chain with an amino acid sequence of SEQ ID NO: 5 and optionally an Fc polypeptide with an amino acid sequence of SEQ ID NO: 65 and a light chain with an amino acid sequence of SEQ ID NO: 6.
27. The pharmaceutical composition of paragraph 22 to 26, wherein the transgene comprises a nucleotide sequence of SEQ ID NO: 26 encoding the heavy chain and a nucleotide sequence of SEQ ID NO: 27 encoding the light chain; a nucleotide sequence of SEQ ID NO: 28 encoding the heavy chain and a nucleotide sequence of SEQ ID NO: 29 encoding the light chain; or a nucleotide sequence of SEQ ID NO: 30 encoding the heavy chain and a nucleotide sequence of SEQ ID NO: 31 encoding the light chain.
28. The pharmaceutical composition of any of paragraphs 22 to 27, wherein the antiIL6 or anti-IL6R antibody is satralizumab, sarilumab, siltuximab, clazakizumab, sirukumab, olokizumab, gerilizumab, or tocilizumab, or an antigen binding fragment thereof.
29. The pharmaceutical composition of any of paragraphs 22 to 24 or 27, wherein the full-length mAb or the antigen-binding fragment comprises a heavy chain with an amino acid sequence of SEQ ID NO: 7 and optionally an Fc polypeptide with an amino acid sequence of SEQ ID NO: 67 and a light chain with an amino acid sequence of SEQ ID NO: 8; a heavy chain with an amino acid sequence of SEQ ID NO: 9 and optionally an Fc polypeptide with an amino acid sequence of SEQ ID NO:185 and a light chain with an amino acid sequence of SEQ ID NO: 10; a heavy chain with an amino acid sequence of SEQ ID NO: 11 and optionally an Fc polypeptide with an amino acid sequence of SEQ ID NO: 68 and a light chain with an amino acid sequence of SEQ ID NO: 12; comprises a heavy chain with an amino acid sequence of SEQ ID NO: 13 and optionally an Fc polypeptide with an amino acid sequence of SEQ ID NO: 69 and a light chain with an amino acid sequence of SEQ ID NO: 14; a heavy chain with an amino acid sequence of SEQ ID NO: 15 and optionally an Fc polypeptide with an amino acid sequence of SEQ ID NO: 70 and a light chain with an amino acid sequence of SEQ ID NO: 16; a heavy chain with an amino acid sequence of SEQ ID NO: 17 and optionally an Fc polypeptide with an amino acid sequence of SEQ ID NO: 71 and a light chain with an amino acid sequence of SEQ ID NO: 18; a heavy chain with an amino acid sequence of SEQ ID NO: 19 and a light chain with an amino acid sequence of SEQ ID NO: 20; or a heavy chain with an amino acid sequence of SEQ ID NO: 21 and optionally an Fc polypeptide with an amino acid sequence of SEQ ID NO: 72 and a light chain with an amino acid sequence of SEQ ID NO: 22.
30. The pharmaceutical composition of any of paragraphs 22-24 or 28-29, wherein the transgene comprises a nucleotide sequence of SEQ ID NO: 32 encoding the heavy chain and a nucleotide sequence of SEQ ID NO: 33 encoding the light chain; a nucleotide sequence of SEQ ID NO: 34 encoding the heavy chain and a nucleotide sequence of SEQ ID NO: 35 encoding the light chain; a nucleotide sequence of SEQ ID NO: 36 encoding the heavy chain and a nucleotide sequence of SEQ ID NO: 37 encoding the light chain; wherein the transgene comprises a nucleotide sequence of SEQ ID NO: 38 encoding the heavy chain and a nucleotide sequence of SEQ ID NO: 39 encoding the light chain; a nucleotide sequence of SEQ ID NO: 40 encoding the heavy chain and a nucleotide sequence of SEQ ID NO: 41 encoding the light chain; a nucleotide sequence of SEQ ID NO: 42 encoding the heavy chain and a nucleotide sequence of SEQ ID NO: 43 encoding the light chain; wherein the transgene comprises a nucleotide sequence of SEQ ID NO: 44 encoding the heavy chain and a nucleotide sequence of SEQ ID NO: 45 encoding the light chain; or a nucleotide sequence of SEQ ID NO:183 encoding the heavy chain and a nucleotide sequence of SEQ ID NO:184 encoding the light chain.
31. The composition of any of paragraphs 22 to 30, wherein the transgene comprises a Furin/2A linker between the nucleotide sequences coding for the heavy and light chains of said mAb.
32. The composition of any of paragraphs 22 to 31, wherein the nucleic acid encoding a Furin 2A linker is incorporated into the expression cassette in between the nucleotide sequences encoding the heavy and light chain sequences, resulting in a construct with the structure:
   Signal sequence-Heavy chain-Furin site-2A site-Signal sequence-Light chain-PolyA.
33. The composition of paragraphs 22 to 32, wherein said Furin 2A linker is a Furin/T2A linker having the amino acid sequence RKRR(GSG) APVKQTLNFDLLKLAGDVESNPGP (SEQ ID NOS:143 or 144).
34. The composition of any of paragraphs 22 to 33, wherein said signal sequence is MYRMQLLLLIALSLALVTNS (SEQ ID NO:85) or a signal sequence from Table 2.
35. The composition of any of paragraphs 22 to 34, wherein the artificial genome is self complementary.
36. The composition of any of paragraphs 22 to 27 or 31 to 35, wherein the artificial genome is the construct EF1ac.Vh4i.Adalimumab.Fab scAAV, mU1a.Vh4i.Adalimumab.Fab scAAV, CAG.Adalimumab.IgG, CAG.Adalimumab.Fab, GRK1.Vh4i.Adalimumab.IgG, CB.VH4i.adalimumab.IgG, CBlong.VH4i.adalimumab.IgG. or Best1/GRK1.VH4i.adalimumab.IgG.

### Method of Treatment

37. A method of treating non-infectious uveitis in a human subject in need thereof, comprising subretinally, intravitreally, intranasally, intracamerally, suprachoroidally, or systemically administering to the subject a therapeutically effective amount of a composition comprising a recombinant AAV comprising a transgene encoding an anti-TNFα mAb, anti-IL6 mAb, or anti-IL6R mAb, or antigen-binding fragment thereof, operably linked to one or more regulatory sequences that control expression of the transgene in ocular tissue cells.
38. A method of treating non-infectious uveitis in a human subject in need thereof, comprising: subretinally, intravitreally, intranasally, intracamerally, suprachoroidally, or systemically administering to said subject a therapeutically effective amount of a recombinant nucleotide expression vector comprising a transgene encoding an anti-TNFα mAb, anti-IL6 mAb, or anti-IL6R mAb, or antigen-binding fragment thereof, operably linked to one or more regulatory sequences that control expression of the transgene in human ocular tissue cells, so that a depot is formed that releases a human post-translationally modified (HuPTM) form of anti-TNFα mAb, anti-IL6 mAb, or anti-IL6R mAb or antigen-binding fragment thereof
39. The method of paragraphs 37 or 38 wherein the anti-TNFα mAb is adalimumab, infliximab or golimumab.
40. The method of paragraphs 37 to 39, wherein the full-length anti-TNFα mAb or the antigen-binding fragment comprises a heavy chain with an amino acid sequence of SEQ ID NO: 1 and optionally an Fc polypeptide with an amino acid sequence of SEQ ID NO: 64 and a light chain with an amino acid sequence of SEQ ID NO: 2; or a heavy chain with an amino acid sequence of SEQ ID NO: 3 and optionally an Fc polypeptide with an amino acid sequence of SEQ ID NO: and a light chain with an amino acid sequence of SEQ ID NO: 4; a heavy chain with an amino acid sequence of SEQ ID NO: 5 and optionally an Fc polypeptide with an amino acid sequence of SEQ ID NO: 65 and a light chain with an amino acid sequence of SEQ ID NO: 6.
41. The method of paragraphs 37 to 40, wherein the transgene comprises a nucleotide sequence of SEQ ID NO: 26 encoding the heavy chain and a nucleotide sequence of SEQ ID NO: 27 encoding the light chain; a nucleotide sequence of SEQ ID NO: 28 encoding the heavy chain and a nucleotide sequence of SEQ ID NO: 29 encoding the light chain; or a nucleotide sequence of SEQ ID NO: 30 encoding the heavy chain and a nucleotide sequence of SEQ ID NO: 31 encoding the light chain.
42. The method of any of paragraphs 37 to 38, wherein the antiIL6 or anti-IL6R antibody is satralizumab, sarilumab, siltuximab, clazakizumab, sirukumab, olokizumab, gerilizumab, or tocilizumab, or an antigen binding fragment thereof
43. The method of any of paragraphs 37 to 38 or 42, wherein the full-length mAb or the antigen-binding fragment comprises a heavy chain with an amino acid sequence of SEQ ID NO: 7 and optionally an Fc polypeptide with an amino acid sequence of SEQ ID NO: 67 and a light chain with an amino acid sequence of SEQ ID NO: 8; a heavy chain with an amino acid sequence of SEQ ID NO: 9 and optionally an Fc polypeptide with an amino acid sequence of SEQ ID NO:185 and a light chain with an amino acid sequence of SEQ ID NO: 10; a heavy chain with an amino acid sequence of SEQ ID NO: 11 and optionally an Fc polypeptide with an amino acid sequence of SEQ ID NO: 68 and a light chain with an amino acid sequence of SEQ ID NO: 12; comprises a heavy chain with an amino acid sequence of SEQ ID NO: 13 and optionally an Fc polypeptide with an amino acid sequence of SEQ ID NO: 69 and a light chain with an amino acid sequence of SEQ ID NO: 14; a heavy chain with an amino acid sequence of SEQ ID NO: 15 and optionally an Fc polypeptide with an amino acid sequence of SEQ ID NO: 70 and a light chain with an amino acid sequence of SEQ ID NO: 16; a heavy chain with an amino acid sequence of SEQ ID NO: 17 and optionally an Fc polypeptide with an amino acid sequence of SEQ ID NO: 71 and a light chain with an amino acid sequence of SEQ ID NO: 18; a heavy chain with an amino acid sequence of SEQ ID NO: 19 and a light chain with an amino acid sequence of SEQ ID NO: 20; or a heavy chain with an amino acid sequence of SEQ ID NO: 21 and optionally an Fc polypeptide with an amino acid sequence of SEQ ID NO: 72 and a light chain with an amino acid sequence of SEQ ID NO: 22.
44. The method of any of paragraphs 37 to 38 or 42 to 43, wherein the transgene comprises a nucleotide sequence of SEQ ID NO: 32 encoding the heavy chain and a nucleotide sequence of SEQ ID NO: 33 encoding the light chain; a nucleotide sequence of SEQ ID NO: 34 encoding the heavy chain and a nucleotide sequence of SEQ ID NO: 35 encoding the light chain; a nucleotide sequence of SEQ ID NO: 36 encoding the heavy chain and a nucleotide sequence of SEQ ID NO: 37 encoding the light chain; wherein the transgene comprises a nucleotide sequence of SEQ ID NO: 38 encoding the heavy chain and a nucleotide sequence of SEQ ID NO: 39 encoding the light chain; a nucleotide sequence of SEQ ID NO: 40 encoding the heavy chain and a nucleotide sequence of SEQ ID NO: 41 encoding the light chain; a nucleotide sequence of SEQ ID NO: 42 encoding the heavy chain and a nucleotide sequence of SEQ ID NO: 43 encoding the light chain; wherein the transgene comprises a nucleotide sequence of SEQ ID NO: 44 encoding the heavy chain and a nucleotide sequence of SEQ ID NO: 45 encoding the light chain; or a nucleotide sequence of SEQ ID NO:183 encoding the heavy chain and a nucleotide sequence of SEQ ID NO:184 encoding the light chain.
45. The method of paragraphs 37 to 44, wherein the ocular tissue cell is a cornea cell, an iris cell, a ciliary body cell, a schlemm's canal cell, a trabecular meshwork cell, a retinal cell, a RPE-choroid tissue cell, or an optic nerve cell.
46. The method of any of paragraphs 37 to 44 wherein the viral capsid is at least 95% identical to the amino acid sequence of an AAV serotype 1 (AAV1), serotype 2 (AAV2), serotype AAV2.7m8 (AAV2.7m8), serotype 3 (AAV3), serotype 3B (AAV3B), serotype 4 (AAV4), serotype 5 (AAV5), serotype 6 (AAV6), serotype 7 (AAV7), serotype 8 (AAV8), serotype rh8 (AAVrh8), serotype 9 (AAV9), serotype 9e (AAV9e), serotype rh10 (AAVrh10), serotype rh20 (AAVrh20), serotype rh39 (AAVrh39), serotype hu.37 (AAVhu.37), serotype rh73 (AAVrh73), or serotype rh74 (AAVrh74), serotype hu51 (AAV.hu51), serotype hu21 (AAV.hu21), serotype hu12 (AAV.hu12), or serotype hu26 (AAV.hu26);
47. The method of any of paragraphs 37 to 46, wherein the AAV capsid is AAV2.7m8, AAV8, AAV3B, or AAVrh73.
48. The method of any of paragraphs 37 to 46, wherein the regulatory sequence includes a regulatory sequence from Table 1.
49. The method of paragraph 48, wherein the regulator sequence is a human rhodopsin kinase (GRK1) promoter (SEQ ID NOS:77 or 217), a mouse cone arresting (CAR) promoter (SEQ ID NOS: 214-216), or a human red opsin (RedO) promoter (SEQ ID NO: 212).
50. The method of any of paragraphs 37 to 49, wherein the transgene comprises a Furin/2A linker between the nucleotide sequences coding for the heavy and light chains of said mAb.
51. The method of paragraph 50, wherein said Furin 2A linker is a Furin/T2A linker having the amino acid sequence RKRR(GSG) APVKQTLNFDLLKLAGDVESNPGP (SEQ ID NOS:143 or 144).
52. The method of any of paragraphs 37 to 51, wherein the transgene encodes a signal sequence at the N-terminus of the heavy chain and the light chain of said antigen-binding fragment that directs secretion and post translational modification in said human ocular tissue cells.
53. The method of paragraph 52, wherein said signal sequence is MYRMQLLLLIALSLALVTNS (SEQ ID NO:85) or a signal sequence from Table 2.
54. The method of any of paragraphs 37 to 53, wherein transgene has the structure: Signal sequence-Heavy chain-Furin site-2A site-Signal sequence-Light chain-PolyA.
55. The method of any of paragraphs 37 to 54, wherein the mAb is a hyperglycosylated mutant or wherein the Fc polypeptide of the mAb is glycosylated or aglycosylated.
56. The method of any of paragraphs 37 to 55 wherein the mAb contains an alpha 2,6-sialylated glycan.
57. The method of any of paragraphs 37 to 56 wherein the mAb is glycosylated but does not contain detectable NeuGc and/or α-Gal.
58. The method of any of paragraphs 37 to 57 wherein the mAb contains a tyrosine sulfation.
59. The method of any of paragraphs 37 to 58 in which production of said HuPTM form of said mAb or antigen-binding fragment thereof is confirmed by transducing human ocular tissue cells in culture with said recombinant nucleotide expression vector and expressing said mAb or antigen-binding fragment thereof.
60. The method of paragraphs 37 or 59, wherein the therapeutically effective amount is determined to be sufficient to maintain a concentration of at least 10 ng/ml in aqueous humor, vitreous humor, RPE, retina, and/or anterior segment/chamber.
61. The method of paragraphs 37 or 60, wherein the therapeutically effective amount is determined to be sufficient to improve best corrected visual acuity (BCVA) by >=2 ETDRS lines or increase in logMAR, reduced inflammatory activity of the anterior and posterior chamber according to the SUN classification, and/or reduction in grade of vitreous haze.
62. The method of any of paragraphs 37 to 61, wherein the rAAV is self complementary.
63. The method of any of paragraphs 37 to 62 wherein the transgene within the construct EF1ac.Vh4i.Adalimumab.Fab scAAV, mU1a.Vh4i.Adalimumab.Fab scAAV, CAG.Adalimumab.IgG, CAG.Adalimumab.Fab, GRK1.Vh4i.Adalimumab.IgG, CB.VH4i.adalimumab.IgG, CBlong.VH4i.adalimumab.IgG or Best1/GRK1.VH4i.adalimumab.IgG.

### Method of Manufacture

64. A method of producing recombinant AAVs comprising:
   (a) culturing a host cell containing:
      (i) an artificial genome comprising a cis expression cassette flanked by AAV ITRs, wherein the cis expression cassette comprises comprising a transgene encoding a substantially full-length or full-length anti-TNFα mAb, anti-IL6, or anti-IL6R or antigen-binding fragment thereof, operably linked to one or more regulatory sequences that promote expression of the transgene in human ocular tissue cells;
      (ii) a trans expression cassette lacking AAV ITRs, wherein the trans expression cassette encodes an AAV rep and an AAV capsid protein operably linked to expression control elements that drive expression of the AAV rep and the AAV capsid protein in the host cell in culture and supply the AAV rep and the AAV capsid protein in trans, wherein the capsid has ocular tissue cell tropism;
      (iii) sufficient adenovirus helper functions to permit replication and packaging of the artificial genome by the AAV capsid protein; and
   (b) recovering recombinant AAV encapsidating the artificial genome from the cell culture.
65. The method of paragraph 64, wherein the transgene encodes a substantially full-length or full-length mAb or antigen binding fragment that comprises the heavy and light chain variable domains of adalimumab, infliximab, golimumab, satralizumab, sarilumab, siltuximab, clazakizumab, sirukumab, olokizumab, gerilizumab, or tocilizumab wherein the AAV capsid protein is an AAV2.7m8, AAV8, AAV3B, or AAVrh73, capsid protein.
66. The method of paragraphs 64 or 65, wherein the ocular tissue cell is a cornea cell, an iris cell, a ciliary body cell, a schlemm's canal cell, a trabecular meshwork cell, a retinal cell, a RPE-choroid tissue cell, or an optic nerve cell.
67. A host cell containing:
   a. an artificial genome comprising a cis expression cassette flanked by AAV ITRs, wherein the cis expression cassette comprises comprising a transgene encoding a substantially full-length or full-length anti-TNFα mAb, anti-IL6 mAb or anti-IL6R mAb, or antigen-binding fragment thereof, operably linked to one or more regulatory sequences that promote expression of the transgene in human ocular tissue cells;
   b. a trans expression cassette lacking AAV ITRs, wherein the trans expression cassette encodes an AAV rep and an AAV capsid protein operably linked to expression control elements that drive expression of the AAV rep and the AAV capsid protein in the host cell in culture and supply the AAV rep and the AAV capsid protein in trans, wherein the capsid has ocular tissue cell tropism;
   c. sufficient adenovirus helper functions to permit replication and packaging of the artificial genome by the AAV capsid protein.
68. The host cell of paragraph 67, wherein the transgene encodes a substantially full-length or full-length mAb or antigen binding fragment that comprises the heavy and light chain variable domains of adalimumab, infliximab, golimumab, satralizumab, sarilumab, siltuximab, clazakizumab, sirukumab, olokizumab, gerilizumab, or tocilizumab.
69. The host cell of paragraphs 67 or 68, wherein the AAV capsid protein is an AAV2.7m8, AAV8, AAV3B, or AAVrh73 capsid protein.
70. The host cell of paragraphs 67 to 69, wherein the ocular tissue cell is a cornea cell, an iris cell, a ciliary body cell, a schlemm's canal cell, a trabecular meshwork cell, a retinal cell, a RPE-choroid tissue cell, or an optic nerve cell.

### 4. BRIEF DESCRIPTION OF THE DRAWINGS

FIGS. 1A-1B. Schematics of rAAV vector genome constructs containing an expression cassette encoding the heavy and light chains of a therapeutic mAb separated by a Furin-2A linker, operably linked to a CAG promoter, controlled by expression elements, flanked by the AAV ITRs. The transgene can comprise nucleotide sequences encoding the full-length heavy and light chains with Fc regions (A) or the heavy and light chains of the Fab portion (B).

FIGS. 2A-2C. The amino acid sequence of a transgene construct for the Fab region of adalimumab (A), infliximab (B), and golimumab (C), therapeutic antibodies to tumor necrosis factor (TNFα). Glycosylation sites are boldface. Glutamine glycosylation sites; asparaginal (N) glycosylation sites, non-consensus asparaginal (N) glycosylation sites; and tyrosine-O-sulfation sites (italics) are as indicated in the legend. Complementarity-determining regions (CDR) are underscored. The hinge region is highlighted in grey.

FIGS. 3A-3H. The amino acid sequence of a transgene construct for the Fab region of satralizumab (A), sarilumab (B), siltuximab (C), Clazakizumab (D), Sirukumab (E), olokizumab (F), gerilizumab (G), or tocilizumab (H). Glycosylation sites are boldface. Glutamine glycosylation sites; asparaginal (N) glycosylation sites, non-consensus asparaginal (N) glycosylation sites; and tyrosine-sites (italics) are as indicated in the legend. Complementarity-determining regions (CDR) are underscored. The hinge region is highlighted in grey.

FIG. 4. Clustal Multiple Sequence Alignment of various capsids with ocular tissue tropism. Amino acid substitutions (shown in bold in the bottom rows) can be made to AAV8 capsids by “recruiting” amino acid residues from the corresponding position of other aligned AAV capsids. Sequence shown in gray=hypervariable regions. The amino acid sequences of the AAV capsids are assigned sequence ID numbers as indicated in FIG. 4.

FIG. 5 Glycans that can be attached to HuGlyFab regions of full length mAbs or the antigen-binding domains. (Adapted from Bondt et al., 2014, Mol & Cell Proteomics 13.1: 3029-3039).

FIG. 6. Clustal Multiple Sequence Alignment of constant heavy chain regions (CH2 and CH3) of IgG1 (SEQ ID NO: 61), IgG2 (SEQ ID NO: 62), and IgG4 (SEQ ID NO: 63). The hinge region, from residue 219 to residue 230 of the heavy chain, is shown in italics. The numbering of the amino acids is in EU-format.

FIG. 7. Expression levels of vectorized adalimumab (AAV8.CAG.adalimumabigG) in ocular tissues (retina, retinal pigment epithelial (RPE), and anterior segment) at three different doses (1e7, 1e8, and 1e9 vg/eye). PBS is used a vehicle control and AAV.GFP as control vector. Adalimumab expression levels (ng) are depicted relative to the total amount of protein (g).

FIG. 8. Expression levels of vectorized adalimumab (AAV8.CAG.adalimumab.IgG) in ocular tissues (retina, retinal pigment epithelial (RPE), and anterior segment) at three different doses (1e7, 1e8, and 1e9 vg/eye). PBS is used a vehicle control and AAV.GFP as control vector. Adalimumab expression levels (ng) are depicted as concentration per ml.

FIGS. 9A and 9B show the alignment of different antibody sequences. A) heavy chain sequences of antibodies. Top to bottom: amino acids 1-229 of SEQ ID NO:23, amino acids 1-228 of SEQ ID NO:3, amino acids 1-237 of SEQ ID NO:5, amino acids 1-224 of SEQ ID NO:7, amino acids 1-224 of SEQ ID NO:9, amino acids 1-227 of SEQ ID NO:11, amino acids 1-228 of SEQ ID NO:13, amino acids 1-227 of SEQ ID NO:15, amino acids 1-224 of SEQ ID NO:17, amino acids 1-230 of SEQ ID NO:19, amino acids 1-228 of SEQ ID NO:21. B) Light chain sequences of antibodies. Top to bottom: amino acids 1-229 of SEQ ID NO:24, amino acids 1-228 of SEQ ID NO:4, amino acids 1-237 of SEQ ID NO:6, amino acids 1-224 of SEQ ID NO:8, amino acids 1-224 of SEQ ID NO:10, amino acids 1-227 of SEQ ID NO:12, amino acids 1-228 of SEQ ID NO:14, amino acids 1-227 of SEQ ID NO:16, amino acids 1-224 of SEQ ID NO:18, amino acids 1-230 of SEQ ID NO:20, amino acids 1-228 of SEQ ID NO:22.

FIGS. 10A and 10B show binding to various concentrations of mouse or human TNFα compared in a competitive ELISA assay for both vector-expressed adalimumab extracted from mouse eye (following subretinal) administration) (10A) and commercial adalimumab (10B).

FIGS. 11A and B show results of dose response studies. A depicts results of an ADCC dose response study with CHO/DG44-tm TNFα cells used as the target cells with E/T ratio at 25:1. B. CHO/DG44-tm TNFα cells were used as the target cells with 5% normal human serum complement (NHSC) in CDC dose-response study. Dose-responses and best-fit values of positive control (Adalimumab), samples (AAV-Adalimumab) and negative control (Human IgG1) are shown in A and B.

FIG. 12 depicts total scores over time for 3 (rat) groups administered with varying doses of hTNFα (50 ng, 100 ng and 170 ng) and a control (vehicle) group and naïve group.

FIG. 13 shows levels of adalimumab (as measured by ELISA with wells coated with recombinant human TNF) in eyes of Lewis Rats 21 days after subretinal injection with AAV8.CAG.Adalimumab at 1.0E+9 GC/eye and 3.0E+8 GC/eye have 86.0 ng/eye and 17.1 ng/eye of adalimumab/eye, respectively.

FIG. 14 depicts adalimumab levels in ocular tissues RPE, Retina and Anterior Segment, from mice following subretinal administration of AAV8.CAG.adalumumab or AAV8.GRK1.adalimumab at doses of 1.0E08 or 1.0E09 and vehicle control 4 to 5 weeks after administration.

### 5. DETAILED DESCRIPTION OF THE INVENTION

Compositions and methods are described for the systemic delivery of a fully human post-translationally modified (HuPTM) therapeutic monoclonal antibody (mAb) or a HuPTM antigen-binding fragment of a therapeutic anti-TNFα, anti-IL6, or anti-IL6R mAb (for example, a fully human-glycosylated Fab (HuGlyFab) of a therapeutic mAb) to a patient (human subject) diagnosed with non-infectious uveitis or other indication indicated for treatment with the therapeutic mAb. Delivery may be advantageously accomplished via gene therapy—e.g., by administering a viral vector or other DNA expression construct encoding a therapeutic mAb or its antigen-binding fragment (or a hyperglycosylated derivative of either) to a patient (human subject) diagnosed with a condition indicated for treatment with the therapeutic mAb—to create a permanent depot in a tissue or organ of the patient, particularly the eye that continuously supplies the HuPTM mAb or antigen-binding fragment of the therapeutic mAb, e.g., a human-glycosylated transgene product, into ocular tissues of the subject to where the mAb or antigen-binding fragment there of exerts its therapeutic effect.

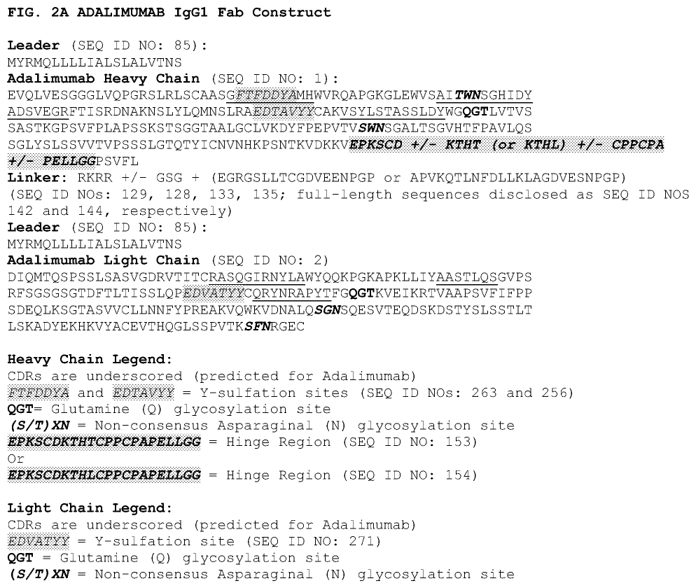

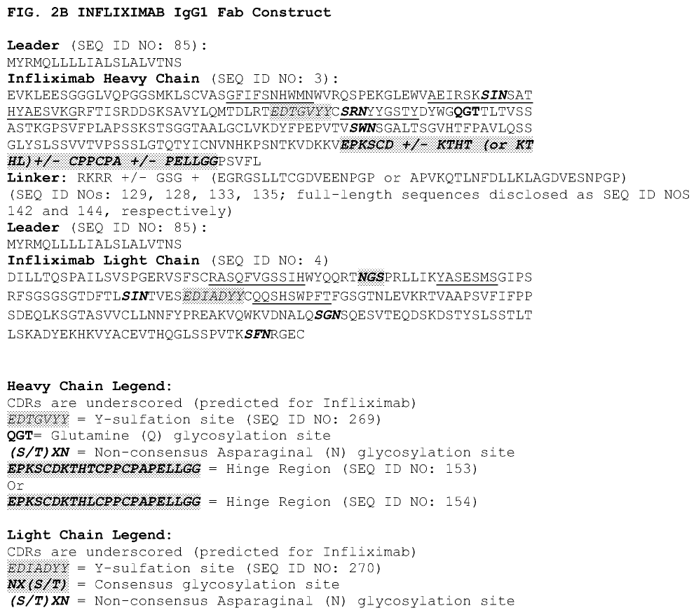

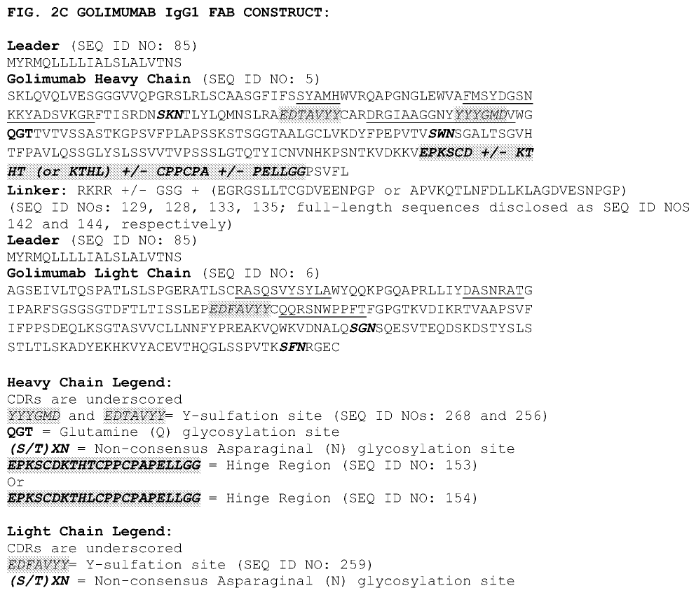

In certain embodiments, the HuPTM mAb or HuPTM antigen-binding fragment encoded by the transgene, but it not limited to, is a full-length or an antigen-binding fragment of a HuPTM mAb or HuPTM that binds TNFα, particularly adalimumab (see FIG. 2A for the heavy and light chain sequences of the Fab portion of adalimumab), IL6, or IL6R.

The compositions and methods provided herein systemically deliver anti-TNFα, particularly, adalimumab, anti-IL6, or anti-IL6R antibodies, from a depot of viral genomes, for example, in the subject's eye, or liver/muscle, at a level either in the ocular tissue (e.g., in the vitreous or aqueous humor, or in the serum that is therapeutically or prophylactically effective to treat or ameliorate the symptoms of non-infectious uveitis or other indication that may be treated with an anti-TNFα, anti-IL6, or anti-IL6R antibody. Identified herein are viral vectors for delivery of transgenes encoding the therapeutic anti-TNFα, anti-IL6 or anti-IL6R antibodies to cells in the human subject, including, in embodiments, one or more ocular tissue cells, and regulatory elements operably linked to the nucleotide sequence encoding the heavy and light chains of the anti-TNFα, anti-IL6, or anti-IL6R antibody that promote the expression of the antibody in the cells, in embodiments, in the ocular tissue cells. Such regulatory elements, including ocular tissue-specific regulatory elements, are provided in Table 1 and Table 1a herein. Accordingly, such viral vectors may be delivered to the human subject at appropriate dosages, such that at least 20, 30, 40, 50 or 60 days after administration, the anti-TNFα, anti-IL6, or anti-IL6R antibody is present at therapeutically effective levels in the serum or in ocular tissues of said human subject. In embodiments, the therapeutically effective level of the anti-TNFα, anti-IL6, or anti-IL6R antibody is determined (in human trials, animal models, etc.) to improve best corrected visual acuity (BCVA) by >=2 ETDRS lines or increase in logMAR, reduced inflammatory activity of the anterior and posterior chamber according to the SUN classification, and/or reduction in grade of vitreous haze.

The HuPTM mAb or HuPTM antigen-binding fragment encoded by the transgene can include, but is not limited to, a full-length or an antigen-binding fragment of a therapeutic antibody that binds to TNFα, including but not limited to, adalimumab, infliximab or golimumab, or to IL6, or IL6R, including but not limited to satralizumab, sarilumab, siltuximab, clazakizumab, sirukumab, olokizumab, gerilizumab, or tocilizumab. The amino acid sequences of the heavy and light chain of antigen binding fragments of the foregoing are provided in Table 7, infra. Heavy chain variable domain having an amino acid sequence of SEQ ID NO: 1, 3, 5, 7, 9, 11, 13, 15, 17, 19, 21, or 23 (encoded by nucleotide sequence SEQ ID NO: 26, 28, 30, 32, 34, 36, 38, 40, 42, or 44, respectively) and light chain variable domain having an amino acid sequence of SEQ ID NO: 2, 4, 6, 8, 10, 12, 14, 16, 18, 20, or 22 (encoded by nucleotide sequence SEQ ID NO: 27, 29, 31, 33, 35, 37, 39, 41, 43, or 45, respectively). The HuPTM mAb or HuPTM antigen-binding fragment encoded by the transgene can include, but is not limited to, a full-length or an antigen-binding fragment of a therapeutic antibody or antigen-binding fragments engineered to contain additional glycosylation sites on the Fab domain (e.g., see Courtois et al., 2016, mAbs 8: 99-112 which is incorporated by reference herein in its entirety for its description of derivatives of antibodies that are hyperglycosylated on the Fab domain of the full-length antibody).

The recombinant vector used for delivering the transgene includes non-replicating recombinant adeno-associated virus vectors (“rAAV”). rAAVs are particularly attractive vectors for a number of reasons-they can be modified to preferentially target a specific organ of choice; and there are hundreds of capsid serotypes to choose from to obtain the desired tissue specificity, and/or to avoid neutralization by pre-existing patient antibodies to some AAVs. The AAV types for use here in preferentially target the eye, i.e., have a tropism for retinal cells. Such rAAVs include but are not limited to AAV based vectors comprising capsid components from one or more of AAV1, AAV2, AAV3, AAV3B, AAV4, AAV5, AAV6, AAV7, AAV8, AAVrh8, AAV9, AAV9e, AAVrh10, AAVrh20, AAVrh39, AAVhu.37, AAVrh73, AAVrh74, AAV.hu51, AAV.hu21, AAV.hu12, or AAV.hu26. In certain embodiments, AAV based vectors provided herein comprise capsids from one or more of AA2.7m8, AAV3B, AAV8, AAV9, AAVrh10, AAV10, or AAVrh73 serotypes.

However, other viral vectors may be used, including but not limited to lentiviral vectors; vaccinia viral vectors, or non-viral expression vectors referred to as “naked DNA” constructs. Expression of the transgene can be controlled by constitutive or tissue-specific expression control elements.

Gene therapy constructs are designed such that both the heavy and light chains are expressed. In certain embodiments, the full length heavy and light chains of the antibody are expressed. In certain embodiments, the coding sequences encode for a Fab or F(ab′)₂ or an scFv. The heavy and light chains should be expressed at about equal amounts, in other words, the heavy and light chains are expressed at approximately a 1:1 ratio of heavy chains to light chains. The coding sequences for the heavy and light chains can be engineered in a single construct in which the heavy and light chains are separated by a cleavable linker or IRES so that separate heavy and light chain polypeptides are expressed. In specific embodiments, the linker separating the heavy and light chains is a Furin-2A linker, for example a Furin-F2A linker RKRR(GSG) APVKQTLNFDLLKLAGDVESNPGP (SEQ ID NOS:143 or 144) or a Furin-T2A linker RKRR(GSG) EGRGSLLTCGDVEENPGP (SEQ ID NOS:141 or 142). In certain embodiments, the construct expresses, from the N-terminus to C-terminus, NH2-VL-linker-VH—COOH or NH2-VH-linker-VL-COOH. In other embodiments, the construct expresses, from the N-terminus to C-terminus, NH2-signal or localization sequence-VL-linker-VH—COOH or NH2-signal or localization sequence-VH-linker-VL-COOH. In other embodiments, the constructs express a scFv in which the heavy and light chain variable domains are connected via a flexible, non-cleavable linker.

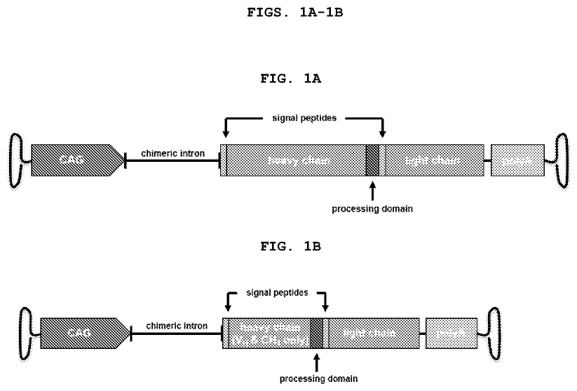

In certain embodiments, nucleic acids (e.g., polynucleotides) and nucleic acid sequences disclosed herein may be codon-optimized, for example, via any codon-optimization technique known to one of skill in the art (see, e.g., review by Quax et al., 2015, Mol Cell 59:149-161) and may also be optimized to reduce CpG dimers. Codon optimized sequences of the adalimumab heavy and light chains are provided in Table 8 (SEQ ID NOs: 46 to 60). Each heavy and light chain requires a signal sequence to ensure proper post-translation processing and secretion (unless expressed as a scFv, in which only the N-terminal chain requires a signal sequence sequence). Useful signal sequences for the expression of the heavy and light chains of the therapeutic antibodies in human cells are disclosed herein. Exemplary recombinant expression constructs are shown in FIGS. 1A and 1B.

The production of HuPTM mAb or HuPTM Fab (including an HuPTM scFv) should result in a “biobetter” molecule for the treatment of disease accomplished via gene therapy—e.g., by administering a viral vector or other DNA expression construct encoding a full-length HuPTM mAb or HuPTM Fab or other antigen binding fragment, such as an scFv, of a therapeutic mAb to a patient (human subject) diagnosed with a disease indication for that mAb, to create a permanent depot in the subject that continuously supplies the human-glycosylated, sulfated transgene product produced by the subject's transduced cells. The cDNA construct for the HuPTM mAb or HuPTM Fab or HuPTM scFv should include a signal peptide that ensures proper co- and post-translational processing (glycosylation and protein sulfation) by the transduced human cells.

Pharmaceutical compositions suitable for administration to human subjects comprise a suspension of the recombinant vector in a formulation buffer comprising a physiologically compatible aqueous buffer, a surfactant and optional excipients. Such formulation buffer can comprise one or more of a polysaccharide, a surfactant, polymer, or oil.

As an alternative, or an additional treatment to gene therapy, the full-length HuPTM mAb or HuPTM Fab or other antigen binding fragment thereof can be produced in human cell lines by recombinant DNA technology, and the glycoprotein can be administered to patients. Human cell lines that can be used for such recombinant glycoprotein production include but are not limited to human embryonic kidney 293 cells (HEK293), fibrosarcoma HT-1080, HKB-11, CAP, HuH-7, and retinal cell lines, PER.C6, or RPE to name a few (e.g., see Dumont et al., 2015, Crit. Rev. Biotechnol. 36(6):1110-1122, which is incorporated by reference in its entirety for a review of the human cell lines that could be used for the recombinant production of the HuPTM mAb, HuPTM Fab or HuPTM scFv product, e.g., HuPTM Fab glycoprotein). To ensure complete glycosylation, especially sialylation, and tyrosine-sulfation, the cell line used for production can be enhanced by engineering the host cells to co-express α-2,6-sialyltransferase (or both α-2,3- and α-2,6-sialyltransferases) and/or TPST-1 and TPST-2 enzymes responsible for tyrosine-O-sulfation in human cells.

It is not essential that every molecule produced either in the gene therapy or protein therapy approach be fully glycosylated and sulfated. Rather, the population of glycoproteins produced should have sufficient glycosylation (including 2,6-sialylation) and sulfation to demonstrate efficacy. The goal of gene therapy treatment of the invention is to slow or arrest the progression of disease.

Combination therapies involving delivery of the full-length HuPTM mAb or HuPTM Fab or antigen binding fragment thereof to the patient accompanied by administration of other available treatments are encompassed by the methods of the invention. The additional treatments may be administered before, concurrently or subsequent to the gene therapy treatment. Such additional treatments can include but are not limited to co-therapy with the therapeutic mAb.

Also provided are methods of manufacturing the viral vectors, particularly the AAV based viral vectors. In specific embodiments, provided are methods of producing recombinant AAVs comprising culturing a host cell containing an artificial genome comprising a cis expression cassette flanked by AAV ITRs, wherein the cis expression cassette comprises a transgene encoding a therapeutic antibody operably linked to expression control elements that will control expression of the transgene in human cells; a trans expression cassette lacking AAV ITRs, wherein the trans expression cassette encodes an AAV rep and capsid protein operably linked to expression control elements that drive expression of the AAV rep and capsid proteins in the host cell in culture and supply the rep and cap proteins in trans; sufficient adenovirus helper functions to permit replication and packaging of the artificial genome by the AAV capsid proteins; and recovering recombinant AAV encapsidating the artificial genome from the cell culture.

5.1 Constructs

Viral vectors or other DNA expression constructs encoding an anti-TNFα, anti-IL6, or anti-IL6 HuPTM mAb or antigen-binding fragment thereof, particularly a HuGlyFab, or a hyperglycosylated derivative of a HuPTM mAb antigen-binding fragment are provided herein. The viral vectors and other DNA expression constructs provided herein include any suitable method for delivery of a transgene to a target cell. The means of delivery of a transgene include viral vectors, liposomes, other lipid-containing complexes, other macromolecular complexes, synthetic modified mRNA, unmodified mRNA, small molecules, non-biologically active molecules (e.g., gold particles), polymerized molecules (e.g., dendrimers), naked DNA, plasmids, phages, transposons, cosmids, or episomes. In some embodiments, the vector is a targeted vector, e.g., a vector targeted ocular tissue cells or a vector that has a tropism for ocular tissue cells.

In some aspects, the disclosure provides for a nucleic acid for use, wherein the nucleic acid comprises a nucleotide sequence that encodes a HuPTM mAb or HuGlyFab or other antigen-binding fragment thereof, as a transgene described herein, operatively linked to an ubiquitous promoter, a ocular tissue-specific promoter, or an inducible promoter, wherein the promoter is selected for expression in tissue targeted for expression of the transgene. Promoters may, for example, be a CB7/CAG promoter (SEQ ID NO: 73) and associated upstream regulatory sequences, cytomegalovirus (CMV) promoter, EF-1 alpha promoter (SEQ ID NO: 76), mU1a (SEQ ID NO: 75), UB6 promoter, chicken beta-actin (CBA) promoter, and ocular-tissue specific promoters, such as human rhodopsin kinase (GRK1) promoter (SEQ ID NOS:77 or 217), a mouse cone arresting (CAR) promoter (SEQ ID NOS: 214-216), or a human red opsin (RedO) promoter (SEQ ID NO: 212). See Tables 1 and 1a for a list of useful promoters.

In certain embodiments, provided herein are recombinant vectors that comprise one or more nucleic acids (e.g., polynucleotides). The nucleic acids may comprise DNA, RNA, or a combination of DNA and RNA. In certain embodiments, the DNA comprises one or more of the sequences selected from the group consisting of promoter sequences, the sequence of the gene of interest (the transgene, e.g., the nucleotide sequences encoding the heavy and light chains of the HuPTMmAb or HuGlyFab or other antigen-binding fragment), untranslated regions, and termination sequences. In certain embodiments, viral vectors provided herein comprise a promoter operably linked to the gene of interest.

In certain embodiments, nucleic acids (e.g., polynucleotides) and nucleic acid sequences disclosed herein may be codon-optimized, for example, via any codon-optimization technique known to one of skill in the art (see, e.g., review by Quax et al., 2015, Mol Cell 59:149-161).

In a specific embodiment, the constructs described herein comprise the following components: (1) AAV2 inverted terminal repeats that flank the expression cassette; (2) one or more control elements, b) optionally, a chicken β-actin or other intron and c) a rabbit β-globin poly A signal; and (3) nucleic acid sequences coding for the heavy and light chains of a mAb or Fab, separated by a self-cleaving furin (F)/(F/T)2A linker (SEQ ID NOS:141-144), ensuring expression of equal amounts of the heavy and the light chain polypeptides. An exemplary construct is shown in FIGS. 1A and 1B.

In a specific embodiment, the constructs described herein comprise the following components: (1) AAV2 inverted terminal repeats that flank the expression cassette; (2) GRK1 promoter (SEQ ID NO:77), b) optionally, a VH4 intron (SEQ ID NO:80) or other intron and c) a rabbit β-globin polyA signal (SEQ ID NO:78); and (3) nucleic acid sequences coding for a full-length antibody comprising the heavy and light chain sequences using sequences that encode the Fab portion of the heavy chain, including the hinge region sequence, plus the Fc polypeptide of the heavy chain for the appropriate isotype and the light chain, wherein heavy and light chain nucleotide sequences are separated by a self-cleaving furin (F)/(F/T)2A linker (SEQ ID NOS:141-144), ensuring expression of equal amounts of the heavy and the light chain polypeptides.

5.1.1 mRNA Vectors

In certain embodiments, as an alternative to DNA vectors, the vectors provided herein are modified mRNA encoding for the gene of interest (e.g., the transgene, for example, HuPTMmAb or HuGlyFab or other antigen binding fragment thereof). The synthesis of modified and unmodified mRNA for delivery of a transgene to retinal pigment epithelial cells is taught, for example, in Hansson et al., J. Biol. Chem., 2015, 290(9):5661-5672, which is incorporated by reference herein in its entirety. In certain embodiments, provided herein is a modified mRNA encoding for a HuPTMmAb, HuPTM Fab, or HuPTM scFv.

5.1.2 Viral Vectors

Viral vectors include adenovirus, adeno-associated virus (AAV, e.g., AAV2.7m8, AAV8, AAV9, AAVrh10, AAV10), lentivirus, helper-dependent adenovirus, herpes simplex virus, poxvirus, hemagglutinin virus of Japan (HVJ), alphavirus, vaccinia virus, and retrovirus vectors. Retroviral vectors include murine leukemia virus (MLU) and human immunodeficiency virus (HIV)-based vectors. Alphavirus vectors include semliki forest virus (SFV) and sindbis virus (SIN). In certain embodiments, the viral vectors provided herein are recombinant viral vectors. In certain embodiments, the viral vectors provided herein are altered such that they are replication-deficient in humans. In certain embodiments, the viral vectors are hybrid vectors, e.g., an AAV vector placed into a “helpless” adenoviral vector. In certain embodiments, provided herein are viral vectors comprising a viral capsid from a first virus and viral envelope proteins from a second virus. In specific embodiments, the second virus is vesicular stomatitus virus (VSV). In more specific embodiments, the envelope protein is VSV-G protein.

In certain embodiments, the viral vectors provided herein are HIV based viral vectors. In certain embodiments, HIV-based vectors provided herein comprise at least two polynucleotides, wherein the gag and pol genes are from an HIV genome and the env gene is from another virus.

In certain embodiments, the viral vectors provided herein are herpes simplex virus-based viral vectors. In certain embodiments, herpes simplex virus-based vectors provided herein are modified such that they do not comprise one or more immediately early (IE) genes, rendering them non-cytotoxic.

In certain embodiments, the viral vectors provided herein are MLU based viral vectors. In certain embodiments, MLV-based vectors provided herein comprise up to 8 kb of heterologous DNA in place of the viral genes.

In certain embodiments, the viral vectors provided herein are lentivirus-based viral vectors. In certain embodiments, lentiviral vectors provided herein are derived from human lentiviruses. In certain embodiments, lentiviral vectors provided herein are derived from non-human lentiviruses. In certain embodiments, lentiviral vectors provided herein are packaged into a lentiviral capsid. In certain embodiments, lentiviral vectors provided herein comprise one or more of the following elements: long terminal repeats, a primer binding site, a polypurine tract, att sites, and an encapsidation site.

In certain embodiments, the viral vectors provided herein are alphavirus-based viral vectors. In certain embodiments, alphavirus vectors provided herein are recombinant, replication-defective alphaviruses. In certain embodiments, alphavirus replicons in the alphavirus vectors provided herein are targeted to specific cell types by displaying a functional heterologous ligand on their virion surface.

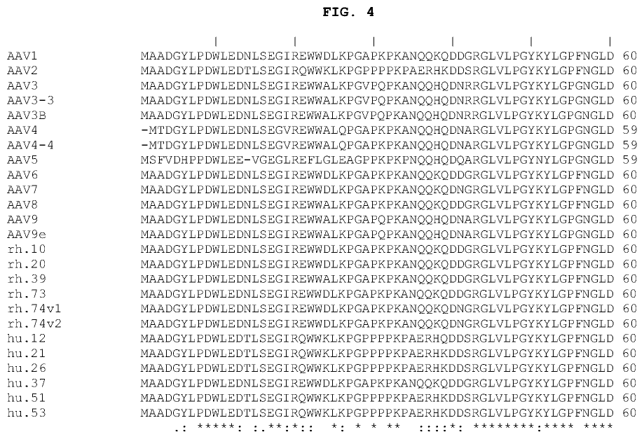

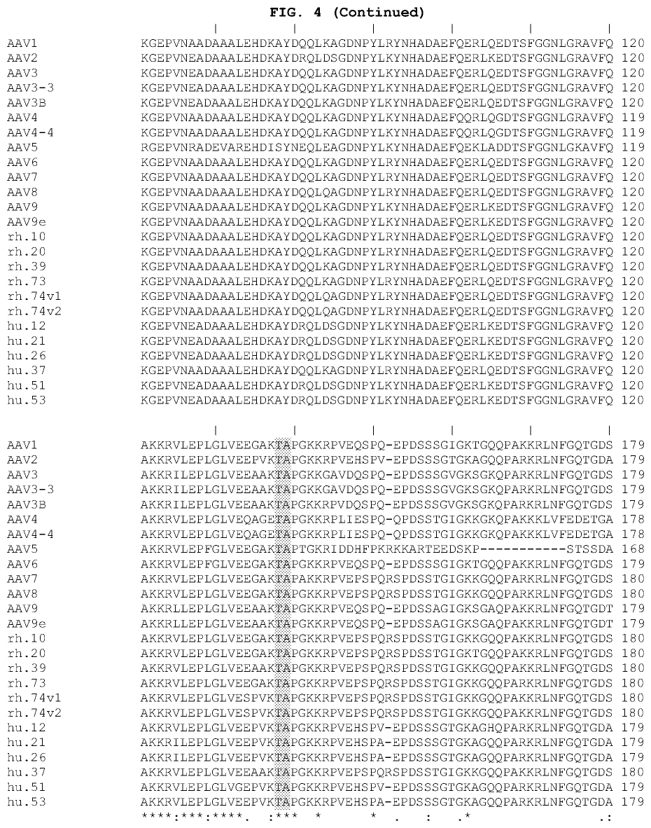

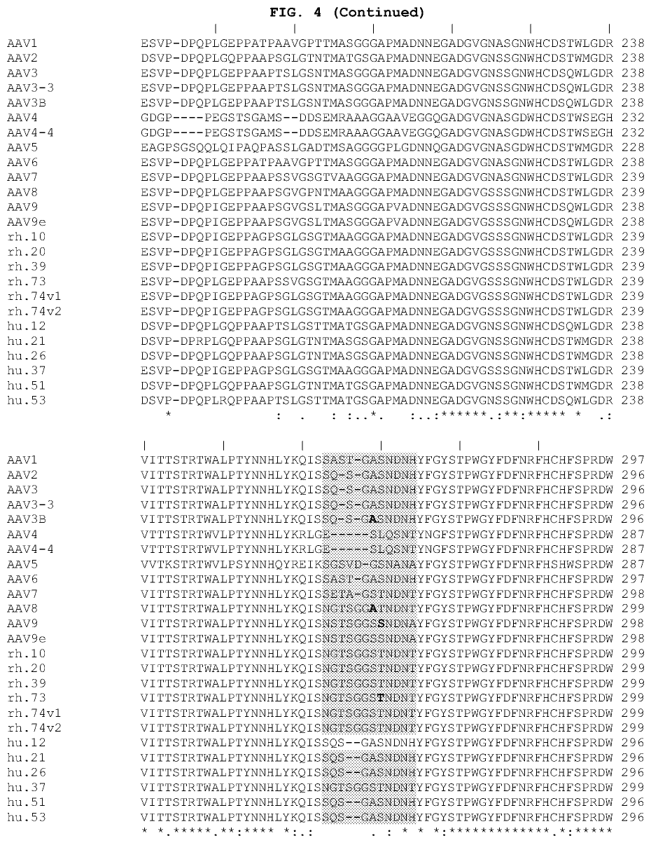

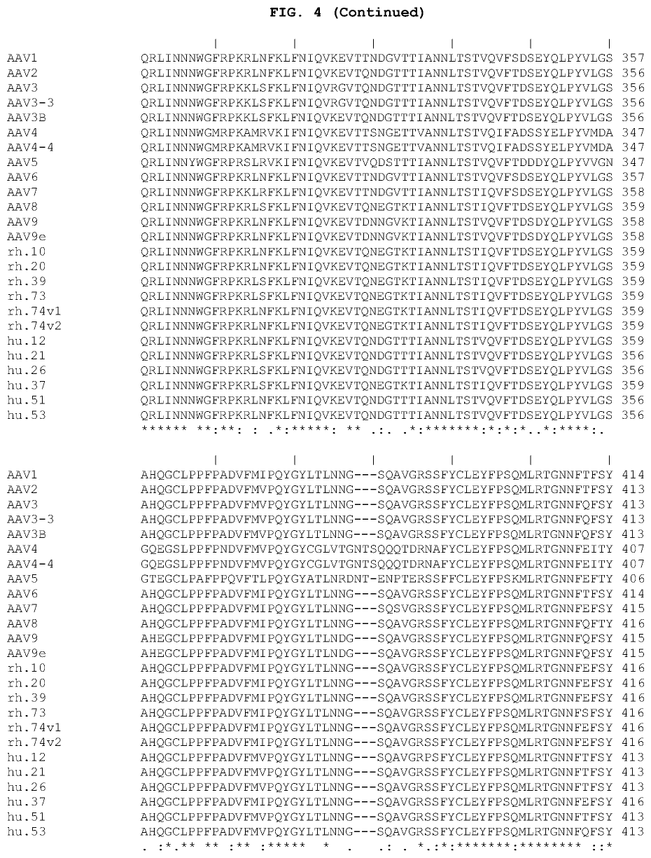

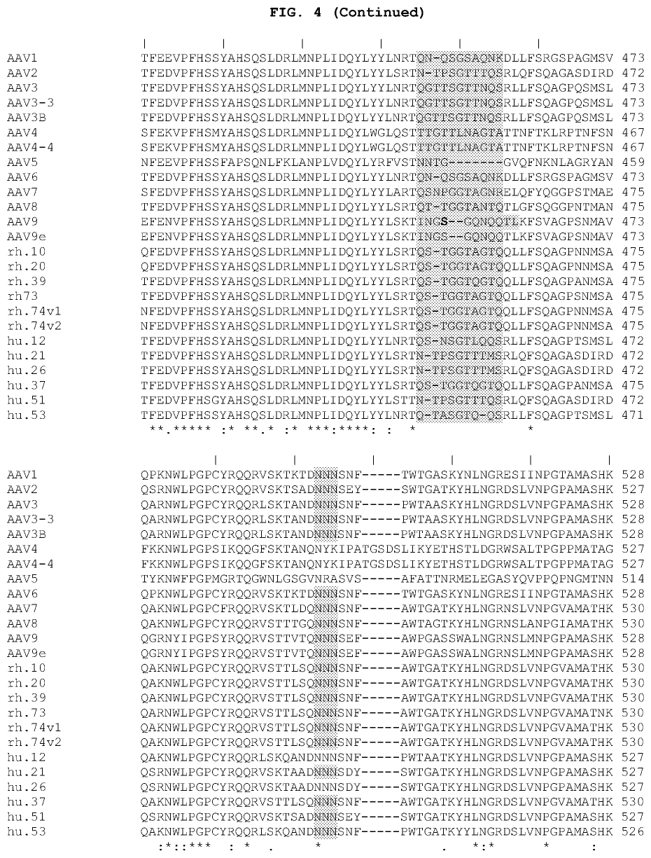

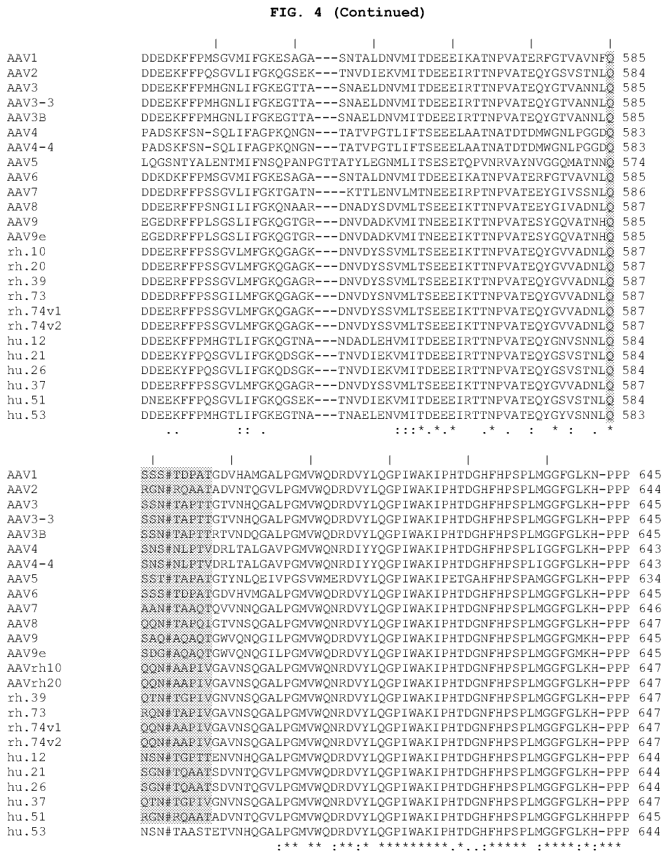

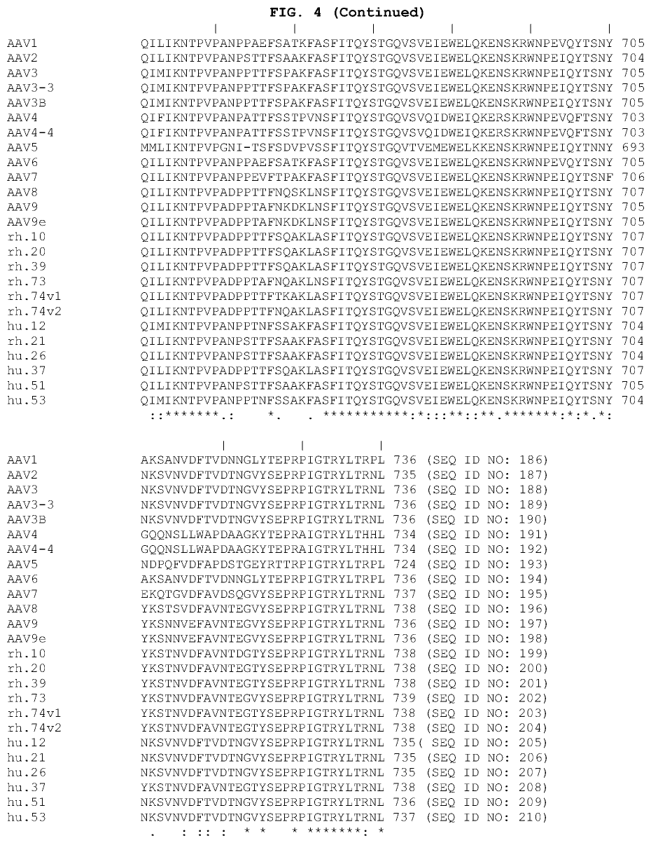

In certain embodiments, the viral vectors provided herein are AAV based viral vectors. In certain embodiments, the AAV-based vectors provided herein do not encode the AAV rep gene (required for replication) and/or the AAV cap gene (required for synthesis of the capsid proteins) (the rep and cap proteins may be provided by the packaging cells in trans). Multiple AAV serotypes have been identified. In certain embodiments, AAV-based vectors provided herein comprise components from one or more serotypes of AAV. In preferred embodiments, AAV-based vectors provided herein comprise components from one or more serotypes of AAV with tropism to ocular tissues, liver and/or muscle. In certain embodiments, AAV based vectors provided herein comprise capsid components from one or more of AAV1, AAV2, AAV3, AAV3B, AAV4, AAV5, AAV6, AAV7, AAV8, AAVrh8, AAV9, AAV9e, AAVrh10, AAVrh20, AAVrh39, AAVhu.37, AAVrh73, AAVrh74, AAV.hu51, AAV.hu21, AAV.hu12, or AAV.hu26. In certain embodiments, AAV based vectors provided herein are or comprise components from one or more of AAV8, AAV3B, AAV9, AAV10, AAVrh73, or AAVrh10 serotypes. Provided are viral vectors in which the capsid protein is a variant of the AAV8 capsid protein (SEQ ID NO:196), AAV3B capsid protein (SEQ ID NO:190), or AAVrh73 capsid protein (SEQ ID NO:202), and the capsid protein is e.g., at least 95%, 96%, 97%, 98%, 99% or 99.9% identical to the amino acid sequence of the AAV8 capsid protein (SEQ ID NO:196), AAV3B capsid protein (SEQ ID NO:190), or AAVrh73 capsid protein (SEQ ID NO:202), while retaining the biological function of the native capsid. In certain embodiments, the encoded AAV capsid has the sequence of SEQ ID NO:196 with 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 amino acid substitutions and retaining the biological function of the AAV8, AAV3B, or AAVrh73 capsid. FIG. 4 provides a comparative alignment of the amino acid sequences of the capsid proteins of different AAV serotypes with potential amino acids that may be substituted at certain positions in the aligned sequences based upon the comparison in the row labeled SUBS. Accordingly, in specific embodiments, the AAV vector comprises an AAV8, AAV3B, or AAVrh73, capsid variant that has 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 amino acid substitutions that are not present at that position in the native AAV capsid sequence as identified in the SUBS row of FIG. 4. Amino acid sequence for AAV8, AAV3B, or AAVrh73 capsids are provided in FIG. 4.

The amino acid sequence of hu37 capsid can be found in international application PCT WO 2005/033321 (SEQ ID NO: 88 thereof) and the amino acid sequence for the rh8 capsid can be found in international application PCT WO 03/042397 (SEQ ID NO:97). The amino acid sequence for the rh64R1 sequence is found in WO2006/110689 (a R697W substitution of the Rh.64 sequence, which is SEQ ID NO: 43 of WO 2006/110689).

In some embodiments, AAV-based vectors comprise components from one or more serotypes of AAV. In some embodiments, AAV based vectors provided herein comprise capsid components from one or more of AAV1, AAV2, AAV3, AAV4, AAV5, AAV6, AAV7, AAV8, AAV9, AAV10, AAV11, AAV12, AAV13, AAV14, AAV15, AAV16, AAVS3, AAV.rh8, AAV.rh10, AAV.rh20, AAV.rh39, AAV.rh46, AAVrh73, AAV.Rh74, AAV.RHM4-1, AAV.hu37, AAV.Anc80, AAV. Anc80L65, AAV.7m8, AAV.PHP.B, AAV.PHP.eB, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, or AAV.HSC16 or other rAAV particles, or combinations of two or more thereof. In some embodiments, AAV based vectors provided herein comprise components from one or more of AAV1, AAV2, AAV3, AAV4, AAV5, AAV6, AAV7, AAV8, AAV9, AAV10, AAV11, AAV12, AAV13, AAV14, AAV15, AAV16, AAVS3, AAV.rh8, AAV.rh10, AAV.rh20, AAV.rh39, AAV.rh46, AAV.rh73, AAV.Rh74, AAV.RHM4-1, AAV.hu37, AAV.Anc80, AAV.Anc80L65, AAV.7m8, AAV.PHP.B, AAV.PHP.eB, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, or AAV.HSC16 or other rAAV particles, or combinations of two or more thereof serotypes. In some embodiments, rAAV particles comprise a capsid protein at least 80% or more identical, e.g., 85%, 85%, 87%, 88%, 89%, 90%, 91%, 92%, 93%, 94%, 95%, 96%, 97%, 98%, 99%, 99.5%, etc., i.e. up to 100% identical, to e.g., VP1, VP2 and/or VP3 sequence of an AAV capsid serotype selected from AAV1, AAV2, AAV3, AAV4, AAV5, AAV6, AAV7, AAV8, AAV9, AAV10, AAV11, AAV12, AAV13, AAV14, AAV15, AAV16, AAVS3, AAV.rh8, AAV.rh10, AAV.rh20, AAV.rh39, AAV.rh46, AAV.rh73, AAV.Rh74, AAV.RHM4-1, AAV.hu37, AAV.Anc80, rAAV.Anc80L65, AAV.7m8, AAV.PHP.B, AAV.PHP.eB, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, or AAV.HSC16, or a derivative, modification, or pseudotype thereof.

In particular embodiments, the recombinant AAV for us in compositions and methods herein is AAVS3 (including variants thereof) (see e.g., US Patent Application No. 20200079821, which is incorporated herein by reference in its entirety). In particular embodiments, rAAV particles comprise the capsids of AAV-LK03 or AAV3B, as described in Puzzo et al., 2017, Sci. Transl. Med. 29(9): 418, which is incorporated by reference in its entirety. In particular embodiments, the AAV for use in compositions and methods herein is any AAV disclosed in U.S. Pat. No. 10,301,648, such as AAV.rh46 or AAV.rh73. In some embodiments, the recombinant AAV for use in compositions and methods herein is Anc80 or Anc80L65 (see, e.g., Zinn et al., 2015, Cell Rep. 12(6): 1056-1068, which is incorporated by reference in its entirety). In particular embodiments, the AAV for use in compositions and methods herein is any AAV disclosed in U.S. Pat. No. 9,585,971, such as AAV-PHP.B. In particular embodiments, the AAV for use in compositions and methods herein is an AAV2/Rec2 or AAV2/Rec3 vector, which has hybrid capsid sequences derived from AAV8 and serotypes cy5, rh20 or rh39 (see, e.g., Issa et al., 2013, PLoS One 8(4): e60361, which is incorporated by reference herein for these vectors). In particular embodiments, the AAV for use in compositions and methods herein is an AAV disclosed in any of the following, each of which is incorporated herein by reference in its entirety: U.S. Pat. Nos. 7,282,199; 7,906,111; 8,524,446; 8,999,678; 8,628,966; 8,927,514; 8,734,809; 9,284,357; 9,409,953; 9,169,299; 9,193,956; 9,458,517; 9,587,282; US 2015/0374803; US 2015/0126588; US 2017/0067908; US 2013/0224836; US 2016/0215024; US 2017/0051257; PCT/US2015/034799; and PCT/EP2015/053335. In some embodiments, rAAV particles have a capsid protein at least 80% or more identical, e.g., 85%, 85%, 87%, 88%, 89%, 90%, 91%, 92%, 93%, 94%, 95%, 96%, 97%, 98%, 99%, 99.5%, etc., i.e. up to 100% identical, to the VP1, VP2 and/or VP3 sequence of an AAV capsid disclosed in any of the following patents and patent applications, each of which is incorporated herein by reference in its entirety: U.S. Pat. Nos. 7,282,199; 7,906,111; 8,524,446; 8,999,678; 8,628,966; 8,927,514; 8,734,809; 9,284,357; 9,409,953; 9,169,299; 9,193,956; 9,458,517; and 9,587,282; US patent application publication nos. 2015/0374803; 2015/0126588; 2017/0067908; 2013/0224836; 2016/0215024; 2017/0051257; and International Patent Application Nos. PCT/US2015/034799; PCT/EP2015/053335.

In some embodiments, rAAV particles comprise any AAV capsid disclosed in U.S. Pat. No. 9,840,719 and WO 2015/013313, such as AAV.Rh74 and RHM4-1, each of which is incorporated herein by reference in its entirety. In some embodiments, rAAV particles comprise any AAV capsid disclosed in WO 2014/172669, such as AAV rh.74, which is incorporated herein by reference in its entirety. In some embodiments, rAAV particles comprise the capsid of AAV2/5, as described in Georgiadis et al., 2016, Gene Therapy 23: 857-862 and Georgiadis et al., 2018, Gene Therapy 25: 450, each of which is incorporated by reference in its entirety. In some embodiments, rAAV particles comprise any AAV capsid disclosed in WO 2017/070491, such as AAV2tYF, which is incorporated herein by reference in its entirety. In some embodiments, rAAV particles comprise any AAV capsid disclosed in U.S. Pat. Nos. 8,628,966; 8,927,514; 9,923,120 and WO 2016/049230, such as HSC1, HSC2, HSC3, HSC4, HSC5, HSC6, HSC7, HSC8, HSC9, HSC10, HSC11, HSC12, HSC13, HSC14, HSC15, or HSC16, each of which is incorporated by reference in its entirety.

In some embodiments, rAAV particles have a capsid protein disclosed in Intl. Appl. Publ. No. WO 2003/052051 (see, e.g., SEQ ID NO: 2 of '051 publication), WO 2005/033321 (see, e.g., SEQ ID NOs: 123 and 88 of '321 publication), WO 03/042397 (see, e.g., SEQ ID NOs: 2, 81, 85, and 97 of '397 publication), WO 2006/068888 (see, e.g., SEQ ID NOs: 1 and 3-6 of '888 publication), WO 2006/110689, (see, e.g., SEQ ID NOs: 5-38 of '689 publication) WO2009/104964 (see, e.g., SEQ ID NOs: 1-5, 7, 9, 20, 22, 24 and 31 of '964 publication), WO 2010/127097 (see, e.g., SEQ ID NOs: 5-38 of '097 publication), and WO 2015/191508 (see, e.g., SEQ ID NOs: 80-294 of '508 publication), and U.S. Appl. Publ. No. 20150023924 (see, e.g., SEQ ID NOs: 1, 5-10 of '924 publication), the contents of each of which is herein incorporated by reference in its entirety. In some embodiments, rAAV particles have a capsid protein at least 80% or more identical, e.g., 85%, 85%, 87%, 88%, 89%, 90%, 91%, 92%, 93%, 94%, 95%, 96%, 97%, 98%, 99%, 99.5%, etc., i.e. up to 100% identical, to the VP1, VP2 and/or VP3 sequence of an AAV capsid disclosed in Intl. Appl. Publ. No. WO 2003/052051 (see, e.g., SEQ ID NO: 2 of '051 publication), WO 2005/033321 (see, e.g., SEQ ID NOs: 123 and 88 of '321 publication), WO 03/042397 (see, e.g., SEQ ID NOs: 2, 81, 85, and 97 of '397 publication), WO 2006/068888 (see, e.g., SEQ ID NOs: 1 and 3-6 of '888 publication), WO 2006/110689 (see, e.g., SEQ ID NOs: 5-38 of '689 publication) WO2009/104964 (see, e.g., SEQ ID NOs: 1-5, 7, 9, 20, 22, 24 and 31 of 964 publication), WO 2010/127097 (see, e.g., SEQ ID NOs: 5-38 of '097 publication), and WO 2015/191508 (see, e.g., SEQ ID NOs: 80-294 of '508 publication), and U.S. Appl. Publ. No. 20150023924 (see, e.g., SEQ ID NOs: 1, 5-10 of '924 publication).

In additional embodiments, rAAV particles comprise a pseudotyped AAV capsid. In some embodiments, the pseudotyped AAV capsids are rAAV2/8 or rAAV2/9 pseudotyped AAV capsids. Methods for producing and using pseudotyped rAAV particles are known in the art (see, e.g., Duan et al., J. Virol., 75:7662-7671 (2001); Halbert et al., J. Virol., 74:1524-1532 (2000); Zolotukhin et al., Methods 28:158-167 (2002); and Auricchio et al., Hum. Molec. Genet. 10:3075-3081, (2001).

AAV8-based, AAV3B-based, and AAVrh73-based viral vectors are used in certain of the methods described herein. Nucleotide sequences of AAV based viral vectors and methods of making recombinant AAV and AAV capsids are taught, for example, in U.S. Pat. No. 7,282,199 B2, U.S. Pat. No. 7,790,449 B2, U.S. Pat. No. 8,318,480 B2, U.S. Pat. No. 8,962,332 B2 and International Patent Application No. PCT/EP2014/076466, each of which is incorporated herein by reference in its entirety. In one aspect, provided herein are AAV (e.g., AAV8, AAV3B, AAVrh73, or AAVrh10)-based viral vectors encoding a transgene (e.g., an HuPTM Fab). The amino acid sequences of AAV capsids, including AAV8, AAV3B, AAVrh73 and AAVrh10 are provided in FIG. 4.

In certain embodiments, a single-stranded AAV (ssAAV) may be used supra. In certain embodiments, a self-complementary vector, e.g., scAAV, may be used (see, e.g., Wu, 2007, Human Gene Therapy, 18(2):171-82, McCarty et al, 2001, Gene Therapy, Vol 8, Number 16, Pages 1248-1254; and U.S. Pat. Nos. 6,596,535; 7,125,717; and 7,456,683, each of which is incorporated herein by reference in its entirety).

In certain embodiments, the viral vectors used in the methods described herein are adenovirus based viral vectors. A recombinant adenovirus vector may be used to transfer in the transgene encoding the HuPTMmAb or HuGlyFab or antigen-binding fragment. The recombinant adenovirus can be a first-generation vector, with an E1 deletion, with or without an E3 deletion, and with the expression cassette inserted into either deleted region. The recombinant adenovirus can be a second-generation vector, which contains full or partial deletions of the E2 and E4 regions. A helper-dependent adenovirus retains only the adenovirus inverted terminal repeats and the packaging signal (phi). The transgene is inserted between the packaging signal and the 3′ITR, with or without stuffer sequences to keep the genome close to wild-type size of approximately 36 kb. An exemplary protocol for production of adenoviral vectors may be found in Alba et al., 2005, “Gutless adenovirus: last generation adenovirus for gene therapy,” Gene Therapy 12:S18-S27, which is incorporated by reference herein in its entirety.

In certain embodiments, the viral vectors used in the methods described herein are lentivirus based viral vectors. A recombinant lentivirus vector may be used to transfer in the transgene encoding the HuPTM mAb antigen binding fragment. Four plasmids are used to make the construct: Gag/pol sequence containing plasmid, Rev sequence containing plasmids, Envelope protein containing plasmid (e.g., VSV-G), and Cis plasmid with the packaging elements and the anti-TNFα antigen-binding fragment gene.

For lentiviral vector production, the four plasmids are co-transfected into cells (e.g., HEK293 based cells), whereby polyethylenimine or calcium phosphate can be used as transfection agents, among others. The lentivirus is then harvested in the supernatant (lentiviruses need to bud from the cells to be active, so no cell harvest needs/should be done). The supernatant is filtered (0.45 μm) and then magnesium chloride and benzonase added. Further downstream processes can vary widely, with using TFF and column chromatography being the most GMP compatible ones. Others use ultracentrifugation with/without column chromatography. Exemplary protocols for production of lentiviral vectors may be found in Lesch et al., 2011, “Production and purification of lentiviral vector generated in 293T suspension cells with baculoviral vectors,” Gene Therapy 18:531-538, and Ausubel et al., 2012, “Production of CGMP-Grade Lentiviral Vectors,” Bioprocess Int. 10(2):32-43, both of which are incorporated by reference herein in their entireties.

In a specific embodiment, a vector for use in the methods described herein is one that encodes an HuPTM mAb, such that, upon introduction of the vector into a relevant cell, a glycosylated and/or tyrosine sulfated variant of the HuPTM mAb is expressed by the cell.

5.1.3 Promoters and Modifiers of Gene Expression

In certain embodiments, the vectors provided herein comprise components that modulate gene delivery or gene expression (e.g., “expression control elements”). In certain embodiments, the vectors provided herein comprise components that modulate gene expression. In certain embodiments, the vectors provided herein comprise components that influence binding or targeting to cells. In certain embodiments, the vectors provided herein comprise components that influence the localization of the polynucleotide (e.g., the transgene) within the cell after uptake. In certain embodiments, the vectors provided herein comprise components that can be used as detectable or selectable markers, e.g., to detect or select for cells that have taken up the polynucleotide.

In certain embodiments, the viral vectors provided herein comprise one or more promoters that control expression of the transgene. These promoters (and other regulatory elements that control transcription, such as enhancers) may be constitutive (promote ubiquitous expression) or may specifically or selectively express in the eye. In certain embodiments, the promoter is a constitutive promoter.

In certain embodiments, the promoter is a CB7 (also referred to as a CAG promoter) (see Dinculescu et al., 2005, Hum Gene Ther 16: 649-663, incorporated by reference herein in its entirety). In some embodiments, the CAG (SEQ ID NO: 74) or CB7 promoter (SEQ ID NO: 73) includes other expression control elements that enhance expression of the transgene driven by the vector. In certain embodiments, the other expression control elements include chicken β-actin intron and/or rabbit β-globin polyA signal (SEQ ID NO:78). In certain embodiments, the promoter comprises a TATA box. In certain embodiments, the promoter comprises one or more elements. In certain embodiments, the one or more promoter elements may be inverted or moved relative to one another. In certain embodiments, the elements of the promoter are positioned to function cooperatively. In certain embodiments, the elements of the promoter are positioned to function independently. In certain embodiments, the viral vectors provided herein comprise one or more promoters selected from the group consisting of the human CMV immediate early gene promoter, the SV40 early promoter, the Rous sarcoma virus (RS) long terminal repeat, and rat insulin promoter. In certain embodiments, the vectors provided herein comprise one or more long terminal repeat (LTR) promoters selected from the group consisting of AAV, MLV, MMTV, SV40, RSV, HIV-1, and HIV-2 LTRs.

In certain embodiments, the vectors provided herein comprise one or more tissue specific promoters (e.g., a retinal-specific promoter). In particular embodiments, the viral vectors provided herein comprises a ocular tissue cell specific promoter, such as, human rhodopsin kinase (GRK1) promoter (SEQ ID NOS:77 or 217), a mouse cone arresting (CAR) promoter (SEQ ID NOS: 214-216), or a human red opsin (RedO) promoter (SEQ ID NO: 212).

Provided are nucleic acid regulatory elements that are chimeric with respect to arrangements of elements in tandem in the expression cassette. Regulatory elements, in general, have multiple functions as recognition sites for transcription initiation or regulation, coordination with cell-specific machinery to drive expression upon signaling, and to enhance expression of the downstream gene.

In certain embodiments, the promoter is an inducible promoter. In certain embodiments the promoter is a hypoxia-inducible promoter. In certain embodiments, the promoter comprises a hypoxia-inducible factor (HIF) binding site. In certain embodiments, the promoter comprises a HIF-1α binding site. In certain embodiments, the promoter comprises a HIF-2a binding site. In certain embodiments, the HIF binding site comprises an RCGTG (SEQ ID NO:227) motif. For details regarding the location and sequence of HIF binding sites, see, e.g., Schödel, et al., Blood, 2011, 117(23):e207-e217, which is incorporated by reference herein in its entirety. In certain embodiments, the promoter comprises a binding site for a hypoxia induced transcription factor other than a HIF transcription factor. In certain embodiments, the viral vectors provided herein comprise one or more IRES sites that is preferentially translated in hypoxia. For teachings regarding hypoxia-inducible gene expression and the factors involved therein, see, e.g., Kenneth and Rocha, Biochem J., 2008, 414:19-29, which is incorporated by reference herein in its entirety. In specific embodiments, the hypoxia-inducible promoter is the human N-WASP promoter, see, e.g., Salvi, 2017, Biochemistry and Biophysics Reports 9:13-21 (incorporated by reference for the teaching of the N-WASP promoter) or is the hypoxia-induced promoter of human Epo, see, e.g., Tsuchiya et al., 1993, J. Biochem. 113:395-400 (incorporated by reference for the disclosure of the Epo hypoxia-inducible promoter). In other embodiments, the promoter is a drug inducible promoter, for example, a promoter that is induced by administration of rapamycin or analogs thereof. See, e.g., the disclosure of rapamycin inducible promoters in PCT publications WO94/18317, WO 96/20951, WO 96/41865, WO 99/10508, WO 99/10510, WO 99/36553, and WO 99/41258, and U.S. Pat. No. 7,067,526, which are hereby incorporated by reference in their entireties for the disclosure of drug inducible promoters.

Provided herein are constructs containing certain ubiquitous and tissue-specific promoters. Such promoters include synthetic and tandem promoters. Examples and nucleotide sequences of promoters are provided in Tables 1 and 1a below. Table 1 also includes the nucleotide sequences of other regulatory elements useful for the expression cassettes provided herein.

TABLE 1
Promoter and Other Regulatory Element Sequences

| Name/ | |
|---|---|
| SEQ ID NO. | Sequence |
| CAG/CB7 | gacattgattattgactagttattaatagtaatcaattacggggtcattagt |
| SEQ ID NO: 73 | tcatagcccatatatggagttccgcgttacataacttacggtaaatggcccg |
| | cctggctgaccgcccaacgacccccgcccattgacgtcaataatgacgtatg |
| | ttcccatagtaacgccaatagggactttccattgacgtcaatgggtggagta |
| | tttacggtaaactgcccacttggcagtacatcaagtgtatcatatgccaagt |
| | acgccccctattgacgtcaatgacggtaaatggcccgcctggcattatgccc |
| | agtacatgaccttatgggactttcctacttggcagtacatctacgtattagt |
| | catcgctattaccatggtcgaggtgagccccacgttctgcttcactctcccc |
| | atctcccccccctccccacccccaattttgtatttatttattttttaattat |
| | tttgtgcagcgatgggggcggggggggggggggggcgcgcgccaggcggggc |
| | ggggcggggcgaggggcggggcggggcgaggcggagaggtgcggcggcagcc |
| | aatcagagcggcgcgctccgaaagtttccttttatggcgaggcggcggcggc |
| | ggcggccctataaaaagcgaagcgcgcggcgggcgggagtcgctgcgcgctg |
| | ccttcgccccgtgccccgctccgccgccgcctcgcgccgcccgccccggctc |
| | tgactgaccgcgttactcccacaggtgagcgggcgggacggcccttctcctc |
| | cgggctgtaattagcgcttggtttaatgacggcttgtttcttttctgtggct |
| | gcgtgaaagccttgaggggctccgggagggccctttgtgcggggggagcggc |
| | tcggggggtgcgtgcgtgtgtgtgtgcgtggggagcgccgcgtgcggctccg |
| | cgctgcccggcggctgtgagcgctgcgggcgcggcgcggggctttgtgcgct |
| | ccgcagtgtgcgcgaggggagcgcggccgggggcggtgccccgcggtgcggg |
| | gggggctgcgaggggaacaaaggctgcgtgcggggtgtgtgcgtgggggggt |
| | gagcagggggtgtgggcgcgtcggtcgggctgcaaccccccctgcacccccc |
| | tccccgagttgctgagcacggcccggcttcgggtgcggggctccgtacgggg |
| | cgtggcgcggggctcgccgtgccgggcggggggtggcggcaggtgggggtgc |
| | cgggcggggcggggccgcctcgggccggggagggctcgggggaggggcgcgg |
| | cggcccccggagcgccggcggctgtcgaggcgcggcgagccgcagccattgc |
| | cttttatggtaatcgtgcgagagggcgcagggacttcctttgtcccaaatct |
| | gtgcggagccgaaatctgggaggcgccgccgcaccccctctagcgggcgcgg |
| | ggcgaagcggtgcggcgccggcaggaaggaaatgggcggggagggccttcgt |
| | gcgtcgccgcgccgccgtccccttctccctctccagcctcggggctgtccgc |
| | ggggggacggctgccttcgggggggacggggcagggcggggttcggcttctg |
| | gcgtgtgaccggcggctctagagcctctgctaaccatgttcatgccttcttc |
| | tttttcctacagctcctgggcaacgtgctggttattgtgctgtctcatcatt |
| | ttggcaaag |
| CAG (CMV- | gacattgattattgactagttattaatagtaatcaattacggggtcattagt |
| Chicken B-Actin | tcatagcccatatatggagttccgcgttacataacttacggtaaatggcccg |
| Promoter- | cctggctgaccgcccaacgacccccgcccattgacgtcaataatgacgtatg |
| chimeric intron) | ttcccatagtaacgccaatagggactttccattgacgtcaatgggtggagta |
| SEQ ID NO: 74 | tttacggtaaactgcccacttggcagtacatcaagtgtatcatatgccaagt |
| | acgccccctattgacgtcaatgacggtaaatggcccgcctggcattatgccc |
| | agtacatgaccttatgggactttcctacttggcagtacatctacgtattagt |
| | catcgctattaccatggtcgaggtgagccccacgttctgcttcactctcccc |
| | atctcccccccctccccacccccaattttgtatttatttattttttaattat |
| | tttgtgcagcgatgggggcggggggggggggggggcgcgcgccaggcggggc |
| | ggggcggggcgaggggcggggcggggcgaggcggagaggtgcggcggcagcc |
| | aatcagagcggcgcgctccgaaagtttccttttatggcgaggcggcggcggc |
| | ggcggccctataaaaagcgaagcgcgcggcgggcgggagtcgctgcgcgctg |
| | ccttcgccccgtgccccgctccgccgccgcctcgcgccgcccgccccggctc |
| | tgactgaccgcgttactcccacaggtgagcgggcgggacggcccttctcctc |
| | cgggctgtaattagcgcttggtttaatgacggcttgtttcttttctgtggct |
| | gcgtgaaagccttgaggggctccgggagggccctttgtgcggggggagcggc |
| | tcggggggtgcgtgcgtgtgtgtgtgcgtggggagcgccgcgtgcggctccg |
| | cgctgcccggcggctgtgagcgctgcgggcgcggcgcggggctttgtgcgct |
| | ccgcagtgtgcgcgaggggagcgcggccgggggcggtgccccgcggtgcggg |
| | gggggctgcgaggggaacaaaggctgcgtgcggggtgtgtgcgtgggggggt |
| | gagcagggggtgtgggcgcgtcggtcgggctgcaaccccccctgcacccccc |
| | tccccgagttgctgagcacggcccggcttcgggtgcggggctccgtacgggg |
| | cgtggcgcggggctcgccgtgccgggcggggggtggcggcaggtgggggtgc |
| | cgggcggggcggggccgcctcgggccggggagggctcgggggaggggcgcgg |
| | cggcccccggagcgccggcggctgtcgaggcgcggcgagccgcagccattgc |
| | cttttatggtaatcgtgcgagagggcgcagggacttcctttgtcccaaatct |
| | gtgcggagccgaaatctgggaggcgccgccgcaccccctctagcgggcgcgg |
| | ggcgaagcggtgcggcgccggcaggaaggaaatgggcggggagggccttcgt |
| | gcgtcgccgcgccgccgtccccttctccctctccagcctcggggctgtccgc |
| | ggggggacggctgccttcgggggggacggggcagggcggggttcggcttctg |
| | gcgtgtgaccggcggctctagagcctctgctaaccatgttcatgccttcttc |
| | tttttcctacag |
| mU1a | atggaggcggtactatgtagatgagaattcaggagcaaactgggaaaagcaactgcttccaaa |
| SEQ ID NO: 75 | tatttgtgatttttacagtgtagttttggaaaaactcttagcctaccaattcttctaagtgtt |
| | ttaaaatgtgggagccagtacacatgaagttatagagtgttttaatgaggcttaaatatttac |
| | cgtaactatgaaatgctacgcatatcatgctgttcaggctccgtggccacgcaactcatact |
| EF-1α (core) | gggcagagcgcacatcgcccacagtccccgagaagttggggggaggggtcggcaattgaacgg |
| SEQ ID NO: 76 | gtgcctagagaaggtggcgcggggtaaactgggaaagtgatgtcgtgtactggctccgccttt |
| | ttcccgagggtgggggagaaccgtatataagtgcagtagtcgccgtgaacgttctttttcgca |
| | acgggtttgccgccagaacacag |
| Human UbC | Gtctaacaaaaaagccaaaaacggccagaatttagcggacaatttactagtc |
| SEQ ID NO: 211 | taacactgaa |
| | Aattacatattgacccaaatgattacatttcaaaaggtgcctaaaaaacttc |
| | acaaaacacactCgccaaccccgagcgcatagttcaaaaccggagcttcagc |
| | tacttaagaagataggtacataaAaccgaccaaagaaactgacgcctcactt |
| | atccctcccctcaccagaggtccggcgcctgtcgaTtcaggagagcctaccc |
| | taggcccgaaccctgcgtcctgcgacggagaaaagcctaccgcacaCctacc |
| | ggcaggtggccccaccctgcattataagccaacagaacgggtgacgtcacga |
| | cacgaCgagggcgcgcgctcccaaaggtacgggtgcactgcccaacggcacc |
| | gccataactgccgcccCcgcaacagacgacaaaccgagttctccagtcagtg |
| | acaaacttcacgtcagggtccccagatgGtgccccagcccatctcacccgaa |
| | taagagctttcccgcattagcgaaggcctcaagaccttgggTtcttgccgcc |
| | caccatgccccccaccttgtttcaacgacctcacagcccgcctcacaagcgt |
| | cttcCattcaagactcgggaacagccgccattttgctgcgctccccccaacc |
| | cccagttcagggcaaccTtgctcgcggacccagactacagcccttggcggtc |
| | tctccacacgcttccgtcccaccgagcggccCggcggccacgaaagccccgg |
| | ccagcccagcagcccgctactcaccaagtgacgatcacagcgaTccacaaac |
| | aagaaccgcgacccaaatcccggctgcgacggaactagctgtgccacacccg |
| | gcgCgtccttatataatcatcggcgttcaccgccccacggagatccctccgc |
| | agaatcgccgagaagggActacttttcctcgcctgttccgctctctggaaag |
| | aaaaccagtgccctagagtcacccaagtcccgtCctaaaatgtccttctgct |
| | gatactggggttctaaggccgagtcttatgagcagcgggccgctgtcctGag |
| | cgtccgggcggaaggatcaggacgctcgctgcgcccttcgtctgacgtggca |
| | gcgctcgccgtgaggaggggggcgcccgcgggaggcgccaaaacccggcgcg |
| | gaggcc |
| CB Promoter | gacgacattgattattgactagttattaatagtaatcaattacggggtcatt |
| SEQ ID NO: 273 | agttcatagcccatatatggagttccgcgttacataacttacggtaaatggc |
| | ccgcctggctgaccgcccaacgacccccgcccattgacgtcaataatgacgt |
| | atgttcccatagtaacgccaatagggactttccattgacgtcaatgggtgga |
| | gtatttacggtaaactgcccacttggcagtacatcaagtgtatcatatgcca |
| | agtacgccccctattgacgtcaatgacggtaaatggcccgcctggcattatg |
| | cccagtacatgaccttatgggactttcctacttggcagtacatctacgtatt |
| | agtcatcgctattaccatggtcgaggtgagccccacgttctgcttcactctc |
| | cccatctcccccccctccccacccccaattttgtatttatttattttttaat |
| | tattttgtgcagcgatgggggcggggggggggggggggcgcgcgccaggcgg |
| | ggcggggcggggcgaggggcggggcggggcgaggcggagaggtgcggcggca |
| | gccaatcagagcggcgcgctccgaaagtttccttttatggcgaggcggcggc |
| | ggcggcggccctataaaaagcgaagcgcgcggcgggcgg |
| CB Long | gacgacattgattattgactagttattaatagtaatcaattacggggtcatt |
| Promoter SEQ | agttcatagcccatatatggagttccgcgttacataacttacggtaaatggc |
| ID NO: 274 | ccgcctggctgaccgcccaacgacccccgcccattgacgtcaataatgacgt |
| | atgttcccatagtaacgccaatagggactttccattgacgtcaatgggtgga |
| | gtatttacggtaaactgcccacttggcagtacatcaagtgtatcatatgcca |
| | agtacgccccctattgacgtcaatgacggtaaatggcccgcctggcattatg |
| | cccagtacatgaccttatgggactttcctacttggcagtacatctacgtatt |
| | agtcatcgctattaccatggtcgaggtgagccccacgttctgcttcactctc |
| | cccatctcccccccctccccacccccaattttgtatttatttattttttaat |
| | tattttgtgcagcgatgggggcggggggggggggggggcgcgcgccaggcgg |
| | ggcggggcggggcgaggggcggggcggggcgaggcggagaggtgcggcggca |
| | gccaatcagagcggcgcgctccgaaagtttccttttatggcgaggcggcggc |
| | ggcggcggccctataaaaagcgaagcgcgcggcgggcgggagtcgctgcgcg |
| | ctgccttcgccccgtgccccgctccgccgccgcctcgcgccgcccgccccgg |
| | ctctgactgaccgcgttactcccacaggtgagcg |
| Human red opsin | ggaggctgaggggggggaaagggcatgggtgtttcatgaggacagagcttc |
| (RedO) | cgtttcat |
| (photoreceptor- | gcaatgaaaagagtttggagacggatggtggtgactggactatacacttaca |
| specific) | cacggtag |
| SEQ ID NO: 212 | cgatggtacactttgtattatgtatattttaccacgatctttttaaagtgtc |
| | aaaggcaa |
| | atggccaaatggttccttgtcctatagctgtagcagccatcggctgttagtg |
| | acaaagcc |
| | cctgagtcaagatgacagcagcccccataactcctaatcggctctcccgcgt |
| | ggagtcat |
| | ttaggagtagtcgcattagagacaagtccaacatctaatcttccaccctggc |
| | cagggccc |
| | cagctggcagcgagggtgggagactccgggcagagcagagggcgctgacatt |
| | ggggcccg |
| | gcctggcttgggtccctctggcctttccccaggggccctctttccttggggc |
| | tttcttgg |
| | gccgccactgctcccgctcctctccccccatcccaccccctcaccccctcgt |
| | tcttcata |
| | tccttctctagtgctccctccactttcatccacccttctgcaagagtgtggg |
| | accacaaa |
| | tgagttttcacctggcctggggacacacgtgcccccacaggtgctgagtgac |
| | tttctagg |
| | acagtaatctgctttaggctaaaatgggacttgatcttctgttagccctaat |
| | catcaatt |
| | agcagagccggtgaaggtgcagaacctaccgcctttccaggcctcctcccac |
| | ctctgcca |
| | cctccactctccttcctgggatgtgggggctggcacacgtgtggcccagggc |
| | attggtgg |
| | gattgcactgagctgggtcattagcgtaatcctggacaagggcagacagggc |
| | gagcggag |
| | ggccagctccggggctcaggcaaggctgggggcttcccccagacaccccact |
| | cctcctct |
| | gctggacccccacttcatagggcacttcgtgttctcaaagggcttccaaata |
| | gcatggtg |
| | gccttggatgcccagggaagcctcagagttgcttatctccctctagacagaa |
| | ggggaatc |
| | tcggtcaagagggagaggtcgccctgttcaaggccacccagccagctcatgg |
| | cggtaatg |
| | ggacaaggctggccagccatcccaccctcagaagggacccggtggggcaggt |
| | gatctcag |
| | aggaggctcacttctgggtctcacattcttggatccggttccaggcctcggc |
| | cctaaata |
| | gtctccctgggctttcaagagaaccacatgagaaaggaggattcgggctctg |
| | agcagttt |
| | caccacccaccccccagtctgcaaatcctgacccgtgggtccacctgcccca |
| | aaggcgga |
| | cgcaggacagtagaagggaacagagaacacataaacacagagagggccacag |
| | cggctccc |
| | acagtcaccgccaccttcctggcggggatgggtggggcgtctgagtttggtt |
| | cccagcaa |
| | atccctctgagccgcccttgcgggctcgcctcaggagcaggggagcaagagg |
| | tgggagga |
| | ggaggtctaagtcccaggcccaattaagagatcaggtagtgtagggtttggg |
| | agctttta |
| | aggtgaagaggcccgggctgatcccacaggccagtataaagcgccgtgaccc |
| | tcaggtga |
| | tgcgccagggccggctgccgtcggggacagggctttccatagcc |
| Human | Agatcttcccc a cctagccacc tggcaaactg ctccttctct |
| rhodopsin (Rho) | caaaggccca aacatggcct cccagactgc aacccccagg |
| (photoreceptor- | cagtcaggcc ctgtctccac aacctcacag ccaccctgga |
| specific) | cggaatctgc ttcttcccac atttgagtcc tcctcagccc |
| SEQ ID NO: 213 | ctgagctcct ctgggcaggg ctgtttcttt ccatctttgt |
| | attcccaggg gcctgcaaat aaatgtttaa tgaacgaaca |
| | agagagtgaa ttccaattcc atgcaacaag gattgggctc |
| | ctgggcccta ggctatgtgt ctggcaccag aaacggaagc |
| | tgcaggttgc agcccctgcc ctcatggagc tcctcctgtc |
| | agaggagtgt ggggactgga tgactccaga ggtaacttgt |
| | gggggaacga acaggtaagg ggctgtgtga cgagatgaga |
| | gactgggaga ataaaccaga aagtctctag ctgtccagag |
| | gacatagcac agaggcccat |
| | ggtccctatt tcaaacccag gccaccagac tgagctggga |
| | ccttgggaca gacaagtcat gcagaagtta ggggaccttc |
| | tcctcccttt tcctggatgg atcctgagta ccttctcctc |
| | cctgacctca ggcttcctcc tagtgtcacc ttggcccctc |
| | ttagaagcca attaggccct cagtttctgc agcggggatt |
| | aatatgatta tgaacacccc caatctccca gatgctgatt |
| | cagccaggag cttaggaggg ggaggtcact ttataagggt |
| | ctgggggggt cagaacccag |
| | agtc |
| | (start of exon 1 at 5339; See also Bennett, J., Sun, D. and |
| | Kariko, K. Sequence analysis of the 5.34-kb 5′ flanking region |
| | of the human rhodopsin-encoding gene. Gene 167 (1-2), 317-320 |
| | (1995)) |
| Mouse cone | TGGCCCTGGCATTCCCCTATACTGGGACATAGAACCTTCACAGGACCAAGGG |
| arrestin promoter | CCTCTCCTCCCATTGATGACTGACTAGGCCATCCTCTAGCTACATAGGTGGT |
| (CAR) | GGAGCCTAGAGTCCCTCCTTGTGTACTCTTTGGTGGTGGTTACTCTGGGGGT |
| (photoreceptor- | ACTAGGTTAGTTCGTATTGTTGTTCCTCCTAGGGGACTGCAAACCCCTTCAG |
| specific) | CTCCTTGGGTCCTTTCTCTAGTTCCTTCTTTGGGGACCCTGTGCTCAGTTCA |
| SEQ ID NO: 214 | ATGGATGGCCAATTTCCTTCTTAAATGCCCCTAGCAGTAACTGTTAGGTCTC |
| | AATCCCAAGACAAATGTCTGAGGTGCCTATTTAACAGATCAAAGCGGACCTG |
| | TCCTCAGGTTAACCCAGTCACTCCCTGTACCTCAGTCCCTACCCATCACAAT |
| | TCTCCAGCCCATGAGCTTCGGGCTGTACTTCCCCAACGGGTTCTCCCATTTT |
| | GGGTACATGGCCTTTTTTTTTTACCTTTTTGGTTCCTTTGGCCTTTTGGCTT |
| | TTGGCTTCCAGGGCTTCTGGATCCCCCCCAACCCCTCCCATACACA |
| | TACACATGTGCACTCGTGCACTCAACCCAGCACAGGATAATGTTCATTCTTG |
| | ACCTTTCCACATACATCTGGCTATGTTCTCTCTCTTATCTACAATAAATCTC |
| | CTCCACTATACTTAGGAGCAGTTATGTTCTTCTTCTTTCTTTCTTTTTTTTT |
| | TTTTTTTCATTCAGTAACATCATCAGAATCCCCTAGCTCTGGCCTACCTCCT |
| | CAGTAACAATCAGCTGATCCCTGGCCACTAATCTGTACTCACTAATCTGTTT |
| | TCCAAACTCTTGGCCCCTGAGCTAATTATAGCAGTGCTTCATGCCACCCACC |
| | CCAACCCTATCCTTGTTCTCTGACTCCCACTAATCTACACATTCAGAGGATT |
| | GTGGATATAAGAGGCTGGGAGGCCAGCTTAGCAACCAGAGCTCGAGGCTGAT |
| | GCGAGCTTCATCTCTTCCCTC |
| Mouse cone | TTTTTTTTTTACCTTTTTGGTTCCTTTGGCCTTTTGGCTTTTGGCTTCCAGG |
| arrestin promoter | GCTTCTGGATCCCCCCCAACCCCTCCCATACACATACACATGTGCACTCGTG |
| (CAR) | CACTCAACCCAGCACAGGATA |
| (photoreceptor- | ATGTTCATTCTTGACCTTTCCACATACATCTGGCTATGTTCTCTCTCTTATC |
| specific) | TACAATAAATCTCCTCCACTATACTTAGGAGCAGTTATGTTCTTCTTCTTTC |
| SEQ ID NO: 215 | TTTCTTTTTTTTTTTTTTTTCATTCAGTAACATCATCAGAATCCCCTAGCTC |
| | TGGCCTACCTCCTCAGTAACAATCAGCTGATCCCTGGCCACTAATCTGTACT |
| | CACTAATCTGTTTTCCAAACTCTTGGCCCCTGAGCTAATTATAGCAGTGCTT |
| | CATGCCACCCACCCCAACCCTATCCTTGTTCTCTGACTCCCACTAATCTACA |
| | CATTCAGAGGATTGTGGATATAAGAGGCTGGGAGGCCAGCTTAGCAACCAGA |
| | GCTCGAGGCTGATGCGAGCTTCATCTCTTCCCTC |
| Mouse cone | TGATCCCTGGCCACTAATCTGTACTCACTAATCTGTTTTCCAAACTCTTGGC |
| arrestin promoter | CCCTGAGCTAATTATAGCAGTGCTTCATGCCACCCACCCCAACCCTATCCTT |
| (CAR) | GTTCTCTGACTCCCACTAATCTACACATTCAGAGGATTGTGGATATAAGAGG |
| (photoreceptor- | CTGGGAGGCCAGCTTAGCAACCAGAGCTCGAGGCTGATGCGAGCTTCATCTC |
| specific) | TTCCCTC |
| SEQ ID NO: 216 | |
| Human | gggcccca gaagcctggt ggttgtttgt ccttctcagg ggaaaagtga |
| rhodopsin kinase | ggcggcccct tggaggaagg ggccgggcag aatgatctaa |
| (RK) or GRK1 | tcggattcca agcagctcag gggattgtct ttttctagca |
| (photoreceptor- | ccttcttgcc actcctaagc gtcctccgtg accccggctg |
| specific) | ggatttagcc tggtgctgtg tcagccccgg |
| SEQ ID NO: 217 | |
| Human | AAGG ACTCCTTTGT GGAGGTCCTG GCTTAGGGAG TCAAGTGACG |
| Bestrophin 1 | GCGGCTCAGC ACTCACGTGG GCAGTGCCAG CCTCTAAGAG |
| (BEST1) | TGGGCAGGGG CACTGGCCAC AGAGTCCCAG GGAGTCCCAC |
| promoter | CAGCCTAGTC GCCAGACC +38 |
| (a.k.a. hVMD2 | |
| promoter) | |
| SEQ ID NO: 218 | |
| Human | GTCAAGTGACG GCGGCTCAGC ACTCACGTGG GCAGTGCCAG |
| Bestrophin 1 | CCTCTAAGAG TGGGCAGGGG CACTGGCCAC AGAGTCCCAG |
| (BEST1) | GGAGTCCCAC CAGCCTAGTC |
| promoter | GCCAGACC |
| (a.k.a. hVMD2 | (transcriptional start site residue underlined at +1) |
| promoter) | |
| SEQ ID NO: 219 | |
| Human | CTCAGC ACTCACGTGG GCAGTGCCAG CCTCTAAGAG TGGGCAGGGG |
| Bestrophin 1 | CACTGGCCAC |
| (BEST1) | AGAGTCCCAG GGAGTCCCAC CAGCCTAGTC GCCAGAC |
| promoter | (transcriptional start site residue underlined at +1) |
| (a.k.a. hVMD2 | |
| promoter) | |
| SEQ ID NO: 220 | |
| Human | TTAATAAACA TTTGGGCGAT TCTTACGGCC TCTAAAGACC |
| Bestrophin 1 | AAGAACCACT GCTGCCTAGA GCTCTGCTCT CTTCATTGAA |
| (BEST1) | CAATACAAGA GGAGTGTGTA GGTAGACACC CACCACTTCC |
| promoter | AACAGCTTAG GAGAGCCCTT GAGTATGGAT TGATGTATTA |
| (a.k.a. hVMD2 | AAATTTATTG AATCACATGC TGAGATTTTC ACCAGCTGCC |
| promoter) | CGTGGGGATC TGGGCATTTA TTCCCATATT GCACTGGCTG |
| SEQ ID NO: 221 | GCTGGAAGCC AGCAGCATAA ACTCCAGGGC TGTTCTGTCA |
| | ACCCCCACCA GACTCACCCC |
| | GCTCCACCAG CCCCGGCAGG CTTCTCCTTC CATCTCTCTG |
| | AAGCAACTTA CTGATGGGCC CTGCCAGCCA ATCACAGCCA |
| | GAATAACGTA TGATGTCACC AGCAGCCAAT CAGAGCTCCT |
| | CGTCAGCATA TGCAGAATTC TGTCATTTTA CTAGGGTGAT |
| | GAAATTCCCA AGCAACACCA TCCTTTTCAGATAAGGGCAC |
| | TGAGGCTGAG AGAGGAGCTG AAACCTACCC GGCGTCACCA |
| | CACACAGGTG GCAAGGCTGG GACCAGAAAC CAGGACTGTT |
| | GACTGCAGCC CGGTATTCAT |
| | TCTTTCCATA GCCCACAGGG CTGTCAAAGA CCCCAGGGCC |
| | TAGTCAGAGG CTCCTCCTTC CTGGAGAGTT CCTGGCACAG |
| | AAGTTGAAGC TCAGCACAGC CCCCTAACCC CCAACTCTCT |
| | CTGCAAGGCC TCAGGGGTCA GAACACTGGT GGAGCAGATC |
| | CTTTAGCCTC TGGATTTTAG GGCCATGGTA GAGGGGGTGT |
| | TGCCCTAAAT TCCAGCCCTG GTCTCAGCCC AACACCCTCC |
| | AAGAAGAAAT TAGAGGGGCC ATGGCCAGGC TGTGCTAGCC |
| | GTTGCTTCTG AGCAGATTAC |
| | AAGAAGGGAC CAAGACAAGG ACTCCTTTGT GGAGGTCCTG |
| | GCTTAGGGAG TCAAGTGACG GCGGCTCAGC ACTCACGTGG |
| | GCAGTGCCAG CCTCTAAGAG TGGGCAGGGG CACTGGCCAC |
| | AGAGTCCCAG GGAGTCCCAC CAGCCTAGTC GCCAGACCTT |
| | CTGTGGGATC ATCGGACCCA |
| | (transcriptional start site residue underlined at |
| | +1; minimal active promoter region in bold) |
| GRK1 promoter | GGGCCCCAGAAGCCTGGTGGTTGTTTGTCCTTCTCAGGGGAAAAGTGAGGCGGCCCCTTGGAG |
| SEQ ID NO: 77 | GAAGGGGCCGGGCAGAATGATCTAATCGGATTCCAAGCAGCTCAGGGGATTGTCTTTTTCTAG |
| | CACCTTCTTGCCACTCCTAAGCGTCCTCCGTGACCCCGGCTGGGATTTAGCCTGGTGCTGTGT |
| | CAGCCCCGGGCTCCCAGGGGCTTCCCAGTGGTCCCCAGGAACCCTCGACAGGGCCAGGGCGTC |
| | TCTCTCGTCCAGCAAGGGCAGGGACGGGCCACAGGCCAAGGGC |
| Best/GRK1 | GTCGACAAATTCTGTCATTTTACTAGGGTGATGAAATTCCCAAGCAACACCATCCTTTTCAGA |
| Tandem | TAAGGGCACTGAGGCTGAGAGAGGAGCTGAAACCTACCCGGGGTCACCACACACAGGTGGCAA |
| Promoter | GGCTGGGACCAGAAACCAGGACTGTTGACTGCAGCCCGGTATTCATTCTTTCCATAGCCCACA |
| SEQ ID NO: 275 | GGGCTGTCAAAGACCCCAGGGCCTAGTCAGAGGCTCCTCCTTCCTGGAGAGTTCCTGGCACAG |
| | AAGTTGAAGCTCAGCACAGCCCCCTAACCCCCAACTCTCTCTGCAAGGCCTCAGGGGTCAGAA |
| | CACTGGTGGAGCAGATCCTTTAGCCTCTGGATTTTAGGGCCATGGTAGAGGGGGTGTTGCCCT |
| | AAATTCCAGCCCTGGTCTCAGCCCAACACCCTCCAAGAAGAAATTAGAGGGGCCATGGCCAGG |
| | CTGTGCTAGCCGTTGCTTCTGAGCAGATTACAAGAAGGGACTAAGACAAGGACTCCTTTGTGG |
| | AGGTCCTGGCTTAGGGAGTCAAGTGACGGCGGCTCAGCACTCACGTGGGCAGTGCCAGCCTCT |
| | AAGAGTGGGCAGGGGCACTGGCCACAGAGTCCCAGGGAGTCCCACCAGCCTAGTCGCCAGAtC |
| | TAGAGGGCCCCAGAAGCCTGGTGGTTGTTTGTCCTTCTCAGGGGAAAAGTGAGGCGGCCCCTT |
| | GGAGGAAGGGGCCGGGCAGAAGATCTAATCGGATTCCAAGCAGCTCAGGGGATTGTCTTTTTC |
| | TAGCACCTTCTTGCCACTCCTAAGCGTCCTCCGTGACCCCGGCTGGGATTTAGCCTGGTGCTG |
| | TGTCAGCCCCGGGCTCCCAGGGGCTTCCCAGTGGTCCCCAGGAACCCTCGACAGGGCCAGGGC |
| | GTCTCTCTCGTCCAGCAAGGGCAGGGACGGGCCACAGGCCAAGGGCCTTAAGC |
| Upstream Kozak | GCCACC |
| sequence | |
| Rabbit beta- | gatctttttccctctgccaaaaattatggggacatcatgaagccccttgagcatctgacttct |
| globin polyA | ggctaataaaggaaatttattttcattgcaatagtgtgttggaattttttgtgtctctcactc |
| SEQ ID NO: 78 | g |
| Chimeric Intron | gtaagtatcaaggttacaagacaggtttaaggagaccaatagaaactgggcttgtcgagacag |
| SEQ ID NO: 79 | agaagactcttgcgtttctgataggcacctattggtcttactgacatccactttgcctttctc |
| | tccacag |
| VH4 Intron | gtgagtatctcagggatccagacatggggatatgggaggtgcctctgatcccagggctcactg |
| SEQ ID NO: 80 | tgggtctctctgttcacag |
| SV40 Intron | gtaagtttagtctttttgtcttttatttcaggtcccggatccggtggtggtgcaaatcaaaga |
| SEQ ID NO: 272 | actgctcctcagtggatgttgcctttacttctag |
| 84 | |
| 5′ITR | ctgcgcgctcgctcgctcactgaggccgcccgggcaaagcccgggcgtcgggcgacctttggt |
| SEQ ID NO: 81 | cgcccggcctcagtgagcgagcgagcgcgcagagagggagtggccaactccatcactaggggt |
| | tcct |
| 5′-ITR | ctgcgcgctcgctcgctcactgaggccgcccgggcaaagcccgggcgtcgggcgacctttggt |
| (Deleted D- | cgcccggcctcagtgagcgagcgagcgcgcagagagggagtgg |
| sequence for | |
| self- | |
| complimentary | |
| AAV) | |
| SEQ ID NO: 82 | |
| 3′-ITR AAV | gaacccctagtgatggagttggccactccctctctgcgcgctcgctcgctcactgaggccgcc |
| SEQ ID NO: 83 | cgggcaaagcccgggcgtcgggcgacctttggtcgcccggcctcagtgagcgagcgagcgcgc |
| | agagagggagtggccaa |
| 3′-ITR | ttggccactccctctctgcgcgctcgctcgctcactgaggccgggcgaccaaaggtcgcccga |
| (Deleted D- | cgcccgggctttgcccgggcggcctcagtgagcgagcgagcgcgcag |
| sequence for | |
| self- | |
| complimentary | |
| AAV) | |
| SEQ ID NO: 84 | |

TABLE 1a
Other regulatory sequences

| | |
|---|---|
| LSPX1 | aggttaatttttaaaaagcagtcaaaagtccaagtggcccttggcagcatttactctctctgt |
| SEQ ID NO: 228 | ttgctctggttaataatctcaggagcacaaacattccagatccaggttaatttttaaaaagca |
| | gtcaaaagtccaagtggcccttggcagcatttactctctctgtttgctctggttaataatctc |
| | aggagcacaaacattccagatccggcgcgccagggctggaagctacctttgtctagaaggctc |
| | agaggcacacaggagtttctgggctcaccctgcccccttccaacccctcagttcccatcctcc |
| | agcagctgtttgtgtgctgcctctgaagtccacactgaacaaacttcagcctactcatgtccc |
| | taaaatgggcaaacattgcaagcagcaaacagcaaacacacagccctccctgcctgctgacct |
| | tggagctggggcagaggtcagagacctctctgggcccatgccacctccaacatccactcgacc |
| | ccttggaatttcggtggagaggagcagaggttgtcctggcgtggtttaggtagtgtgagaggg |
| | gtacccggggatcttgctaccagtggaacagccactaaggattctgcagtgagagcagagggc |
| | cagctaagtggtactctcccagagactgtctgactcacgccaccccctccaccttggacacag |
| | gacgctgtggtttctgagccaggtacaatgactcctttcggtaagtgcagtggaagctgtaca |
| | ctgcccaggcaaagcgtccgggcagcgtaggcgggcgactcagatcccagccagtggacttag |
| | cccctgtttgctcctccgataactggggtgaccttggttaatattcaccagcagcctcccccg |
| | ttgcccctctggatccactgcttaaatacggacgaggacagggccctgtctcctcagcttcag |
| | gcaccaccactgacctgggacagt |
| LSXP2 | aggctcagaggcacacaggagtttctgggctcaccctgcccccttccaaccc |
| SEQ ID NO: 229 | ctcagttcccatcctccagcagctgtttgtgtgctgcctctgaagtccacac |
| | tgaacaaacttcagcctactcatgtccctaaaatgggcaaacattgcaagca |
| | gcaaacagcaaacacacagccctccctgcctgctgaccttggagctggggca |
| | gaggtcagagacctctctgggcccatgccacctccaacatccactcgacccc |
| | ttggaatttcggtggagaggagcagaggttgtcctggcgtggtttaggtagt |
| | gtgagagggtctagaaggctcagaggcacacaggagtttctgggctcaccct |
| | gcccccttccaacccctcagttcccatcctccagcagctgtttgtgtgctgc |
| | ctctgaagtccacactgaacaaacttcagcctactcatgtccctaaaatggg |
| | caaacattgcaagcagcaaacagcaaacacacagccctccctgcctgctgac |
| | cttggagctggggcagaggtcagagacctctctgggcccatgccacctccaa |
| | catccactcgaccccttggaatttcggtggagaggagcagaggttgtcctgg |
| | cgtggtttaggtagtgtgagaggggtacccggggatcttgctaccagtggaa |
| | cagccactaaggattctgcagtgagagcagagggccagctaagtggtactct |
| | cccagagactgtctgactcacgccaccccctccaccttggacacaggacgct |
| | gtggtttctgagccaggtacaatgactcctttcggtaagtgcagtggaagct |
| | gtacactgcccaggcaaagcgtccgggcagcgtaggcgggcgactcagatcc |
| | cagccagtggacttagcccctgtttgctcctccgataactggggtgaccttg |
| | gttaatattcaccagcagcctcccccgttgcccctctggatccactgcttaa |
| | atacggacgaggacagggccctgtctcctcagcttcaggcaccaccactgac |
| | ctgggacagt |
| LTP1 | aggttaatttttaaaaagcagtcaaaagtccaagtggcccttggcagcattt |
| SEQ ID NO: 230 | actctctctgtttgctctggttaataatctcaggagcacaaacattccagat |
| | ccaggttaatttttaaaaagcagtcaaaagtccaagtggcccttggcagcat |
| | ttactctctctgtttgctctggttaataatctcaggagcacaaacattccag |
| | atccggcgcgccagggctggaagctacctttgacatcatttcctctgcgaat |
| | gcatgtataatttctacagaacctattagaaaggatcacccagcctctgctt |
| | ttgtacaactttcccttaaaaaactgccaattccactgctgtttggcccaat |
| | agtgagaactttttcctgctgcctcttggtgcttttgcctatggcccctatt |
| | ctgcctgctgaagacactcttgccagcatggacttaaacccctccagctctg |
| | acaatcctctttctcttttgttttacatgaagggtctggcagccaaagcaat |
| | cactcaaagttcaaaccttatcattttttgctttgttcctcttggccttggt |
| | tttgtacatcagctttgaaaataccatcccagggttaatgctggggttaatt |
| | tataactaagagtgctctagttttgcaatacaggacatgctataaaaatgga |
| | aagatgttgctttctgagaggatcttgctaccagtggaacagccactaagga |
| | ttctgcagtgagagcagagggccagctaagtggtactctcccagagactgtc |
| | tgactcacgccaccccctccaccttggacacaggacgctgtggtttctgagc |
| | caggtacagtgactcctttcggtaagtgcagtggaagctgtacactgcccag |
| | gcaaagcgtccgggcagcgtaggcgggcgactcagatcccagccagtggact |
| | tagcccctgtttgctcctccgataactggggtgaccttggttaatattcacc |
| | agcagcctcccccgttgcccctctggatccactgcttaaatacggacgagga |
| | cagggccctgtctcctcagcttcaggcaccaccactgacctgggacagt |
| LTP2 | aggctcagaggcacacaggagtttctgggctcaccctgcccccttccaaccc |
| SEQ ID NO: 231 | ctcagttcccatcctccagcagctgtttgtgtgctgcctctgaagtccacac |
| | tgaacaaacttcagcctactcatgtccctaaaatgggcaaacattgcaagca |
| | gcaaacagcaaacacacagccctccctgcctgctgaccttggagctggggca |
| | gaggtcagagacctctctgggcccatgccacctccaacatccactcgacccc |
| | ttggaatttcggtggagaggagcagaggttgtcctggcgtggtttaggtagt |
| | gtgagagggtctagagcccttaagctagcaggttaatttttaaaaagcagtc |
| | aaaagtccaagtggcccttggcagcatttactctctctgtttgctctggtta |
| | ataatctcaggagcacaaacattccagatccaggttaatttttaaaaagcag |
| | tcaaaagtccaagtggcccttggcagcatttactctctctgtttgctctggt |
| | taataatctcaggagcacaaacattccagatccggcgcgccagggctggaag |
| | ctacctttgacatcatttcctctgcgaatgcatgtataatttctacagaacc |
| | tattagaaaggatcacccagcctctgcttttgtacaactttcccttaaaaaa |
| | ctgccaattccactgctgtttggcccaatagtgagaactttttcctgctgcc |
| | tcttggtgcttttgcctatggcccctattctgcctgctgaagacactcttgc |
| | cagcatggacttaaacccctccagctctgacaatcctctttctcttttgttt |
| | tacatgaagggtctggcagccaaagcaatcactcaaagttcaaaccttatca |
| | ttttttgctttgttcctcttggccttggttttgtacatcagctttgaaaata |
| | ccatcccagggttaatgctggggttaatttataactaagagtgctctagttt |
| | tgcaatacaggacatgctataaaaatggaaagatgttgctttctgagaggat |
| | cttgctaccagtggaacagccactaaggattctgcagtgagagcagagggcc |
| | agctaagtggtactctcccagagactgtctgactcacgccaccccctccacc |
| | ttggacacaggacgctgtggtttctgagccaggtacagtgactcctttcggt |
| | aagtgcagtggaagctgtacactgcccaggcaaagcgtccgggcagcgtagg |
| | cgggcgactcagatcccagccagtggacttagcccctgtttgctcctccgat |
| | aactggggtgaccttggttaatattcaccagcagcctcccccgttgcccctc |
| | tggatccactgcttaaatacggacgaggacagggccctgtctcctcagcttc |
| | aggcaccaccactgacctgggacagt |
| LTP3 | aggttaatttttaaaaagcagtcaaaagtccaagtggcccttggcagcattt |
| SEQ ID NO: 232 | actctctctgtttgctctggttaataatctcaggagcacaaacattccagat |
| | ccaggttaatttttaaaaagcagtcaaaagtccaagtggcccttggcagcat |
| | ttactctctctgtttgctctggttaataatctcaggagcacaaacattccag |
| | atccggcgcgccagggctggaagctacctttgacatcatttcctctgcgaat |
| | gcatgtataatttctacagaacctattagaaaggatcacccagcctctgctt |
| | ttgtacaactttcccttaaaaaactgccaattccactgctgtttggcccaat |
| | agtgagaactttttcctgctgcctcttggtgcttttgcctatggcccctatt |
| | ctgcctgctgaagacactcttgccagcatggacttaaacccctccagctctg |
| | acaatcctctttctcttttgttttacatgaagggtctggcagccaaagcaat |
| | cactcaaagttcaaaccttatcattttttgctttgttcctcttggccttggt |
| | tttgtacatcagctttgaaaataccatcccagggttaatgctggggttaatt |
| | tataactaagagtgctctagttttgcaatacaggacatgctataaaaatgga |
| | aagatgttgctttctgagaggatcttgctaccagtggaacagccactaagga |
| | ttctgcagtgagagcagagggccagctaagtggtactctcccagagactgtc |
| | tgactcacgccaccccctccaccttggacacaggacgctgtggtttctgagc |
| | caggtacagtgactcctttcggtaagtgcagtggaagctgtacactgcccag |
| | gcaaagcgtccgggcagcgtaggcgggcgactcagatcccagccagtggact |
| | tagcccctgtttgctcctccgataactggggtgaccttggttaatattcacc |
| | agcagcctcccccgttgcccctctggatccactgcttaaatacggacgagga |
| | cagggccctgtctcctcagcttcaggcaccaccactgacctgggacagtaaa |
| | acaggtaagtccgctgtttgtgtgctgcctctgaagtccacactgaacaaac |
| | ttcagcctactcatgtccctaaaatgggcaaacattgcaagcagcaaacagc |
| | aaacacacagccctccctgcctgctgaccttggagctggggcagaggtcaga |
| | gacctctctggcctctactaaccatgttcatgttttctttttttttctacag |
| | gtcctgggtgacgaacag |
| LMTP6 | aggctcagaggcacacaggagtttctgggctcaccctgcccccttccaaccc |
| SEQ ID NO: 233 | ctcagttcccatcctccagcagctgtttgtgtgctgcctctgaagtccacac |
| | tgaacaaacttcagcctactcatgtccctaaaatgggcaaacattgcaagca |
| | gcaaacagcaaacacacagccctccctgcctgctgaccttggagctggggca |
| | gaggtcagagacctctctgggcccatgccacctccaacatccactcgacccc |
| | ttggaatttcggtggagaggagcagaggttgtcctggcgtggtttaggtagt |
| | gtgagagggccactacgggtttaggctgcccatgtaaggaggcaaggcctgg |
| | ggacacccgagatgcctggttataattaacccagacatgtggctgccccccc |
| | cccccccaacacctgctgcctctaaaaataaccctgtccctggtggatccca |
| | ctacgggtttaggctgcccatgtaaggaggcaaggcctggggacacccgaga |
| | tgcctggttataattaacccagacatgtggctgccccccccccccccaacac |
| | ctgctgcctctaaaaataaccctgtccctggtggatcccactacgggtttag |
| | gctgcccatgtaaggaggcaaggcctggggacacccgagatgcctggttata |
| | attaacccagacatgtggctgccccccccccccccaacacctgctgcctcta |
| | aaaataaccctgtccctggtggatcccctgcatgcgaagatcttcgaacaag |
| | gctgtgggggactgagggcaggctgtaacaggcttgggggccagggcttata |
| | cgtgcctgggactcccaaagtattactgttccatgttcccggcgaagggcca |
| | gctgtcccccgccagctagactcagcacttagtttaggaaccagtgagcaag |
| | tcagcccttggggcagcccatacaaggccatggggctgggcaagctgcacgc |
| | ctgggtccggggtgggcacggtgcccgggcaacgagctgaaagctcatctgc |
| | tctcaggggcccctccctggggacagcccctcctggctagtcacaccctgta |
| | ggctcctctatataacccaggggcacaggggctgccctcattctaccaccac |
| | ctccacagcacagacagacactcaggagccagccagcgtcgagatcttgcta |
| | ccagtggaacagccactaaggattctgcagtgagagcagagggccagctaag |
| | tggtactctcccagagactgtctgactcacgccaccccctccaccttggaca |
| | caggacgctgtggtttctgagccaggtacagtgactcctttcggtaagtgca |
| | gtggaagctgtacactgcccaggcaaagcgtccgggcagcgtaggcgggcga |
| | ctcagatcccagccagtggacttagcccctgtttgctcctccgataactggg |
| | gtgaccttggttaatattcaccagcagcctcccccgttgcccctctggatcc |
| | actgcttaaatacggacgaggacagggccctgtctcctcagcttcaggcacc |
| | accactgacctgggacagt |
| LMTP13 | aggctcagaggcacacaggagtttctgggctcaccctgcccccttccaaccc |
| SEQ ID NO: 234 | ctcagttcccatcctccagcagctgtttgtgtgctgcctctgaagtccacac |
| | tgaacaaacttcagcctactcatgtccctaaaatgggcaaacattgcaagca |
| | gcaaacagcaaacacacagccctccctgcctgctgaccttggagctggggca |
| | gaggtcagagacctctctgggcccatgccacctccaacatccactcgacccc |
| | ttggaatttcggtggagaggagcagaggttgtcctggcgtggtttaggtagt |
| | gtgagaggggtacccggggatcttgctaccagtctagaggccgtccgccctc |
| | ggcaccatcctcacgacacccaaatatggcgacgggtgaggaatggtgggga |
| | gttatttttagagcggtgaggaaggtgggcaggcagcaggtgttggcgctct |
| | aaaaataactcccgggagttatttttagagcggaggaatggtggacacccaa |
| | atatggcgacggttcctcacccgtcgccatatttgggtgtccgccctcggcc |
| | ggggccgcattcctgggggccgggcggtgctcccgcccgcctcgataaaagg |
| | ctccggggccggcggcggcccacgagctacccggaggagcgggaggcgccaa |
| | gcgtgagtatcgatcttgctaccagtggaacagccactaaggattctgcagt |
| | gagagcagagggccagctaagtggtactctcccagagactgtctgactcacg |
| | ccaccccctccaccttggacacaggacgctgtggtttctgagccaggtacag |
| | tgactcctttcggtaagtgcagtggaagctgtacactgcccaggcaaagcgt |
| | ccgggcagcgtaggcgggcgactcagatcccagccagtggacttagcccctg |
| | tttgctcctccgataactggggtgaccttggttaatattcaccagcagcctc |
| | ccccgttgcccctctggatccactgcttaaatacggacgaggacagggccct |
| | gtctcctcagcttcaggcaccaccactgacctgggacagt |
| LMTP14 | gaatggtggacacccaaatatggcgacggttcctcacccgtcgccatatttg |
| SEQ ID NO: 235 | ggtgtccgccctcggccggggccgcattcctgggggccgggcggtgctcccg |
| | cccgcctcgataaaaggctccggggccggcggcggcccacgagctacccgga |
| | ggagcgggaggcgccaagcgatcttgctaccagtggaacagccactaaggat |
| | tctgcagtgagagcagagggccagctaagtggtactctcccagagactgtct |
| | gactcacgccaccccctccaccttggacacaggacgctgtggtttctgagcc |
| | aggtacagtgactcctttcggtaagtgcagtggaagctgtacactgcccagg |
| | caaagcgtccgggcagcgtaggcgggcgactcagatcccagccagtggactt |
| | agcccctgtttgctcctccgataactggggtgaccttggttaatattcacca |
| | gcagcctcccccgttgcccctctggatccactgcttaaatacggacgaggac |
| | agggccctgtctcctcagcttcaggcaccaccactgacctgggacagt |
| LMTP15 | aggctcagaggcacacaggagtttctgggctcaccctgcccccttccaaccc |
| SEQ ID NO: 236 | ctcagttcccatcctccagcagctgtttgtgtgctgcctctgaagtccacac |
| | tgaacaaacttcagcctactcatgtccctaaaatgggcaaacattgcaagca |
| | gcaaacagcaaacacacagccctccctgcctgctgaccttggagctggggca |
| | gaggtcagagacctctctgggcccatgccacctccaacatccactcgacccc |
| | ttggaatttcggtggagaggagcagaggttgtcctggcgtggtttaggtagt |
| | gtgagagggtctagagaatggtggacacccaaatatggcgacggttcctcac |
| | ccgtcgccatatttgggtgtccgccctcggccggggccgcattcctgggggc |
| | cgggcggtgctcccgcccgcctcgataaaaggctccggggccggcggcggcc |
| | cacgagctacccggaggagcgggaggcgccaagcgatcttgctaccagtgga |
| | acagccactaaggattctgcagtgagagcagagggccagctaagtggtactc |
| | tcccagagactgtctgactcacgccaccccctccaccttggacacaggacgc |
| | tgtggtttctgagccaggtacagtgactcctttcggtaagtgcagtggaagc |
| | tgtacactgcccaggcaaagcgtccgggcagcgtaggcgggcgactcagatc |
| | ccagccagtggacttagcccctgtttgctcctccgataactggggtgacctt |
| | ggttaatattcaccagcagcctcccccgttgcccctctggatccactgctta |
| | aatacggacgaggacagggccctgtctcctcagcttcaggcaccaccactga |
| | cctgggacagt |
| LMTP18 | aggctcagaggcacacaggagtttctgggctcaccctgcccccttccaaccc |
| SEQ ID NO: 237 | ctcagttcccatcctccagcagctgtttgtgtgctgcctctgaagtccacac |
| | tgaacaaacttcagcctactcatgtccctaaaatgggcaaacattgcaagca |
| | gcaaacagcaaacacacagccctccctgcctgctgaccttggagctggggca |
| | gaggtcagagacctctctgggcccatgccacctccaacatccactcgacccc |
| | ttggaatttcggtggagaggagcagaggttgtcctggcgtggtttaggtagt |
| | gtgagagggccactacgggtttaggctgcccatgtaaggaggcaaggcctgg |
| | ggacacccgagatgcctggttataattaacccagacatgtggctgccccccc |
| | cccccccaacacctgctgcctctaaaaataaccctgtccctggtggatcccc |
| | tgcatgcgaagatcttcgaacaaggctgtgggggactgagggcaggctgtaa |
| | caggcttgggggccagggcttatacgtgcctgggactcccaaagtattactg |
| | ttccatgttcccggcgaagggccagctgtcccccgccagctagactcagcac |
| | ttagtttaggaaccagtgagcaagtcagcccttggggcagcccatacaaggc |
| | catggggctgggcaagctgcacgcctgggtccggggtgggcacggtgcccgg |
| | gcaacgagctgaaagctcatctgctctcaggggcccctccctggggacagcc |
| | cctcctggctagtcacaccctgtaggctcctctatataacccaggggcacag |
| | gggctgccctcattctaccaccacctccacagcacagacagacactcaggag |
| | ccagccagcgtcgagatcttgctaccagtggaacagccactaaggattctgc |
| | agtgagagcagagggccagctaagtggtactctcccagagactgtctgactc |
| | acgccaccccctccaccttggacacaggacgctgtggtttctgagccaggta |
| | cagtgactcctttcggtaagtgcagtggaagctgtacactgcccaggcaaag |
| | cgtccgggcagcgtaggcgggcgactcagatcccagccagtggacttagccc |
| | ctgtttgctcctccgataactggggtgaccttggttaatattcaccagcagc |
| | ctcccccgttgcccctctggatccactgcttaaatacggacgaggacagggc |
| | cctgtctcctcagcttcaggcaccaccactgacctgggacagt |
| LMTP19 | aggctcagaggcacacaggagtttctgggctcaccctgcccccttccaaccc |
| SEQ ID NO: 238 | ctcagttcccatcctccagcagctgtttgtgtgctgcctctgaagtccacac |
| | tgaacaaacttcagcctactcatgtccctaaaatgggcaaacattgcaagca |
| | gcaaacagcaaacacacagccctccctgcctgctgaccttggagctggggca |
| | gaggtcagagacctctctgggcccatgccacctccaacatccactcgacccc |
| | ttggaatttcggtggagaggagcagaggttgtcctggcgtggtttaggtagt |
| | gtgagagggccctgcatgcgaagatcttcgaacaaggctgtgggggactgag |
| | ggcaggctgtaacaggcttgggggccagggcttatacgtgcctgggactccc |
| | aaagtattactgttccatgttcccggcgaagggccagctgtcccccgccagc |
| | tagactcagcacttagtttaggaaccagtgagcaagtcagcccttggggcag |
| | cccatacaaggccatggggctgggcaagctgcacgcctgggtccggggtggg |
| | cacggtgcccgggcaacgagctgaaagctcatctgctctcaggggcccctcc |
| | ctggggacagcccctcctggctagtcacaccctgtaggctcctctatataac |
| | ccaggggcacaggggctgccctcattctaccaccacctccacagcacagaca |
| | gacactcaggagccagccagcgtcgagatcttgctaccagtggaacagccac |
| | taaggattctgcagtgagagcagagggccagctaagtggtactctcccagag |
| | actgtctgactcacgccaccccctccaccttggacacaggacgctgtggttt |
| | ctgagccaggtacagtgactcctttcggtaagtgcagtggaagctgtacact |
| | gcccaggcaaagcgtccgggcagcgtaggcgggcgactcagatcccagccag |
| | tggacttagcccctgtttgctcctccgataactggggtgaccttggttaata |
| | ttcaccagcagcctcccccgttgcccctctggatccactgcttaaatacgga |
| | cgaggacagggccctgtctcctcagcttcaggcaccaccactgacctgggac |
| | agt |
| LMTP20 | aggctcagaggcacacaggagtttctgggctcaccctgcccccttccaaccc |
| SEQ ID NO: 239 | ctcagttcccatcctccagcagctgtttgtgtgctgcctctgaagtccacac |
| | tgaacaaacttcagcctactcatgtccctaaaatgggcaaacattgcaagca |
| | gcaaacagcaaacacacagccctccctgcctgctgaccttggagctggggca |
| | gaggtcagagacctctctgggcccatgccacctccaacatccactcgacccc |
| | ttggaatttcggtggagaggagcagaggttgtcctggcgtggtttaggtagt |
| | gtgagagggcccttcagattaaaaataactgaggtaagggcctgggtagggg |
| | aggtggtgtgagacgctcctgtctctcctctatctgcccatcggccctttgg |
| | ggaggaggaatgtgcccaaggactaaaaaaaggccatggagccagaggggcg |
| | agggcaacagacctttcatgggcaaaccttggggccctgctgaagctttggc |
| | ccactacgggtttaggctgcccatgtaaggaggcaaggcctggggacacccg |
| | agatgcctggttataattaacccagacatgtggctgccccccccccccccaa |
| | cacctgctgcctctaaaaataaccctgtccctggtggatcccctgcatgcga |
| | agatcttcgaacaaggctgtgggggactgagggcaggctgtaacaggcttgg |
| | gggccagggcttatacgtgcctgggactcccaaagtattactgttccatgtt |
| | cccggcgaagggccagctgtcccccgccagctagactcagcacttagtttag |
| | gaaccagtgagcaagtcagcccttggggcagcccatacaaggccatggggct |
| | gggcaagctgcacgcctgggtccggggtgggcacggtgcccgggcaacgagc |
| | tgaaagctcatctgctctcaggggcccctccctggggacagcccctcctggc |
| | tagtcacaccctgtaggctcctctatataacccaggggcacaggggctgccc |
| | tcattctaccaccacctccacagcacagacagacactcaggagccagccagc |
| | gtcgagatcttgctaccagtggaacagccactaaggattctgcagtgagagc |
| | agagggccagctaagtggtactctcccagagactgtctgactcacgccaccc |
| | cctccaccttggacacaggacgctgtggtttctgagccaggtacagtgactc |
| | ctttcggtaagtgcagtggaagctgtacactgcccaggcaaagcgtccgggc |
| | agcgtaggcgggcgactcagatcccagccagtggacttagcccctgtttgct |
| | cctccgataactggggtgaccttggttaatattcaccagcagcctcccccgt |
| | tgcccctctggatccactgcttaaatacggacgaggacagggccctgtctcc |
| | tcagcttcaggcaccaccactgacctgggacagt |
| ApoE.hAAT | aggctcagaggcacacaggagtttctgggctcaccctgcccccttccaaccc |
| SEQ ID NO: 240 | ctcagttcccatcctccagcagctgtttgtgtgctgcctctgaagtccacac |
| | tgaacaaacttcagcctactcatgtccctaaaatgggcaaacattgcaagca |
| | gcaaacagcaaacacacagccctccctgcctgctgaccttggagctggggca |
| | gaggtcagagacctctctgggcccatgccacctccaacatccactcgacccc |
| | ttggaatttcggtggagaggagcagaggttgtcctggcgtggtttaggtagt |
| | gtgagaggggtacccggggatcttgctaccagtggaacagccactaaggatt |
| | ctgcagtgagagcagagggccagctaagtggtactctcccagagactgtctg |
| | actcacgccaccccctccaccttggacacaggacgctgtggtttctgagcca |
| | ggtacaatgactcctttcggtaagtgcagtggaagctgtacactgcccaggc |
| | aaagcgtccgggcagcgtaggcgggcgactcagatcccagccagtggactta |
| | gcccctgtttgctcctccgataactggggtgaccttggttaatattcaccag |
| | cagcctcccccgttgcccctctggatccactgcttaaatacggacgaggaca |
| | gggccctgtctcctcagcttcaggcaccaccactgacctgggacagt |
| Alpha-Mic/Bik | aggttaatttttaaaaagcagtcaaaagtccaagtggcccttggcagcattt |
| Enhancer | actctctctgtttgctctggttaataatctcaggagcacaaacattcc |
| (Mic/BikE) | |
| SEQ ID NO: 241 | |
| Tandem (2) | aggttaatttttaaaaagcagtcaaaagtccaagtggcccttggcagcattt |
| alpha-Mic/Bik | actctctctgtttgctctggttaataatctcaggagcacaaacattccaggt |
| Enhancers | taatttttaaaaagcagtcaaaagtccaagtggcccttggcagcatttactc |
| (2 Mic/BikE) | tctctgtttgctctggttaataatctcaggagcacaaacattcc |
| SEQ ID NO: 242 | |
| ApoE Hepatic | aggctcagaggcacacaggagtttctgggctcaccctgcccccttccaaccc |
| Control Region | ctcagttcccatcctccagcagctgtttgtgtgctgcctctgaagtccacac |
| containing ApoE | tgaacaaacttcagcctactcatgtccctaaaatgggcaaacattgcaagca |
| Enhancer | gcaaacagcaaacacacagccctccctgcctgctgaccttggagctggggca |
| SEQ ID NO: 243 | gaggtcagagacctctctgggcccatgccacctccaacatccactcgacccc |
| | ttggaatttcggtggagaggagcagaggttgtcctggcgtggtttaggtagt |
| | gtgagaggg |
| Tandem (2) | aggctcagaggcacacaggagtttctgggctcaccctgcccccttccaaccc |
| ApoE Enhancers | ctcagttcccatcctccagcagctgtttgtgtgctgcctctgaagtccacac |
| SEQ ID NO: 244 | tgaacaaacttcagcctactcatgtccctaaaatgggcaaacattgcaagca |
| | gcaaacagcaaacacacagccctccctgcctgctgaccttggagctggggca |
| | gaggtcagagacctctctgggcccatgccacctccaacatccactcgacccc |
| | ttggaatttcggtggagaggagcagaggttgtcctggcgtggtttaggtagt |
| | gtgagagggtctagaaggctcagaggcacacaggagtttctgggctcaccct |
| | gcccccttccaacccctcagttcccatcctccagcagctgtttgtgtgctgc |
| | ctctgaagtccacactgaacaaacttcagcctactcatgtccctaaaatggg |
| | caaacattgcaagcagcaaacagcaaacacacagccctccctgcctgctgac |
| | cttggagctggggcagaggtcagagacctctctg |
| hAAT Promoter | gatcttgctaccagtggaacagccactaaggattctgcagtgagagcagagg |
| SEQ ID NO: 245 | gccagctaagtggtactctcccagagactgtctgactcacgccaccccctcc |
| | accttggacacaggacgctgtggtttctgagccaggtacaatgactcctttc |
| | ggtaagtgcagtggaagctgtacactgcccaggcaaagcgtccgggcagcgt |
| | aggcgggcgactcagatcccagccagtggacttagcccctgtttgctcctcc |
| | gataactggggtgaccttggttaatattcaccagcagcctcccccgttgccc |
| | ctctggatccactgcttaaatacggacgaggacagggccctgtctcctcagc |
| | ttcaggcaccaccactgacctgggacagt |
| hAAT(ΔATG) | gatcttgctaccagtggaacagccactaaggattctgcagtgagagcagagg |
| Promoter | gccagctaagtggtactctcccagagactgtctgactcacgccaccccctcc |
| SEQ ID NO:246 | accttggacacaggacgctgtggtttctgagccaggtacagtgactcctttc |
| | ggtaagtgcagtggaagctgtacactgcccaggcaaagcgtccgggcagcgt |
| | aggcgggcgactcagatcccagccagtggacttagcccctgtttgctcctcc |
| | gataactggggtgaccttggttaatattcaccagcagcctcccccgttgccc |
| | ctctggatccactgcttaaatacggacgaggacagggccctgtctcctcagc |
| | ttcaggcaccaccactgacctgggacagt |
| Mck Enhancer | ccactacgggtttaggctgcccatgtaaggaggcaaggcctggggacacccg |
| (MckE) | agatgcctggttataattaacccagacatgtggctgccccccccccccccaa |
| SEQ ID NO: 247 | cacctgctgcctctaaaaataaccctgtccctggtggatc |
| Tandem (2) Mck | ccactacgggtttaggctgcccatgtaaggaggcaaggcctggggacacccg |
| Enhancers | agatgcctggttataattaacccagacatgtggctgccccccccccccccaa |
| (2 MckE) | cacctgctgcctctaaaaataaccctgtccctggtggatcccactacgggtt |
| SEQ ID NO: 248 | taggctgcccatgtaaggaggcaaggcctggggacacccgagatgcctggtt |
| | ataattaacccagacatgtggctgccccccccccccccaacacctgctgcct |
| | ctaaaaataaccctgtccctggtggatc |
| Tandem Mck (3) | ccactacgggtttaggctgcccatgtaaggaggcaaggcctggggacacccg |
| Enhancers | agatgcctggttataattaacccagacatgtggctgccccccccccccccaa |
| (3 MckE) | cacctgctgcctctaaaaataaccctgtccctggtggatcccactacgggtt |
| SEQ ID NO: 249 | taggctgcccatgtaaggaggcaaggcctggggacacccgagatgcctggtt |
| | ataattaacccagacatgtggctgccccccccccccccaacacctgctgcct |
| | ctaaaaataaccctgtccctggtggatcccactacgggtttaggctgcccat |
| | gtaaggaggcaaggcctggggacacccgagatgcctggttataattaaccca |
| | gacatgtggctgccccccccccccccaacacctgctgcctctaaaaataacc |
| | ctgtccctggtggatc |
| Myosin heavy | cccttcagattaaaaataactgaggtaagggcctgggtaggggaggtggtgt |
| chain enhancer | gagacgctcctgtctctcctctatctgcccatcggccctttggggaggagga |
| (MhcE) | atgtgcccaaggactaaaaaaaggccatggagccagaggggcgagggcaaca |
| SEQ ID NO: 250 | gacctttcatgggcaaaccttggggccctgctgaagctttggc |

In certain embodiments, the viral vectors provided herein comprise one or more regulatory elements other than a promoter. In certain embodiments, the viral vectors provided herein comprise an enhancer. In certain embodiments, the viral vectors provided herein comprise a repressor. In certain embodiments, the viral vectors provided herein comprise an intron (e.g. V_{H}4 intron (SEQ ID NO:80), SV40 intron (SEQ ID NO:272), or a chimeric intron ((3-globin/Ig Intron) (SEQ ID NO:79). The viral vectors may also include a Kozak sequence to promote translation of the transgene product, for example GCCACC.

In certain embodiments, the viral vectors provided herein comprise a polyadenylation sequence downstream of the coding region of the transgene. Any polyA site that signals termination of transcription and directs the synthesis of a polyA tail is suitable for use in AAV vectors of the present disclosure. Exemplary polyA signals are derived from, but not limited to, the following: the SV40 late gene, the rabbit β-globin gene (SEQ ID NO:78), the bovine growth hormone (BPH) gene, the human growth hormone (hGH) gene, the synthetic polyA (SPA) site, and the bovine growth hormone (bGH) gene. See, e.g., Powell and Rivera-Soto, 2015, Discov. Med., 19(102):49-57.

5.1.4 Signal Peptides

In certain embodiments, the vectors provided herein comprise components that modulate protein delivery. In certain embodiments, the viral vectors provided herein comprise one or more signal peptides. Signal peptides (also referred to as “signal sequences”) may also be referred to herein as “leader sequences” or “leader peptides”. In certain embodiments, the signal peptides allow for the transgene product to achieve the proper packaging (e.g., glycosylation) in the cell. In certain embodiments, the signal peptides allow for the transgene product to achieve the proper localization in the cell. In certain embodiments, the signal peptides allow for the transgene product to achieve secretion from the cell.

There are two general approaches to select a signal sequence for protein production in a gene therapy context or in cell culture. One approach is to use a signal peptide from proteins homologous to the protein being expressed. For example, a human antibody signal peptide may be used to express IgGs in CHO or other cells. Another approach is to identify signal peptides optimized for the particular host cells used for expression. Signal peptides may be interchanged between different proteins or even between proteins of different organisms, but usually the signal sequences of the most abundant secreted proteins of that cell type are used for protein expression. For example, the signal peptide of human albumin, the most abundant protein in plasma, was found to substantially increase protein production yield in CHO cells. However, certain signal peptides may retain function and exert activity after being cleaved from the expressed protein as “post-targeting functions”. Thus, in specific embodiments, the signal peptide is selected from signal peptides of the most abundant proteins secreted by the cells used for expression to avoid the post-targeting functions. In a certain embodiment, the signal sequence is fused to both the heavy and light chain sequences. An exemplary sequence is MYRMQLLLLIALSLALVTNS (SEQ ID NO:85) which can be encoded by a nucleotide sequence of SEQ ID NO: 90 (see Table 2, FIGS. 2A-2C and 3A-31I). Alternatively, signal sequences that are appropriate for expression, and may cause selective expression or directed expression of the HuPTM mAb or Fab or scFv in the eye/CNS, muscle, or liver are provided in Tables 2, 3, and 4, respectively, below.

TABLE 2
Signal peptides for expression in eye/CNS

| Signal | SEQ ID | |
|---|---|---|
| Peptide Origin | NO: | Sequence |
| Mutant interleukin | 85 | MYRMQLLLLIALSLALVINS |
| 2 signal peptide | | |
| Mutant interleukin | 86 | atgtataggatgcaactgct |
| 2 signal peptide | | cctcctgattgctctgagcc |
| coding sequence | | tggctcttgtgaccaactct |
| VEGF-A signal | 87 | MNFLLSWVHWSLALLLYLHH |
| peptide | | AKWSQA |
| Fibulin-1 signal | 88 | MERAAPSRRVPLPLLLLGGL |
| peptide | | ALLAAGVDA |
| Vitronectin signal | 89 | MAPLRPLLILALLAWVALA |
| peptide | | |
| Complement Factor H | 90 | MRLLAKIICLMLWAICVA |
| signal peptide | | |
| Opticin signal | 91 | MRLLAFLSLLALVLQETGT |
| peptide | | |
| Albumin signal | 92 | MKWVTFISLLFLFSSAYS |
| peptide | | |
| Chymotrypsinogen | 93 | MAFLWLLSCWALLGTTFG |
| signal peptide | | |
| Interleukin-2 | 94 | MYRMQLLSCIALILALVTNS |
| signal peptide | | |
| Trypsinogen-2 | 95 | MNLLLILTFVAAAVA |
| signal peptide | | |

TABLE 3
Signal peptides for expression in liver cells.

| Signal | SEQ ID | |
|---|---|---|
| Peptide Origin | NO: | Sequence |
| Human Serum albumin | 96 | MKWVTFISLLFLFSSAYS |
| Human α-1 Antitrypsin | 97 | MPSSVSWGILLLAGLCCL |
| (SERPINA1) | | VPVSLA |
| Human Apolipoprotein | 98 | MKAAVLTLAVLFLTGSQA |
| A-1 | | |
| Human Apolipoprotein | 99 | MKLLAATVLLLTICSLEG |
| A-2 | | |
| Human Apolipoprotein | 100 | MDPPRPALLALLALPALL |
| B-100 | | LLLLAGARA |
| Human Coagulation | 101 | MQRVNMIMAESPGLITIC |
| Factor IX | | LLGYLLSAEC |
| Human Complement | 102 | MGPLMVLFCLLFLYPGLA |
| C2 | | DS |
| Human Complement | 103 | MWLLVSVILISRISSVGG |
| Factor H-related | | |
| Protein 2 (CFHR2) | | |
| Human Complement | 104 | MLLLFSVILISWVSTVGG |
| Factor H-related | | |
| Protein 5 (CFHR5) | | |
| Human Fibrinogen α- | 105 | MFSMRIVCLVLSVVGTAW |
| chain (FGA) | | T |
| Human Fibrinogen β- | 106 | MKRMVSWSFHKLKTMKHL |
| chain (FGB) | | LLLLLCVFLVKS |
| Human Fibrinogen γ- | 107 | MSWSLHPRNLILYFYALL |
| chain (FGG) | | FLSSTCVA |
| Human α-2-HS- | 108 | MKSLVLLLCLAQLWGCHS |
| Glycoprotein (AHSG) | | |
| Human Hemopexin | 109 | MARVLGAPVALGLWSLCW |
| (HPX) | | SLAIA |
| Human Kininogen-1 | 110 | MKLITILFLCSRLLLSLT |
| Human Mannose- | 111 | MSLFPSLPLLLLSMVAAS |
| binding protein C | | YS |
| (MBL2) | | |
| Human Plasminogen | 112 | MEHKEVVLLLLLFLKSGQ |
| (PLMN) | | G |
| Human Prothrombin | 113 | MAHVRGLQLPGCLALAAL |
| (Coagulation | | CSLVHS |
| Factor II) | | |
| Human Secreted | 114 | MISRMEKMTMMMKILIMF |
| Phosphoprotein 24 | | ALGMNYWSCSG |
| Human Anti-thrombin- | 115 | MYSNVIGTVTSGKRKVYL |
| III (SERPINC1) | | LSLLLIGFWDCVTC |
| Human Serotransferrin | 116 | MRLAVGALLVCAVLGLCL |
| (TF) | | A |

TABLE 4
Signal peptides for expression in muscle cells.

| Signal | SEQ ID | |
|---|---|---|
| Peptide Origin | NO: | Sequence |
| Human SPARC | 117 | MRAWIFFLLCLAGRALA |
| Human Collagen | 118 | MFSFVDLRLLLLLAATA |
| alpha-1(I) | | LLTHG |
| chain | | |
| Human | 119 | MKLVFLVLLFLGALGLC |
| Lactotransferrin | | LA |
| Human Complement C3 | 120 | MGPTSGPSLLLLLLTHL |
| | | PLALG |
| Human Lumican | 121 | MSLSAFTLFLALIGGTS |
| | | G |
| Human Gelsolin | 122 | MAPHRPAPALLCALSLA |
| isoform 1 | | LCALSLPVRA |
| Human Pro- | 123 | MWATLPLLCAGAWLLGV |
| cathepsin H | | PVCGA |
| Human SERPINF1 | 124 | MQALVLLLCIGALLGHS |
| | | SC |
| Human SERPINE1 | 125 | MQMSPALTCLVLGLALV |
| | | FGEGSA |
| Human Cathepsin D | 126 | MQPSSLLPLALCLLAAP |
| | | ASA |
| Human TIMP1 | 127 | MAPFEPLASGILLLLWL |
| | | IAPSRA |
| Human Fibronectin | 226 | MLRGPGPGLLLLAVQCL |
| | | GTAVPSTGASKSKR |
| Human Complement C1s | 178 | MWCIVLFSLLAWVYA |
| subcomponent | | |
| Human Cathepsin L1 | 179 | MNPTLILAAFCLGIASA |
| Human Cathepsin B | 180 | MWQLWASLCCLLVLANA |
| Human Salivary acidic | 181 | MLLILLSVALLAFSSA |
| proline-rich | | |
| phosphoprotein ½ | | |
| Human Follistatin- | 182 | MWKRWLALALALVAVAW |
| related protein 1 | | VRA |

5.1.5 Polycistronic Messages—IRES and 2A Linkers and scFv Constructs

Internal ribosome entry sites. A single construct can be engineered to encode both the heavy and light chains separated by a cleavable linker or IRES so that separate heavy and light chain polypeptides are expressed by the transduced cells. In certain embodiments, the viral vectors provided herein provide polycistronic (e.g., bicistronic) messages. For example, the viral construct can encode the heavy and light chains separated by an internal ribosome entry site (IRES) elements (for examples of the use of IRES elements to create bicistronic vectors see, e.g., Gurtu et al., 1996, Biochem. Biophys. Res. Comm. 229(1):295-8, which is herein incorporated by reference in its entirety). IRES elements bypass the ribosome scanning model and begin translation at internal sites. The use of IRES in AAV is described, for example, in Furling et al., 2001, Gene Ther 8(11): 854-73, which is herein incorporated by reference in its entirety. In certain embodiments, the bicistronic message is contained within a viral vector with a restraint on the size of the polynucleotide(s) therein. In certain embodiments, the bicistronic message is contained within an AAV virus-based vector (e.g., an AAV8-based, AAV3B-based or AAVrh73-based vector).

Furin-2A linkers. In other embodiments, the viral vectors provided herein encode the heavy and light chains separated by a cleavable linker such as the self-cleaving 2A and 2A-like peptides, with or without upstream furin cleavage sites, e.g. Furin/2A linkers, such as furin/F2A (F/F2A) or furin/T2A (F/T2A) linkers (Fang et al., 2005, Nature Biotechnology 23: 584-590, Fang, 2007, Mol Ther 15: 1153-9, and Chang, J. et al, MAbs 2015, 7(2):403-412, each of which is incorporated by reference herein in its entirety). For example, a furin/2A linker may be incorporated into an expression cassette to separate the heavy and light chain coding sequences, resulting in a construct with the structure:

Signal sequence-Heavy chain-Furin site-2A site-Signal Sequence-Light chain-PolyA.

A 2A site or 2A-like site, such as an F2A site comprising the amino acid sequence RKRR(GSG) APVKQTLNFDLLKLAGDVESNPGP (SEQ ID NOS:143 or 144) or a T2A site comprising the amino acid sequence RKRR(GSG) EGRGSLLTCGDVEENPGP (SEQ ID NOS:141 or 142), is self-processing, resulting in “cleavage” between the final G and P amino acid residues. Several linkers, with or without an upstream flexible Gly-Ser-Gly (GSG) linker sequence (SEQ ID NO:128), that could be used include but are not limited to:

T2A:
(SEQ ID NOS: 133 or 134)
(GSG)EGRGSLLTCGDVEENPGP;
P2A:
(SEQ ID NOS: 135 or 136)
(GSG)ATNFSLLKQAGDVEENPGP;
E2A:
(SEQ ID NOS: 137 or 138)
(GSG)QCTNYALLKLAGDVESNPGP;
F2A:
(SEQ ID NOS: 139 or 140)
(GSG)APVKQTLNFDLLKLAGDVESNPGP

(see also, e.g., Szymczak, et al., 2004, Nature Biotechnol 22(5):589-594, and Donnelly, et al., 2001, J Gen Virol, 82:1013-1025, each of which is incorporated herein by reference). Exemplary amino acid and nucleotide sequences encoding different parts of the flexible linker are described in Table 4.

TABLE 4
Linker Sequences

| | SEQ | |
|---|---|---|
| ID | ID NO: | Sequence |
| GSG linker | 128 | GSG |
| Furin linker | 129 | RKRR |
| Furin linker | 130 | RRRR |
| Furin linker | 131 | RRKR |
| Furin linker | 132 | RKKR |
| T2A | 133 | EGRGSLLTCGDVEENPGP |
| T2A | 134 | GSGEGRGSLLTCGDVEEN |
| | | PGP |
| P2A | 135 | ATNFSLLKQAGDVEENPG |
| | | P |
| P2A | 136 | GSGATNFSLLKQAGDVEE |
| | | NPGP |
| E2A | 137 | QCTNYALLKLAGDVESNP |
| | | GP |
| E2A | 138 | GSGQCTNYALLKLAGDVE |
| | | SNPGP |
| F2A | 139 | APVKQTLNFDLLKLAGDV |
| | | ESNPGP |
| F2A | 140 | GSGAPVKQTLNFDLLKLA |
| | | GDVESNPGP |
| Furin-T2A | 141 | RKRREGRGSLLTCGDVEE |
| | | NPGP |
| Furin-GSG-T2A | 142 | RKRRGSGEGRGSLLTCGD |
| | | VEENPGP |
| Furin-F2A | 143 | RKRRAPVKQTLNFDLLKL |
| | | AGDVESNPGP |
| Furin-GSG-F2A | 144 | RKRRGSGAPVKQTLNFDL |
| | | LKLAGDVESNPGP |
| Furin-GSG-T2A | 145 | agaaagagaagaggctct |
| | | ggagaaggcagaggctcc |
| | | ctgctgacatgtggggat |
| | | gttgaagagaatcctggg |
| | | cct |
| Furin | 146 | agaaagagaaga |
| Furin-GSG | 147 | agaaagagaagaggctct |
| linker | | gga |
| GSG linker | 148 | ggctctgga |
| T2A | 149 | gaaggcagaggctccctg |
| | | ctgacatgtggggatgtt |
| | | gaagagaatcctgggcct |

In certain embodiments an additional proteolytic cleavage site, e.g. a furin cleavage site, is incorporated into the expression construct adjacent to the self-processing cleavage site (e.g. 2A or 2A like sequence), thereby providing a means to remove additional amino acids that remain following cleavage by the self processing cleavage sequence. Without being bound to any one theory, a peptide bond is skipped when the ribosome encounters the 2A sequence in the open reading frame, resulting in the termination of translation, or continued translation of the downstream sequence (the light chain). This self-processing sequence results in a string of additional amino acids at the end of the C-terminus of the heavy chain. However, such additional amino acids can then be cleaved by host cell Furin at the furin cleavage site(s), e.g. located immediately prior to the 2A site and after the heavy chain sequence, and further cleaved by carboxypeptidases. The resultant heavy chain may have one, two, three, or more additional amino acids included at the C-terminus, or it may not have such additional amino acids, depending on the sequence of the Furin linker used and the carboxypeptidase that cleaves the linker in vivo (See, e.g., Fang et al., 17 Apr. 2005, Nature Biotechnol. Advance Online Publication; Fang et al., 2007, Molecular Therapy 15(6):1153-1159; Luke, 2012, Innovations in Biotechnology, Ch. 8, 161-186). Furin linkers that may be used comprise a series of four basic amino acids, for example, RKRR (SEQ ID NO:129), RRRR (SEQ ID NO:130), RRKR (SEQ ID NO:131), or RKKR (SEQ ID NO:132). Once this linker is cleaved by a carboxypeptidase, additional amino acids may remain, such that an additional zero, one, two, three or four amino acids may remain on the C-terminus of the heavy chain, for example, R, RR, RK, RKR, RRR, RRK, RKK, RKRR (SEQ ID NO:129), RRRR (SEQ ID NO:130), RRKR (SEQ ID NO:131), or RKKR (SEQ ID NO:132). In certain embodiments, once the linker is cleaved by a carboxypeptidase, no additional amino acids remain. In certain embodiments, 0.5% to 1%, 1% to 2%, 5%, 10%, 15%, or 20% of the antibody, e.g., antigen-binding fragment, population produced by the constructs for use in the methods described herein has one, two, three, or four amino acids remaining on the C-terminus of the heavy chain after cleavage. In certain embodiments, the furin linker has the sequence R-X-K/R-R, such that the additional amino acids on the C-terminus of the heavy chain are R, RX, RXK, RXR, RXKR (SEQ ID NO:251), or RXRR (SEQ ID NO:252), where X is any amino acid, for example, alanine (A). In certain embodiments, no additional amino acids may remain on the C-terminus of the heavy chain.

Flexible peptide linker. In some embodiments, a single construct can be engineered to encode both the heavy and light chains (e.g. the heavy and light chain variable domains) separated by a flexible peptide linker such as those encoding a scFv. A flexible peptide linker can be composed of flexible residues like glycine and serine so that the adjacent heavy chain and light chain domains are free to move relative to one another. The construct may be arranged such that the heavy chain variable domain is at the N-terminus of the scFv, followed by the linker and then the light chain variable domain. Alternatively, the construct may be arranged such that the light chain variable domain is at the N-terminus of the scFv, followed by the linker and then the heavy chain variable domain. That is, the components may be arranged as NH₂-V_{L}-linker-V_{H}-COOH or NH₂-V_{H}-linker-V_{L}-COOH.

In certain embodiments, an expression cassette described herein is contained within a viral vector with a restraint on the size of the polynucleotide(s) therein. In certain embodiments, the expression cassette is contained within an AAV virus-based vector. Due to the size restraints of certain vectors, the vector may or may not accommodate the coding sequences for the full heavy and light chains of the therapeutic antibody but may accommodate the coding sequences of the heavy and light chains of antigen binding fragments, such as the heavy and light chains of a Fab or F(ab′)₂ fragment or an scFv. In particular, the AAV vectors described herein may accommodate a transgene of approximately 4.7 kilobases. Substitution of smaller expression elements would permit the expression of larger protein products, such as full-length therapeutic antibodies.

5.1.6 Untranslated Regions

In certain embodiments, the viral vectors provided herein comprise one or more untranslated regions (UTRs), e.g., 3′ and/or 5′ UTRs. In certain embodiments, the UTRs are optimized for the desired level of protein expression. In certain embodiments, the UTRs are optimized for the mRNA half-life of the transgene. In certain embodiments, the UTRs are optimized for the stability of the mRNA of the transgene. In certain embodiments, the UTRs are optimized for the secondary structure of the mRNA of the transgene.

5.1.7 Inverted Terminal Repeats

In certain embodiments, the viral vectors provided herein comprise one or more inverted terminal repeat (ITR) sequences. ITR sequences may be used for packaging the recombinant gene expression cassette into the virion of the viral vector. In certain embodiments, the ITR is from an AAV, e.g., AAV8 or AAV2 (see, e.g., Yan et al., 2005, J. Virol., 79(1):364-379; U.S. Pat. No. 7,282,199 B2, U.S. Pat. No. 7,790,449 B2, U.S. Pat. No. 8,318,480 B2, U.S. Pat. No. 8,962,332 B2 and International Patent Application No. PCT/EP2014/076466, each of which is incorporated herein by reference in its entirety). In preferred embodiments, nucleotide sequences encoding the ITRs may, for example, comprise the nucleotide sequences of SEQ ID NOS:81 (5′-ITR) or 82 (3′-ITR). In certain embodiments, the modified ITRs used to produce self-complementary vector, e.g., scAAV, may be used (see, e.g., Wu, 2007, Human Gene Therapy, 18(2):171-82, McCarty et al, 2001, Gene Therapy, Vol 8, Number 16, Pages 1248-1254; and U.S. Pat. Nos. 6,596,535; 7,125,717; and 7,456,683, each of which is incorporated herein by reference in its entirety). In preferred embodiments, nucleotide sequences encoding the modified ITRs may, for example, comprise the nucleotide sequences of SEQ ID NOS:81 (5′-ITR) or 83 (3′-ITR) or modified for scAAV, SEQ ID NO:82 (m 5′ITR) or SEQ ID NO:84 (m 3′ ITR).

5.1.8 Transgenes

The transgenes encode a HuPTM mAb, either as a full-length antibody or an antigen binding fragment thereof, e.g. a Fab fragment (an HuGlyFab) or a F(ab′)₂, nanobody, or an scFv based upon a therapeutic antibody disclosed herein. In specific embodiments, the HuPTM mAb or antigen binding fragment, particularly the HuGlyFab, are engineered to contain additional glycosylation sites on the Fab domain (e.g., see Courtois et al., 2016, mAbs 8: 99-112 which is incorporated by reference herein in its entirety for it description of sites of hyperglycosylation on a Fab domain). In addition, for the HuPTM mAb comprising an Fc domain, the Fc domain may be engineered to alter the glycosylation site at N297 to prevent glycosylation at that site (for example, a substitution at N297 for another amino acid and/or a substitution at T297 for a residue that is not a T or S to knock out the glycosylation site). Such Fc domains are “aglycosylated”.

5.1.8.1 Constructs for Expression of Full Length HuPTM mAb

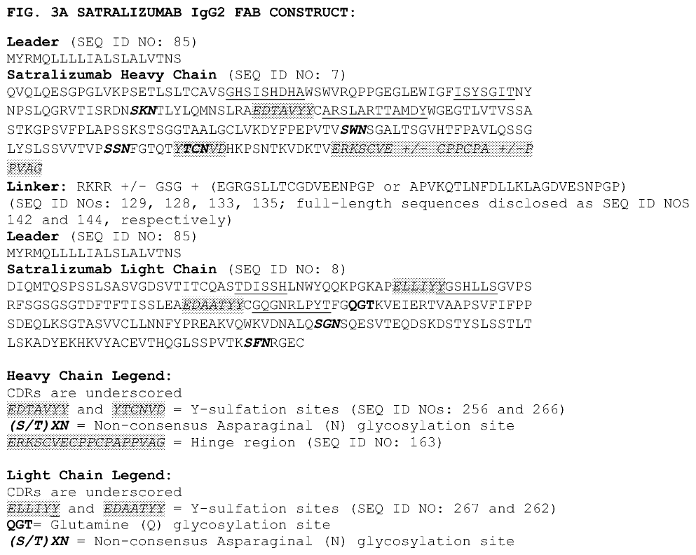

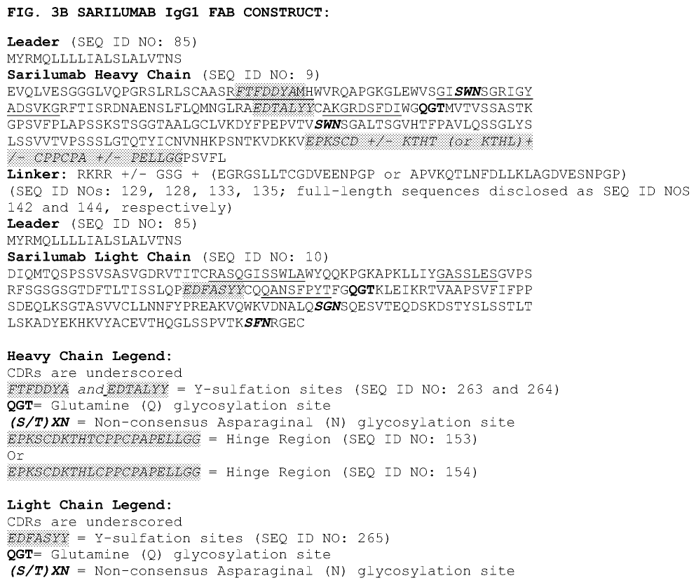

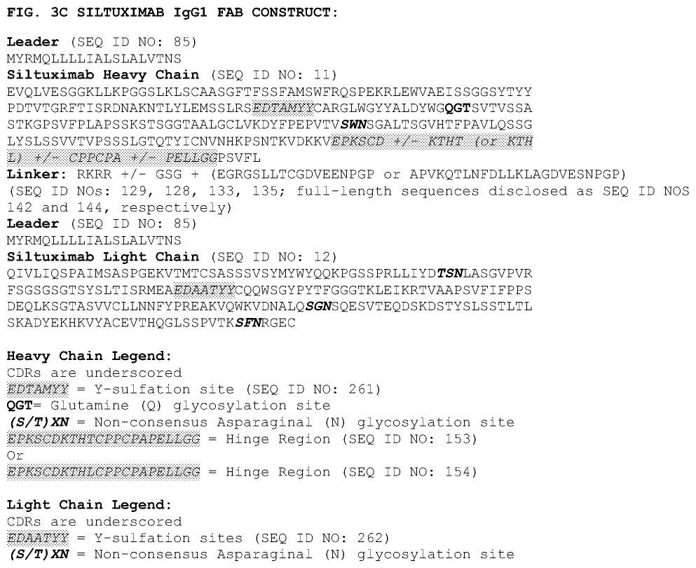

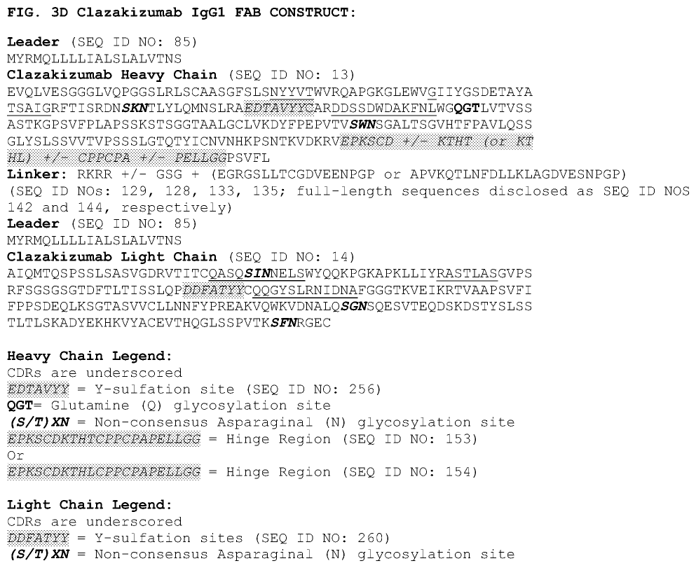

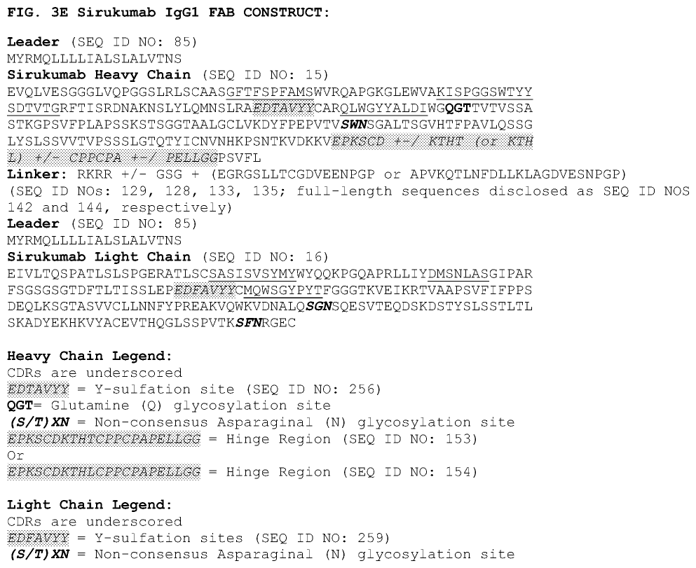

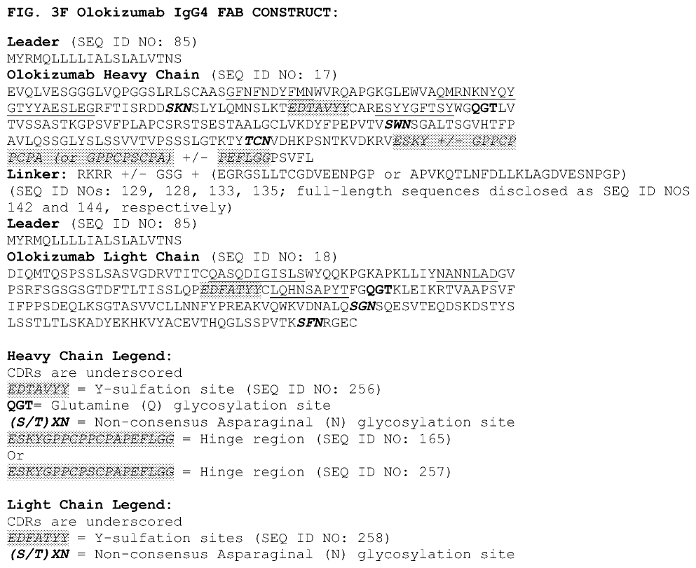

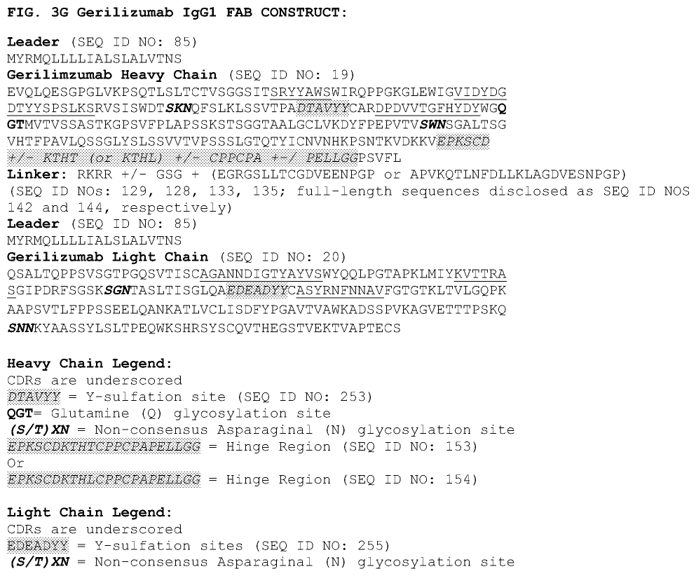

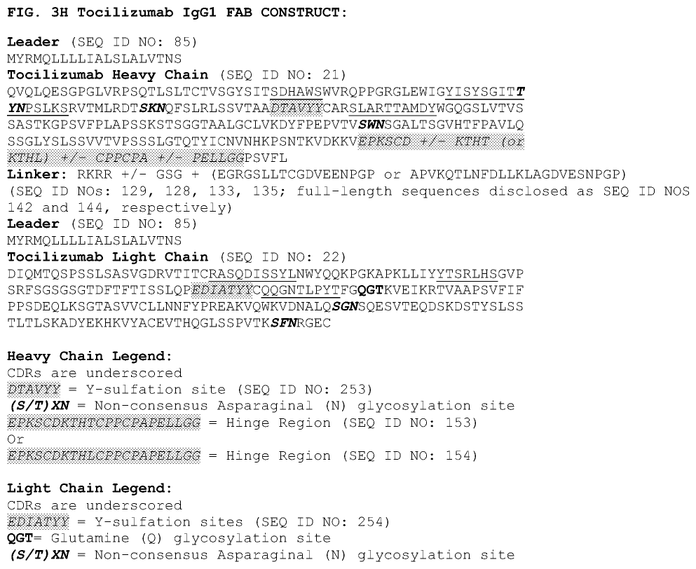

In certain embodiments, the transgenes encode a full length heavy chain (including the heavy chain variable domain, the heavy chain constant domain 1 (CH1), the hinge and Fc domain) and a full length light chain (light chain variable domain and light chain constant domain) that upon expression associate to form antigen-binding antibodies with Fc domains. The recombinant AAV constructs express the intact (i.e., full length) or substantially intact HuPTM mAb in a cell, cell culture, or in a subject. (“Substantially intact” refers to mAb having a sequence that is at least 95% identical to the full-length mAb sequence.) The nucleotide sequences encoding the heavy and light chains may be codon optimized for expression in human cells and have reduced incidence of CpG dimers in the sequence to promote expression in human cells. See for example, the codon optimized sequences of adalimumab (SEQ ID NOs: 46 to 60) of Table 8. The transgenes may encode any full-length antibody. In preferred embodiments, the transgenes encode a full-length form of any of the therapeutic antibodies disclosed herein, for example, the Fab fragment of which depicted in FIGS. 2A-2C or 3A-3H herein and including, in certain embodiments, the associated Fc domain provided in Table 6.

The full length mAb encoded by the transgene described herein preferably have the Fc domain of the full-length therapeutic antibody or is an Fc domain of the same type of immunoglobulin as the therapeutic antibody to be expressed. In certain embodiments, the Fc region is an IgG Fc region, but in other embodiments, the Fc region may be an IgA, IgD, IgE, or IgM. The Fc domain is preferably of the same isotype as the therapeutic antibody to be expressed, for example, if the therapeutic antibody is an IgG1 isotype, then the antibody expressed by the transgene comprises an IgG1 Fc domain. The antibody expressed from the transgene may have an IgG1, IgG2, IgG3 or IgG4 Fc domain.

The Fc region of the intact mAb has one or more effector functions that vary with the antibody isotype. The effector functions can be the same as that of the wild-type or the therapeutic antibody or can be modified therefrom to add, enhance, modify, or inhibit one or more effector functions using the Fc modifications disclosed in Section 5.1.9, infra. In certain embodiments, the HuPTM mAb transgene encodes a mAb comprising an Fc polypeptide comprising an amino acid sequence that is at least 85%, 86%, 87%, 88%, 89%, 90%, 91%, 92%, 93%, 94%, 95%, 96%, 97%, 98% or 99% identical to the sequence set forth in the Fc domain polypeptides of the therapeutic antibodies described herein as set forth in Table 6 for adalimumab, infliximab, and golimumab, or an exemplary Fc domain of an IgG1, IgG2 or IgG4 isotype as set forth in Table 6. In some embodiments, the HuPTM mAb comprises a Fc polypeptide of a sequence that is a variant of the Fc polypeptide sequence in Table 6 in that the sequence has been modified with one or more of the techniques described in Section 5.1.9, infra, to alter the Fc polypeptide's effector function.

In some embodiments, provided are exemplary recombinant AAV constructs such as the constructs shown in FIGS. 1A and 1B, for gene therapy administration to a human subject in order to express an intact or substantially intact HuPTM mAb in the subject. Gene therapy constructs are designed such that both the heavy and light chains are expressed in tandem from the vector including the Fc domain polypeptide of the heavy chain. In certain embodiments, the transgene encodes a transgene with heavy and light chain Fab fragment polypeptides as shown in Table 7, yet have a heavy chain that further comprises an Fc domain polypeptide C terminal to the hinge region of the heavy chain (including an IgG1, IgG2 or IgG4 Fc domain or the adalimumab, infliximab, or golimumab Fc as in Table 6). In specific embodiments, the transgene is a nucleotide sequence that encodes the following: Signal sequence-heavy chain Fab portion (including hinge region)-heavy chain Fc polypeptide-Furin-2A linker-signal sequence-light chain Fab portion.

In specific embodiments for expressing an intact or substantially intact mAb in ocular tissue cell types, the constructs described herein comprise the following components: (1) AAV2 inverted terminal repeats that flank the expression cassette; (2) Control elements, which include a) an inducible promoter, preferably a ocular-tissue specific promoter, b) optionally an intron, such as a chicken β-actin intron or VH4 intron and c) a rabbit β-globin poly A signal; and (3) nucleic acid sequences coding for the heavy chain Fab of an anti-TNFα, anti-IL6, or anti-IL6R mAb (e.g. adalimumab, infliximab, golimumab, satralizumab, sarilumab, tocilizumab, siltuximab, clazakizumab, sirukumab, olokizumab, gerilizumab); an Fc polypeptide associated with the therapeutic antibody (Table 6) or of the same isotype as the native form of the therapeutic antibody, such as an IgG isotype amino acid sequence from Table 6; and the light chain of an anti-TNFα, anti-IL6, or anti-IL6R mAb (e.g. adalimumab, infliximab, golimumab, satralizumab, sarilumab, tocilizumab, siltuximab, clazakizumab, sirukumab, olokizumab, gerilizumab), wherein the heavy chain (Fab and Fc region) and the light chain are separated by a self-cleaving furin (F)/F2A or T2A or flexible linker, ensuring expression of equal amounts of the heavy and the light chain polypeptides. Exemplary constructs are provided in FIGS. 1A and 1B.

In specific embodiments, provided are AAV vectors comprising a viral capsid that is at least 95% identical to the amino acid sequence of an AAV8 capsid (SEQ ID NO:196); and an artificial genome comprising an expression cassette flanked by AAV inverted terminal repeats (ITRs), wherein the expression cassette comprises a transgene encoding an intact or substantially intact anti-TNFα, anti-IL6, or anti-IL6R mAb; operably linked to one or more regulatory sequences that control expression of the transgene in ocular tissue type cells.

The rAAV vectors that encode and express the full-length therapeutic antibodies may be administered to treat or prevent or ameliorate symptoms of a disease or condition amenable to treatment, prevention or amelioration of symptoms with the therapeutic antibodies. Also provided are methods of expressing HuPTM mAbs in human cells using the rAAV vectors and constructs encoding them.

5.1.8.2 Constructs for Expression of Antigen Binding Fragments

In some embodiments, the transgenes express antigen binding fragments, e.g. a Fab fragment (an HuGlyFab) or a F(ab′)₂, nanobody, or an scFv based upon a therapeutic antibody disclosed herein. FIGS. 2A-2C and 3A-3H and section 5.4. provide the amino acid sequence of the heavy and light chains of the Fab fragments of the therapeutic antibodies (see also Table 7, which provides the amino acid sequences of the Fab heavy and light chains of the therapeutic antibodies).

Certain of these nucleotide sequences are codon optimized for expression in human cells. See for example, the codon optimized sequences of adalimumab (SEQ ID NOs: 46 to 60) in Table 8. The transgene may encode a Fab fragment using nucleotide sequences encoding the amino acid sequences provided in Table 7, but not including the portion of the hinge region on the heavy chain that forms interchain di-sulfide bonds (e.g., the portion containing the sequence CPPCPA (SEQ ID NO:150)). Heavy chain Fab domain sequences that do not contain a CPPCP (SEQ ID NO:151) sequence of the hinge region at the C-terminus will not form intrachain disulfide bonds and, thus, will form Fab fragments with the corresponding light chain Fab domain sequences, whereas those heavy chain Fab domain sequences with a portion of the hinge region at the C-terminus containing the sequence CPPCP (SEQ ID NO:151) will form intrachain disulfide bonds and, thus, will form Fab₂ fragments. For example, in some embodiments, the transgene may encode a scFv comprising a light chain variable domain and a heavy chain variable domain connected by a flexible linker in between (where the heavy chain variable domain may be either at the N-terminal end or the C-terminal end of the scFv), and optionally, may further comprise a Fc polypeptide (e.g., IgG1, IgG2, IgG3, or IgG4) on the C-terminal end of the heavy chain. Alternatively, in other embodiments, the transgene may encode F(ab′)₂ fragments comprising a nucleotide sequence that encodes the light chain and the heavy chain sequence that includes at least the sequence CPPCA (SEQ ID NO:152) of the hinge region, as depicted in FIGS. 2A-2C and 3A-31I which depict various regions of the hinge region that may be included at the C-terminus of the heavy chain sequence. Pre-existing anti-hinge antibodies (AHA) may cause immunogenicity and reduce efficacy. Thus, in certain embodiments, for the IgG1 isotype, C-terminal ends with D221 or ends with a mutation T225L or with L242 can reduce binding to AHA. (See, e.g., Brerski, 2008, J Immunol 181: 3183-92 and Kim, 2016, 8: 1536-1547). For IgG2, the risk of AHA is lower since the hinge region of IgG2 is not as susceptible to enzymatic cleavage required to generate endogenous AHA. (See, e.g., Brerski, 2011, MAbs 3: 558-567).

TABLE 5
Hinge Regions

| SEQ ID NO: | Sequence |
|---|---|
| 150 | CPPCP |
| 151 | CPPCPA |
| 152 | CPPCA |
| 153 | EPKSCDKTHTCPPCPAPELLGG |
| 154 | EPKSCDKTHLCPPCPAPELLGG |
| 155 | EPKSCDKTHL |
| 156 | EPKSCDKTHT |
| 157 | EPKSCDKTHTCPPCPA |
| 158 | EPKSCDKTHLCPPCPA |
| 159 | EPKSCDKTHTCPPCPAPELLGGPSVFL |
| 160 | EPKSCDKTHLCPPCPAPELLGGPSVFL |
| 161 | EPKSCDKTHTCPPCPAPEAAGG |
| 162 | EPKSCDKTHTCPPCPAPEAAGGPSVFL |
| 163 | EPKSCDKTHLCPPCPAPEAAGGPSVFL |
| 164 | ERKSCVECPPCPAPPVAG |
| 165 | ERKSCVECPPCPA |
| 166 | ESKYGPPCPPCPAPEFLGG |
| 167 | ESKYGPPCPPCPA |
| 168 | ESKYGPPCPSCPA |
| 150 | ESKYGPPCPSCPAPEFLGGPSVFL |
| 169 | ESKYGPPCPPCPAPEFLGGPSVFL |
| 170 | ERKCCVECPPCPAPPVAG |
| 171 | ERKCCVECPPCPA |
| 172 | EPKSCDKTHTCPPCPAPELAGA |
| 173 | EPKSCDKTHTCPPCPAPELAGAPSVFL |
| 174 | EPKSCDKTHLCPPCPAPELAGAPSVFL |
| 175 | EPKSCDKTHTCPPCPAPEFEGG |
| 176 | EPKSCDKTHTCPPCPAPEFEGGPSVFL |
| 177 | EPKSCDKTHLCPPCPAPEFEGGPSVFL |

In certain embodiments, the viral vectors provided herein comprise the following elements in the following order: a) a constitutive or inducible (e.g., hypoxia-inducible or rifamycin-inducible) promoter sequence or a tissue specific promoter/regulatory region, for example, one of the regulatory regions provided in Table 1 or 1a, and b) a sequence encoding the transgene (e.g., a HuGlyFab). In certain embodiments, the sequence encoding the transgene comprises multiple ORFs separated by IRES elements. In certain embodiments, the ORFs encode the heavy and light chain domains of the HuGlyFab. In certain embodiments, the sequence encoding the transgene comprises multiple subunits in one ORF separated by F/F2A sequences or F/T2A sequences. In certain embodiments, the sequence comprising the transgene encodes the heavy and light chain domains of the HuGlyFab separated by an F/F2A sequence or a F/T2A sequence. In certain embodiments, the sequence comprising the transgene encodes the heavy and light chain variable domains of the HuGlyFab separated by a flexible peptide linker (as an scFv). In certain embodiments, the viral vectors provided herein comprise the following elements in the following order: a) a constitutive or an inducible promoter sequence or a tissue specific promoter, such as one of the promoters or regulatory regions in Table 1 or 1a, and b) a sequence encoding the transgene (e.g., a HuGlyFab), wherein the transgene comprises a nucleotide sequence encoding a signal peptide, a light chain and a heavy chain Fab portion separated by an IRES element. In certain embodiments, the viral vectors provided herein comprise the following elements in the following order: a) a constitutive or a hypoxia-inducible promoter sequence or regulatory element listed in Table 1 or 1a, and b) a sequence encoding the transgene comprising a signal peptide, a light chain and a heavy chain sequence separated by a cleavable F/F2A sequence (SEQ ID NOS:143 or 144) or a F/T2A sequence (SEQ ID NOS:141 or 142) or a flexible peptide linker.

In certain embodiments, the viral vectors provided herein comprise the following elements in the following order: a) a first ITR sequence, b) a first linker sequence, c) a constitutive or an inducible promoter sequence or a tissue specific promoter or regulatory region, d) a second linker sequence, e) an intron sequence, f) a third linker sequence, g) a first UTR sequence, h) a sequence encoding the transgene (e.g., a HuGlyFab), i) a second UTR sequence, j) a fourth linker sequence, k) a poly A sequence, l) a fifth linker sequence, and m) a second ITR sequence.

In certain embodiments, the viral vectors provided herein comprise the following elements in the following order: a) a first ITR sequence, b) a first linker sequence, c) a constitutive or an inducible promoter sequence or a tissue specific regulatory region, d) a second linker sequence, e) an intron sequence, f) a third linker sequence, g) a first UTR sequence, h) a sequence encoding the transgene (e.g., HuGlyFab), i) a second UTR sequence, j) a fourth linker sequence, k) a poly A sequence, l) a fifth linker sequence, and m) a second ITR sequence, wherein the transgene comprises a signal, and wherein the transgene encodes a light chain and a heavy chain sequence separated by a cleavable F/2A sequence.

5.1.9. Fc Region Modifications

In certain embodiments, the transgenes encode full length or substantially full length heavy and light chains that associate to form a full length or intact antibody. (“Substantially intact” or “substantially full length” refers to a mAb having a heavy chain sequence that is at least 95% identical to the full-length heavy chain mAb amino acid sequence and a light chain sequence that is at least 95% identical to the full-length light chain mAb amino acid sequence). Accordingly, the transgenes comprise nucleotide sequences that encode, for example, the light and heavy chains of the Fab fragments including the hinge region of the heavy chain and C-terminal of the heavy chain of the Fab fragment, an Fc domain peptide. Table 6 provides the amino acid sequence of the Fc polypeptides for adalimumab, infliximab, golimumab, satralizumab, sarilumab, siltuximab, clazakizumab, sirukumab, olokizumab, and tocilizumab. Alternatively, an IgG1, IgG2, or IgG4 Fc domain, the sequences of which are provided in Table 6 may be utilized.

The term “Fc region” refers to a dimer of two “Fc polypeptides” (or “Fc domains”), each “Fc polypeptide” comprising the heavy chain constant region of an antibody excluding the first constant region immunoglobulin domain. In some embodiments, an “Fc region” includes two Fc polypeptides linked by one or more disulfide bonds, chemical linkers, or peptide linkers. “Fc polypeptide” refers to at least the last two constant region immunoglobulin domains of IgA, IgD, and IgG, or the last three constant region immunoglobulin domains of IgE and IgM and may also include part or all of the flexible hinge N-terminal to these domains. For IgG, e.g., “Fc polypeptide” comprises immunoglobulin domains Cgamma2 (Cy2, often referred to as CH2 domain) and Cgamma3 (Cy3, also referred to as CH3 domain) and may include the lower part of the hinge domain between Cgamma1 (Cγ1, also referred to as CH1 domain) and CH2 domain. Although the boundaries of the Fc polypeptide may vary, the human IgG heavy chain Fc polypeptide is usually defined to comprise residues starting at T223 or C226 or P230, to its carboxyl-terminus, wherein the numbering is according to the EU index as in Kabat et al. (1991, NIH Publication 91-3242, National Technical Information Services, Springfield, Va.). For IgA, e.g., Fc polypeptide comprises immunoglobulin domains Calpha2 (Cα2) and Calpha3 (Cα3) and may include the lower part of the hinge between Calpha1 (Cα1) and Cα2.

In certain embodiments, the Fc polypeptide is that of the therapeutic antibody or is the Fc polypeptide corresponding to the isotype of the therapeutic antibody). In still other embodiments, the Fc polypeptide is an IgG Fc polypeptide. The Fc polypeptide may be from the IgG1, IgG2, or IgG4 isotype (see Table 6) or may be an IgG3 Fc domain, depending, for example, upon the desired effector activity of the therapeutic antibody. In some embodiments, the engineered heavy chain constant region (CH), which includes the Fc domain, is chimeric. As such, a chimeric CH region combines CH domains derived from more than one immunoglobulin isotype and/or subtype. For example, the chimeric (or hybrid) CH region comprises part or all of an Fc region from IgG, IgA and/or IgM. In other examples, the chimeric CH region comprises part or all a CH2 domain derived from a human IgG1, human IgG2, or human IgG4 molecule, combined with part or all of a CH3 domain derived from a human IgG1, human IgG2, or human IgG4 molecule. In other embodiments, the chimeric CH region contains a chimeric hinge region.

TABLE 6
Table of Fc Domain Amino Acid Sequences

| | Chain/ | |
|---|---|---|
| | SEQ ID | |
| Fc | NO. | Sequence |
| IgG1 | Fc | FPPKPKDTLM ISRTPEVTCV VVDVSHEDPE VKFNWYVDGV |
| | domain/ | EVHNAKTKPR EEQYNSTYRV VSVLTVLHQD WLNGKEYKCK |
| | SEQ ID | VSNKALPAPI EKTISKAKGQ PREPQVYTLP PSRDELTKNQ |
| | NO: 61 | VSLTCLVKGF YPSDIAVEWE SNGQPENNYK TTPPVLDSDG |
| | | SFFLYSKLTV DKSRWQQGNV FSCSVMHEAL |
| | | HNHYTQKSLSLSPGK |
| IgG2 | Fc | FPPKPKDTLM ISRTPEVTCV VVDVSHEDPE VQFNWYVDGV |
| | domain/ | EVHNAKTKPR EEQFNSTFRV VSVLTVVHQD WLNGKEYKCK |
| | SEQ ID | VSNKGLPAPI EKTISKTKGQ PREPQVYTLP PSREEMTKNQ |
| | NO: 62 | VSLTCLVKGF YPSDISVEWE SNGQPENNYK TTPPMLDSDG |
| | | SFFLYSKLTV DKSRWQQGNV FSCSVMHEAL HNHYTQKSLS |
| | | LSPGK |
| IgG4 | Fc | FPPKPKDTLM ISRTPEVTCV VVDVSQEDPE VQFNWYVDGV |
| | domain/ | EVHNAKTKPR EEQFNSTYRV VSVLTVLHQD WLNGKEYKCK |
| | SEQ ID | VSNKGLPSSI EKTISKAKGQ PREPQVYTLP PSQEEMTKNQ |
| | NO: 63 | VSLTCLVKGF YPSDIAVEWE SNGQPENNYK TTPPVLDSDG |
| | | SFFLYSRLTV DKSRWQEGNV FSCSVMHEAL HNHYTQKSLS |
| | | LSLGK |
| Adalimumab | Fc | FPPKPKDTLM ISRTPEVTCV VVDVSHEDPE VKFNWYVDGV |
| | domain/ | EVHNAKTKPR EEQYNSTYRV VSVLTVLHQD WLNGKEYKCK |
| | SEQ ID | VSNKALPAPI EKTISKAKGQ PREPQVYTLP TTPPVLDSDG |
| | NO: 64 | VSLTCLVKGF YPSDIAVEWE SNGQPENNYK HNHYTQKSLS |
| | | SFFLYSKLTV DKSRWQQGNV FSCSVMHEAL PSRDELTKNQ |
| | | LSPGK |
| Infliximab | Fc | FPPKPKDTLM ISRTPEVTCV VVDVSHEDPE VKFNWYVDGV |
| | domain/ | EVHNAKTKPR EEQYNSTYRV VSVLTVLHQD WLNGKEYKCK |
| | SEQ ID | VSNKALPAPI EKTISKAKGQ PREPQVYTLP PSRDELTKNQ |
| | NO: 65 | VSLTCLVKGF YPSDIAVEWE SNGQPENNYK TTPPVLDSDG |
| | | SFFLYSKLTV DKSRWQQGNV FSCSVMHEAL HNHYTQKSLS |
| | | LSPGK |
| Golimumab | Fc | FPPKPKDTLM ISRTPEVTCV VVDVSHEDPE VKFNWYVDGV |
| | domain/ | EVHNAKTKPR EEQYNSTYRV VSVLTVLHQD WLNGKEYKCK |
| | SEQ ID | VSNKALPAPI EKTISKAKGQ PREPQVYTLP PSRDELTKNQ |
| | NO: 66 | VSLTCLVKGF YPSDIAVEWE SNGQPENNYK TTPPVLDSDG |
| | | SFFLYSKLTV DKSRWQQGNV FSCSVMHEAL HNHYTQKSLS |
| | | LSPGK |
| Satralizumab | Fc | FPPKPKDTLM ISRTPEVTCV VVDVSQEDPE VQFNWYVDGV |
| | domain/ | EVHNAKTKPR EEQFNSTFRV VSVLTVVHQD WLNGKEYKCK |
| | SEQ ID | VSNKGLPAPI EKTISKTKGQ PREPQVYTLP PSQEEMTKNQ |
| | NO: 67 | VSLTCLVKGF YPSDIAVEWE SNGQPENNYK TTPPMLDSDG |
| | | SFFLYSKLTV DKSRWQEGNV FSCSVMHEAL HAHYTQKSLS |
| | | LSP |
| Sarilumab | Fc | FPPKPKDTLM ISRTPEVTCV VVDVSHEDPE VKFNWYVDGV |
| | domain/ | EVHNAKTKPR EEQYNSTYRV VSVLTVLHQD WLNGKEYKCK |
| | SEQ ID | VSNKALPAPI EKTISKAKGQ PREPQVYTLP PSRDELTKNQ |
| | NO: 185 | VSLTCLVKGF YPSDIAVEWE SNGQPENNYK TTPPVLDSDG |
| | | SFFLYSKLTV DKSRWQQGNV FSCSVMHEAL HNHYTQKSLS |
| | | LSPGK |
| Siltuximab | Fc | FPPKPKDTLM ISRTPEVTCV VVDVSHEDPE VKFNWYVDGV |
| | domain/ | EVHNAKTKPR EEQYNSTYRV VSVLTVLHQD WLNGKEYKCK |
| | SEQ ID | VSNKALPAPI EKTISKAKGQ PREPQVYTLP PSRDELTKNQ |
| | NO: 68 | VSLTCLVKGF YPSDIAVEWE SNGQPENNYK TTPPVLDSDG |
| | | SFFLYSKLTV DKSRWQQGNV FSCSVMHEAL HNHYTQKSLS |
| | | LSPGK |
| Clazakizumab | Fc | FPPKPKDTLM ISRTPEVTCV VVDVSHEDPE VKFNWYVDGV |
| | domain/ | EVHNAKTKPR EEQYASTYRV VSVLTVLHQD WINGKEYKCK |
| | SEQ ID | VSNKALPAPI EKTISKAKGQ PREPQVYTLP PSREEMTKNQ |
| | NO: 69 | VSLTCLVKGF YPSDIAVEWE SNGQPENNYK TTPPVLDSDG |
| | | SFFLYSKLTV DKSRWQQGNV FSCSVMHEAL HNHYTQKSLS |
| | | LSPGK |
| Sirukumab | Fc | FPPKPKDTLM ISRTPEVTCV VVDVSHEDPE VKFNWYVDGV |
| | domain/ | EVHNAKTKPR EEQYNSTYRV VSVLTVLHQD WLNGKEYKCK |
| | SEQ ID | VSNKALPAPI EKTISKAKGQ PREPQVYTLP PSRDELTKNQ |
| | NO: 70 | VSLTCLVKGF YPSDIAVEWE SNGQPENNYK TTPPVLDSDG |
| | | SFFLYSKLTV DKSRWQQGNV FSCSVMHEAL HNHYTQKSLS |
| | | LSPGK |
| Olokizumab | Fc | FPPKPKDTLM ISRTPEVTCV VVDVSQEDPE VQFNWYVDGV |
| | domain/ | EVHNAKTKPR EEQFNSTYRV VSVLTVLHQD WINGKEYKCK |
| | SEQ ID | VSNKGLPSSI EKTISKAKGQ PREPQVYTLP PSQEEMTKNQ |
| | NO: 71 | VSLTCLVKGF YPSDIAVEWE SNGQPENNYK TTPPVLDSDG |
| | | SFFLYSRLTV DKSRWQEGNV FSCSVMHEAL HNHYTQKSLS |
| | | LSLGK |
| Tocilizumab | Fc | FPPKPKDTLM ISRTPEVTCV VVDVSHEDPE VKFNWYVDGV |
| | domain/ | EVHNAKTKPR EEQYNSTYRV VSVLTVLHQD WLNGKEYKCK |
| | SEQ ID | VSNKALPAPI EKTISKAKGQ PREPQVYTLP PSRDELTKNQ |
| | NO: 72 | VSLTCLVKGF YPSDIAVEWE SNGQPENNYK TTPPVLDSDG |
| | | SFFLYSKLTV DKSRWQQGNV FSCSVMHEAL HNHYTQKSLS |
| | | LSPG |

In some embodiments, the recombinant vectors encode therapeutic antibodies comprising an engineered (mutant) Fc regions, e.g. engineered Fc regions of an IgG constant region. Modifications to an antibody constant region, Fc region or Fc fragment of an IgG antibody may alter one or more effector functions such as Fc receptor binding or neonatal Fc receptor (FcRn) binding and thus half-life, CDC activity, ADCC activity, and/or ADPC activity, compared to a corresponding antibody having a wild-type IgG constant region, or an IgG heavy chain constant region without the recited modification(s). Accordingly, in some embodiments, the antibody may be engineered to provide an antibody constant region, Fc region or Fc fragment of an IgG antibody that exhibits altered binding (as compared to a reference or wild-type constant region without the recited modification(s)) to one or more Fc receptors (e.g., FcγRI, FcγRIIA, FcγRIIB, FcγRIIIA, FcγRIIIB, FcγRIV, or FcRn receptor). In some embodiments, the antibody an antibody constant region, Fc region or Fc fragment of an IgG antibody that exhibits a one or more altered effector functions such as CDC, ADCC, or ADCP activity, compared to a corresponding antibody having a wild-type IgG constant region, or an IgG constant without the recited modification(s).

“Effector function” refers to a biochemical event that results from the interaction of an antibody Fc region with an Fc receptor or ligand. Effector functions include FcγR-mediated effector functions such as ADCC and ADCP and complement-mediated effector functions such as CDC.

An “effector cell” refers to a cell of the immune system that expresses one or more Fc receptors and mediates one or more effector functions. Effector cells include but are not limited to monocytes, macrophages, neutrophils, dendritic cells, eosinophils, mast cells, platelets, B cells, large granular lymphocytes, Langerhans' cells, natural killer (NK) cells, and T cells, and may be from any organism including but not limited to humans, mice, rats, rabbits, and monkeys.

“ADCC” or “antibody dependent cell-mediated cytotoxicity” refers to the cell-mediated reaction wherein nonspecific cytotoxic effector (immune) cells that express FcγRs recognize bound antibody on a target cell and subsequently cause lysis of the target cell.

“ADCP” or “antibody dependent cell-mediated phagocytosis” refers to the cell-mediated reaction wherein nonspecific cytotoxic effector (immune) cells that express FcγRs recognize bound antibody on a target cell and subsequently cause phagocytosis of the target cell.

“CDC” or “complement-dependent cytotoxicity” refers to the reaction wherein one or more complement protein components recognize bound antibody on a target cell and subsequently cause lysis of the target cell.

In some embodiments, the modifications of the Fc domain include, but are not limited to, the following modifications and combinations thereof, with reference to EU numbering of an IgG constant region (see FIG. 6): 233, 234, 235, 236, 237, 238, 239, 248, 249, 250, 252, 254, 255, 256, 258, 265, 267, 268, 269, 270, 272, 276, 278, 280, 283, 285, 286, 289, 290, 292, 293, 294, 295, 296, 297, 298, 301, 303, 305, 307, 308, 309, 311, 312, 315, 318, 320, 322, 324, 326, 327, 328, 329, 330, 331, 332, 333, 334, 335, 337, 338, 339, 340, 342, 344, 356, 358, 359, 360, 361, 362, 373, 375, 376, 378, 380, 382, 383, 384, 386, 388, 389, 398, 414, 416, 419, 428, 430, 433, 434, 435, 437, 438, and 439.

In certain embodiments, the Fc region comprises an amino acid addition, deletion, or substitution of one or more of amino acid residues 251-256, 285-290, 308-314, 385-389, and 428-436 of the IgG. In some embodiments, 251-256, 285-290, 308-314, 385-389, and 428-436 (EU numbering of Kabat; see FIG. 6) is substituted with histidine, arginine, lysine, aspartic acid, glutamic acid, serine, threonine, asparagine, or glutamine. In some embodiments, a non-histidine residue is substituted with a histidine residue. In some embodiments, a histidine residue is substituted with a non-histidine residue.

Enhancement of FcRn binding by an antibody having an engineered Fc leads to preferential binding of the affinity-enhanced antibody to FcRn as compared to antibody having wild-type Fc, and thus leads to a net enhanced recycling of the FcRn-affinity-enhanced antibody, which results in further increased antibody half-life. An enhanced recycling approach allows highly effective targeting and clearance of antigens, including e.g. “high titer” circulating antigens, such as C5, cytokines, or bacterial or viral antigens.

Provided in certain embodiments are modified constant region, Fc region or Fc fragment of an IgG antibody with enhanced binding to FcRn in serum as compared to a wild-type Fc region (without engineered modifications). In some instances, antibodies, e.g. IgG antibodies, are engineered to bind to FcRn at a neutral pH, e.g., at or above pH 7.4, to enhance pH-dependence of binding to FcRn as compared to a wild-type Fc region (without engineered modifications). In some instances, antibodies, e.g. IgG antibodies, are engineered to exhibit enhanced binding (e.g. increased affinity or K_{D}) to FcRn in endosomes (e.g., at an acidic pH, e.g., at or below pH 6.0) relative to a wild-type IgG and/or reference antibody binding to FcRn at an acidic pH, as well as in comparison to binding to FcRn in serum (e.g., at a neutral pH, e.g., at or above pH 7.4). Provided are antibodies with an engineered antibody constant region, Fc region or Fc fragment of an IgG antibody that exhibits an improved serum or resident tissue half-life, compared to a corresponding antibody having a wild-type IgG constant region, or an IgG constant without the recited modification(s);

Non-limiting examples of such Fc modifications include, e.g., a modification at position 250 (e.g., E or Q); 250 and 428 (e.g., L or F); 252 (e.g., LN/Y/W or T), 254 (e.g., S or T), and 256 (e.g., S/R/Q/E/D or T); or a modification at position 428 and/or 433 (e.g., H/L/R/S/P/Q or K) and/or 434 (e.g., H/F or Y); or a modification at position 250 and/or 428; or a modification at position 307 or 308 (e.g., 308F, V308F), and 434. In one embodiment, the modification comprises a 428L (e.g., M428L) and 434S (e.g., N434S) modification; a 428L, 2591 (e.g., V2591), and 308F (e.g., V308F) modification; a 433K (e.g., H433K) and a 434 (e.g., 434Y) modification; a 252, 254, and 256 (e.g., 252Y, 254T, and 256E) modification; a 250Q and 428L modification (e.g., T250Q and M428L); and a 307 and/or 308 modification (e.g., 308F or 308P) (EU numbering; see FIG. 6).

In some embodiments, the Fc region can be a mutant form such as hIgG1 Fc including M252 mutations, e.g. M252Y and S254T and T256E (“YTE mutation”) exhibit enhanced affinity for human FcRn (Dall'Acqua, et al., 2002, J Immunol 169:5171-5180) and subsequent crystal structure of this mutant antibody bound to hFcRn resulting in the creation of two salt bridges (Oganesyan, et al. 2014, JBC 289(11): 7812-7824). Antibodies having the YTE mutation have been administered to monkeys and humans, and have significantly improved pharmacokinetic properties (Haraya, et al., 2019, Drug Metabolism and Pharmacokinetics, 34(1):25-41).

In some embodiments, modifications to one or more amino acid residues in the Fc region may reduce half-life in systemic circulation (serum), however result in improved retainment in tissues (e.g. in the eye) by disabling FcRn binding (e.g. H435A, EU numbering of Kabat) (Ding et al., 2017, MAbs 9:269-284; and Kim, 1999, Eur J Immunol 29:2819).

In some embodiments, the Fc domain may be engineered to activate all, some, or none of the normal Fc effector functions, without affecting the Fc polypeptide's (e.g. antibody's) desired pharmacokinetic properties. Fc polypeptides having altered effector function may be desirable as they may reduce unwanted side effects, such as activation of effector cells, by the therapeutic protein.

Methods to alter or even ablate effector function may include mutation(s) or modification(s) to the hinge region amino acid residues of an antibody. For example, IgG Fc domain mutants comprising 234A, 237A, and 238S substitutions, according to the EU numbering system, exhibit decreased complement dependent lysis and/or cell mediated destruction. Deletions and/or substitutions in the lower hinge, e.g. where positions 233-236 within a hinge domain (EU numbering) are deleted or modified to glycine, have been shown in the art to significantly reduce ADCC and CDC activity.

In specific embodiments, the Fc domain is an aglycosylated Fc domain that has a substitution at residue 297 or 299 to alter the glycosylation site at 297 such that the Fc domain is not glycosylated. Such aglycosylated Fc domains may have reduced ADCC or other effector activity.

Non-limiting examples of proteins comprising mutant and/or chimeric CH regions having altered effector functions, and methods of engineering and testing mutant antibodies, are described in the art, e.g. K. L. Amour, et al., Eur. J. Immunol. 1999, 29:2613-2624; Lazar et al., Proc. Natl. Acad. Sci. USA 2006, 103:4005; US Patent Application Publication No. 20070135620A1 published Jun. 14, 2007; US Patent Application Publication No. 20080154025 A1, published Jun. 26, 2008; US Patent Application Publication No. 20100234572 A1, published Sep. 16, 2010; US Patent Application Publication No. 20120225058 A1, published Sep. 6, 2012; US Patent Application Publication No. 20150337053 A1, published Nov. 26, 2015; International Publication No. WO20/16161010A2 published Oct. 6, 2016; U.S. Pat. No. 9,359,437, issued Jun. 7, 2016; and U.S. Pat. No. 10,053,517, issued Aug. 21, 2018, all of which are herein incorporated by reference.

The C-terminal lysines (−K) conserved in the heavy chain genes of all human IgG subclasses are generally absent from antibodies circulating in serum—the C-terminal lysines are cleaved off in circulation, resulting in a heterogeneous population of circulating IgGs. (van den Bremer et al., 2015, mAbs 7:672-680). In the vectored constructs for full length mAbs, the DNA encoding the C-terminal lysine (−K) or glycine-lysine (−GK) of the Fc terminus can be deleted to produce a more homogeneous antibody product in situ. (See, Hu et al., 2017 Biotechnol. Prog. 33: 786-794 which is incorporated by reference herein in its entirety).

5.1.10 Manufacture and Testing of Vectors

The viral vectors provided herein may be manufactured using host cells. The viral vectors provided herein may be manufactured using mammalian host cells, for example, A549, WEHI, BHK, MDCK, COS1, COS7, BSC 1, BSC 40, BMT 10, VERO, W138, HeLa, 293, Saos, C2C12, L, HT1080, HepG2, primary fibroblast, hepatocyte, and myoblast cells. The viral vectors provided herein may be manufactured using host cells from human, monkey, mouse, rat, rabbit, or hamster.

The host cells are stably transformed with the sequences encoding the transgene and associated elements (e.g., the vector genome), and the means of producing viruses in the host cells, for example, the replication and capsid genes (e.g., the rep and cap genes of AAV). For a method of producing recombinant AAV vectors with AAV8 capsids, see Section IV of the Detailed Description of U.S. Pat. No. 7,282,199 B2, which is incorporated herein by reference in its entirety. Genome copy titers of said vectors may be determined, for example, by TAQMAN® analysis. Virions may be recovered, for example, by CsCl₂ sedimentation.

Alternatively, baculovirus expression systems in insect cells may be used to produce AAV vectors. For a review, see Aponte-Ubillus et al., 2018, Appl. Microbiol. Biotechnol. 102:1045-1054 which is incorporated by reference herein in its entirety for manufacturing techniques.

In vitro assays, e.g., cell culture assays, can be used to measure transgene expression from a vector described herein, thus indicating, e.g., potency of the vector. In addition, in vitro neutralization assays can be used to measure the activity of the transgene expressed from a vector described herein. For example, Vero-E6 cells, a cell line derived from the kidney of an African green monkey, or HeLa cells engineered to stably express the ACE2 receptor (HeLa-ACE2), can be used to assess neutralization activity of transgenes expressed from a vector described herein. In addition, other characteristics of the expressed product can be determined, for example determination of the glycosylation and tyrosine sulfation patterns associated with the HuGlyFab. Glycosylation patterns and methods of determining the same are discussed in Section 5.3, while tyrosine sulfation patterns and methods of determining the same are discussed in Section 5.3. In addition, benefits resulting from glycosylation/sulfation of the cell-expressed HuGlyFab can be determined using assays known in the art, e.g., the methods described in Section 5.3.

Vector genome concentration (GC) or vector genome copies can be evaluated using digital PCR (dPCR) or ddPCR™ (BioRad Technologies, Hercules, CA, USA). In one example, ocular tissue samples, such as aqueous and/or vitreous humor samples, are obtained at several timepoints. In another example, several mice are sacrificed at various timepoints post injection. Ocular tissue samples are subjected to total DNA extraction and dPCR assay for vector copy numbers. Copies of vector genome (transgene) per gram of tissue may be measured in a single biopsy sample, or measured in various tissue sections at sequential timepoints will reveal spread of AAV throughout the eye. Total DNA from collected ocular fluid or tissue is extracted with the DNeasy Blood & Tissue Kit and the DNA concentration measured using a Nanodrop spectrophotometer. To determine the vector copy numbers in each tissue sample, digital PCR is performed with Naica Crystal Digital PCR system (Stilla technologies). Two color multiplexing system is applied to simultaneously measure the transgene AAV and an endogenous control gene. In brief, the transgene probe can be labelled with FAM (6-carboxyfluorescein) dye while the endogenous control probe can be labelled with VIC fluorescent dye. The copy number of delivered vector in a specific tissue section per diploid cell is calculated as: (vector copy number)/(endogenous control)×2. Vector copy in specific cell types or tissues, such as cornea, iris, ciliary body, schlemm's canal cells, trabecular meshwork, retinal cells, RPE cells, RPE-choroid tissue, or optic nerve cells, over time may indicate sustained expression of the transgene by the tissue.

5.1.11 Compositions

Pharmaceutical compositions suitable for administration to human subjects comprise a suspension of the recombinant vector in a formulation buffer comprising a physiologically compatible aqueous buffer, a surfactant and optional excipients. Such formulation buffer can comprise one or more of a polysaccharide, a surfactant, polymer, or oil. In some embodiments, the pharmaceutical composition comprises rAAV combined with a pharmaceutically acceptable carrier for administration to a subject. In one embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant (e.g., Freund's complete and incomplete adjuvant), excipient, or vehicle with which the agent is administered. Such pharmaceutical carriers can be sterile liquids, such as water and oils, including those of petroleum, animal, vegetable, or synthetic origin, including, e.g., peanut oil, soybean oil, mineral oil, sesame oil and the like. Water is a common carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. Additional examples of pharmaceutically acceptable carriers, excipients, and stabilizers include, but are not limited to, buffers such as phosphate, citrate, and other organic acids; antioxidants including ascorbic acid; low molecular weight polypeptides; proteins, such as serum albumin and gelatin; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN′, polyethylene glycol (PEG), and PLURONICS' as known in the art. The pharmaceutical composition of the present invention can also include a lubricant, a wetting agent, a sweetener, a flavoring agent, an emulsifier, a suspending agent, and a preservative, in addition to the above ingredients. These compositions can take the form of solutions, suspensions, emulsion, tablets, pills, capsules, powders, sustained-release formulations and the like.

5.2 Methods of Treating Non-Infectious Uveitis

In another aspect, methods for treating non-infectious uveitis or other indication that can be treated with an anti-TNFα, anti-IL6, or anti-IL6R antibody in a subject in need thereof comprising the administration of recombinant AAV particles comprising an expression cassette encoding anti-TNFα, anti-IL6, or anti-IL6R antibodies and antibody-binding fragments and variants thereof, or peptides, are provided. A subject in need thereof includes a subject suffering from non-infectious uveitis, or a subject pre-disposed thereto, e.g., a subject at risk of developing or having a recurrence of the non-infectious uveitis, or other indication that may be treated with an anti-TNFα antibody. Subjects to whom such gene therapy is administered can be those responsive to anti-TNFα, e.g. adalimumab, infliximab, or golimumab, or anti-IL6, or anti-IL6R therapy e.g., satralizumab, sarilumab, siltuximab, clazakizumab, sirukumab, olokizumab, gerilizumab, or tocilizumab. In particular embodiments, the methods encompass treating patients who have been diagnosed with non-infectious uveitis, and, in certain embodiments, identified as responsive to treatment with an anti-TNFα, anti-IL6, or anti-IL6R antibody or considered a good candidate for therapy with an anti-TNFα, anti-IL6, or anti-IL6R antibody. In specific embodiments, the patients have previously been treated with an anti-TNFα, anti-IL6, or anti-IL6R antibody. To determine responsiveness, the anti-TNFα, anti-IL6, or anti-IL6R antibody or antigen-binding fragment transgene product (e.g., produced in human cell culture, bioreactors, etc.) may be administered directly to the subject.

In specific embodiments, provided are methods of treating non-infectious uveitis or other indication amenable to treatment with an anti-TNFα, anti-IL6, or anti-IL6R antibody in a human subject in need thereof comprising: administering to the eye (or liver and/or muscle) of said subject a therapeutically effective amount of a recombinant nucleotide expression vector comprising a transgene encoding a substantially full-length or full-length anti-TNFα, anti-IL6, or anti-IL6R mAb having an Fc region, or an antigen-binding fragment thereof, operably linked to one or more regulatory sequences that control expression of the transgene in human ocular tissue cells, so that a depot is formed that releases a HuPTM form of mAb or antigen-binding fragment thereof. Subretinal, intravitreal, intracamerally, or suprachoroidal administration should result in expression of the soluble transgene product in one or more of the following retinal cell types: human photoreceptor cells (cone cells, rod cells); horizontal cells; bipolar cells; amarcrine cells; retina ganglion cells (midget cell, parasol cell, bistratified cell, giant retina ganglion cell, photosensitive ganglion cell, and muller glia); and retinal pigment epithelial cells or other ocular tissue cell: cornea cells, iris cells, ciliary body cells, a schlemm's canal cells, a trabecular meshwork cells, RPE-choroid tissue cells, or optic nerve cells.

Recombinant vectors and pharmaceutical compositions for treating diseases or disorders in a subject in need thereof are described in Section 5.1. Such vectors should have a tropism for human ocular tissue, or liver and/or muscle cells and can include non-replicating rAAV, particularly those bearing an AAV2.7m8, AAV3B, AAV8, AAAV9, AAV10, AAVrh10, or AAVrh73 capsid. The recombinant vectors can be administered in any manner such that the recombinant vector enters ocular tissue cells, e.g., by introducing the recombinant vector into the eye. Such vectors should further comprise one or more regulatory sequences that control expression of the transgene in human ocular tissue cells and/or human liver and muscle cells include, but are not limited to, human rhodopsin kinase (GRK1) promoter (SEQ ID NOS:77 or 217), a mouse cone arresting (CAR) promoter (SEQ ID NOS: 214-216), a human red opsin (RedO) promoter (SEQ ID NO: 212), a CAG promoter (SEQ ID NO: 74), a CB promoter or CBlong promoter (SEQ ID NO: 273 or 274) or a Best1/GRK1 tandem promoter (SEQ ID NO: 275) (see also Tables 1 and 1a).

5.3.N-Glycosylation, Tyrosine Sulfation, and O-Glycosylation

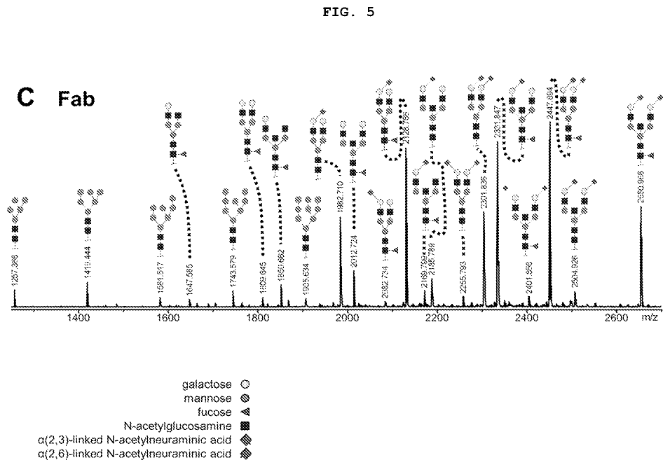

The amino acid sequence (primary sequence) of HuGlyFabs or HuPTM Fabs, HuPTMmAbs, and HuPTM scFvs disclosed herein each comprises at least one site at which N-glycosylation or tyrosine sulfation takes place (see exemplary FIG. 5) for glycosylation and/or sulfation positions within the amino acid sequences of the Fab fragments of the therapeutic antibodies). Post-translational modification also occurs in the Fc domain of full length antibodies, particularly at residue N297 (by EU numbering, see Table 6).

Alternatively, mutations may be introduced into the Fc domain to alter the glycosylation site at residue N297 (EU numbering, see Table 6), in particular substituting another amino acid for the asparagine at 297 or the threonine at 299 to remove the glycosylation site resulting in an aglycosylated Fc domain.

5.3.1. N-Glycosylation

Reverse Glycosylation Sites

The canonical N-glycosylation sequence is known in the art to be Asn-X-Ser (or Thr), wherein X can be any amino acid except Pro. However, it recently has been demonstrated that asparagine (Asn) residues of human antibodies can be glycosylated in the context of a reverse consensus motif, Ser (or Thr)-X-Asn, wherein X can be any amino acid except Pro. See Valliere-Douglass et al., 2009, J. Biol. Chem. 284:32493-32506; and Valliere-Douglass et al., 2010, J. Biol. Chem. 285:16012-16022. As disclosed herein, certain HuGlyFabs and HuPTM scFvs disclosed herein comprise such reverse consensus sequences.

Non-Consensus Glycosylation Sites

In addition to reverse N-glycosylation sites, it recently has been demonstrated that glutamine (Gln) residues of human antibodies can be glycosylated in the context of a non-consensus motif, Gln-Gly-Thr. See Valliere-Douglass et al., 2010, J. Biol. Chem. 285:16012-16022. Surprisingly, certain of the HuGlyFab fragments disclosed herein comprise such non-consensus sequences. In addition, O-glycosylation comprises the addition of N-acetyl-galactosamine to serine or threonine residues by the enzyme. It has been demonstrated that amino acid residues present in the hinge region of antibodies can be O-glycosylated. The possibility of O-glycosylation confers another advantage to the therapeutic antibodies provided herein, as compared to, e.g., antigen-binding fragments produced in E. coli, again because the E. coli naturally does not contain machinery equivalent to that used in human O-glycosylation. (Instead, O-glycosylation in E. coli has been demonstrated only when the bacteria is modified to contain specific O-glycosylation machinery. See, e.g., Farid-Moayer et al., 2007, J. Bacteriol. 189:8088-8098.)

Engineered N-Glycosylation Sites

In certain embodiments, a nucleic acid encoding a HuPTM mAb, HuGlyFab or HuPTM scFv is modified to include 1, 2, 3, 4, 5, 6, 7, 8, 9, 10, or more N-glycosylation sites (including the canonical N-glycosylation consensus sequence, reverse N-glycosylation site, and non-consensus N-glycosylation sites) than would normally be associated with the HuPTM mAb, HuGlyFab or HuPTM scFv (e.g., relative to the number of N-glycosylation sites associated with the HuPTM mAb, HuGlyFab or HuPTM scFv in its unmodified state). In specific embodiments, introduction of glycosylation sites is accomplished by insertion of N-glycosylation sites (including the canonical N-glycosylation consensus sequence, reverse N-glycosylation site, and non-consensus N-glycosylation sites) anywhere in the primary structure of the antigen-binding fragment, so long as said introduction does not impact binding of the antibody or antigen-binding fragment to its antigen. Introduction of glycosylation sites can be accomplished by, e.g., adding new amino acids to the primary structure of the antigen-binding fragment, or the antibody from which the antigen-binding fragment is derived (e.g., the glycosylation sites are added, in full or in part), or by mutating existing amino acids in the antigen-binding fragment, or the antibody from which the antigen-binding fragment is derived, in order to generate the N-glycosylation sites (e.g., amino acids are not added to the antigen-binding fragment/antibody, but selected amino acids of the antigen-binding fragment/antibody are mutated so as to form N-glycosylation sites). Those of skill in the art will recognize that the amino acid sequence of a protein can be readily modified using approaches known in the art, e.g., recombinant approaches that include modification of the nucleic acid sequence encoding the protein.

In a specific embodiment, a HuGlyMab or antigen-binding fragment is modified such that, when expressed in mammalian cells, such as retina, CNS, liver or muscle cells, it can be hyperglycosylated. See Courtois et al., 2016, mAbs 8:99-112 which is incorporated by reference herein in its entirety.

N-Glycosylation of HuPTM mAbs and HuPTM Antigen-Binding Fragments

Unlike small molecule drugs, biologics usually comprise a mixture of many variants with different modifications or forms that could have a different potency, pharmacokinetics, and/or safety profile. It is not essential that every molecule produced either in the gene therapy or protein therapy approach be fully glycosylated and sulfated. Rather, the population of glycoproteins produced should have sufficient glycosylation (including 2,6-sialylation) and sulfation to demonstrate efficacy. The goal of gene therapy treatment provided herein can be, for example, to slow or arrest the progression of a disease or abnormal condition or to reduce the severity of one or more symptoms associated with the disease or abnormal condition.

When a HuPTM mAb, HuGlyFab or HuPTM scFv is expressed in a human cell, the N-glycosylation sites of the antigen-binding fragment can be glycosylated with various different glycans. N-glycans of antigen-binding fragments and the Fc domain have been characterized in the art. For example, Bondt et al., 2014, Mol. & Cell. Proteomics 13.11:3029-3039 (incorporated by reference herein in its entirety for its disclosure of Fab-associated N-glycans; see also, FIG. 22) characterizes glycans associated with Fabs, and demonstrates that Fab and Fc portions of antibodies comprise distinct glycosylation patterns, with Fab glycans being high in galactosylation, sialylation, and bisection (e.g., with bisecting GlcNAc) but low in fucosylation with respect to Fc glycans. Like Bondt, Huang et al., 2006, Anal. Biochem. 349:197-207 (incorporated by reference herein in its entirety for it disclosure of Fab-associated N-glycans) found that most glycans of Fabs are sialylated. However, in the Fab of the antibody examined by Huang (which was produced in a murine cell background), the identified sialic residues were N-Glycolylneuraminic acid (“Neu5Gc” or “NeuGc”) (which is not natural to humans) instead of N-acetylneuraminic acid (“Neu5Ac,” the predominant human sialic acid). In addition, Song et al., 2014, Anal. Chem. 86:5661-5666 (incorporated by reference herein in its entirety for its disclosure of Fab-associated N-glycans) describes a library of N-glycans associated with commercially available antibodies.

Glycosylation of the Fc domain has been characterized and is a single N-linked glycan at asparagine 297 (EU numbering; see Table 6). The glycan plays an integral structural and functional role, impacting antibody effector function, such as binding to Fc receptor (see, for example, Jennewein and Alter, 2017, Trends In Immunology 38:358 for a discussion of the role of Fc glycosylation in antibody function). Removal of the Fc region glycan almost completely ablates effector function (Jennewien and Alter at 362). The composition of the Fc glycan has been shown to impact effector function, for example hypergalactosylation and reduction in fucosylation have been shown to increase ADCC activity while sialylation correlates with anti-inflammatory effects (Id. at 364). Disease states, genetics and even diet can impact the composition of the Fc glycan in vivo. For recombinantly expressed antibodies, the glycan composition can differ significantly by the type of host cell used for recombinant expression and strategies are available to control and modify the composition of the glycan in therapeutic antibodies recombinantly expressed in cell culture, such as CHO to alter effector function (see, for example, US 2014/0193404 by Hansen et al.). Accordingly, the HuPTM mAbs provided herein may advantageously have a glycan at N297 that is more like the native, human glycan composition than antibodies expressed in non-human host cells.

Importantly, when the HuPTM mAb, HuGlyFab or HuPTM scFv are expressed in human cells, the need for in vitro production in prokaryotic host cells (e.g., E. coli) or eukaryotic host cells (e.g., CHO cells or NS0 cells) is circumvented. Instead, as a result of the methods described herein, N-glycosylation sites of the HuPTM mAb, HuGlyFab or HuPTM scFv are advantageously decorated with glycans relevant to and beneficial to treatment of humans. Such an advantage is unattainable when CHO cells, NS0 cells, or E. coli are utilized in antibody/antigen-binding fragment production, because e.g., CHO cells (1) do not express 2,6 sialyltransferase and thus cannot add 2,6 sialic acid during N-glycosylation; (2) can add Neu5Gc as sialic acid instead of Neu5Ac; and (3) can also produce an immunogenic glycan, the α-Gal antigen, which reacts with anti-α-Gal antibodies present in most individuals, which at high concentrations can trigger anaphylaxis; and because (4) E. coli does not naturally contain components needed for N-glycosylation.

Assays for determining the glycosylation pattern of antibodies, including antigen-binding fragments are known in the art. For example, hydrazinolysis can be used to analyze glycans. First, polysaccharides are released from their associated protein by incubation with hydrazine (the Ludger Liberate Hydrazinolysis Glycan Release Kit, Oxfordshire, UK can be used). The nucleophile hydrazine attacks the glycosidic bond between the polysaccharide and the carrier protein and allows release of the attached glycans. N-acetyl groups are lost during this treatment and have to be reconstituted by re-N-acetylation. Glycans may also be released using enzymes such as glycosidases or endoglycosidases, such as PNGase F and Endo H, which cleave cleanly and with fewer side reactions than hydrazines. The free glycans can be purified on carbon columns and subsequently labeled at the reducing end with the fluorophor 2-amino benzamide. The labeled polysaccharides can be separated on a GlycoSep-N column (GL Sciences) according to the HPLC protocol of Royle et al, Anal Biochem 2002, 304(1):70-90. The resulting fluorescence chromatogram indicates the polysaccharide length and number of repeating units. Structural information can be gathered by collecting individual peaks and subsequently performing MS/MS analysis. Thereby the monosaccharide composition and sequence of the repeating unit can be confirmed and additionally in homogeneity of the polysaccharide composition can be identified. Specific peaks of low or high molecular weight can be analyzed by MALDI-MS/MS and the result used to confirm the glycan sequence. Each peak in the chromatogram corresponds to a polymer, e.g., glycan, consisting of a certain number of repeat units and fragments, e.g., sugar residues, thereof. The chromatogram thus allows measurement of the polymer, e.g., glycan, length distribution. The elution time is an indication for polymer length, while fluorescence intensity correlates with molar abundance for the respective polymer, e.g., glycan. Other methods for assessing glycans associated with antigen-binding fragments include those described by Bondt et al., 2014, Mol. & Cell. Proteomics 13.11:3029-3039, Huang et al., 2006, Anal. Biochem. 349:197-207, and/or Song et al., 2014, Anal. Chem. 86:5661-5666.

Homogeneity or heterogeneity of the glycan patterns associated with antibodies (including antigen-binding fragments), as it relates to both glycan length or size and numbers glycans present across glycosylation sites, can be assessed using methods known in the art, e.g., methods that measure glycan length or size and hydrodynamic radius. HPLC, such as size exclusion, normal phase, reversed phase, and anion exchange HPLC, as well as capillary electrophoresis, allows the measurement of the hydrodynamic radius. Higher numbers of glycosylation sites in a protein lead to higher variation in hydrodynamic radius compared to a carrier with less glycosylation sites. However, when single glycan chains are analyzed, they may be more homogenous due to the more controlled length. Glycan length can be measured by hydrazinolysis, SDS PAGE, and capillary gel electrophoresis. In addition, homogeneity can also mean that certain glycosylation site usage patterns change to a broader/narrower range. These factors can be measured by Glycopeptide LC-MS/MS.

In certain embodiments, the HuPTM mAbs, or antigen binding fragments thereof, also do not contain detectable NeuGc and/or α-Gal. By “detectable NeuGc” or “detectable α-Gal” or “does not contain or does not have NeuGc or α-Gal” means herein that the HuPTM mAb or antigen-binding fragment, does not contain NeuGc or α-Gal moieties detectable by standard assay methods known in the art. For example, NeuGc may be detected by HPLC according to Hara et al., 1989, “Highly Sensitive Determination of N-Acetyl-and N-Glycolylneuraminic Acids in Human Serum and Urine and Rat Serum by Reversed-Phase Liquid Chromatography with Fluorescence Detection.” J. Chromatogr., B: Biomed. 377, 111-119, which is hereby incorporated by reference for the method of detecting NeuGc. Alternatively, NeuGc may be detected by mass spectrometry. The α-Gal may be detected using an ELISA, see, for example, Galili et al., 1998, “A sensitive assay for measuring α-Gal epitope expression on cells by a monoclonal anti-Gal antibody.” Transplantation. 65(8):1129-32, or by mass spectrometry, see, for example, Ayoub et al., 2013, “Correct primary structure assessment and extensive glyco-profiling of cetuximab by a combination of intact, middle-up, middle-down and bottom-up ESI and MALDI mass spectrometry techniques.” Landes Bioscience. 5(5):699-710. See also the references cited in Platts-Mills et al., 2015, “Anaphylaxis to the Carbohydrate Side-Chain Alpha-gal” Immunol Allergy Clin North Am. 35(2): 247-260.

Benefits of N-Glycosylation

N-glycosylation confers numerous benefits on the HuPTM mAb, HuGlyFab or HuPTM scFv described herein. Such benefits are unattainable by production of antigen-binding fragments in E. coli, because E. coli does not naturally possess components needed for N-glycosylation. Further, some benefits are unattainable through antibody production in, e.g., CHO cells (or murine cells such as NS0 cells), because CHO cells lack components needed for addition of certain glycans (e.g., 2,6 sialic acid and bisecting GlcNAc) and because either CHO or murine cell lines add N-N-Glycolylneuraminic acid (“Neu5Gc” or “NeuGc”) which is not natural to humans (and potentially immunogenic), instead of N-Acetylneuraminic acid (“Neu5Ac”) the predominant human sialic acid. See, e.g., Dumont et al., 2015, Crit. Rev. Biotechnol. 36(6):1110-1122; Huang et al., 2006, Anal. Biochem. 349:197-207 (NeuGc is the predominant sialic acid in murine cell lines such as SP2/0 and NS0); and Song et al., 2014, Anal. Chem. 86:5661-5666, each of which is incorporated by reference herein in its entirety). Moreover, CHO cells can also produce an immunogenic glycan, the α-Gal antigen, which reacts with anti-α-Gal antibodies present in most individuals, which at high concentrations can trigger anaphylaxis. See, e.g., Bosques, 2010, Nat. Biotech. 28:1153-1156. The human glycosylation pattern of the HuGlyFab of HuPTM scFv described herein should reduce immunogenicity of the transgene product and improve efficacy.

While non-canonical glycosylation sites usually result in low level glycosylation (e.g., 1-5%) of the antibody population, the functional benefits may be significant (See, e.g., van de Bovenkamp et al., 2016, J. Immunol. 196:1435-1441). For example, Fab glycosylation may affect the stability, half-life, and binding characteristics of an antibody. To determine the effects of Fab glycosylation on the affinity of the antibody for its target, any technique known to one of skill in the art may be used, for example, enzyme linked immunosorbent assay (ELISA), or surface plasmon resonance (SPR). To determine the effects of Fab glycosylation on the half-life of the antibody, any technique known to one of skill in the art may be used, for example, by measurement of the levels of radioactivity in the blood or organs in a subject to whom a radiolabelled antibody has been administered. To determine the effects of Fab glycosylation on the stability, for example, levels of aggregation or protein unfolding, of the antibody, any technique known to one of skill in the art may be used, for example, differential scanning calorimetry (DSC), high performance liquid chromatography (HPLC), e.g., size exclusion high performance liquid chromatography (SEC-HPLC), capillary electrophoresis, mass spectrometry, or turbidity measurement.

The presence of sialic acid on HuPTM mAb, HuGlyFab or HuPTM scFv used in the methods described herein can impact clearance rate of the HuPTM mAb, HuGlyFab or HuPTM scFv. Accordingly, sialic acid patterns of a HuPTM mAb, HuGlyFab or HuPTM scFv can be used to generate a therapeutic having an optimized clearance rate. Methods of assessing antigen-binding fragment clearance rate are known in the art. See, e.g., Huang et al., 2006, Anal. Biochem. 349:197-207.

In another specific embodiment, a benefit conferred by N-glycosylation is reduced aggregation. Occupied N-glycosylation sites can mask aggregation prone amino acid residues, resulting in decreased aggregation. Such N-glycosylation sites can be native to an antigen-binding fragment used herein or engineered into an antigen-binding fragment used herein, resulting in HuGlyFab or HuPTM scFv that is less prone to aggregation when expressed, e.g., expressed in human cells. Methods of assessing aggregation of antibodies are known in the art. See, e.g., Courtois et al., 2016, mAbs 8:99-112 which is incorporated by reference herein in its entirety.

In another specific embodiment, a benefit conferred by N-glycosylation is reduced immunogenicity. Such N-glycosylation sites can be native to an antigen-binding fragment used herein or engineered into an antigen-binding fragment used herein, resulting in HuPTM mAb, HuGlyFab or HuPTM scFv that is less prone to immunogenicity when expressed, e.g., expressed in human ocular tissue cells, human CNS cells, human liver cells or human muscle cells.

In another specific embodiment, a benefit conferred by N-glycosylation is protein stability. N-glycosylation of proteins is well-known to confer stability on them, and methods of assessing protein stability resulting from N-glycosylation are known in the art. See, e.g., Sola and Griebenow, 2009, J Pharm Sci., 98(4): 1223-1245.

In another specific embodiment, a benefit conferred by N-glycosylation is altered binding affinity. It is known in the art that the presence of N-glycosylation sites in the variable domains of an antibody can increase the affinity of the antibody for its antigen. See, e.g., Bovenkamp et al., 2016, J. Immunol. 196:1435-1441. Assays for measuring antibody binding affinity are known in the art. See, e.g., Wright et al., 1991, EMBO J. 10:2717-2723; and Leibiger et al., 1999, Biochem. J. 338:529-538.

5.3.2 Tyrosine Sulfation

Tyrosine sulfation occurs at tyrosine (Y) residues with glutamate (E) or aspartate (D) within +5 to −5 position of Y, and where position −1 of Y is a neutral or acidic charged amino acid, but not a basic amino acid, e.g., arginine (R), lysine (K), or histidine (H) that abolishes sulfation. The HuGlyFabs and HuPTM scFvs described herein comprise tyrosine sulfation sites (see exemplary FIG. 2).

Importantly, tyrosine-sulfated antigen-binding fragments cannot be produced in E. coli, which naturally does not possess the enzymes required for tyrosine-sulfation. Further, CHO cells are deficient for tyrosine sulfation—they are not secretory cells and have a limited capacity for post-translational tyrosine-sulfation. See, e.g., Mikkelsen & Ezban, 1991, Biochemistry 30: 1533-1537. Advantageously, the methods provided herein call for expression of HuPTM Fab in human cells that are secretory and have capacity for tyrosine sulfation.

Tyrosine sulfation is advantageous for several reasons. For example, tyrosine-sulfation of the antigen-binding fragment of therapeutic antibodies against targets has been shown to dramatically increase avidity for antigen and activity. See, e.g., Loos et al., 2015, PNAS 112: 12675-12680, and Choe et al., 2003, Cell 114: 161-170. Assays for detection tyrosine sulfation are known in the art. See, e.g., Yang et al., 2015, Molecules 20:2138-2164.

5.3.3 O-Glycosylation

O-glycosylation comprises the addition of N-acetyl-galactosamine to serine or threonine residues by the enzyme. It has been demonstrated that amino acid residues present in the hinge region of antibodies can be O-glycosylated. In certain embodiments, the HuGlyFab comprise all or a portion of their hinge region, and thus are capable of being O-glycosylated when expressed in human cells. The possibility of O-glycosylation confers another advantage to the HuGlyFab provided herein, as compared to, e.g., antigen-binding fragments produced in E. coli, again because the E. coli naturally does not contain machinery equivalent to that used in human O-glycosylation. (Instead, O-glycosylation in E. coli has been demonstrated only when the bacteria is modified to contain specific machinery. See, e.g., Farid-Moayer et al., 2007, J. Bacteriol. 189:8088-8098.) O-glycosylated HuGlyFab, by virtue of possessing glycans, shares advantageous characteristics with N-glycosylated HuGlyFab (as discussed above).

5.4 Anti-TNFα HuPTM Constructs and Formulations for Non-Infectious Uveitis

Compositions and methods are described for the delivery of HuPTM mAb or the antigen-binding fragment thereof, such as HuPTM Fab, that bind to TNFα, derived from an anti-TNFα antibody and indicated for treating non-infectious uveitis. In certain embodiments, the HuPTM mAb has the amino acid sequence of adalimumab, infliximab, or golimumab or an antigen binding fragment thereof. The amino acid sequence of Fab fragment of these antibodies is provided in FIGS. 2A-2C. Delivery may be accomplished via gene therapy—e.g., by administering a viral vector or other DNA expression construct encoding an TNFα-binding HuPTM mAb (or an antigen binding fragment and/or a hyperglycosylated derivative or other derivative, thereof) to patients (human subjects) diagnosed with non-infectious uveitis to create a permanent depot that continuously supplies the human PTM, e.g., human-glycosylated, transgene product.

Transgenes

Provided are recombinant vectors containing a transgene encoding a HuPTM mAb or HuPTM Fab (or other antigen binding fragment of the HuPTM mAb) that binds to TNFα that can be administered to deliver the HuPTM mAb or antigen binding fragment in a patient. The transgene is a nucleic acid comprising the nucleotide sequences encoding an antigen binding fragment of an antibody that binds to TNFα, such as adalimumab, infliximab, or golimumab, or variants thereof as detailed herein. The transgene may also encode an anti-TNFα antigen binding fragment that contains additional glycosylation sites (e.g., see Courtois et al.).

In certain embodiments, the anti-TNFα antigen-binding fragment transgene comprises the nucleotide sequences encoding the heavy and light chains of the Fab portion of adalimumab (having amino acid sequences of SEQ ID NOs. 1 and 2, respectively, see Table 7 and FIG. 2A). The nucleotide sequences may be codon optimized for expression in human cells. Nucleotide sequences may, for example, comprise the nucleotide sequences of SEQ ID NO: 26 (encoding the adalimumab heavy chain Fab portion) and SEQ ID NO: 27 (encoding the adalimumab light chain Fab portion) as set forth in Table 8. The heavy and light chain sequences both have a signal or leader sequence at the N-terminus appropriate for expression and secretion in human cells, in particular, human ocular tissue cells (e.g., retinal cells) or liver and/or muscle cells. The signal sequence may have the amino acid sequence of MYRMQLLLLIALSLALVTNS (SEQ ID NO:85). Alternatively, the signal sequence may have an amino acid sequence selected from any one of the signal sequences set forth in Table 2 that correspond to the proteins secreted by ocular tissue cell types. Alternatively, the signal sequence may be appropriate for expression in muscle or liver cells, such as those listed in Tables 3 and 4 infra.

In addition to the heavy and light chain variable domain and CH1 and CL domain sequences, the transgenes may comprise, at the C-terminus of the heavy chain CH1 domain sequence, all or a portion of the hinge region. In specific embodiments, the anti-TNFα-antigen binding domain has a heavy chain Fab domain of SEQ ID NO: 1 with additional hinge region sequence starting after the C-terminal valine (V), contains all or a portion of the amino acid sequence EPKSCDKTHTCPPCPAPELLGG (SEQ ID NO:153), and specifically, EPKSCDKTHL (SEQ ID NO:155), EPKSCDKTHT (SEQ ID NO:156), EPKSCDKTHTCPPCPA (SEQ ID NO:157), EPKSCDKTHLCPPCPA (SEQ ID NO:158), EPKSCDKTHTCPPCPAPELLGGPSVFL (SEQ ID NO:159) or EPKSCDKTHLCPPCPAPELLGGPSVFL (SEQ ID NO:160) as set forth in FIG. 2A. These hinge regions may be encoded by nucleotide sequences at the 3′ end of SEQ ID NO: 26 by the hinge region encoding sequences set forth in Table 7 (SEQ ID NO: 26). In another embodiment, the transgenes comprise the amino acid sequences encoding the full length (or substantially full length) heavy and light chains of the antibody, comprising the Fc domain at the C terminus of the heavy chain, e.g., having an amino acid sequence of SEQ ID NO: 64 (Table 6) or an IgG1 Fc domain, such as SEQ ID No. 61 or as depicted in FIG. 6, or a mutant or variant thereof. The Fc domain may be engineered for altered binding to one or more Fc receptors and/or effector function as disclosed in Section 5.1.9, infra.

In specific embodiments, provided are constructs encoding a full length adalimumab, including the Fc domain, particularly nucleotide sequence of CAG.adalimumab.IgG (SEQ ID NOs: 46, 47, or 48), GRK1.adalimumab.IgG (SEQ ID NOs: 52 or 53), CB.VH4.adalimumab (SEQ ID NO: 276 or 277), Best1.GRK1.VH4.adalimumab, or an antigen-binding fragment of adalimumab, particularly CAG.adalimumab.Fab (SEQ ID NOS: 49 or 50), mU1a.adalimumab.Fab (SEQ ID NOS:224 or 225), and EF1a.adalimumab.Fab (SEQ ID NOs:222 or 223) as set forth in Table 8, herein, in certain cases depleted for CpG dimers. The transgene may also comprises a nucleotide sequence that encodes a signal peptide MYRMQLLLLIALSLALVTNS (SEQ ID NO:85); for example at the N-terminal of the heavy and/or the light chain) which may be encoded by the nucleotide sequence of SEQ ID NO:86. The nucleotide sequences encoding the light chain and heavy chain may be separated by a Furin-2A linker (SEQ ID NOs:146-149, see also amino acid sequences of SEQ ID NOs:142 and 144) to create a bicistronic vector. Alternatively, the nucleotide sequences of the light chain and heavy chain are separated by a Furin-T2A linker, such as SEQ ID NO:145. Expression of the adalimumab may be directed by a constitutive or a tissue specific promoter. In certain embodiments, the transgene contains a CAG promoter (SEQ ID NO: 74), a CB promoter or CB long promoter (SEQ ID NO: 273 or 274), a GRK1 (SEQ ID NO:77) promoter. Alternatively, the promoter may be a tissue specific promoter (or regulatory sequence including promoter and enhancer elements) such as the GRK1 promoter (SEQ ID NO:77 or 217), (a mouse cone arresting (CAR) promoter (SEQ ID NOS: 214-216), a human red opsin (RedO) promoter (SEQ ID NO: 212) or a Best1/GRK1 tandem promoter (SEQ ID NO: 275). In embodiments, a intron sequence is positioned between the promoter and the coding sequence, for example a VH4 intron sequence (SEQ ID NO: 70). The transgenes may contain elements provided in Table 1 or 1a. Exemplary transgenes encoding full length adalimumab are provided in Table 8 and include CAG.Adalimumab.T2A (SEQ ID NO: 46 to 48); GRK1.Adalimumab (SEQ ID NO: 52 and 53). ITR sequences are added to the 5′ and 3′ ends of the constructs to generate the genomes. including pAAV.CB.VH4.adalimumab (SEQ ID NO: 277), pAAV.CBlong.VH4.adalimumab, or pAAV.Best1.GRK1.VH4 adalimumab. The transgenes may be packaged into AAV, particularly AAV8.

In certain embodiments, the anti-TNFα antigen-binding fragment transgene encodes an TNFα antigen-binding fragment comprising a light chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 2. In certain embodiments, the anti-TNFα antigen-binding fragment transgene encodes an TNFα antigen-binding fragment comprising a heavy chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 1. In certain embodiments, the anti-TNFα antigen-binding fragment transgene encodes an antigen-binding fragment comprising a light chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 2 and a heavy chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 1. In specific embodiments, the TNFα antigen binding fragment comprises a heavy chain comprising an amino acid sequence of SEQ ID NO: 1 with 1, 2, 3, 4, 5, 6, 7, 8, 9, 10, 11, 12, 13, 14, 15 or more amino acid substitutions, insertions or deletions, and the substitutions, insertions or deletions are made, e.g., in the framework regions (e.g., those regions outside of the CDRs, which CDRs are underlined in FIG. 2A). In specific embodiments, the TNFα antigen binding fragment comprises a light chain comprising an amino acid sequence of SEQ ID NO: 2 with 1, 2, 3, 4, 5, 6, 7, 8, 9, 10, 11, 12, 13, 14, 15 or more amino acid substitutions, insertions or deletions, and the substitutions, insertions or deletions are made, e.g., in the framework regions (e.g., those regions outside of the CDRs, which CDRs are underlined in FIG. 2A).

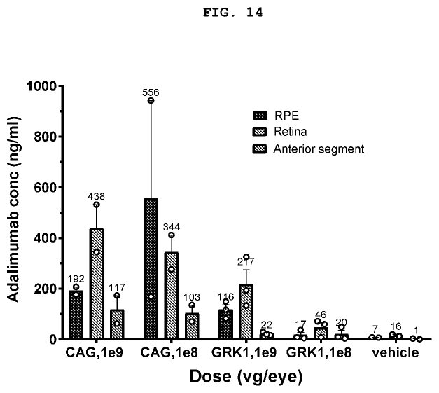

In certain embodiments, the anti-TNFα antigen-binding fragment transgene encodes a hyperglycosylated adalimumab Fab, comprising a heavy chain and a light chain of SEQ ID NOs: 1 and 2, respectively, with one or more of the following mutations: L116N (heavy chain), Q160N or Q160S (light chain), and/or E195N (light chain) (see FIGS. 14A (heavy chain) and 14B (light chain)).

In certain embodiments, the anti-TNFα antigen-binding fragment transgene encodes an antigen-binding fragment and comprises the nucleotide sequences encoding the six adalimumab CDRs which are underlined in the heavy and light chain variable domain sequences of FIG. 2A which are spaced between framework regions, generally human framework regions, and associated with constant domains depending upon the form of the antigen-binding molecule, as is known in the art to form the heavy and/or light chain variable domain of an anti-TNFα antibody or antigen-binding fragment thereof.

In certain embodiments, the anti-TNFα antigen-binding fragment transgene comprises the nucleotide sequences encoding the heavy and light chains of the Fab portion of infliximab (having amino acid sequences of SEQ ID NOs. 3 and 4, respectively, see Table 7 and FIG. 2B). The nucleotide sequences may be codon optimized for expression in human cells. Nucleotide sequences may, for example, comprise the nucleotide sequences of SEQ ID NO: 28 (encoding the infliximab heavy chain Fab portion) and SEQ ID NO: 29 (encoding the infliximab light chain Fab portion) as set forth in Table 8. The heavy and light chain sequences both have a signal or leader sequence at the N-terminus appropriate for expression and secretion in human cells, in particular, human ocular tissue cells (e.g., retinal cells) or liver and/or muscle cells. The signal sequence may have the amino acid sequence of MYRMQLLLLIALSLALVTNS (SEQ ID NO:85). Alternatively, the signal sequence may have an amino acid sequence selected from any one of the signal sequences set forth in Table 2 that correspond to the proteins secreted by ocular tissue cell types. Alternatively, the signal sequence may be appropriate for expression in muscle or liver cells, such as those listed in Tables 3 and 4 infra.

In addition to the heavy and light chain variable domain and CH1 and CL domain sequences, the transgenes may comprise, at the C-terminus of the heavy chain CH1 domain sequence, all or a portion of the hinge region. In specific embodiments, the anti-TNFα-antigen binding domain has a heavy chain Fab domain of SEQ ID NO: 3 with additional hinge region sequence starting after the C-terminal valine (V), contains all or a portion of the amino acid sequence EPKSCDKTHTCPPCPAPELLGG (SEQ ID NO:153), and specifically, EPKSCDKTHL (SEQ ID NO:155), EPKSCDKTHT (SEQ ID NO:156), EPKSCDKTHTCPPCPA (SEQ ID NO:157), EPKSCDKTHLCPPCPA (SEQ ID NO:158), EPKSCDKTHTCPPCPAPELLGGPSVFL (SEQ ID NO:159) or EPKSCDKTHLCPPCPAPELLGGPSVFL (SEQ ID NO:160) as set forth in FIG. 2B. These hinge regions may be encoded by nucleotide sequences at the 3′ end of SEQ ID NO: 28 by the hinge region encoding sequences set forth in Table 8 (SEQ ID NO: 28). In another embodiment, the transgenes comprise the amino acid sequences encoding the full length (or substantially full length) heavy and light chains of the antibody, comprising the Fc domain at the C terminus of the heavy chain, e.g. having an amino acid sequence of SEQ ID NO: 65 (Table 7) or an IgG1 Fc domain, such as SEQ ID No. 61 or as depicted in FIG. 6, or a mutant or variant thereof. The Fc domain may be engineered for altered binding to one or more Fc receptors and/or effector function as disclosed in Section 5.1.9, infra.

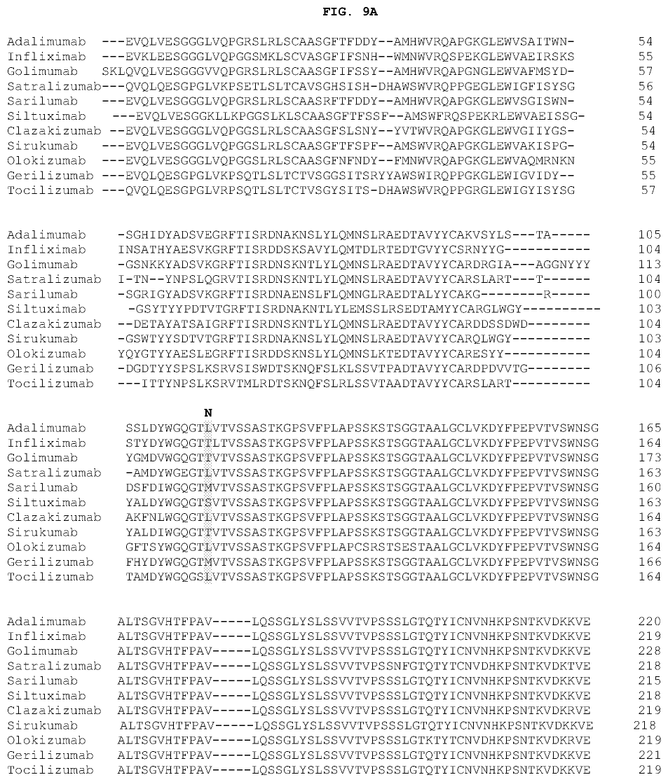

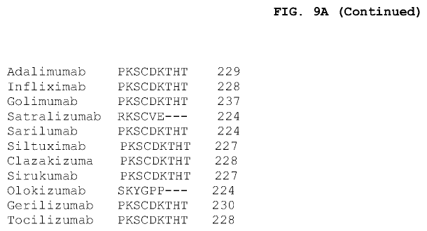

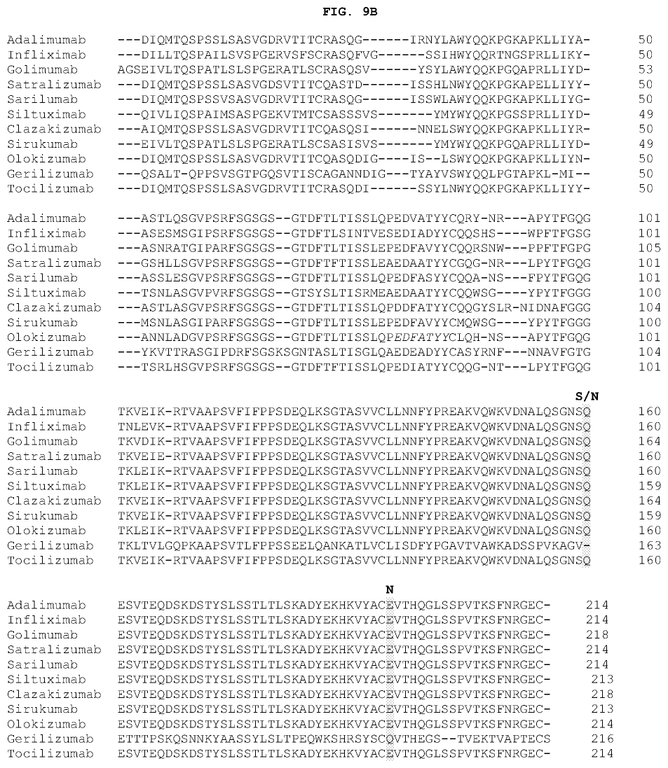

In certain embodiments, the anti-TNFα antigen-binding fragment transgene encodes an TNFα antigen-binding fragment comprising a light chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 4. In certain embodiments, the anti-TNFα antigen-binding fragment transgene encodes an TNFα antigen-binding fragment comprising a heavy chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 3. In certain embodiments, the anti-TNFα antigen-binding fragment transgene encodes an antigen-binding fragment comprising a light chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 4 and a heavy chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 3. In specific embodiments, the TNFα antigen binding fragment comprises a heavy chain comprising an amino acid sequence of SEQ ID NO: 3 with 1, 2, 3, 4, 5, 6, 7, 8, 9, 10, 11, 12, 13, 14, 15 or more amino acid substitutions, insertions or deletions, and the substitutions, insertions or deletions are made, e.g., in the framework regions (e.g., those regions outside of the CDRs, which CDRs are underlined in FIG. 2B) or are substitutions with an amino acid present at that position in the heavy chain of one or more of the other therapeutic antibodies, for example, as identified by the alignment in FIG. 9A. In specific embodiments, the TNFα antigen binding fragment comprises a light chain comprising an amino acid sequence of SEQ ID NO: 4 with 1, 2, 3, 4, 5, 6, 7, 8, 9, 10, 11, 12, 13, 14, 15 or more amino acid substitutions, insertions or deletions, and the substitutions, insertions or deletions are made, for example, in the framework regions (e.g., those regions outside of the CDRs, which CDRs are underlined in FIG. 2B) or are substitutions with an amino acid present at that position in the light chain of one or more of the other therapeutic antibodies, for example, as identified by the alignment in FIG. 9B.

In certain embodiments, the anti-TNFα antigen-binding fragment transgene encodes a hyperglycosylated infliximab Fab, comprising a heavy chain and a light chain of SEQ ID NOs: 3 and 4, respectively, with one or more of the following mutations: T115N (heavy chain), Q160N or Q160S (light chain), and/or E195N (light chain) (see FIGS. 9A (heavy chain) and 9B (light chain)).

In certain embodiments, the anti-TNFα antigen-binding fragment transgene encodes an antigen-binding fragment and comprises the nucleotide sequences encoding the six infliximab CDRs which are underlined in the heavy and light chain variable domain sequences of FIG. 2B which are spaced between framework regions, generally human framework regions, and associated with constant domains depending upon the form of the antigen-binding molecule, as is known in the art to form the heavy and/or light chain variable domain of an anti-TNFα antibody or antigen-binding fragment thereof.

In certain embodiments, the anti-TNFα antigen-binding fragment transgene comprises the nucleotide sequences encoding the heavy and light chains of the Fab portion of golimumab (having amino acid sequences of SEQ ID NOs. 5 and 6, respectively, see Table 7 and FIG. 2C). The nucleotide sequences may be codon optimized for expression in human cells. Nucleotide sequences may, for example, comprise the nucleotide sequences of SEQ ID NO: 30 (encoding the golimumab heavy chain Fab portion) and SEQ ID NO: 31 (encoding the golimumab light chain Fab portion) as set forth in Table 6. The heavy and light chain sequences both have a signal or leader sequence at the N-terminus appropriate for expression and secretion in human cells, in particular, human ocular tissue cells (e.g., retinal cells) or liver and/or muscle cells. The signal sequence may have the amino acid sequence of MYRMQLLLLIALSLALVTNS (SEQ ID NO:85). Alternatively, the signal sequence may have an amino acid sequence selected from any one of the signal sequences set forth in Table 2 that correspond to the proteins secreted by ocular tissue cell types. Alternatively, the signal sequence may be appropriate for expression in muscle or liver cells, such as those listed in Tables 3 and 4 infra.

In addition to the heavy and light chain variable domain and CH1 and CL domain sequences, the transgenes may comprise, at the C-terminus of the heavy chain CH1 domain sequence, all or a portion of the hinge region. In specific embodiments, the anti-TNFα-antigen binding domain has a heavy chain variable domain of SEQ ID NO: 5 with additional hinge region sequence starting after the C-terminal valine (V), contains all or a portion of the amino acid sequence EPKSCDKTHTCPPCPAPELLGG (SEQ ID NO:153), and specifically, EPKSCDKTHL (SEQ ID NO:155), EPKSCDKTHT (SEQ ID NO:156), EPKSCDKTHTCPPCPA (SEQ ID NO:157), EPKSCDKTHLCPPCPA (SEQ ID NO:158), EPKSCDKTHTCPPCPAPELLGGPSVFL (SEQ ID NO:159) or EPKSCDKTHLCPPCPAPELLGGPSVFL (SEQ ID NO:160) as set forth in FIG. 2C. These hinge regions may be encoded by nucleotide sequences at the 3′ end of SEQ ID NO: 30 by the hinge region encoding sequences set forth in Table 8 (SEQ ID NO: 30). In another embodiment, the transgenes comprise the amino acid sequences encoding the full length (or substantially full length) heavy and light chains of the antibody, comprising the Fc domain at the C terminus of the heavy chain, e.g., having an amino acid sequence of SEQ ID NO: 66 (Table 6) or an IgG1 Fc domain, such as SEQ ID No. 61 or as depicted in FIG. 6, or a mutant or variant thereof. The Fc domain may be engineered for altered binding to one or more Fc receptors and/or effector function as disclosed in Section 5.1.9, infra.

In certain embodiments, the anti-TNFα antigen-binding fragment transgene encodes an TNFα antigen-binding fragment comprising a light chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 6. In certain embodiments, the anti-TNFα antigen-binding fragment transgene encodes an TNFα antigen-binding fragment comprising a heavy chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 5. In certain embodiments, the anti-TNFα antigen-binding fragment transgene encodes an antigen-binding fragment comprising a light chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 6 and a heavy chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 5. In specific embodiments, the TNFα antigen binding fragment comprises a heavy chain comprising an amino acid sequence of SEQ ID NO: 5 with 1, 2, 3, 4, 5, 6, 7, 8, 9, 10, 11, 12, 13, 14, 15 or more amino acid substitutions, insertions or deletions, and the substitutions, insertions or deletions are made, for example, in the framework regions (e.g., those regions outside of the CDRs, which CDRs are underlined in FIG. 2C) or are substitutions with an amino acid present at that position in the heavy chain of one or more of the other therapeutic antibodies, for example, as identified by the alignment in FIG. 149A. In specific embodiments, the TNFα antigen binding fragment comprises a light chain comprising an amino acid sequence of SEQ ID NO: 6 with 1, 2, 3, 4, 5, 6, 7, 8, 9, 10, 11, 12, 13, 14, 15 or more amino acid substitutions, insertions or deletions, and the substitutions, insertions or deletions are made, e.g., in the framework regions (e.g., those regions outside of the CDRs, which CDRs are underlined in FIG. 2C) or are substitutions with an amino acid present at that position in the light chain of one or more of the other therapeutic antibodies, for example, as identified by the alignment in FIG. 9B.

In certain embodiments, the anti-TNFα antigen-binding fragment transgene encodes a hyperglycosylated golimumab Fab, comprising a heavy chain and a light chain of SEQ ID NOs: 5 and 6, respectively, with one or more of the following mutations: T124N (heavy chain), Q164N or Q164S (light chain), and/or E199N (light chain) (see FIGS. 9A (heavy chain) and 9B (light chain)).

In certain embodiments, the anti-TNFα antigen-binding fragment transgene encodes an antigen-binding fragment and comprises the nucleotide sequences encoding the six golimumab CDRs which are underlined in the heavy and light chain variable domain sequences of FIG. 2C which are spaced between framework regions, generally human framework regions, and associated with constant domains depending upon the form of the antigen-binding molecule, as is known in the art to form the heavy and/or light chain variable domain of an anti-TNFα antibody or antigen-binding fragment thereof. Table 7 provides the amino acid sequences of Fab heavy and light chains, the full length heavy chain for adalimumab and the amino acid sequence for the translation product of full length and Fab adalimumab (SEQ ID Nos: 1, 2, 23, 24, 25). Table 8 provides a nucleotide sequence encoding the Fab heavy and light chains of the antibodies disclosed herein, adalimumab full length heavy chain, expression cassettes and genomes.

TABLE 7
Amino Acid Sequences of Heavy and Light Chains

| | Chain/ | |
|---|---|---|
| | SEQ ID | |
| mAb | NO: | Sequences |
| Adalimumab | Heavy/ | EVQLVESGGG LVQPGRSLRL SCAASGFTFD DYAMHWVRQA |
| | SEQ ID | PGKGLEWVSA ITWNSGHIDY ADSVEGRFTI SRDNAKNSLY |
| | NO: 1 | LQMNSLRAED TAVYYCAKVS YLSTASSLDY WGQGTLVTVS |
| | | SASTKGPSVF PLAPSSKSTS GGTAALGCLV KDYFPEPVTV |
| | | SWNSGALTSG VHTFPAVLQS SGLYSLSSVV TVPSSSLGTQ |
| | | TYICNVNHKP SNTKVDKKVE PKSCD +/− KTHT (or |
| | | KTHL) +/− CPPCPA +/− PELLGGPSVFL |
| Adalimumab | Light/ | DIQMTQSPSS LSASVGDRVT ITCRASQGIR NYLAWYQQKP |
| | SEQ ID | GKAPKLLIYA ASTLQSGVPS RFSGSGSGTD FTLTISSLQP |
| | NO: 2 | EDVATYYCQR YNRAPYTFGQ GTKVEIKRTV AAPSVFIFPP |
| | | SDEQLKSGTA SVVCLLNNFY PREAKVQWKV DNALQSGNSQ |
| | | ESVTEQDSKD STYSLSSTLT LSKADYEKHK VYACEVTHQG |
| | | LSSPVTKSFN RGEC |
| Infliximab | Heavy/ | EVKLEESGGG LVQPGGSMKL SCVASGFIFS NHWMNWVRQS |
| | SEQ ID | PEKGLEWVAE IRSKSINSAT HYAESVKGRF TISRDDSKSA |
| | NO: 3 | VYLQMTDLRT EDTGVYYCSR NYYGSTYDYW GQGTTLTVSS |
| | | ASTKGPSVFP LAPSSKSTSG GTAALGCLVK DYFPEPVTVS |
| | | WNSGALTSGV HTFPAVLQSS GLYSLSSVVT VPSSSLGTQT |
| | | YICNVNHKPS NTKVDKKVEP KSCD +/− KTHT (or KT |
| | | HL) +/− CPPCPA +/− PELLGGPSVFL |
| Infliximab | Light/ | DILLTQSPAI LSVSPGERVS FSCRASQFVG SSIHWYQQRT |
| | SEQ ID | NGSPRLLIKY ASESMSGIPS RFSGSGSGTD FTLSINTVES |
| | NO: 4 | EDIADYYCQQ SHSWPFTFGS GTNLEVKRTV AAPSVFIFPP |
| | | SDEQLKSGTA SVVCLLNNFY PREAKVQWKV DNALQSGNSQ |
| | | ESVTEQDSKD STYSLSSTLT LSKADYEKHK VYACEVTHQG |
| | | LSSPVTKSFN RGEC |
| Golimumab | Heavy/ | SKLQVQLVES GGGVVQPGRS LRLSCAASGF IFSSYAMHWV |
| | SEQ ID | RQAPGNGLEW VAFMSYDGSN KKYADSVKGR FTISRDNSKN |
| | NO: 5 | TLYLQMNSLR AEDTAVYYCA RDRGIAAGGN YYYYGMDVWG |
| | | QGTTVTVSSA STKGPSVFPL APSSKSTSGG TAALGCLVKD |
| | | YFPEPVTVSW NSGALTSGVH TFPAVLQSSG LYSLSSVVTV |
| | | PSSSLGTQTY ICNVNHKPSN TKVDKKVEPK SCD +/− KT |
| | | HT (or KTHL) +/− CPPCPA +/− PELLGGPSVFL |
| Golimumab | Light/ | AGSEIVLTQS PATLSLSPGE RATLSCRASQ SVYSYLAWYQ |
| | SEQ ID | QKPGQAPRLL IYDASNRATG IPARFSGSGS GTDFTLTISS |
| | NO: 6 | LEPEDFAVYY CQQRSNWPPF TFGPGTKVDI KRTVAAPSVF |
| | | IFPPSDEQLK SGTASVVCLL NNFYPREAKV QWKVDNALQS |
| | | GNSQESVTEQ DSKDSTYSLS STLTLSKADY EKHKVYACEV |
| | | THQGLSSPVT KSFNRGEC |
| Satralizumab | Heavy/ | QVQLQESGPG LVKPSETLSL TCAVSGHSIS HDHAWSWVRQ |
| | SEQ ID | PPGEGLEWIG FISYSGITNY NPSLQGRVTI SRDNSKNTLY |
| | NO: 7 | LQMNSLRAED TAVYYCARSL ARTTAMDYWG EGTLVTVSSA |
| | | STKGPSVFPL APSSKSTSGG TAALGCLVKD YFPEPVTVSW |
| | | NSGALTSGVH TFPAVLQSSG LYSLSSVVTV PSSNFGTQTY |
| | | TCNVDHKPSN TKVDKTVERK SCVE +/− CPPCPA +/−P |
| | | PVAG |
| Satralizumab | Light/ | DIQMTQSPSS LSASVGDSVT ITCQASTDIS SHLNWYQQKP |
| | SEQ ID | GKAPELLIYY GSHLLSGVPS RFSGSGSGTD FTFTISSLEA |
| | NO: 8 | EDAATYYCGQ GNRLPYTFGQ GTKVEIERTV AAPSVFIFPP |
| | | SDEQLKSGTA SVVCLLNNFY PREAKVQWKV DNALQSGNSQ |
| | | ESVTEQDSKD STYSLSSTLT LSKADYEKHK VYACEVTHQG |
| | | LSSPVTKSFN RGEC |
| Sarilumab | Heavy/ | EVQLVESGGG LVQPGRSLRL SCAASRFTFD |
| | SEQ ID | DYAMHWVRQA PGKGLEWVSG ISWNSGRIGY ADSVKGRFTI |
| | NO: 9 | SRDNAENSLF LQMNGLRAED TALYYCAKGR DSFDIWGQGT |
| | | MVTVSSASTK GPSVFPLAPS SKSTSGGTAA LGCLVKDYFP |
| | | EPVTVSWNSG ALTSGVHTFP AVLQSSGLYS LSSVVTVPSS |
| | | SLGTQTYICN VNHKPSNTKV DKKVEPKSCD +/− KTHT |
| | | (or KTHL) +/− CPPCPA +/− PELLGGPSVFL |
| Sarilumab | Light/ | DIQMTQSPSS VSASVGDRVT ITCRASQGIS |
| | SEQ ID | SWLAWYQQKP GKAPKLLIYG ASSLESGVPS RTSGSGSGTD |
| | NO: 10 | FTLTISSLQP EDFASYYCQQ ANSFPYTFGQ GTKLEIKRTV |
| | | AAPSVFIFPP SDEQLKSGTA SVVCLLNNFY PREAKVQWKV |
| | | DNALQSGNSQ ESVTEQDSKD STYSLSSTLT LSKADYEKHK |
| | | VYACEVTHQG LSSPVTKSFN RGEC |
| Siltuximab | Heavy/ | EVQLVESGGK LLKPGGSLKL SCAASGFTFS SFAMSWFRQS |
| | SEQ ID | PEKRLEWVAE ISSGGSYTYY PDTVTGRFTI SRDNAKNTLY |
| | NO: 11 | LEMSSLRSED TAMYYCARGL WGYYALDYWG QGTSVTVSSA |
| | | STKGPSVFPL APSSKSTSGG TAALGCLVKD YFPEPVTVSW |
| | | NSGALTSGVH TFPAVLQSSG LYSLSSVVTV PSSSLGTQTY |
| | | ICNVNHKPSN TKVDKKVEPK SCD +/− KTHT (or |
| | | KTHL) +/− CPPCPA +/− PELLGGPSVFL |
| Siltuximab | Heavy/ | QIVLIQSPAI MSASPGEKVT MTCSASSSVS YMYWYQQKPG |
| | SEQ ID | SSPRLLIYDT SNLASGVPVR FSGSGSGTSY SLTISRMEAE |
| | NO: 12 | DAATYYCQQW SGYPYTFGGG TKLEIKRTVA APSVFIFPPS |
| | | DEQLKSGTAS VVCLLNNFYP REAKVQWKVD NALQSGNSQE |
| | | SVTEQDSKDS TYSLSSTLTL SKADYEKHKV YACEVTHQGL |
| | | SSPVTKSFNR GEC |
| Clazakizumab | Heavy/ | EVQLVESGGG LVQPGGSLRL SCAASGFSLS NYYVTWVRQA |
| | SEQ ID | PGKGLEWVGI IYGSDETAYA TSAIGRFTIS RDNSKNTLYL |
| | NO: 13 | QMNSLRAEDT AVYYCARDDS SDWDAKFNLW GQGTLVTVSS |
| | | ASTKGPSVFP LAPSSKSTSG GTAALGCLVK DYFPEPVTVS |
| | | WNSGALTSGV HTFPAVLQSS GLYSLSSVVT VPSSSLGTQT |
| | | YICNVNHKPS NTKVDKRVEP KSCD +/− KTHT (or |
| | | KTHL) +/− CPPCPA +/− PELLGGPSVFL |
| Clazakizumab | Light/ | AIQMTQSPSS LSASVGDRVT ITCQASQSIN NELSWYQQKP |
| | SEQ ID | GKAPKLLIYR ASTLASGVPS RFSGSGSGTD FTLTISSLQP |
| | NO: 14 | DDFATYYCQQ GYSLRNIDNA FGGGTKVEIK RTVAAPSVFI |
| | | FPPSDEQLKS GTASVVCLLN NFYPREAKVQ WKVDNALQSG |
| | | NSQESVTEQD SKDSTYSLSS TLTLSKADYE KHKVYACEVT |
| | | HQGLSSPVTK SFNRGEC |
| Sirukumab | Heavy/ | EVQLVESGGG LVQPGGSLRL SCAASGFTFS PFAMSWVRQA |
| | SEQ ID | PGKGLEWVAK ISPGGSWTYY SDTVTGRFTI SRDNAKNSLY |
| | NO: 15 | LQMNSLRAED TAVYYCARQL WGYYALDIWG QGTTVTVSSA |
| | | STKGPSVFPL APSSKSTSGG TAALGCLVKD YFPEPVTVSW |
| | | NSGALTSGVH TFPAVLQSSG LYSLSSVVTV PSSSLGTQTY |
| | | ICNVNHKPSN TKVDKKVEPK SCD +−/ KTHT (or |
| | | KTHL) +/− CPPCPA +−/ PELLGGPSVFL |
| Sirukumab | Light/ | EIVLTQSPAT LSLSPGERAT LSCSASISVS YMYWYQQKPG |
| | SEQ ID | QAPRLLIYDM SNLASGIPAR FSGSGSGTDF TLTISSLEPE |
| | NO: 16 | DFAVYYCMQW SGYPYTFGGG TKVEIKRTVA APSVFIFPPS |
| | | DEQLKSGTAS VVCLLNNFYP REAKVQWKVD NALQSGNSQE |
| | | SVTEQDSKDS TYSLSSTLTL SKADYEKHKV YACEVTHQGL |
| | | SSPVTKSFNR GEC |
| Olokizumab | Heavy/ | EVQLVESGGG LVQPGGSLRL SCAASGFNFN DYFMNWVRQA |
| | SEQ ID | PGKGLEWVAQ MRNKNYQYGT YYAESLEGRF TISRDDSKNS |
| | NO: 17 | LYLQMNSLKT EDTAVYYCAR ESYYGFTSYW GQGTLVTVSS |
| | | ASTKGPSVFP LAPCSRSTSE STAALGCLVK DYFPEPVTVS |
| | | WNSGALTSGV HTFPAVLQSS GLYSLSSVVT VPSSSLGTKT |
| | | YTCNVDHKPS NTKVDKRVES KY +/− GPPCPPCPA (or |
| | | GPPCPSCPA) +/− PEFLGGPSVFL |
| Olokizumab | Light/ | DIQMTQSPSS LSASVGDRVT ITCQASQDIG ISLSWYQQKP |
| | SEQ ID | GKAPKLLIYN ANNLADGVPS RFSGSGSGTD FTLTISSLQP |
| | NO: 18 | EDFATYYCLQ HNSAPYTFGQ GTKLEIKRTV AAPSVFIFPP |
| | | SDEQLKSGTA SVVCLLNNFY PREAKVQWKV DNALQSGNSQ |
| | | ESVTEQDSKD STYSLSSTLT LSKADYEKHK VYACEVTHQG |
| | | LSSPVTKSFN RGEC |
| Gerilizumab | Heavy/ | EVQLQESGPG LVKPSQTLSL TCTVSGGSIT SRYYAWSWIR |
| | SEQ ID | QPPGKGLEWI GVIDYDGDTY YSPSLKSRVS ISWDTSKNQF |
| | NO: 19 | SLKLSSVTPA DTAVYYCARD PDVVTGFHYD YWGQGTMVTV |
| | | SSASTKGPSV FPLAPSSKST SGGTAALGCL VKDYFPEPVT |
| | | VSWNSGALTS GVHTFPAVLQ SSGLYSLSSV VTVPSSSLGT |
| | | QTYICNVNHK PSNTKVDKKV EPKSCD +/− KTHT (or |
| | | KTHL) +/− CPPCPA +−/ PELLGGPSVFL |
| Gerilizumab | Light/ | QSALTQPPSV SGTPGQSVTI SCAGANNDIG TYAYVSWYQQ |
| | SEQ ID | LPGTAPKLMI YKVTTRASGI PDRFSGSKSG NTASLTISGL |
| | NO: 20 | QAEDEADYYC ASYRNFNNAV FGTGTKLTVL GQPKAAPSVT |
| | | LFPPSSEELQ ANKATLVCLI SDFYPGAVTV AWKADSSPVK |
| | | AGVETTTPSK QSNNKYAASS YLSLTPEQWK SHRSYSCQVT |
| | | HEGSTVEKTV APTECS |
| Tocilizumab | Heavy/ | QVQLQESGPG LVRPSQTLSL TCTVSGYSIT SDHAWSWVRQ |
| | SEQ ID | PPGRGLEWIG YISYSGITTY NPSLKSRVTM LRDTSKNQFS |
| | NO: 21 | LRLSSVTAAD TAVYYCARSL ARTTAMDYWG QGSLVTVSSA |
| | | STKGPSVFPL APSSKSTSGG TAALGCLVKD YFPEPVTVSW |
| | | NSGALTSGVH TFPAVLQSSG LYSLSSVVTV PSSSLGTQTY |
| | | ICNVNHKPSN TKVDKKVEPK SCD +/− KTHT (or |
| | | KTHL) +/− CPPCPA +/− PELLGGPSVFL |
| Tocilizumab | Light/ | DIQMTQSPSS LSASVGDRVT ITCRASQDIS SYLNWYQQKP |
| | SEQ ID | GKAPKLLIYY TSRLHSGVPS RFSGSGSGTD FTFTISSLQP |
| | NO: 22 | EDIATYYCQQ GNTLPYTFGQ GTKVEIKRTV AAPSVFIFPP |
| | | SDEQLKSGTA SVVCLLNNFY PREAKVQWKV DNALQSGNSQ |
| | | ESVTEQDSKD STYSLSSTLT LSKADYEKHK VYACEVTHQG |
| | | LSSPVTKSFN RGEC |
| Adalimumab | Heavy/ | EVQLVESGGGLVQPGRSLRLSCAASGFTFDDYAMHWVRQAPGK |
| Heavy Chain | SEQ ID | GLEWVSAITWNSGHIDYADSVEGRFTISRDNAKNSLYLQMNSL |
| (IgG1 Fc: CH1- | NO: 23 | RAEDTAVYYCAKVSYLSTASSLDYWGQGTLVTVSSASTKGPSV |
| hinge-CH2) | | FPLAPSSKSTSGGTAALGCLVKDYFPEPVTVSWNSGALTSGVH |
| | | TFPAVLQSSGLYSLSSVVTVPSSSLGTQTYICNVNHKPSNTKV |
| | | DKKVEPKSCDKTHTCPPCPAPELLGGPSVFLFPPKPKDTLMIS |
| | | RTPEVTCVVVDVSHEDPEVKFNWYVDGVEVHNAKTKPREEQYN |
| | | STYRVVSVLTVLHQDWLNGKEYKCKVSNKALPAPIEKTISKAK |
| | | GQPREPQVYTLPPSRDELTKNQVSLTCLVKGFYPSDIAVEWES |
| | | NGQPENNYKTTPPVLDSDGSFFLYSKLTVDKSRWQQGNVFSCS |
| | | VMHEALHNHYTQKSLSLSPGK |
| Adalimumab | SEQ ID | MYRMQLLLLIALSLALVTNSEVQLVESGGGLVQPGRSLRLSCA |
| Vectorized IgG- | NO: 24 | ASGFTFDDYAMHWVRQAPGKGLEWVSAITWNSGHIDYADSVEG |
| fully encoded | | RFTISRDNAKNSLYLQMNSLRAEDTAVYYCAKVSYLSTASSLD |
| protein | | YWGQGTLVTVSSASTKGPSVFPLAPSSKSTSGGTAALGCLVKD |
| | | YFPEPVTVSWNSGALTSGVHTFPAVLQSSGLYSLSSVVTVPSS |
| | | SLGTQTYICNVNHKPSNTKVDKKVEPKSCDKTHTCPPCPAPEL |
| | | LGGPSVFLFPPKPKDTLMISRTPEVTCVVVDVSHEDPEVKFNW |
| | | YVDGVEVHNAKTKPREEQYNSTYRVVSVLTVLHQDWLNGKEYK |
| | | CKVSNKALPAPIEKTISKAKGQPREPQVYTLPPSRDELTKNQV |
| | | SLTCLVKGFYPSDIAVEWESNGQPENNYKTTPPVLDSDGSFFL |
| | | YSKLTVDKSRWQQGNVFSCSVMHEALHNHYTQKSLSLSPGKRK |
| | | RRGSGEGRGSLLTCGDVEENPGPMYRMQLLLLIALSLALVTNS |
| | | DIQMTQSPSSLSASVGDRVTITCRASQGIRNYLAWYQQKPGKA |
| | | PKLLIYAASTLQSGVPSRFSGSGSGTDFTLTISSLQPEDVATY |
| | | YCQRYNRAPYTFGQGTKVEIKRTVAAPSVFIFPPSDEQLKSGT |
| | | ASVVCLLNNFYPREAKVQWKVDNALQSGNSQESVTEQDSKDST |
| | | YSLSSTLTLSKADYEKHKVYACEVTHQGLSSPVTKSFNRGEC |
| | | Leaders are shown in bold; Furin-2A linker |
| | | is underlined; hinge region is shown in |
| | | italic |
| Adalimumab | SEQ ID | MYRMQLLLLIALSLALVTNSEVQLVESGGGLVQPGRSLRLSCA |
| Vectorized Fab- | NO: 25 | ASGFTEDDYAMHWVRQAPGKGLEWVSAITWNSGHIDYADSVEG |
| fully encoded | | RFTISRDNAKNSLYLQMNSLRAEDTAVYYCAKVSYLSTASSLD |
| protein | | YWGQGTLVTVSSASTKGPSVFPLAPSSKSTSGGTAALGCLVKD |
| | | YFPEPVTVSWNSGALTSGVHTFPAVLQSSGLYSLSSVVTVPSS |
| | | SLGTQTYICNVNHKPSNTKVDKKVEPKSCDKTHRKRRGSGEGR |
| | | GSLLTCGDVEENPGPMYRMQLLLLIALSLALVTNSDIQMTQSP |
| | | SSLSASVGDRVTITCRASQGIRNYLAWYQQKPGKAPKLLIYAA |
| | | STLQSGVPSRFSGSGSGTDFTLTISSLQPEDVATYYCQRYNRA |
| | | PYTFGQGTKVEIKRTVAAPSVFIFPPSDEQLKSGTASVVCLLN |
| | | NFYPREAKVQWKVDNALQSGNSQESVTEQDSKDSTYSLSSTLT |
| | | LSKADYEKHKVYACEVTHQGLSSPVTKSFNRGEC |
| | | Leaders are shown in bold; Furin-2A linker |
| | | is underlined; hinge region is shown in |
| | | italic |

TABLE 8
Nucleotide Sequences of Heavy and Light Chains,
Expression Cassettes and Genomes

| | Chain/ | |
|---|---|---|
| mAb or | SEQ ID | |
| construct name | NO: | Sequences |
| Adalimumab | Heavy/ | GAGGTGCAGCTGGTGGAGAGCGGCGGCGGCCTGGTGCAGCCCG |
| | SEQ ID | GCAGGAGCCTGAGGCTGAGCTGCGCCGCCAGCGGCTTCACCTT |
| | NO: 26 | CGACGACTACGCCATGCACTGGGTGAGGCAGGCCCCCGGCAAG |
| | | GGCCTGGAGTGGGTGAGCGCCATCACCTGGAACAGCGGCCACA |
| | | TCGACTACGCCGACAGCGTGGAGGGCAGGTTCACCATCAGCAG |
| | | GGACAACGCCAAGAACAGCCTGTACCTGCAGATGAACAGCCTG |
| | | AGGGCCGAGGACACCGCCGTGTACTACTGCGCCAAGGTGAGCT |
| | | ACCTGAGCACCGCCAGCAGCCTGGACTACTGGGGCCAGGGCAC |
| | | CCTGGTGACCGTGAGCAGCGCCAGCACCAAGGGCCCCAGCGTG |
| | | TTCCCCCTGGCCCCCAGCAGCAAGAGCACCAGCGGCGGCACCG |
| | | CCGCCCTGGGCTGCCTGGTGAAGGACTACTTCCCCGAGCCCGT |
| | | GACCGTGAGCTGGAACAGCGGCGCCCTGACCAGCGGCGTGCAC |
| | | ACCTTCCCCGCCGTGCTGCAGAGCAGCGGCCTGTACAGCCTGA |
| | | GCAGCGTGGTGACCGTGCCCAGCAGCAGCCTGGGCACCCAGAC |
| | | CTACATCTGCAACGTGAACCACAAGCCCAGCAACACCAAGGTG |
| | | GACAAGAAGGTGGAGCCCAAGAGCTGCGAC+/− |
| | | AAGACCCACACC (or AAGACCCACCTG) +/− |
| | | TGCCCCCCCTGCCCCGCC+/− |
| | | CCCGAGCTGCTGGGCGGCCCCAGCGTGTT CCTG |
| Adalimumab | Light/ | GACATCCAGATGACCCAGAGCCCCAGCAGCCTGAGCGCCAGCG |
| | SEQ ID | TGGGCGACAGGGTGACCATCACCTGCAGGGCCAGCCAGGGCAT |
| | NO: 27 | CAGGAACTACCTGGCCTGGTACCAGCAGAAGCCCGGCAAGGCC |
| | | CCCAAGCTGCTGATCTACGCCGCCAGCACCCTGCAGAGCGGCG |
| | | TGCCCAGCAGGTTCAGCGGCAGCGGCAGCGGCACCGACTTCAC |
| | | CCTGACCATCAGCAGCCTGCAGCCCGAGGACGTGGCCACCTAC |
| | | TACTGCCAGAGGTACAACAGGGCCCCCTACACCTTCGGCCAGG |
| | | GCACCAAGGTGGAGATCAAGAGGACCGTGGCCGCCCCCAGCGT |
| | | GTTCATCTTCCCCCCCAGCGACGAGCAGCTGAAGAGCGGCACC |
| | | GCCAGCGTGGTGTGCCTGCTGAACAACTTCTACCCCAGGGAGG |
| | | CCAAGGTGCAGTGGAAGGTGGACAACGCCCTGCAGAGCGGCAA |
| | | CAGCCAGGAGAGCGTGACCGAGCAGGACAGCAAGGACAGCACC |
| | | TACAGCCTGAGCAGCACCCTGACCCTGAGCAAGGCCGACTACG |
| | | AGAAGCACAAGGTGTACGCCTGCGAGGTGACCCACCAGGGCCT |
| | | GAGCAGCCCCGTGACCAAGAGCTTCAACAGGGGCGAGTGC |
| Infliximab | Heavy/ | GAGGTGAAGCTGGAGGAGAGCGGCGGCGGCCTGGTGCAGCCCG |
| | SEQ ID | GCGGCAGCATGAAGCTGAGCTGCGTGGCCAGCGGCTTCATCTT |
| | NO: 28 | CAGCAACCACTGGATGAACTGGGTGAGGCAGAGCCCCGAGAAG |
| | | GGCCTGGAGTGGGTGGCCGAGATCAGGAGCAAGAGCATCAACA |
| | | GCGCCACCCACTACGCCGAGAGCGTGAAGGGCAGGTTCACCAT |
| | | CAGCAGGGACGACAGCAAGAGCGCCGTGTACCTGCAGATGACC |
| | | GACCTGAGGACCGAGGACACCGGCGTGTACTACTGCAGCAGGA |
| | | ACTACTACGGCAGCACCTACGACTACTGGGGCCAGGGCACCAC |
| | | CCTGACCGTGAGCAGCGCCAGCACCAAGGGCCCCAGCGTGTTC |
| | | CCCCTGGCCCCCAGCAGCAAGAGCACCAGCGGCGGCACCGCCG |
| | | CCCTGGGCTGCCTGGTGAAGGACTACTTCCCCGAGCCCGTGAC |
| | | CGTGAGCTGGAACAGCGGCGCCCTGACCAGCGGCGTGCACACC |
| | | TTCCCCGCCGTGCTGCAGAGCAGCGGCCTGTACAGCCTGAGCA |
| | | GCGTGGTGACCGTGCCCAGCAGCAGCCTGGGCACCCAGACCTA |
| | | CATCTGCAACGTGAACCACAAGCCCAGCAACACCAAGGTGGAC |
| | | AAGAAGGTGGAGCCCAAGAGCTGCGAC+/− AAGACCCACACC |
| | | (or AAGACCCACCTG) +/− TGCCCCCCCTGCCCCGCC+/− |
| | | CCCGAGCTGCTGGGCGGCCCCAGCG TGTTCCTG |
| Infliximab | Light/ | GACATCCTGCTGACCCAGAGCCCCGCCATCCTGAGCGTGAGCC |
| | SEQ ID | CCGGCGAGAGGGTGAGCTTCAGCTGCAGGGCCAGCCAGTTCGT |
| | NO: 29 | GGGCAGCAGCATCCACTGGTACCAGCAGAGGACCAACGGCAGC |
| | | CCCAGGCTGCTGATCAAGTACGCCAGCGAGAGCATGAGCGGCA |
| | | TCCCCAGCAGGTTCAGCGGCAGCGGCAGCGGCACCGACTTCAC |
| | | CCTGAGCATCAACACCGTGGAGAGCGAGGACATCGCCGACTAC |
| | | TACTGCCAGCAGAGCCACAGCTGGCCCTTCACCTTCGGCAGCG |
| | | GCACCAACCTGGAGGTGAAGAGGACCGTGGCCGCCCCCAGCGT |
| | | GTTCATCTTCCCCCCCAGCGACGAGCAGCTGAAGAGCGGCACC |
| | | GCCAGCGTGGTGTGCCTGCTGAACAACTTCTACCCCAGGGAGG |
| | | CCAAGGTGCAGTGGAAGGTGGACAACGCCCTGCAGAGCGGCAA |
| | | CAGCCAGGAGAGCGTGACCGAGCAGGACAGCAAGGACAGCACC |
| | | TACAGCCTGAGCAGCACCCTGACCCTGAGCAAGGCCGACTACG |
| | | AGAAGCACAAGGTGTACGCCTGCGAGGTGACCCACCAGGGCCT |
| | | GAGCAGCCCCGTGACCAAGAGCTTCAACAGGGGCGAGTGC |
| Golimumab | Heavy/ | AGCAAGCTGCAGGTGCAGCTGGTGGAGAGCGGCGGCGGCGTGG |
| | SEQ ID | TGCAGCCCGGCAGGAGCCTGAGGCTGAGCTGCGCCGCCAGCGG |
| | NO: 30 | CTTCATCTTCAGCAGCTACGCCATGCACTGGGTGAGGCAGGCC |
| | | CCCGGCAACGGCCTGGAGTGGGTGGCCTTCATGAGCTACGACG |
| | | GCAGCAACAAGAAGTACGCCGACAGCGTGAAGGGCAGGTTCAC |
| | | CATCAGCAGGGACAACAGCAAGAACACCCTGTACCTGCAGATG |
| | | AACAGCCTGAGGGCCGAGGACACCGCCGTGTACTACTGCGCCA |
| | | GGGACAGGGGCATCGCCGCCGGCGGCAACTACTACTACTACGG |
| | | CATGGACGTGTGGGGCCAGGGCACCACCGTGACCGTGAGCAGC |
| | | GCCAGCACCAAGGGCCCCAGCGTGTTCCCCCTGGCCCCCAGCA |
| | | GCAAGAGCACCAGCGGCGGCACCGCCGCCCTGGGCTGCCTGGT |
| | | GAAGGACTACTTCCCCGAGCCCGTGACCGTGAGCTGGAACAGC |
| | | GGCGCCCTGACCAGCGGCGTGCACACCTTCCCCGCCGTGCTGC |
| | | AGAGCAGCGGCCTGTACAGCCTGAGCAGCGTGGTGACCGTGCC |
| | | CAGCAGCAGCCTGGGCACCCAGACCTACATCTGCAACGTGAAC |
| | | CACAAGCCCAGC+/− AAGACCCACACC (or |
| | | AAGACCCACCTG) +/− TGCCCCCCCTGCCCCGCC+/− |
| | | CCCGAGCTG CTGGGCGGCCCCAGCGTGTTCCTG |
| Golimumab | Light/ | GCCGGCAGCGAGATCGTGCTGACCCAGAGCCCCGCCACCCTGA |
| | SEQ ID | GCCTGAGCCCCGGCGAGAGGGCCACCCTGAGCTGCAGGGCCAG |
| | NO: 31 | CCAGAGCGTGTACAGCTACCTGGCCTGGTACCAGCAGAAGCCC |
| | | GGCCAGGCCCCCAGGCTGCTGATCTACGACGCCAGCAACAGGG |
| | | CCACCGGCATCCCCGCCAGGTTCAGCGGCAGCGGCAGCGGCAC |
| | | CGACTTCACCCTGACCATCAGCAGCCTGGAGCCCGAGGACTTC |
| | | GCCGTGTACTACTGCCAGCAGAGGAGCAACTGGCCCCCCTTCA |
| | | CCTTCGGCCCCGGCACCAAGGTGGACATCAAGAGGACCGTGGC |
| | | CGCCCCCAGCGTGTTCATCTTCCCCCCCAGCGACGAGCAGCTG |
| | | AAGAGCGGCACCGCCAGCGTGGTGTGCCTGCTGAACAACTTCT |
| | | ACCCCAGGGAGGCCAAGGTGCAGTGGAAGGTGGACAACGCCCT |
| | | GCAGAGCGGCAACAGCCAGGAGAGCGTGACCGAGCAGGACAGC |
| | | AAGGACAGCACCTACAGCCTGAGCAGCACCCTGACCCTGAGCA |
| | | AGGCCGACTACGAGAAGCACAAGGTGTACGCCTGCGAGGTGAC |
| | | CCACCAGGGCCTGAGCAGCCCCGTGACCAAGAGCTTCAACAGG |
| | | GGCGAGTGC |
| Satralizumab | Heavy/ | CAGGTGCAGCTGCAGGAGAGCGGCCCCGGCCTGGTGAAGCCCA |
| | SEQ ID | GCGAGACCCTGAGCCTGACCTGCGCCGTGAGCGGCCACAGCAT |
| | NO: 32 | CAGCCACGACCACGCCTGGAGCTGGGTGAGGCAGCCCCCCGGC |
| | | GAGGGCCTGGAGTGGATCGGCTTCATCAGCTACAGCGGCATCA |
| | | CCAACTACAACCCCAGCCTGCAGGGCAGGGTGACCATCAGCAG |
| | | GGACAACAGCAAGAACACCCTGTACCTGCAGATGAACAGCCTG |
| | | AGGGCCGAGGACACCGCCGTGTACTACTGCGCCAGGAGCCTGG |
| | | CCAGGACCACCGCCATGGACTACTGGGGCGAGGGCACCCTGGT |
| | | GACCGTGAGCAGCGCCAGCACCAAGGGCCCCAGCGTGTTCCCC |
| | | CTGGCCCCCAGCAGCAAGAGCACCAGCGGCGGCACCGCCGCCC |
| | | TGGGCTGCCTGGTGAAGGACTACTTCCCCGAGCCCGTGACCGT |
| | | GAGCTGGAACAGCGGCGCCCTGACCAGCGGCGTGCACACCTTC |
| | | CCCGCCGTGCTGCAGAGCAGCGGCCTGTACAGCCTGAGCAGCG |
| | | TGGTGACCGTGCCCAGCAGCAACTTCGGCACCCAGACCTACAC |
| | | CTGCAACGTGGACCACAAGCCCAGCAACACCAAGGTGGACAAG |
| | | ACCGTGGAGAGGAAGAGCTGCGTGGAG |
| | | +/− TGCCCCCCCTGCCCCGCC +/− CCCCCCGTGGCCGGC |
| Satralizumab | Light/ | GACATCCAGATGACCCAGAGCCCCAGCAGCCTGAGCGCCAGCG |
| | SEQ ID | TGGGCGACAGCGTGACCATCACCTGCCAGGCCAGCACCGACAT |
| | NO: 33 | CAGCAGCCACCTGAACTGGTACCAGCAGAAGCCCGGCAAGGCC |
| | | CCCGAGCTGCTGATCTACTACGGCAGCCACCTGCTGAGCGGCG |
| | | TGCCCAGCAGGTTCAGCGGCAGCGGCAGCGGCACCGACTTCAC |
| | | CTTCACCATCAGCAGCCTGGAGGCCGAGGACGCCGCCACCTAC |
| | | TACTGCGGCCAGGGCAACAGGCTGCCCTACACCTTCGGCCAGG |
| | | GCACCAAGGTGGAGATCGAGAGGACCGTGGCCGCCCCCAGCGT |
| | | GTTCATCTTCCCCCCCAGCGACGAGCAGCTGAAGAGCGGCACC |
| | | GCCAGCGTGGTGTGCCTGCTGAACAACTTCTACCCCAGGGAGG |
| | | CCAAGGTGCAGTGGAAGGTGGACAACGCCCTGCAGAGCGGCAA |
| | | CAGCCAGGAGAGCGTGACCGAGCAGGACAGCAAGGACAGCACC |
| | | TACAGCCTGAGCAGCACCCTGACCCTGAGCAAGGCCGACTACG |
| | | AGAAGCACAAGGTGTACGCCTGCGAGGTGACCCACCAGGGCCT |
| | | GAGCAGCCCCGTGACCAAGAGCTTCAACAGGGGCGAGTGC |
| Sarilumab | Heavy/ | GAGGTGCAGCTGGTGGAGAGCGGCGGCGGCCTGGTGCAGCCCG |
| | SEQ ID | GCAGGAGCCTGAGGCTGAGCTGCGCCGCCAGCAGGTTCACCTT |
| | NO: 34 | CGACGACTACGCCATGCACTGGGTGAGGCAGGCCCCCGGCAAG |
| | | GGCCTGGAGTGGGTGAGCGGCATCAGCTGGAACAGCGGCAGGA |
| | | TCGGCTACGCCGACAGCGTGAAGGGCAGGTTCACCATCAGCAG |
| | | GGACAACGCCGAGAACAGCCTGTTCCTGCAGATGAACGGCCTG |
| | | AGGGCCGAGGACACCGCCCTGTACTACTGCGCCAAGGGCAGGG |
| | | ACAGCTTCGACATCTGGGGCCAGGGCACCATGGTGACCGTGAG |
| | | CAGCGCCAGCACCAAGGGCCCCAGCGTGTTCCCCCTGGCCCCC |
| | | AGCAGCAAGAGCACCAGCGGCGGCACCGCCGCCCTGGGCTGCC |
| | | TGGTGAAGGACTACTTCCCCGAGCCCGTGACCGTGAGCTGGAA |
| | | CAGCGGCGCCCTGACCAGCGGCGTGCACACCTTCCCCGCCGTG |
| | | CTGCAGAGCAGCGGCCTGTACAGCCTGAGCAGCGTGGTGACCG |
| | | TGCCCAGCAGCAGCCTGGGCACCCAGACCTACATCTGCAACGT |
| | | GAACCACAAGCCCAGCAACACCAAGGTGGACAAGAAGGTGGAG |
| | | CCCAAGAGCTGCGAC+/− AAGACCCACACC (or |
| | | AAGACCCACCTG) +/− TGCCCCCCCTGCCCCGCC+/− |
| | | CCCGAGCTGCTGGGCGGCCCCAGCGTGTTCCTG |
| Sarilumab | Light/ | GACATCCAGATGACCCAGAGCCCCAGCAGCGTGAGCGCCAGCG |
| | SEQ ID | TGGGCGACAGGGTGACCATCACCTGCAGGGCCAGCCAGGGCAT |
| | NO: 35 | CAGCAGCTGGCTGGCCTGGTACCAGCAGAAGCCCGGCAAGGCC |
| | | CCCAAGCTGCTGATCTACGGCGCCAGCAGCCTGGAGAGCGGCG |
| | | TGCCCAGCAGGTTCAGCGGCAGCGGCAGCGGCACCGACTTCAC |
| | | CCTGACCATCAGCAGCCTGCAGCCCGAGGACTTCGCCAGCTAC |
| | | TACTGCCAGCAGGCCAACAGCTTCCCCTACACCTTCGGCCAGG |
| | | GCACCAAGCTGGAGATCAAGAGGACCGTGGCCGCCCCCAGCGT |
| | | GTTCATCTTCCCCCCCAGCGACGAGCAGCTGAAGAGCGGCACC |
| | | GCCAGCGTGGTGTGCCTGCTGAACAACTTCTACCCCAGGGAGG |
| | | CCAAGGTGCAGTGGAAGGTGGACAACGCCCTGCAGAGCGGCAA |
| | | CAGCCAGGAGAGCGTGACCGAGCAGGACAGCAAGGACAGCACC |
| | | TACAGCCTGAGCAGCACCCTGACCCTGAGCAAGGCCGACTACG |
| | | AGAAGCACAAGGTGTACGCCTGCGAGGTGACCCACCAGGGCCT |
| | | GAGCAGCCCCGTGACCAAGAGCTTCAACAGGGGCGAGTGC |
| Siltuximab | Heavy/ | gaagtgcagctggtggaaagcggcggcaaactgctgaaaccgg |
| | SEQ ID | gcggcagcctgaaactgagctgcgcggcgagcggctttacctt |
| | NO: 36 | tagcagctttgcgatgagctggtttcgccagagcccggaaaaa |
| | | cgcctggaatgggtggcggaaattagcagcggcggcagctata |
| | | cctattatccggataccgtgaccggccgctttaccattagccg |
| | | cgataacgcgaaaaacaccctgtatctggaaatgagcagcctg |
| | | cgcagcgaagataccgcgatgtattattgcgcgcgcggcctgt |
| | | ggggctattatgcgctggattattggggccagggcaccagcgt |
| | | gaccgtgagcagcgcgagcaccaaaggcccgagcgtgtttccg |
| | | ctggcgccgagcagcaaaagcaccagcggcggcaccgcggcgc |
| | | tgggctgcctggtgaaagattattttccggaaccggtgaccgt |
| | | gagctggaacagcggcgcgctgaccagcggcgtgcataccttt |
| | | ccggcggtgctgcagagcagcggcctgtatagcctgagcagcg |
| | | tggtgaccgtgccgagcagcagcctgggcacccagacctatat |
| | | ttgcaacgtgaaccataaaccgagcaacaccaaagtggataaa |
| | | aaagtggaaccgaaaagctgcgat |
| | | +/− AAGACCCACACC (or AAGACCCACCTG) +/− |
| | | TGCCCCCCCTGCCCCGCC+/− |
| | | CCCGAGCTGCTGGGCGGCCCCAGCGTGTTCCTG |
| Siltuximab | Light/ | cagattgtgctgattcagagcccggcgattatgagcgcgagcc |
| | SEQ ID | cgggcgaaaaagtgaccatgacctgcagcgcgagcagcagcgt |
| | NO: 37 | gagctatatgtattggtatcagcagaaaccgggcagcagcccg |
| | | cgcctgctgatttatgataccagcaacctggcgagcggcgtgc |
| | | cggtgcgctttagcggcagcggcagcggcaccagctatagcct |
| | | gaccattagccgcatggaagcggaagatgcggcgacctattat |
| | | tgccagcagtggagcggctatccgtatacctttggcggcggca |
| | | ccaaactggaaattaaacgcaccgtggcggcgccgagcgtgtt |
| | | tatttttccgccgagcgatgaacagctgaaaagcggcaccgcg |
| | | agcgtggtgtgcctgctgaacaacttttatccgcgcgaagcga |
| | | aagtgcagtggaaagtggataacgcgctgcagagcggcaacag |
| | | ccaggaaagcgtgaccgaacaggatagcaaagatagcacctat |
| | | agcctgagcagcaccctgaccctgagcaaagcggattatgaaa |
| | | aacataaagtgtatgcgtgcgaagtgacccatcagggcctgag |
| | | cagcccggtgaccaaaagctttaaccgcggcgaatgc |
| Clazakizumab | Heavy/ | gaagtgcagctggtggaaagcggcggcggcctggtgcagccgg |
| | SEQ ID | gcggcagcctgcgcctgagctgcgcggcgagcggctttagcct |
| | NO: 38 | gagcaactattatgtgacctgggtgcgccaggcgccgggcaaa |
| | | ggcctggaatgggtgggcattatttatggcagcgatgaaaccg |
| | | cgtatgcgaccagcgcgattggccgctttaccattagccgcga |
| | | taacagcaaaaacaccctgtatctgcagatgaacagcctgcgc |
| | | gcggaagataccgcggtgtattattgcgcgcgcgatgatagca |
| | | gcgattgggatgcgaaatttaacctgtggggccagggcaccct |
| | | ggtgaccgtgagcagcgcgagcaccaaaggcccgagcgtgttt |
| | | ccgctggcgccgagcagcaaaagcaccagcggcggcaccgcgg |
| | | cgctgggctgcctggtgaaagattattttccggaaccggtgac |
| | | cgtgagctggaacagcggcgcgctgaccagcggcgtgcatacc |
| | | tttccggcggtgctgcagagcagcggcctgtatagcctgagca |
| | | gcgtggtgaccgtgccgagcagcagcctgggcacccagaccta |
| | | tatttgcaacgtgaaccataaaccgagcaacaccaaagtggat |
| | | aaacgcgtggaaccgaaaagctgcgat |
| | | +/− AAGACCCACACC (or AAGACCCACCTG) +/− |
| | | TGCCCCCCCTGCCCCGCC+/− |
| | | CCCGAGCTGCTGGGCGGCCCCAGCGTGTTCCTG |
| Clazakizumab | Light/ | gcgattcagatgacccagagcccgagcagcctgagcgcgagcg |
| | SEQ ID | tgggcgatcgcgtgaccattacctgccaggcgagccagagcat |
| | NO: 39 | taacaacgaactgagctggtatcagcagaaaccgggcaaagcg |
| | | ccgaaactgctgatttatcgcgcgagcaccctggcgagcggcg |
| | | tgccgagccgctttagcggcagcggcagcggcaccgattttac |
| | | cctgaccattagcagcctgcagccggatgattttgcgacctat |
| | | tattgccagcagggctatagcctgcgcaacattgataacgcgt |
| | | ttggcggcggcaccaaagtggaaattaaacgcaccgtggcggc |
| | | gccgagcgtgtttatttttccgccgagcgatgaacagctgaaa |
| | | agcggcaccgcgagcgtggtgtgcctgctgaacaacttttatc |
| | | cgcgcgaagcgaaagtgcagtggaaagtggataacgcgctgca |
| | | gagcggcaacagccaggaaagcgtgaccgaacaggatagcaaa |
| | | gatagcacctatagcctgagcagcaccctgaccctgagcaaag |
| | | cggattatgaaaaacataaagtgtatgcgtgcgaagtgaccca |
| | | tcagggcctgagcagcccggtgaccaaaagctttaaccgcggc |
| | | gaatgcgaaccgaaaagctgcgat |
| | | +/− AAGACCCACACC (or AAGACCCACCTG) +/− |
| | | TGCCCCCCCTGCCCCGCC+/− |
| | | CCCGAGCTGCTGGGCGGCCCCAGCGTGTTCCTG |
| Sirukumab | Heavy/ | Gaagtgcagctggtggaaagcggcggcggcctggtgcagccgg |
| | SEQ ID | gcggcagcctgcgcctgagctgcgcggcgagcggctttacctt |
| | NO: 40 | tagcccgtttgcgatgagctgggtgcgccaggcgccgggcaaa |
| | | ggcctggaatgggtggcgaaaattagcccgggcggcagctgga |
| | | cctattatagcgataccgtgaccggccgctttaccattagccg |
| | | cgataacgcgaaaaacagcctgtatctgcagatgaacagcctg |
| | | cgcgcggaagataccgcggtgtattattgcgcgcgccagctgt |
| | | ggggctattatgcgctggatatttggggccagggcaccaccgt |
| | | gaccgtgagcagcgcgagcaccaaaggcccgagcgtgtttccg |
| | | ctggcgccgagcagcaaaagcaccagcggcggcaccgcggcgc |
| | | tgggctgcctggtgaaagattattttccggaaccggtgaccgt |
| | | gagctggaacagcggcgcgctgaccagcggcgtgcataccttt |
| | | ccggcggtgctgcagagcagcggcctgtatagcctgagcagcg |
| | | tggtgaccgtgccgagcagcagcctgggcacccagacctatat |
| | | ttgcaacgtgaaccataaaccgagcaacaccaaagtggataaa |
| | | aaagtggaaccgaaaagctgcgat |
| | | +/− AAGACCCACACC (or AAGACCCACCTG) +/− |
| | | TGCCCCCCCTGCCCCGCC+/− |
| | | CCCGAGCTGCTGGGCGGCCCCAGCGTGTTCCTG |
| Sirukumab | Light/ | gaaattgtgctgacccagagcccggcgaccctgagcctgagcc |
| | SEQ ID | cgggcgaacgcgcgaccctgagctgcagcgcgagcattagcgt |
| | NO: 41 | gagctatatgtattggtatcagcagaaaccgggccaggcgccg |
| | | cgcctgctgatttatgatatgagcaacctggcgagcggcattc |
| | | cggcgcgctttagcggcagcggcagcggcaccgattttaccct |
| | | gaccattagcagcctggaaccggaagattttgcggtgtattat |
| | | tgcatgcagtggagcggctatccgtatacctttggcggcggca |
| | | ccaaagtggaaattaaacgcaccgtggcggcgccgagcgtgtt |
| | | tatttttccgccgagcgatgaacagctgaaaagcggcaccgcg |
| | | agcgtggtgtgcctgctgaacaacttttatccgcgcgaagcga |
| | | aagtgcagtggaaagtggataacgcgctgcagagcggcaacag |
| | | ccaggaaagcgtgaccgaacaggatagcaaagatagcacctat |
| | | agcctgagcagcaccctgaccctgagcaaagcggattatgaaa |
| | | aacataaagtgtatgcgtgcgaagtgacccatcagggcctgag |
| | | cagcccggtgaccaaaagctttaaccgcggcgaatgc |
| Olokizumab | Heavy/ | Gaagtgcagctggtggaaagcggcggcggcctggtgcagccgg |
| | SEQ ID | gcggcagcctgcgcctgagctgcgcggcgagcggctttaactt |
| | NO: 42 | taacgattattttatgaactgggtgcgccaggcgccgggcaaa |
| | | ggcctggaatgggtggcgcagatgcgcaacaaaaactatcagt |
| | | atggcacctattatgcggaaagcctggaaggccgctttaccat |
| | | tagccgcgatgatagcaaaaacagcctgtatctgcagatgaac |
| | | agcctgaaaaccgaagataccgcggtgtattattgcgcgcgcg |
| | | aaagctattatggctttaccagctattggggccagggcaccct |
| | | ggtgaccgtgagcagcgcgagcaccaaaggcccgagcgtgttt |
| | | ccgctggcgccgtgcagccgcagcaccagcgaaagcaccgcgg |
| | | cgctgggctgcctggtgaaagattattttccggaaccggtgac |
| | | cgtgagctggaacagcggcgcgctgaccagcggcgtgcatacc |
| | | tttccggcggtgctgcagagcagcggcctgtatagcctgagca |
| | | gcgtggtgaccgtgccgagcagcagcctgggcaccaaaaccta |
| | | tacctgcaacgtggatcataaaccgagcaacaccaaagtggat |
| | | aaacgcgtgGAGAGCAAGTAC +/− |
| | | GGCCCCCCCTGCCCCCCCTG CCCCGCC |
| | | (GGCCCCCCCTGCCCCAGCTGCCCCGCC)+/− |
| | | CCCGAGTTCCTGGGCGGCCCCAGCGTGTTCCTG |
| Olokizumab | Light/ | gatattcagatgacccagagcccgagcagcctgagcgcgagcg |
| | SEQ ID | tgggcgatcgcgtgaccattacctgccaggcgagccaggatat |
| | NO: 43 | tggcattagcctgagctggtatcagcagaaaccgggcaaagcg |
| | | ccgaaactgctgatttataacgcgaacaacctggcggatggcg |
| | | tgccgagccgctttagcggcagcggcagcggcaccgattttac |
| | | cctgaccattagcagcctgcagccggaagattttgcgacctat |
| | | tattgcctgcagcataacagcgcgccgtatacctttggccagg |
| | | gcaccaaactggaaattaaacgcaccgtggcggcgccgagcgt |
| | | gtttatttttccgccgagcgatgaacagctgaaaagcggcacc |
| | | gcgagcgtggtgtgcctgctgaacaacttttatccgcgcgaag |
| | | cgaaagtgcagtggaaagtggataacgcgctgcagagcggcaa |
| | | cagccaggaaagcgtgaccgaacaggatagcaaagatagcacc |
| | | tatagcctgagcagcaccctgaccctgagcaaagcggattatg |
| | | aaaaacataaagtgtatgcgtgcgaagtgacccatcagggcct |
| | | gagcagcccggtgaccaaaagctttaaccgcggcgaatgc |
| Gerilimzumab | Heavy/ | Gaagtgcagctgcaggaaagcggcccgggcctggtgaaaccga |
| | SEQ ID | gccagaccctgagcctgacctgcaccgtgagcggcggcagcat |
| | NO: 44 | taccagccgctattatgcgtggagctggattcgccagccgccg |
| | | ggcaaaggcctggaatggattggcgtgattgattatgatggcg |
| | | atacctattatagcccgagcctgaaaagccgcgtgagcattag |
| | | ctgggataccagcaaaaaccagtttagcctgaaactgagcagc |
| | | gtgaccccggcggataccgcggtgtattattgcgcgcgcgatc |
| | | cggatgtggtgaccggctttcattatgattattggggccaggg |
| | | caccatggtgaccgtgagcagcgcgagcaccaaaggcccgagc |
| | | gtgtttccgctggcgccgagcagcaaaagcaccagcggcggca |
| | | ccgcggcgctgggctgcctggtgaaagattattttccggaacc |
| | | ggtgaccgtgagctggaacagcggcgcgctgaccagcggcgtg |
| | | catacctttccggcggtgctgcagagcagcggcctgtatagcc |
| | | tgagcagcgtggtgaccgtgccgagcagcagcctgggcaccca |
| | | gacctatatttgcaacgtgaaccataaaccgagcaacaccaaa |
| | | gtggataaaaaagtggaaccgaaaagctgcgat +/− |
| | | AAGACCCACACC (or AAGACCCACCTG) +/− |
| | | TGCCCCCCCTGCCCCGCC+/− |
| | | CCCGAGCTGCTGGGCGGCCCCAGCGTGTTCCTG |
| Gerilimzumab | Light/ | cagagcgcgctgacccagccgccgagcgtgagcggcaccccgg |
| | SEQ ID | gccagagcgtgaccattagctgcgcgggcgcgaacaacgatat |
| | NO: 45 | tggcacctatgcgtatgtgagctggtatcagcagctgccgggc |
| | | accgcgccgaaactgatgatttataaagtgaccacccgcgcga |
| | | gcggcattccggatcgctttagcggcagcaaaagcggcaacac |
| | | cgcgagcctgaccattagcggcctgcaggcggaagatgaagcg |
| | | gattattattgcgcgagctatcgcaactttaacaacgcggtgt |
| | | ttggcaccggcaccaaactgaccgtgctgggccagccgaaagc |
| | | ggcgccgagcgtgaccctgtttccgccgagcagcgaagaactg |
| | | caggcgaacaaagcgaccctggtgtgcctgattagcgattttt |
| | | atccgggcgcggtgaccgtggcgtggaaagcggatagcagccc |
| | | ggtgaaagcgggcgtggaaaccaccaccccgagcaaacagagc |
| | | aacaacaaatatgcggcgagcagctatctgagcctgaccccgg |
| | | aacagtggaaaagccatcgcagctatagctgccaggtgaccca |
| | | tgaaggcagcaccgtggaaaaaaccgtggcgccgaccgaatgc |
| | | agc |
| Tocilizumab | Heavy/ | Caggtgcagctgcaggaaagcggcccgggcctggtgcgcccga |
| | SEQ ID | gccagaccctgagcctgacctgcaccgtgagcggctatagcat |
| | NO:183 | taccagcgatcatgcgtggagctgggtgcgccagccgccgggc |
| | | cgcggcctggaatggattggctatattagctatagcggcatta |
| | | ccacctataacccgagcctgaaaagccgcgtgaccatgctgcg |
| | | cgataccagcaaaaaccagtttagcctgcgcctgagcagcgtg |
| | | accgcggcggataccgcggtgtattattgcgcgcgcagcctgg |
| | | cgcgcaccaccgcgatggattattggggccagggcagcctggt |
| | | gaccgtgagcagcgcgagcaccaaaggcccgagcgtgtttccg |
| | | ctggcgccgagcagcaaaagcaccagcggcggcaccgcggcgc |
| | | tgggctgcctggtgaaagattattttccggaaccggtgaccgt |
| | | gagctggaacagcggcgcgctgaccagcggcgtgcataccttt |
| | | ccggcggtgctgcagagcagcggcctgtatagcctgagcagcg |
| | | tggtgaccgtgccgagcagcagcctgggcacccagacctatat |
| | | ttgcaacgtgaaccataaaccgagcaacaccaaagtggataaa |
| | | aaagtggaaccgaaaagctgcgat +/− AAGACCCACACC |
| | | (or AAGACCCACCTG) +/− TGCCCCCCCTGCCCCGCC+/− |
| | | CCCGAGCTGCTGGGCGGCCCCAGCGTGTTCCTG |
| Tocilizumab | Light/ | gatattcagatgacccagagcccgagcagcctgagcgcgagcg |
| | SEQ ID | tgggcgatcgcgtgaccattacctgccgcgcgagccaggatat |
| | NO: 184 | tagcagctatctgaactggtatcagcagaaaccgggcaaagcg |
| | | ccgaaactgctgatttattataccagccgcctgcatagcggcg |
| | | tgccgagccgctttagcggcagcggcagcggcaccgattttac |
| | | ctttaccattagcagcctgcagccggaagatattgcgacctat |
| | | tattgccagcagggcaacaccctgccgtatacctttggccagg |
| | | gcaccaaagtggaaattaaacgcaccgtggcggcgccgagcgt |
| | | gtttatttttccgccgagcgatgaacagctgaaaagcggcacc |
| | | gcgagcgtggtgtgcctgctgaacaacttttatccgcgcgaag |
| | | cgaaagtgcagtggaaagtggataacgcgctgcagagcggcaa |
| | | cagccaggaaagcgtgaccgaacaggatagcaaagatagcacc |
| | | tatagcctgagcagcaccctgaccctgagcaaagcggattatg |
| | | aaaaacataaagtgtatgcgtgcgaagtgacccatcagggcct |
| | | gagcagcccggtgaccaaaagctttaaccgcggcgaatgc |
| pAAV.CAG. | SEQ ID | ctgcgcgctcgctcgctcactgaggccgcccgggcaaagcccg |
| Adalimumab. | NO: 46 | ggcgtcgggcgacctttggtcgcccggcctcagtgagcgagcg |
| IgG | | agcgcgcagagagggagtggccaactccatcactaggggttcc |
| (5′-ITR to | | ttgtagttaatgattaacccgccatgctacttatctaccaggg |
| 3--ITR) | | taatggggatcctctagaactatagctagtcgacattgattat |
| | | tgactagttattaatagtaatcaattacggggtcattagttca |
| | | tagcccatatatggagttccgcgttacataacttacggtaaat |
| | | ggcccgcctggctgaccgcccaacgacccccgcccattgacgt |
| | | caataatgacgtatgttcccatagtaacgccaatagggacttt |
| | | ccattgacgtcaatgggtggagtatttacggtaaactgcccac |
| | | ttggcagtacatcaagtgtatcatatgccaagtacgcccccta |
| | | ttgacgtcaatgacggtaaatggcccgcctggcattatgccca |
| | | gtacatgaccttatgggactttcctacttggcagtacatctac |
| | | gtattagtcatcgctattaccatggtcgaggtgagccccacgt |
| | | tctgcttcactctccccatctcccccccctccccacccccaat |
| | | tttgtatttatttattttttaattattttgtgcagcgatgggg |
| | | gcggggggggggggggggcgcgcgccaggcggggcggggcggg |
| | | gcgaggggcggggcggggcgaggcggagaggtgcggcggcagc |
| | | caatcagagcggcgcgctccgaaagtttccttttatggcgagg |
| | | cggcggcggcggcggccctataaaaagcgaagcgcgcggcggg |
| | | cgggagtcgctgcgcgctgccttcgccccgtgccccgctccgc |
| | | cgccgcctcgcgccgcccgccccggctctgactgaccgcgtta |
| | | ctcccacaggtgagcgggcgggacggcccttctcctccgggct |
| | | gtaattagcgcttggtttaatgacggcttgtttcttttctgtg |
| | | gctgcgtgaaagccttgaggggctccgggagggccctttgtgc |
| | | ggggggagcggctcggggggtgcgtgcgtgtgtgtgtgcgtgg |
| | | ggagcgccgcgtgcggctccgcgctgcccggcggctgtgagcg |
| | | ctgcgggcgcggcgcggggctttgtgcgctccgcagtgtgcgc |
| | | gaggggagcgcggccgggggcggtgccccgcggtgcggggggg |
| | | gctgcgaggggaacaaaggctgcgtgcggggtgtgtgcgtggg |
| | | ggggtgagcagggggtgtgggcgcgtcggtcgggctgcaaccc |
| | | cccctgcacccccctccccgagttgctgagcacggcccggctt |
| | | cgggtgcggggctccgtacggggcgtggcgcggggctcgccgt |
| | | gccgggggggggtggcggcaggtgggggtgccgggcggggcg |
| | | gggccgcctcgggccggggagggctcgggggaggggcgcggcg |
| | | gcccccggagcgccggcggctgtcgaggcgcggcgagccgcag |
| | | ccattgccttttatggtaatcgtgcgagagggcgcagggactt |
| | | cctttgtcccaaatctgtgcggagccgaaatctgggaggcgcc |
| | | gccgcaccccctctagcgggcgcggggcgaagcggtgcggcgc |
| | | cggcaggaaggaaatgggcggggagggccttcgtgcgtcgccg |
| | | cgccgccgtccccttctccctctccagcctcggggctgtccgc |
| | | ggggggacggctgccttcgggggggacggggcagggggggtt |
| | | cggcttctggcgtgtgaccggcggctctagagcctctgctaac |
| | | catgttcatgccttcttctttttcctacagctcctgggcaacg |
| | | tgctggttattgtgctgtctcatcattttggcaaagAATTCGC |
| | | CACCATGTACAGAATGCAGCTGCTGCTGCTCATTGCCCTGTCT |
| | | CTGGCCCTGGTCACCAATTCTGAAGTGCAGCTGGTGGAAAGTG |
| | | GTGGTGGACTGGTGCAGCCTGGCAGAAGCCTGAGACTGTCTTG |
| | | TGCTGCCTCTGGCTTCACCTTTGATGACTATGCCATGCACTGG |
| | | GTCAGACAGGCCCCTGGCAAAGGACTGGAATGGGTGTCAGCCA |
| | | TCACCTGGAACTCTGGCCACATTGACTATGCTGACTCTGTGGA |
| | | AGGCAGATTCACCATCAGCAGAGACAATGCCAAGAACAGCCTG |
| | | TACCTGCAGATGAACTCCCTGAGAGCTGAGGACACAGCAGTGT |
| | | ACTACTGTGCCAAGGTGTCCTACCTGAGCACAGCCAGCAGCCT |
| | | GGATTATTGGGGCCAGGGCACACTGGTTACAGTGTCCTCTGCC |
| | | AGCACAAAGGGCCCCTCTGTTTTTCCACTGGCTCCCAGCAGCA |
| | | AGAGCACCAGTGGTGGAACAGCTGCCCTGGGCTGTCTGGTCAA |
| | | GGATTACTTCCCTGAGCCTGTGACAGTGTCTTGGAACTCAGGG |
| | | GCTCTGACCTCTGGGGTGCACACATTTCCAGCTGTGCTGCAGT |
| | | CCTCTGGCCTGTACTCTCTGTCCTCTGTGGTCACAGTGCCTAG |
| | | CTCTAGCCTGGGCACCCAGACCTACATCTGCAATGTGAACCAC |
| | | AAGCCTAGCAACACCAAGGTGGACAAGAAGGTGGAACCCAAGA |
| | | GCTGTGACAAGACCCACACCTGTCCTCCATGTCCTGCTCCAGA |
| | | ACTGCTTGGAGGCCCTTCTGTGTTCCTGTTTCCTCCAAAGCCT |
| | | AAGGACACCCTGATGATCAGCAGAACCCCTGAAGTGACCTGTG |
| | | TGGTGGTTGATGTGTCCCATGAGGACCCAGAAGTGAAGTTCAA |
| | | TTGGTATGTGGATGGGGTTGAAGTGCACAATGCTAAGACCAAG |
| | | CCTAGAGAGGAACAGTACAACAGCACCTACAGAGTGGTGTCTG |
| | | TGCTGACAGTGCTGCATCAGGACTGGCTGAATGGCAAAGAGTA |
| | | CAAGTGCAAAGTGTCCAACAAGGCCCTGCCTGCTCCTATTGAG |
| | | AAAACCATCTCCAAGGCCAAGGGCCAGCCAAGAGAACCCCAGG |
| | | TTTACACACTGCCACCTAGCAGAGATGAGCTGACCAAGAACCA |
| | | GGTGTCCCTGACCTGCCTGGTTAAGGGCTTCTACCCCTCTGAC |
| | | ATTGCTGTGGAATGGGAGAGCAATGGCCAGCCTGAGAACAACT |
| | | ACAAGACAACCCCTCCTGTGCTGGACTCTGATGGCTCATTCTT |
| | | CCTGTACAGCAAGCTGACTGTGGACAAGTCCAGATGGCAGCAG |
| | | GGGAATGTGTTCAGCTGCTCTGTGATGCATGAGGCCCTGCACA |
| | | ACCACTACACCCAGAAAAGTCTGAGTCTGAGCCCTGGCAAGAG |
| | | AAAGAGAAGAGGCTCTGGAGAAGGCAGAGGCTCCCTGCTGACA |
| | | TGTGGGGATGTTGAAGAGAATCCTGGGCCTATGTATAGGATGC |
| | | AACTGCTCCTCCTGATTGCTCTGAGCCTGGCTCTTGTGACCAA |
| | | CTCTGACATCCAGATGACACAGAGCCCTAGCAGCCTGTCTGCT |
| | | TCTGTGGGAGACAGAGTGACCATCACATGCAGAGCCAGCCAGG |
| | | GAATCAGAAACTACCTGGCCTGGTATCAGCAAAAGCCTGGCAA |
| | | GGCCCCTAAGCTGCTGATCTATGCAGCCAGCACACTGCAGTCA |
| | | GGGGTGCCAAGCAGATTTTCAGGCTCTGGCTCTGGCACAGACT |
| | | TCACCCTGACCATTICTAGCCTGCAGCCTGAGGATGTGGCCAC |
| | | CTACTACTGCCAGAGATACAACAGAGCCCCATACACCTTTGGA |
| | | CAGGGCACAAAGGTGGAAATCAAGAGAACAGTGGCTGCCCCAT |
| | | CTGTGTTCATCTTCCCACCATCTGATGAACAGCTGAAGTCTGG |
| | | CACTGCCTCTGTTGTGTGCCTGCTGAACAACTTCTACCCTAGA |
| | | GAAGCCAAGGTGCAGTGGAAGGTTGACAATGCCCTGCAGTCTG |
| | | GCAATAGCCAAGAATCTGTGACAGAGCAGGACTCCAAGGATTC |
| | | CACCTACAGCCTGAGCAGCACCCTGACACTGAGCAAGGCTGAC |
| | | TATGAGAAGCACAAAGTGTATGCCTGTGAAGTGACACACCAGG |
| | | GACTGAGCAGCCCAGTGACCAAGAGCTTCAACAGGGGAGAGTG |
| | | CTGATAACtcgaggacggggtgaactacgcctgaggatccgat |
| | | ctttttccctctgccaaaaattatggggacatcatgaagcccc |
| | | ttgagcatctgacttctggctaataaaggaaatttattttcat |
| | | tgcaatagtgtgttggaattttttgtgtctctcactcggaagc |
| | | aattcgttgatctgaatttcgaccacccataatacccattacc |
| | | ctggtagataagtagcatggcgggttaatcattaactacaagg |
| | | aacccctagtgatggagttggccactccctctctgcgcgctcg |
| | | ctcgctcactgaggccgggcgaccaaaggtcgcccgacgcccg |
| | | ggctttgcccgggcggcctcagtgagcgagcgagcgcgcag |
| pAAV.CAG. | SEQ ID | gacattgattattgactagttattaatagtaatcaattacggg |
| Adalimumab. | NO: 47 | gtcattagttcatagcccatatatggagttccgcgttacataa |
| IgG (promoter | | cttacggtaaatggcccgcctggctgaccgcccaacgaccccc |
| to polyA) | | gcccattgacgtcaataatgacgtatgttcccatagtaacgcc |
| | | aatagggactttccattgacgtcaatgggtggagtatttacgg |
| | | taaactgcccacttggcagtacatcaagtgtatcatatgccaa |
| | | gtacgccccctattgacgtcaatgacggtaaatggcccgcctg |
| | | gcattatgcccagtacatgaccttatgggactttcctacttgg |
| | | cagtacatctacgtattagtcatcgctattaccatggtcgagg |
| | | tgagccccacgttctgcttcactctccccatctcccccccctc |
| | | cccacccccaattttgtatttatttattttttaattattttgt |
| | | gcagcgatgggggcggggggggggggggggcgcgcgccaggcg |
| | | gggcggggcggggcgaggggcggggcggggcgaggcggagagg |
| | | tgcggcggcagccaatcagagcggcgcgctccgaaagtttcct |
| | | tttatggcgaggcggcggcggcggcggccctataaaaagcgaa |
| | | gcgcgcggcgggcgggagtcgctgcgcgctgccttcgccccgt |
| | | gccccgctccgccgccgcctcgcgccgcccgccccggctctga |
| | | ctgaccgcgttactcccacaggtgagcgggcgggacggccctt |
| | | ctcctccgggctgtaattagcgcttggtttaatgacggcttgt |
| | | ttcttttctgtggctgcgtgaaagccttgaggggctccgggag |
| | | ggccctttgtgcggggggagcggctcggggggtgcgtgcgtgt |
| | | gtgtgtgcgtggggagcgccgcgtgcggctccgcgctgcccgg |
| | | cggctgtgagcgctgcgggcgcggcgcggggctttgtgcgctc |
| | | cgcagtgtgcgcgaggggagcgcggccgggggcggtgccccgc |
| | | ggtgcggggggggctgcgaggggaacaaaggctgcgtgcgggg |
| | | tgtgtgcgtgggggggtgagcagggggtgtgggcgcgtcggtc |
| | | gggctgcaaccccccctgcacccccctccccgagttgctgagc |
| | | acggcccggcttcgggtgcggggctccgtacggggcgtggcgc |
| | | ggggctcgccgtgccgggcggggggtggcggcaggtgggggtg |
| | | ccgggcggggcggggccgcctcgggccggggagggctcggggg |
| | | aggggcgcggcggcccccggagcgccggcggctgtcgaggcgc |
| | | ggcgagccgcagccattgccttttatggtaatcgtgcgagagg |
| | | gcgcagggacttcctttgtcccaaatctgtgcggagccgaaat |
| | | ctgggaggcgccgccgcaccccctctagcgggcgcggggcgaa |
| | | gcggtgcggcgccggcaggaaggaaatgggcggggagggcctt |
| | | cgtgcgtcgccgcgccgccgtccccttctccctctccagcctc |
| | | ggggctgtccgcggggggacggctgccttcgggggggacgggg |
| | | cagggcggggttcggcttctggcgtgtgaccggcggctctaga |
| | | gcctctgctaaccatgttcatgccttcttctttttcctacagc |
| | | tcctgggcaacgtgctggttattgtgctgtctcatcattttgg |
| | | caaagAATTCGCCACCATGTACAGAATGCAGCTGCTGCTGCTC |
| | | ATTGCCCTGTCTCTGGCCCTGGTCACCAATTCTGAAGTGCAGC |
| | | TGGTGGAAAGTGGTGGTGGACTGGTGCAGCCTGGCAGAAGCCT |
| | | GAGACTGTCTTGTGCTGCCTCTGGCTTCACCTTTGATGACTAT |
| | | GCCATGCACTGGGTCAGACAGGCCCCTGGCAAAGGACTGGAAT |
| | | GGGTGTCAGCCATCACCTGGAACTCTGGCCACATTGACTATGC |
| | | TGACTCTGTGGAAGGCAGATTCACCATCAGCAGAGACAATGCC |
| | | AAGAACAGCCTGTACCTGCAGATGAACTCCCTGAGAGCTGAGG |
| | | ACACAGCAGTGTACTACTGTGCCAAGGTGTCCTACCTGAGCAC |
| | | AGCCAGCAGCCTGGATTATTGGGGCCAGGGCACACTGGTTACA |
| | | GTGTCCTCTGCCAGCACAAAGGGCCCCTCTGTTTTTCCACTGG |
| | | CTCCCAGCAGCAAGAGCACCAGTGGTGGAACAGCTGCCCTGGG |
| | | CTGTCTGGTCAAGGATTACTTCCCTGAGCCTGTGACAGTGTCT |
| | | TGGAACTCAGGGGCTCTGACCTCTGGGGTGCACACATTTCCAG |
| | | CTGTGCTGCAGTCCTCTGGCCTGTACTCTCTGTCCTCTGTGGT |
| | | CACAGTGCCTAGCTCTAGCCTGGGCACCCAGACCTACATCTGC |
| | | AATGTGAACCACAAGCCTAGCAACACCAAGGTGGACAAGAAGG |
| | | TGGAACCCAAGAGCTGTGACAAGACCCACACCTGTCCTCCATG |
| | | TCCTGCTCCAGAACTGCTTGGAGGCCCTTCTGTGTTCCTGTTT |
| | | CCTCCAAAGCCTAAGGACACCCTGATGATCAGCAGAACCCCTG |
| | | AAGTGACCTGTGTGGTGGTTGATGTGTCCCATGAGGACCCAGA |
| | | AGTGAAGTTCAATTGGTATGTGGATGGGGTTGAAGTGCACAAT |
| | | GCTAAGACCAAGCCTAGAGAGGAACAGTACAACAGCACCTACA |
| | | GAGTGGTGTCTGTGCTGACAGTGCTGCATCAGGACTGGCTGAA |
| | | TGGCAAAGAGTACAAGTGCAAAGTGTCCAACAAGGCCCTGCCT |
| | | GCTCCTATTGAGAAAACCATCTCCAAGGCCAAGGGCCAGCCAA |
| | | GAGAACCCCAGGTTTACACACTGCCACCTAGCAGAGATGAGCT |
| | | GACCAAGAACCAGGTGTCCCTGACCTGCCTGGTTAAGGGCTTC |
| | | TACCCCTCTGACATTGCTGTGGAATGGGAGAGCAATGGCCAGC |
| | | CTGAGAACAACTACAAGACAACCCCTCCTGTGCTGGACTCTGA |
| | | TGGCTCATTCTTCCTGTACAGCAAGCTGACTGTGGACAAGTCC |
| | | AGATGGCAGCAGGGGAATGTGTTCAGCTGCTCTGTGATGCATG |
| | | AGGCCCTGCACAACCACTACACCCAGAAAAGTCTGAGTCTGAG |
| | | CCCTGGCAAGAGAAAGAGAAGAGGCTCTGGAGAAGGCAGAGGC |
| | | TCCCTGCTGACATGTGGGGATGTTGAAGAGAATCCTGGGCCTA |
| | | TGTATAGGATGCAACTGCTCCTCCTGATTGCTCTGAGCCTGGC |
| | | TCTTGTGACCAACTCTGACATCCAGATGACACAGAGCCCTAGC |
| | | AGCCTGTCTGCTTCTGTGGGAGACAGAGTGACCATCACATGCA |
| | | GAGCCAGCCAGGGAATCAGAAACTACCTGGCCTGGTATCAGCA |
| | | AAAGCCTGGCAAGGCCCCTAAGCTGCTGATCTATGCAGCCAGC |
| | | ACACTGCAGTCAGGGGTGCCAAGCAGATTTTCAGGCTCTGGCT |
| | | CTGGCACAGACTTCACCCTGACCATTTCTAGCCTGCAGCCTGA |
| | | GGATGTGGCCACCTACTACTGCCAGAGATACAACAGAGCCCCA |
| | | TACACCTTTGGACAGGGCACAAAGGTGGAAATCAAGAGAACAG |
| | | TGGCTGCCCCATCTGTGTTCATCTTCCCACCATCTGATGAACA |
| | | GCTGAAGTCTGGCACTGCCTCTGTTGTGTGCCTGCTGAACAAC |
| | | TTCTACCCTAGAGAAGCCAAGGTGCAGTGGAAGGTTGACAATG |
| | | CCCTGCAGTCTGGCAATAGCCAAGAATCTGTGACAGAGCAGGA |
| | | CTCCAAGGATTCCACCTACAGCCTGAGCAGCACCCTGACACTG |
| | | AGCAAGGCTGACTATGAGAAGCACAAAGTGTATGCCTGTGAAG |
| | | TGACACACCAGGGACTGAGCAGCCCAGTGACCAAGAGCTTCAA |
| | | CAGGGGAGAGTGCTGATAACtcgaggacggggtgaactacgcc |
| | | tgaggatccgatctttttccctctgccaaaaattatggggaca |
| | | tcatgaagccccttgagcatctgacttctggctaataaaggaa |
| | | atttattttcattgcaatagtgtgttggaattttttgtgtctc |
| | | tcactcg |
| Adalimumab | SEQ ID | ATGTACAGAATGCAGCTGCTGCTGCTCATTGCCCTGTCTCTGG |
| Full-length mAb | NO: 48 | CCCTGGTCACCAATTCTGAAGTGCAGCTGGTGGAAAGTGGTGG |
| single chain | | TGGACTGGTGCAGCCTGGCAGAAGCCTGAGACTGTCTTGTGCT |
| construct | | GCCTCTGGCTTCACCTTTGATGACTATGCCATGCACTGGGTCA |
| transgene | | GACAGGCCCCTGGCAAAGGACTGGAATGGGTGTCAGCCATCAC |
| (HC-Furin | | CTGGAACTCTGGCCACATTGACTATGCTGACTCTGTGGAAGGC |
| linker-T2A-LC) | | AGATTCACCATCAGCAGAGACAATGCCAAGAACAGCCTGTACC |
| | | TGCAGATGAACTCCCTGAGAGCTGAGGACACAGCAGTGTACTA |
| | | CTGTGCCAAGGTGTCCTACCTGAGCACAGCCAGCAGCCTGGAT |
| | | TATTGGGGCCAGGGCACACTGGTTACAGTGTCCTCTGCCAGCA |
| | | CAAAGGGCCCCTCTGTTTTTCCACTGGCTCCCAGCAGCAAGAG |
| | | CACCAGTGGTGGAACAGCTGCCCTGGGCTGTCTGGTCAAGGAT |
| | | TACTTCCCTGAGCCTGTGACAGTGTCTTGGAACTCAGGGGCTC |
| | | TGACCTCTGGGGTGCACACATTTCCAGCTGTGCTGCAGTCCTC |
| | | TGGCCTGTACTCTCTGTCCTCTGTGGTCACAGTGCCTAGCTCT |
| | | AGCCTGGGCACCCAGACCTACATCTGCAATGTGAACCACAAGC |
| | | CTAGCAACACCAAGGTGGACAAGAAGGTGGAACCCAAGAGCTG |
| | | TGACAAGACCCACACCTGTCCTCCATGTCCTGCTCCAGAACTG |
| | | CTTGGAGGCCCTTCTGTGTTCCTGTTTCCTCCAAAGCCTAAGG |
| | | ACACCCTGATGATCAGCAGAACCCCTGAAGTGACCTGTGTGGT |
| | | GGTTGATGTGTCCCATGAGGACCCAGAAGTGAAGTTCAATTGG |
| | | TATGTGGATGGGGTTGAAGTGCACAATGCTAAGACCAAGCCTA |
| | | GAGAGGAACAGTACAACAGCACCTACAGAGTGGTGTCTGTGCT |
| | | GACAGTGCTGCATCAGGACTGGCTGAATGGCAAAGAGTACAAG |
| | | TGCAAAGTGTCCAACAAGGCCCTGCCTGCTCCTATTGAGAAAA |
| | | CCATCTCCAAGGCCAAGGGCCAGCCAAGAGAACCCCAGGTTTA |
| | | CACACTGCCACCTAGCAGAGATGAGCTGACCAAGAACCAGGTG |
| | | TCCCTGACCTGCCTGGTTAAGGGCTTCTACCCCTCTGACATTG |
| | | CTGTGGAATGGGAGAGCAATGGCCAGCCTGAGAACAACTACAA |
| | | GACAACCCCTCCTGTGCTGGACTCTGATGGCTCATTCTTCCTG |
| | | TACAGCAAGCTGACTGTGGACAAGTCCAGATGGCAGCAGGGGA |
| | | ATGTGTTCAGCTGCTCTGTGATGCATGAGGCCCTGCACAACCA |
| | | CTACACCCAGAAAAGTCTGAGTCTGAGCCCTGGCAAGAGAAAG |
| | | AGAAGAGGCTCTGGAGAAGGCAGAGGCTCCCTGCTGACATGTG |
| | | GGGATGTTGAAGAGAATCCTGGGCCTATGTATAGGATGCAACT |
| | | GCTCCTCCTGATTGCTCTGAGCCTGGCTCTTGTGACCAACTCT |
| | | GACATCCAGATGACACAGAGCCCTAGCAGCCTGTCTGCTTCTG |
| | | TGGGAGACAGAGTGACCATCACATGCAGAGCCAGCCAGGGAAT |
| | | CAGAAACTACCTGGCCTGGTATCAGCAAAAGCCTGGCAAGGCC |
| | | CCTAAGCTGCTGATCTATGCAGCCAGCACACTGCAGTCAGGGG |
| | | TGCCAAGCAGATTTTCAGGCTCTGGCTCTGGCACAGACTTCAC |
| | | CCTGACCATTTCTAGCCTGCAGCCTGAGGATGTGGCCACCTAC |
| | | TACTGCCAGAGATACAACAGAGCCCCATACACCTTTGGACAGG |
| | | GCACAAAGGTGGAAATCAAGAGAACAGTGGCTGCCCCATCTGT |
| | | GTTCATCTTCCCACCATCTGATGAACAGCTGAAGTCTGGCACT |
| | | GCCTCTGTTGTGTGCCTGCTGAACAACTTCTACCCTAGAGAAG |
| | | CCAAGGTGCAGTGGAAGGTTGACAATGCCCTGCAGTCTGGCAA |
| | | TAGCCAAGAATCTGTGACAGAGCAGGACTCCAAGGATTCCACC |
| | | TACAGCCTGAGCAGCACCCTGACACTGAGCAAGGCTGACTATG |
| | | AGAAGCACAAAGTGTATGCCTGTGAAGTGACACACCAGGGACT |
| | | GAGCAGCCCAGTGACCAAGAGCTTCAACAGGGGAGAGTGCTGA |
| pAAV.CAG. | SEQ ID | ctgcgcgctcgctcgctcactgaggccgcccgggcaaagcccg |
| Adalimumab. | NO: 49 | ggcgtcgggcgacctttggtcgcccggcctcagtgagcgagcg |
| Fab cassette | | agcgcgcagagagggagtggccaactccatcactaggggttcc |
| (5′-ITR to | | ttgtagttaatgattaacccgccatgctacttatctaccaggg |
| 3′-ITR) | | taatggggatcctctagaactatagctagtcgacattgattat |
| | | tgactagttattaatagtaatcaattacggggtcattagttca |
| | | tagcccatatatggagttccgcgttacataacttacggtaaat |
| | | ggcccgcctggctgaccgcccaacgacccccgcccattgacgt |
| | | caataatgacgtatgttcccatagtaacgccaatagggacttt |
| | | ccattgacgtcaatgggtggagtatttacggtaaactgcccac |
| | | ttggcagtacatcaagtgtatcatatgccaagtacgcccccta |
| | | ttgacgtcaatgacggtaaatggcccgcctggcattatgccca |
| | | gtacatgaccttatgggactttcctacttggcagtacatctac |
| | | gtattagtcatcgctattaccatggtcgaggtgagccccacgt |
| | | tctgcttcactctccccatctcccccccctccccacccccaat |
| | | tttgtatttatttattttttaattattttgtgcagcgatgggg |
| | | gcggggggggggggggggcgcgcgccaggcggggcggggcggg |
| | | gcgaggggcggggcggggcgaggcggagaggtgcggcggcagc |
| | | caatcagagcggcgcgctccgaaagtttccttttatggcgagg |
| | | cggcggcggcggcggccctataaaaagcgaagcgcgcggcggg |
| | | cgggagtcgctgcgcgctgccttcgccccgtgccccgctccgc |
| | | cgccgcctcgcgccgcccgccccggctctgactgaccgcgtta |
| | | ctcccacaggtgagcgggcgggacggcccttctcctccgggct |
| | | gtaattagcgcttggtttaatgacggcttgtttcttttctgtg |
| | | gctgcgtgaaagccttgaggggctccgggagggccctttgtgc |
| | | ggggggagcggctcggggggtgcgtgcgtgtgtgtgtgcgtgg |
| | | ggagcgccgcgtgcggctccgcgctgcccggcggctgtgagcg |
| | | ctgcgggcgcggcgcggggctttgtgcgctccgcagtgtgcgc |
| | | gaggggagcgcggccgggggcggtgccccgcggtgcggggggg |
| | | gctgcgaggggaacaaaggctgcgtgcggggtgtgtgcgtggg |
| | | ggggtgagcagggggtgtgggcgcgtcggtcgggctgcaaccc |
| | | cccctgcacccccctccccgagttgctgagcacggcccggctt |
| | | cgggtgcggggctccgtacggggcgtggcgcggggctcgccgt |
| | | gccgggcggggggtggcggcaggtgggggtgccgggcggggcg |
| | | gggccgcctcgggccggggagggctcgggggaggggcgcggcg |
| | | gcccccggagcgccggcggctgtcgaggcgcggcgagccgcag |
| | | ccattgccttttatggtaatcgtgcgagagggcgcagggactt |
| | | cctttgtcccaaatctgtgcggagccgaaatctgggaggcgcc |
| | | gccgcaccccctctagcgggcgcggggcgaagcggtgcggcgc |
| | | cggcaggaaggaaatgggcggggagggccttcgtgcgtcgccg |
| | | cgccgccgtccccttctccctctccagcctcggggctgtccgc |
| | | ggggggacggctgccttcgggggggacggggcagggcggggtt |
| | | cggcttctggcgtgtgaccggcggctctagagcctctgctaac |
| | | catgttcatgccttcttctttttcctacagctcctgggcaacg |
| | | tgctggttattgtgctgtctcatcattttggcaaagAATTCGC |
| | | CACCATGTACAGAATGCAGCTGCTGCTGCTCATTGCCCTGTCT |
| | | CTGGCCCTGGTCACCAATTCTGAAGTGCAGCTGGTGGAAAGTG |
| | | GTGGTGGACTGGTGCAGCCTGGCAGAAGCCTGAGACTGTCTTG |
| | | TGCTGCCTCTGGCTTCACCTTTGATGACTATGCCATGCACTGG |
| | | GTCAGACAGGCCCCTGGCAAAGGACTGGAATGGGTGTCAGCCA |
| | | TCACCTGGAACTCTGGCCACATTGACTATGCTGACTCTGTGGA |
| | | AGGCAGATTCACCATCAGCAGAGACAATGCCAAGAACAGCCTG |
| | | TACCTGCAGATGAACTCCCTGAGAGCTGAGGACACAGCAGTGT |
| | | ACTACTGTGCCAAGGTGTCCTACCTGAGCACAGCCAGCAGCCT |
| | | GGATTATTGGGGCCAGGGCACACTGGTTACAGTGTCCTCTGCC |
| | | AGCACAAAGGGCCCCTCTGTTTTTCCACTGGCTCCCAGCAGCA |
| | | AGAGCACCAGTGGTGGAACAGCTGCCCTGGGCTGTCTGGTCAA |
| | | GGATTACTTCCCTGAGCCTGTGACAGTGTCTTGGAACTCAGGG |
| | | GCTCTGACCTCTGGGGTGCACACATTTCCAGCTGTGCTGCAGT |
| | | CCTCTGGCCTGTACTCTCTGTCCTCTGTGGTCACAGTGCCTAG |
| | | CTCTAGCCTGGGCACCCAGACCTACATCTGCAATGTGAACCAC |
| | | AAGCCTAGCAACACCAAGGTGGACAAGAAGGTGGAACCCAAGA |
| | | GCTGTGACAAGACCCACAGAAAGAGAAGAGGCTCTGGAGAAGG |
| | | CAGAGGCTCCCTGCTGACATGTGGGGATGTTGAAGAGAATCCT |
| | | GGGCCTATGTATAGGATGCAACTGCTCCTCCTGATTGCTCTGA |
| | | GCCTGGCTCTTGTGACCAACTCTGACATCCAGATGACACAGAG |
| | | CCCTAGCAGCCTGTCTGCTTCTGTGGGAGACAGAGTGACCATC |
| | | ACATGCAGAGCCAGCCAGGGCATCAGAAACTACCTGGCCTGGT |
| | | ATCAGCAGAAGCCAGGCAAGGCCCCTAAGCTGCTGATCTATGC |
| | | AGCCAGCACACTGCAGAGTGGGGTGCCAAGCAGATTTTCAGGC |
| | | TCTGGCTCTGGCACAGACTTCACCCTGACCATTTCTAGCCTGC |
| | | AGCCTGAGGATGTGGCCACCTACTACTGCCAGAGATACAACAG |
| | | AGCCCCATACACCTTTGGACAGGGCACAAAGGTGGAAATCAAG |
| | | AGAACAgttgcagcaccctcagttttcatcttccccccctcag |
| | | atgaacagCTGAAGTCTGGCACTGCCTCTGTTGTGTGCCTGCT |
| | | GAACAACTTCTACCCCAGAGAAGCCAAGGTGCAGTGGAAGGTT |
| | | GACAATGCCCTGCAGTCAGGCAACAGCCAAGAATCTGTGACTG |
| | | AACAGGATTCCAAGGATAGCACCTACAGCCTGAGCAGCACCCT |
| | | GACACTGAGCAAGGCTGACTATGAGAAGCACAAAGTGTATGCC |
| | | TGTGAAGTGACCCACCAGGGACTGAGCAGCCCAGTGACCAAGA |
| | | GCTTCAACAGGGGAGAGTGCTGATAACtcgaggacggggtgaa |
| | | ctacgcctgaggatccgatctttttccctctgccaaaaattat |
| | | ggggacatcatgaagccccttgagcatctgacttctggctaat |
| | | aaaggaaatttattttcattgcaatagtgtgttggaatttttt |
| | | gtgtctctcactcggaagcaattcgttgatctgaatttcgacc |
| | | acccataatacccattaccctggtagataagtagcatggcggg |
| | | ttaatcattaactacaaggaacccctagtgatggagttggcca |
| | | ctccctctctgcgcgctcgctcgctcactgaggccgggcgacc |
| | | aaaggtcgcccgacgcccgggctttgcccgggcggcctcagtg |
| | | agcgagcgagcgcgcag |
| pAAV.CAG. | SEQ ID | gacattgattattgactagttattaatagtaatcaattacggg |
| Adalimumab. | NO: 50 | gtcattagttcatagcccatatatggagttccgcgttacataa |
| Fab cassette | | cttacggtaaatggcccgcctggctgaccgcccaacgaccccc |
| (promoter to | | gcccattgacgtcaataatgacgtatgttcccatagtaacgcc |
| polyA) | | aatagggactttccattgacgtcaatgggtggagtatttacgg |
| | | taaactgcccacttggcagtacatcaagtgtatcatatgccaa |
| | | gtacgccccctattgacgtcaatgacggtaaatggcccgcctg |
| | | gcattatgcccagtacatgaccttatgggactttcctacttgg |
| | | cagtacatctacgtattagtcatcgctattaccatggtcgagg |
| | | tgagccccacgttctgcttcactctccccatctcccccccctc |
| | | cccacccccaattttgtatttatttattttttaattattttgt |
| | | gcagcgatgggggcggggggggggggggggcgcgcgccaggcg |
| | | gggcggggcggggcgaggggcggggcggggcgaggcggagagg |
| | | tgcggcggcagccaatcagagcggcgcgctccgaaagtttcct |
| | | tttatggcgaggcggcggcggcggcggccctataaaaagcgaa |
| | | gcgcgcggcgggcgggagtcgctgcgcgctgccttcgccccgt |
| | | gccccgctccgccgccgcctcgcgccgcccgccccggctctga |
| | | ctgaccgcgttactcccacaggtgagcgggcgggacggccctt |
| | | ctcctccgggctgtaattagcgcttggtttaatgacggcttgt |
| | | ttcttttctgtggctgcgtgaaagccttgaggggctccgggag |
| | | ggccctttgtgcggggggagcggctcggggggtgcgtgcgtgt |
| | | gtgtgtgcgtggggagcgccgcgtgcggctccgcgctgcccgg |
| | | cggctgtgagcgctgcgggcgcggcgcggggctttgtgcgctc |
| | | cgcagtgtgcgcgaggggagcgcggccgggggcggtgccccgc |
| | | ggtgcggggggggctgcgaggggaacaaaggctgcgtgcgggg |
| | | tgtgtgcgtgggggggtgagcagggggtgtgggcgcgtcggtc |
| | | gggctgcaaccccccctgcacccccctccccgagttgctgagc |
| | | acggcccggcttcgggtgcggggctccgtacggggcgtggcgc |
| | | ggggctcgccgtgccgggcggggggtggcggcaggtgggggtg |
| | | ccgggcggggcggggccgcctcgggccggggagggctcggggg |
| | | aggggcgcggcggcccccggagcgccggcggctgtcgaggcgc |
| | | ggcgagccgcagccattgccttttatggtaatcgtgcgagagg |
| | | gcgcagggacttcctttgtcccaaatctgtgcggagccgaaat |
| | | ctgggaggcgccgccgcaccccctctagcgggcgcggggcgaa |
| | | gcggtgcggcgccggcaggaaggaaatgggcggggagggcctt |
| | | cgtgcgtcgccgcgccgccgtccccttctccctctccagcctc |
| | | ggggctgtccgcggggggacggctgccttcgggggggacgggg |
| | | cagggcggggttcggcttctggcgtgtgaccggcggctctaga |
| | | gcctctgctaaccatgttcatgccttcttctttttcctacagc |
| | | tcctgggcaacgtgctggttattgtgctgtctcatcattttgg |
| | | caaagAATTCGCCACCATGTACAGAATGCAGCTGCTGCTGCTC |
| | | ATTGCCCTGTCTCTGGCCCTGGTCACCAATTCTGAAGTGCAGC |
| | | TGGTGGAAAGTGGTGGTGGACTGGTGCAGCCTGGCAGAAGCCT |
| | | GAGACTGTCTTGTGCTGCCTCTGGCTTCACCTTTGATGACTAT |
| | | GCCATGCACTGGGTCAGACAGGCCCCTGGCAAAGGACTGGAAT |
| | | GGGTGTCAGCCATCACCTGGAACTCTGGCCACATTGACTATGC |
| | | TGACTCTGTGGAAGGCAGATTCACCATCAGCAGAGACAATGCC |
| | | AAGAACAGCCTGTACCTGCAGATGAACTCCCTGAGAGCTGAGG |
| | | ACACAGCAGTGTACTACTGTGCCAAGGTGTCCTACCTGAGCAC |
| | | AGCCAGCAGCCTGGATTATTGGGGCCAGGGCACACTGGTTACA |
| | | GTGTCCTCTGCCAGCACAAAGGGCCCCTCTGTTTTTCCACTGG |
| | | CTCCCAGCAGCAAGAGCACCAGTGGTGGAACAGCTGCCCTGGG |
| | | CTGTCTGGTCAAGGATTACTTCCCTGAGCCTGTGACAGTGTCT |
| | | TGGAACTCAGGGGCTCTGACCTCTGGGGTGCACACATTTCCAG |
| | | CTGTGCTGCAGTCCTCTGGCCTGTACTCTCTGTCCTCTGTGGT |
| | | CACAGTGCCTAGCTCTAGCCTGGGCACCCAGACCTACATCTGC |
| | | AATGTGAACCACAAGCCTAGCAACACCAAGGTGGACAAGAAGG |
| | | TGGAACCCAAGAGCTGTGACAAGACCCACAGAAAGAGAAGAGG |
| | | CTCTGGAGAAGGCAGAGGCTCCCTGCTGACATGTGGGGATGTT |
| | | GAAGAGAATCCTGGGCCTATGTATAGGATGCAACTGCTCCTCC |
| | | TGATTGCTCTGAGCCTGGCTCTTGTGACCAACTCTGACATCCA |
| | | GATGACACAGAGCCCTAGCAGCCTGTCTGCTTCTGTGGGAGAC |
| | | AGAGTGACCATCACATGCAGAGCCAGCCAGGGCATCAGAAACT |
| | | ACCTGGCCTGGTATCAGCAGAAGCCAGGCAAGGCCCCTAAGCT |
| | | GCTGATCTATGCAGCCAGCACACTGCAGAGTGGGGTGCCAAGC |
| | | AGATTTTCAGGCTCTGGCTCTGGCACAGACTTCACCCTGACCA |
| | | TTTCTAGCCTGCAGCCTGAGGATGTGGCCACCTACTACTGCCA |
| | | GAGATACAACAGAGCCCCATACACCTTTGGACAGGGCACAAAG |
| | | GTGGAAATCAAGAGAACAgttgcagcaccctcagttttcatct |
| | | tccccccctcagatgaacagCTGAAGTCTGGCACTGCCTCTGT |
| | | TGTGTGCCTGCTGAACAACTTCTACCCCAGAGAAGCCAAGGTG |
| | | CAGTGGAAGGTTGACAATGCCCTGCAGTCAGGCAACAGCCAAG |
| | | AATCTGTGACTGAACAGGATTCCAAGGATAGCACCTACAGCCT |
| | | GAGCAGCACCCTGACACTGAGCAAGGCTGACTATGAGAAGCAC |
| | | AAAGTGTATGCCTGTGAAGTGACCCACCAGGGACTGAGCAGCC |
| | | CAGTGACCAAGAGCTTCAACAGGGGAGAGTGCTGATAACtcga |
| | | ggacggggtgaactacgcctgaggatccgatctttttccctct |
| | | gccaaaaattatggggacatcatgaagccccttgagcatctga |
| | | cttctggctaataaaggaaatttattttcattgcaatagtgtg |
| | | ttggaattttttgtgtctctcactcg |
| Adalimumab | SEQ ID | ATGTACAGAATGCAGCTGCTGCTGCTCATTGCCCTGTCTCTGG |
| Fab single chain | NO: 51 | CCCTGGTCACCAATTCTGAAGTGCAGCTGGTGGAAAGTGGTGG |
| construct | | TGGACTGGTGCAGCCTGGCAGAAGCCTGAGACTGTCTTGTGCT |
| transgene | | GCCTCTGGCTTCACCTTTGATGACTATGCCATGCACTGGGTCA |
| (HC-Furin- | | GACAGGCCCCTGGCAAAGGACTGGAATGGGTGTCAGCCATCAC |
| linker-T2A-LC) | | CTGGAACTCTGGCCACATTGACTATGCTGACTCTGTGGAAGGC |
| | | AGATTCACCATCAGCAGAGACAATGCCAAGAACAGCCTGTACC |
| | | TGCAGATGAACTCCCTGAGAGCTGAGGACACAGCAGTGTACTA |
| | | CTGTGCCAAGGTGTCCTACCTGAGCACAGCCAGCAGCCTGGAT |
| | | TATTGGGGCCAGGGCACACTGGTTACAGTGTCCTCTGCCAGCA |
| | | CAAAGGGCCCCTCTGTTTTTCCACTGGCTCCCAGCAGCAAGAG |
| | | CACCAGTGGTGGAACAGCTGCCCTGGGCTGTCTGGTCAAGGAT |
| | | TACTTCCCTGAGCCTGTGACAGTGTCTTGGAACTCAGGGGCTC |
| | | TGACCTCTGGGGTGCACACATTTCCAGCTGTGCTGCAGTCCTC |
| | | TGGCCTGTACTCTCTGTCCTCTGTGGTCACAGTGCCTAGCTCT |
| | | AGCCTGGGCACCCAGACCTACATCTGCAATGTGAACCACAAGC |
| | | CTAGCAACACCAAGGTGGACAAGAAGGTGGAACCCAAGAGCTG |
| | | TGACAAGACCCACAGAAAGAGAAGAGGCTCTGGAGAAGGCAGA |
| | | GGCTCCCTGCTGACATGTGGGGATGTTGAAGAGAATCCTGGGC |
| | | CTATGTATAGGATGCAACTGCTCCTCCTGATTGCTCTGAGCCT |
| | | GGCTCTTGTGACCAACTCTGACATCCAGATGACACAGAGCCCT |
| | | AGCAGCCTGTCTGCTTCTGTGGGAGACAGAGTGACCATCACAT |
| | | GCAGAGCCAGCCAGGGCATCAGAAACTACCTGGCCTGGTATCA |
| | | GCAGAAGCCAGGCAAGGCCCCTAAGCTGCTGATCTATGCAGCC |
| | | AGCACACTGCAGAGTGGGGTGCCAAGCAGATTTTCAGGCTCTG |
| | | GCTCTGGCACAGACTTCACCCTGACCATTTCTAGCCTGCAGCC |
| | | TGAGGATGTGGCCACCTACTACTGCCAGAGATACAACAGAGCC |
| | | CCATACACCTTTGGACAGGGCACAAAGGTGGAAATCAAGAGAA |
| | | CAgttgcagcaccctcagttttcatcttccccccctcagatga |
| | | acagCTGAAGTCTGGCACTGCCTCTGTTGTGTGCCTGCTGAAC |
| | | AACTTCTACCCCAGAGAAGCCAAGGTGCAGTGGAAGGTTGACA |
| | | ATGCCCTGCAGTCAGGCAACAGCCAAGAATCTGTGACTGAACA |
| | | GGATTCCAAGGATAGCACCTACAGCCTGAGCAGCACCCTGACA |
| | | CTGAGCAAGGCTGACTATGAGAAGCACAAAGTGTATGCCTGTG |
| | | AAGTGACCCACCAGGGACTGAGCAGCCCAGTGACCAAGAGCTT |
| | | CAACAGGGGAGAGTGCTGA |
| pAAV.GRK1. | SEQ ID | ctgcgcgctcgctcgctcactgaggccgcccgggcaaagcccg |
| Vh4i. | NO: 52 | ggcgtcgggcgacctttggtcgcccggcctcagtgagcgagcg |
| Adalimumab. | | agcgcgcagagagggagtggccaactccatcactaggggttcc |
| IgG (5′-ITR to | | ttgtagttaatgattaacccgccatgctacttatctaccaggg |
| 3--ITR) | | taatggggatcctCTAGAGGGCCCCAGAAGCCTGGTGGTTGTT |
| | | TGTCCTTCTCAGGGGAAAAGTGAGGCGGCCCCTTGGAGGAAGG |
| | | GGCCGGGCAGAATGATCTAATCGGATTCCAAGCAGCTCAGGGG |
| | | ATTGTCTTTTTCTAGCACCTTCTTGCCACTCCTAAGCGTCCTC |
| | | CGTGACCCCGGCTGGGATTTAGCCTGGTGCTGTGTCAGCCCCG |
| | | GGCTCCCAGGGGCTTCCCAGTGGTCCCCAGGAACCCTCGACAG |
| | | GGCCAGGGCGTCTCTCTCGTCCAGCAAGGGCAGGGACGGGCCA |
| | | CAGGCCAAGGGCCAGGTGAGTATCTCAGGGATCCAGACATGGG |
| | | GATATGGGAGGTGCCTCTGATCCCAGGGCTCACTGTGGGTCTC |
| | | TCTGTTCACAGGTTGAATTCGCCACCATGTACAGAATGCAGCT |
| | | GCTGCTGCTCATTGCCCTGTCTCTGGCCCTGGTCACCAATTCT |
| | | GAAGTGCAGCTGGTGGAAAGTGGTGGTGGACTGGTGCAGCCTG |
| | | GCAGAAGCCTGAGACTGTCTTGTGCTGCCTCTGGCTTCACCTT |
| | | TGATGACTATGCCATGCACTGGGTCAGACAGGCCCCTGGCAAA |
| | | GGACTGGAATGGGTGTCAGCCATCACCTGGAACTCTGGCCACA |
| | | TTGACTATGCTGACTCTGTGGAAGGCAGATTCACCATCAGCAG |
| | | AGACAATGCCAAGAACAGCCTGTACCTGCAGATGAACTCCCTG |
| | | AGAGCTGAGGACACAGCAGTGTACTACTGTGCCAAGGTGTCCT |
| | | ACCTGAGCACAGCCAGCAGCCTGGATTATTGGGGCCAGGGCAC |
| | | ACTGGTTACAGTGTCCTCTGCCAGCACAAAGGGCCCCTCTGTT |
| | | TTTCCACTGGCTCCCAGCAGCAAGAGCACCAGTGGTGGAACAG |
| | | CTGCCCTGGGCTGTCTGGTCAAGGATTACTTCCCTGAGCCTGT |
| | | GACAGTGTCTTGGAACTCAGGGGCTCTGACCTCTGGGGTGCAC |
| | | ACATTTCCAGCTGTGCTGCAGTCCTCTGGCCTGTACTCTCTGT |
| | | CCTCTGTGGTCACAGTGCCTAGCTCTAGCCTGGGCACCCAGAC |
| | | CTACATCTGCAATGTGAACCACAAGCCTAGCAACACCAAGGTG |
| | | GACAAGAAGGTGGAACCCAAGAGCTGTGACAAGACCCACACCT |
| | | GTCCTCCATGTCCTGCTCCAGAACTGCTTGGAGGCCCTTCTGT |
| | | GTTCCTGTTTCCTCCAAAGCCTAAGGACACCCTGATGATCAGC |
| | | AGAACCCCTGAAGTGACCTGTGTGGTGGTTGATGTGTCCCATG |
| | | AGGACCCAGAAGTGAAGTTCAATTGGTATGTGGATGGGGTTGA |
| | | AGTGCACAATGCTAAGACCAAGCCTAGAGAGGAACAGTACAAC |
| | | AGCACCTACAGAGTGGTGTCTGTGCTGACAGTGCTGCATCAGG |
| | | ACTGGCTGAATGGCAAAGAGTACAAGTGCAAAGTGTCCAACAA |
| | | GGCCCTGCCTGCTCCTATTGAGAAAACCATCTCCAAGGCCAAG |
| | | GGCCAGCCAAGAGAACCCCAGGTTTACACACTGCCACCTAGCA |
| | | GAGATGAGCTGACCAAGAACCAGGTGTCCCTGACCTGCCTGGT |
| | | TAAGGGCTTCTACCCCTCTGACATTGCTGTGGAATGGGAGAGC |
| | | AATGGCCAGCCTGAGAACAACTACAAGACAACCCCTCCTGTGC |
| | | TGGACTCTGATGGCTCATTCTTCCTGTACAGCAAGCTGACTGT |
| | | GGACAAGTCCAGATGGCAGCAGGGGAATGTGTTCAGCTGCTCT |
| | | GTGATGCATGAGGCCCTGCACAACCACTACACCCAGAAAAGTC |
| | | TGAGTCTGAGCCCTGGCAAGAGAAAGAGAAGAGGCTCTGGAGA |
| | | AGGCAGAGGCTCCCTGCTGACATGTGGGGATGTTGAAGAGAAT |
| | | CCTGGGCCTATGTATAGGATGCAACTGCTCCTCCTGATTGCTC |
| | | TGAGCCTGGCTCTTGTGACCAACTCTGACATCCAGATGACACA |
| | | GAGCCCTAGCAGCCTGTCTGCTTCTGTGGGAGACAGAGTGACC |
| | | ATCACATGCAGAGCCAGCCAGGGAATCAGAAACTACCTGGCCT |
| | | GGTATCAGCAAAAGCCTGGCAAGGCCCCTAAGCTGCTGATCTA |
| | | TGCAGCCAGCACACTGCAGTCAGGGGTGCCAAGCAGATTTTCA |
| | | GGCTCTGGCTCTGGCACAGACTTCACCCTGACCATTTCTAGCC |
| | | TGCAGCCTGAGGATGTGGCCACCTACTACTGCCAGAGATACAA |
| | | CAGAGCCCCATACACCTTTGGACAGGGCACAAAGGTGGAAATC |
| | | AAGAGAACAGTGGCTGCCCCATCTGTGTTCATCTTCCCACCAT |
| | | CTGATGAACAGCTGAAGTCTGGCACTGCCTCTGTTGTGTGCCT |
| | | GCTGAACAACTTCTACCCTAGAGAAGCCAAGGTGCAGTGGAAG |
| | | GTTGACAATGCCCTGCAGTCTGGCAATAGCCAAGAATCTGTGA |
| | | CAGAGCAGGACTCCAAGGATTCCACCTACAGCCTGAGCAGCAC |
| | | CCTGACACTGAGCAAGGCTGACTATGAGAAGCACAAAGTGTAT |
| | | GCCTGTGAAGTGACACACCAGGGACTGAGCAGCCCAGTGACCA |
| | | AGAGCTTCAACAGGGGAGAGTGCTGATAACtcgaggacggggt |
| | | gaactacgcctgaggatccgatctttttccctctgccaaaaat |
| | | tatggggacatcatgaagccccttgagcatctgacttctggct |
| | | aataaaggaaatttattttcattgcaatagtgtgttggaattt |
| | | tttgtgtctctcactcggaagcaattcgttgatctgaatttcg |
| | | accacccataatacccattaccctggtagataagtagcatggc |
| | | gggttaatcattaactacaaggaacccctagtgatggagttgg |
| | | ccactccctctctgcgcgctcgctcgctcactgaggccgggcg |
| | | accaaaggtcgcccgacgcccgggctttgcccgggcggcctca |
| | | gtgagcgagcgagcgcgcag |
| pAAV.GRK1. | SEQ ID | GGGCCCCAGAAGCCTGGTGGTTGTTTGTCCTTCTCAGGGGAAA |
| Vh4i. | NO: 53 | AGTGAGGCGGCCCCTTGGAGGAAGGGGCCGGGCAGAATGATCT |
| Adalimumab. | | AATCGGATTCCAAGCAGCTCAGGGGATTGTCTTTTTCTAGCAC |
| IgG (promoter | | CTTCTTGCCACTCCTAAGCGTCCTCCGTGACCCCGGCTGGGAT |
| to polyA) | | TTAGCCTGGTGCTGTGTCAGCCCCGGGCTCCCAGGGGCTTCCC |
| | | AGTGGTCCCCAGGAACCCTCGACAGGGCCAGGGCGTCTCTCTC |
| | | GTCCAGCAAGGGCAGGGACGGGCCACAGGCCAAGGGCCAGGTG |
| | | AGTATCTCAGGGATCCAGACATGGGGATATGGGAGGTGCCTCT |
| | | GATCCCAGGGCTCACTGTGGGTCTCTCTGTTCACAGGTTGAAT |
| | | TCGCCACCATGTACAGAATGCAGCTGCTGCTGCTCATTGCCCT |
| | | GTCTCTGGCCCTGGTCACCAATTCTGAAGTGCAGCTGGTGGAA |
| | | AGTGGTGGTGGACTGGTGCAGCCTGGCAGAAGCCTGAGACTGT |
| | | CTTGTGCTGCCTCTGGCTTCACCTTTGATGACTATGCCATGCA |
| | | CTGGGTCAGACAGGCCCCTGGCAAAGGACTGGAATGGGTGTCA |
| | | GCCATCACCTGGAACTCTGGCCACATTGACTATGCTGACTCTG |
| | | TGGAAGGCAGATTCACCATCAGCAGAGACAATGCCAAGAACAG |
| | | CCTGTACCTGCAGATGAACTCCCTGAGAGCTGAGGACACAGCA |
| | | GTGTACTACTGTGCCAAGGTGTCCTACCTGAGCACAGCCAGCA |
| | | GCCTGGATTATTGGGGCCAGGGCACACTGGTTACAGTGTCCTC |
| | | TGCCAGCACAAAGGGCCCCTCTGTTTTTCCACTGGCTCCCAGC |
| | | AGCAAGAGCACCAGTGGTGGAACAGCTGCCCTGGGCTGTCTGG |
| | | TCAAGGATTACTTCCCTGAGCCTGTGACAGTGTCTTGGAACTC |
| | | AGGGGCTCTGACCTCTGGGGTGCACACATTTCCAGCTGTGCTG |
| | | CAGTCCTCTGGCCTGTACTCTCTGTCCTCTGTGGTCACAGTGC |
| | | CTAGCTCTAGCCTGGGCACCCAGACCTACATCTGCAATGTGAA |
| | | CCACAAGCCTAGCAACACCAAGGTGGACAAGAAGGTGGAACCC |
| | | AAGAGCTGTGACAAGACCCACACCTGTCCTCCATGTCCTGCTC |
| | | CAGAACTGCTTGGAGGCCCTTCTGTGTTCCTGTTTCCTCCAAA |
| | | GCCTAAGGACACCCTGATGATCAGCAGAACCCCTGAAGTGACC |
| | | TGTGTGGTGGTTGATGTGTCCCATGAGGACCCAGAAGTGAAGT |
| | | TCAATTGGTATGTGGATGGGGTTGAAGTGCACAATGCTAAGAC |
| | | CAAGCCTAGAGAGGAACAGTACAACAGCACCTACAGAGTGGTG |
| | | TCTGTGCTGACAGTGCTGCATCAGGACTGGCTGAATGGCAAAG |
| | | AGTACAAGTGCAAAGTGTCCAACAAGGCCCTGCCTGCTCCTAT |
| | | TGAGAAAACCATCTCCAAGGCCAAGGGCCAGCCAAGAGAACCC |
| | | CAGGTTTACACACTGCCACCTAGCAGAGATGAGCTGACCAAGA |
| | | ACCAGGTGTCCCTGACCTGCCTGGTTAAGGGCTTCTACCCCTC |
| | | TGACATTGCTGTGGAATGGGAGAGCAATGGCCAGCCTGAGAAC |
| | | AACTACAAGACAACCCCTCCTGTGCTGGACTCTGATGGCTCAT |
| | | TCTTCCTGTACAGCAAGCTGACTGTGGACAAGTCCAGATGGCA |
| | | GCAGGGGAATGTGTTCAGCTGCTCTGTGATGCATGAGGCCCTG |
| | | CACAACCACTACACCCAGAAAAGTCTGAGTCTGAGCCCTGGCA |
| | | AGAGAAAGAGAAGAGGCTCTGGAGAAGGCAGAGGCTCCCTGCT |
| | | GACATGTGGGGATGTTGAAGAGAATCCTGGGCCTATGTATAGG |
| | | ATGCAACTGCTCCTCCTGATTGCTCTGAGCCTGGCTCTTGTGA |
| | | CCAACTCTGACATCCAGATGACACAGAGCCCTAGCAGCCTGTC |
| | | TGCTTCTGTGGGAGACAGAGTGACCATCACATGCAGAGCCAGC |
| | | CAGGGAATCAGAAACTACCTGGCCTGGTATCAGCAAAAGCCTG |
| | | GCAAGGCCCCTAAGCTGCTGATCTATGCAGCCAGCACACTGCA |
| | | GTCAGGGGTGCCAAGCAGATTTTCAGGCTCTGGCTCTGGCACA |
| | | GACTTCACCCTGACCATTTCTAGCCTGCAGCCTGAGGATGTGG |
| | | CCACCTACTACTGCCAGAGATACAACAGAGCCCCATACACCTT |
| | | TGGACAGGGCACAAAGGTGGAAATCAAGAGAACAGTGGCTGCC |
| | | CCATCTGTGTTCATCTTCCCACCATCTGATGAACAGCTGAAGT |
| | | CTGGCACTGCCTCTGTTGTGTGCCTGCTGAACAACTTCTACCC |
| | | TAGAGAAGCCAAGGTGCAGTGGAAGGTTGACAATGCCCTGCAG |
| | | TCTGGCAATAGCCAAGAATCTGTGACAGAGCAGGACTCCAAGG |
| | | ATTCCACCTACAGCCTGAGCAGCACCCTGACACTGAGCAAGGC |
| | | TGACTATGAGAAGCACAAAGTGTATGCCTGTGAAGTGACACAC |
| | | CAGGGACTGAGCAGCCCAGTGACCAAGAGCTTCAACAGGGGAG |
| | | AGTGCTGATAACtcgaggacggggtgaactacgcctgaggatc |
| | | cgatctttttccctctgccaaaaattatggggacatcatgaag |
| | | ccccttgagcatctgacttctggctaataaaggaaatttattt |
| | | tcattgcaatagtgtgttggaattttttgtgtctctcactcg |
| pAAV.sc.EF1ac. | SEQ ID | ctgcgcgctcgctcgctcactgaggccgcccgggcaaagcccg |
| Vh4i.Adalimumab. | NO: 222 | ggcgtcgggcgacctttggtcgcccggcctcagtgagcgagcg |
| Fab | | agcgcgcagagagggagtggaattcacgcgtGGGCAGAGCGCA |
| (5′-ITR to | | CATCGCCCACAGTCCCCGAGAAGTTGGGGGGAGGGGTCGGCAA |
| 3′-ITR) | | TTGAACGGGTGCCTAGAGAAGGTGGCGCGGGGTAAACTGGGAA |
| | | AGTGATGTCGTGTACTGGCTCCGCCTTTTTCCCGAGGGTGGGG |
| | | GAGAACCGTATATAAGTGCAGTAGTCGCCGTGAACGTTCTTTT |
| | | TCGCAACGGGTTTGCCGCCAGAACACAGCAGGTGAGTATCTCA |
| | | GGGATCCAGACATGGGGATATGGGAGGTGCCTCTGATCCCAGG |
| | | GCTCACTGTGGGTCTCTCTGTTCACAGGTTaccggtGCCACCA |
| | | TGTACAGAATGCAGCTGCTGCTGCTCATTGCCCTGTCTCTGGC |
| | | CCTGGTCACCAATTCTGAAGTGCAGCTGGTGGAAAGTGGTGGT |
| | | GGACTGGTGCAGCCTGGCAGAAGCCTGAGACTGTCTTGTGCTG |
| | | CCTCTGGCTTCACCTTTGATGACTATGCCATGCACTGGGTCAG |
| | | ACAGGCCCCTGGCAAAGGACTGGAATGGGTGTCAGCCATCACC |
| | | TGGAACTCTGGCCACATTGACTATGCTGACTCTGTGGAAGGCA |
| | | GATTCACCATCAGCAGAGACAATGCCAAGAACAGCCTGTACCT |
| | | GCAGATGAACTCCCTGAGAGCTGAGGACACAGCAGTGTACTAC |
| | | TGTGCCAAGGTGTCCTACCTGAGCACAGCCAGCAGCCTGGATT |
| | | ATTGGGGCCAGGGCACACTGGTTACAGTGTCCTCTGCCAGCAC |
| | | AAAGGGCCCCTCTGTTTTTCCACTGGCTCCCAGCAGCAAGAGC |
| | | ACCAGTGGTGGAACAGCTGCCCTGGGCTGTCTGGTCAAGGATT |
| | | ACTTCCCTGAGCCTGTGACAGTGTCTTGGAACTCAGGGGCTCT |
| | | GACCTCTGGGGTGCACACATTTCCAGCTGTGCTGCAGTCCTCT |
| | | GGCCTGTACTCTCTGTCCTCTGTGGTCACAGTGCCTAGCTCTA |
| | | GCCTGGGCACCCAGACCTACATCTGCAATGTGAACCACAAGCC |
| | | TAGCAACACCAAGGTGGACAAGAAGGTGGAACCCAAGAGCTGT |
| | | GACAAGACCCACAGAAAGAGAAGAGGCTCTGGAGAAGGCAGAG |
| | | GCTCCCTGCTGACATGTGGGGATGTTGAAGAGAATCCTGGGCC |
| | | TATGTATAGGATGCAACTGCTCCTCCTGATTGCTCTGAGCCTG |
| | | GCTCTTGTGACCAACTCTGACATCCAGATGACACAGAGCCCTA |
| | | GCAGCCTGTCTGCTTCTGTGGGAGACAGAGTGACCATCACATG |
| | | CAGAGCCAGCCAGGGCATCAGAAACTACCTGGCCTGGTATCAG |
| | | CAGAAGCCAGGCAAGGCCCCTAAGCTGCTGATCTATGCAGCCA |
| | | GCACACTGCAGAGTGGGGTGCCAAGCAGATTTTCAGGCTCTGG |
| | | CTCTGGCACAGACTTCACCCTGACCATTTCTAGCCTGCAGCCT |
| | | GAGGATGTGGCCACCTACTACTGCCAGAGATACAACAGAGCCC |
| | | CATACACCTTTGGACAGGGCACAAAGGTGGAAATCAAGAGAAC |
| | | Agttgcagcaccctcagttttcatcttccccccctcagatgaa |
| | | cagCTGAAGTCTGGCACTGCCTCTGTTGTGTGCCTGCTGAACA |
| | | ACTTCTACCCCAGAGAAGCCAAGGTGCAGTGGAAGGTTGACAA |
| | | TGCCCTGCAGTCAGGCAACAGCCAAGAATCTGTGACTGAACAG |
| | | GATTCCAAGGATAGCACCTACAGCCTGAGCAGCACCCTGACAC |
| | | TGAGCAAGGCTGACTATGAGAAGCACAAAGTGTATGCCTGTGA |
| | | AGTGACCCACCAGGGACTGAGCAGCCCAGTGACCAAGAGCTTC |
| | | AACAGGGGAGAGTGCTGATAAaagcttgatctttttccctctg |
| | | ccaaaaattatggggacatcatgaagccccttgagcatctgac |
| | | ttctggctaataaaggaaatttattttcattgcaatagtgtgt |
| | | tggaattttttgtgtctctcactcggctagcgaagcaattcta |
| | | gcaggcatgctggggagagatcgatctgaggaacccctagtga |
| | | tggagttggccactccctctctgcgcgctcgctcgctcactga |
| | | ggccgcccgggcaaagcccgggcgtcgggcgacctttggtcgc |
| | | ccggcctcagtgagcgagcgagcgcgcagagagggagtggcca |
| | | a |
| pAAV.sc.EF1ac. | SEQ ID | GGGCAGAGCGCACATCGCCCACAGTCCCCGAGAAGTTGGGGGG |
| Vh4i. | NO: 223 | AGGGGTCGGCAATTGAACGGGTGCCTAGAGAAGGTGGCGCGGG |
| Adalimumab. | | GTAAACTGGGAAAGTGATGTCGTGTACTGGCTCCGCCTTTTTC |
| Fab | | CCGAGGGTGGGGGAGAACCGTATATAAGTGCAGTAGTCGCCGT |
| (promoter to | | GAACGTTCTTTTTCGCAACGGGTTTGCCGCCAGAACACAGCAG |
| polyA) | | GTGAGTATCTCAGGGATCCAGACATGGGGATATGGGAGGTGCC |
| | | TCTGATCCCAGGGCTCACTGTGGGTCTCTCTGTTCACAGGTTa |
| | | ccggtGCCACCATGTACAGAATGCAGCTGCTGCTGCTCATTGC |
| | | CCTGTCTCTGGCCCTGGTCACCAATTCTGAAGTGCAGCTGGTG |
| | | GAAAGTGGTGGTGGACTGGTGCAGCCTGGCAGAAGCCTGAGAC |
| | | TGTCTTGTGCTGCCTCTGGCTTCACCTTTGATGACTATGCCAT |
| | | GCACTGGGTCAGACAGGCCCCTGGCAAAGGACTGGAATGGGTG |
| | | TCAGCCATCACCTGGAACTCTGGCCACATTGACTATGCTGACT |
| | | CTGTGGAAGGCAGATTCACCATCAGCAGAGACAATGCCAAGAA |
| | | CAGCCTGTACCTGCAGATGAACTCCCTGAGAGCTGAGGACACA |
| | | GCAGTGTACTACTGTGCCAAGGTGTCCTACCTGAGCACAGCCA |
| | | GCAGCCTGGATTATTGGGGCCAGGGCACACTGGTTACAGTGTC |
| | | CTCTGCCAGCACAAAGGGCCCCTCTGTTTTTCCACTGGCTCCC |
| | | AGCAGCAAGAGCACCAGTGGTGGAACAGCTGCCCTGGGCTGTC |
| | | TGGTCAAGGATTACTTCCCTGAGCCTGTGACAGTGTCTTGGAA |
| | | CTCAGGGGCTCTGACCTCTGGGGTGCACACATTTCCAGCTGTG |
| | | CTGCAGTCCTCTGGCCTGTACTCTCTGTCCTCTGTGGTCACAG |
| | | TGCCTAGCTCTAGCCTGGGCACCCAGACCTACATCTGCAATGT |
| | | GAACCACAAGCCTAGCAACACCAAGGTGGACAAGAAGGTGGAA |
| | | CCCAAGAGCTGTGACAAGACCCACAGAAAGAGAAGAGGCTCTG |
| | | GAGAAGGCAGAGGCTCCCTGCTGACATGTGGGGATGTTGAAGA |
| | | GAATCCTGGGCCTATGTATAGGATGCAACTGCTCCTCCTGATT |
| | | GCTCTGAGCCTGGCTCTTGTGACCAACTCTGACATCCAGATGA |
| | | CACAGAGCCCTAGCAGCCTGTCTGCTTCTGTGGGAGACAGAGT |
| | | GACCATCACATGCAGAGCCAGCCAGGGCATCAGAAACTACCTG |
| | | GCCTGGTATCAGCAGAAGCCAGGCAAGGCCCCTAAGCTGCTGA |
| | | TCTATGCAGCCAGCACACTGCAGAGTGGGGTGCCAAGCAGATT |
| | | TTCAGGCTCTGGCTCTGGCACAGACTTCACCCTGACCATTTCT |
| | | AGCCTGCAGCCTGAGGATGTGGCCACCTACTACTGCCAGAGAT |
| | | ACAACAGAGCCCCATACACCTTTGGACAGGGCACAAAGGTGGA |
| | | AATCAAGAGAACAgttgcagcaccctcagttttcatcttcccc |
| | | ccctcagatgaacagCTGAAGTCTGGCACTGCCTCTGTTGTGT |
| | | GCCTGCTGAACAACTTCTACCCCAGAGAAGCCAAGGTGCAGTG |
| | | GAAGGTTGACAATGCCCTGCAGTCAGGCAACAGCCAAGAATCT |
| | | GTGACTGAACAGGATTCCAAGGATAGCACCTACAGCCTGAGCA |
| | | GCACCCTGACACTGAGCAAGGCTGACTATGAGAAGCACAAAGT |
| | | GTATGCCTGTGAAGTGACCCACCAGGGACTGAGCAGCCCAGTG |
| | | ACCAAGAGCTTCAACAGGGGAGAGTGCTGATAAaagcttgatc |
| | | tttttccctctgccaaaaattatggggacatcatgaagcccct |
| | | tgagcatctgacttctggctaataaaggaaatttattttcatt |
| | | gcaatagtgtgttggaattttttgtgtctctcactcg |
| pAAV.sc.mU1a. | SEQ ID | ctgcgcgctcgctcgctcactgaggccgcccgggcaaagcccg |
| Vh4i.Adalimumab. | NO: 224 | ggcgtcgggcgacctttggtcgcccggcctcagtgagcgagcg |
| Fab | | agcgcgcagagagggagtggaattcaCGCGTATGGAGGCGGTA |
| (5′-ITR to | | CTATGTAGATGAGAATTCAGGAGCAAACTGGGAAAAGCAACTG |
| 3′-ITR) | | CTTCCAAATATTTGTGATTTTTACAGTGTAGTTTTGGAAAAAC |
| | | TCTTAGCCTACCAATTCTTCTAAGTGTTTTAAAATGTGGGAGC |
| | | CAGTACACATGAAGTTATAGAGTGTTTTAATGAGGCTTAAATA |
| | | TTTACCGTAACTATGAAATGCTACGCATATCATGCTGTTCAGG |
| | | CTCCGTGGCCACGCAACTCATACTCAGGTGAGTATCTCAGGGA |
| | | TCCAGACATGGGGATATGGGAGGTGCCTCTGATCCCAGGGCTC |
| | | ACTGTGGGTCTCTCTGTTCACAGGTTaccggtGCCACCATGTA |
| | | CAGAATGCAGCTGCTGCTGCTCATTGCCCTGTCTCTGGCCCTG |
| | | GTCACCAATTCTGAAGTGCAGCTGGTGGAAAGTGGTGGTGGAC |
| | | TGGTGCAGCCTGGCAGAAGCCTGAGACTGTCTTGTGCTGCCTC |
| | | TGGCTTCACCTTTGATGACTATGCCATGCACTGGGTCAGACAG |
| | | GCCCCTGGCAAAGGACTGGAATGGGTGTCAGCCATCACCTGGA |
| | | ACTCTGGCCACATTGACTATGCTGACTCTGTGGAAGGCAGATT |
| | | CACCATCAGCAGAGACAATGCCAAGAACAGCCTGTACCTGCAG |
| | | ATGAACTCCCTGAGAGCTGAGGACACAGCAGTGTACTACTGTG |
| | | CCAAGGTGTCCTACCTGAGCACAGCCAGCAGCCTGGATTATTG |
| | | GGGCCAGGGCACACTGGTTACAGTGTCCTCTGCCAGCACAAAG |
| | | GGCCCCTCTGTTTTTCCACTGGCTCCCAGCAGCAAGAGCACCA |
| | | GTGGTGGAACAGCTGCCCTGGGCTGTCTGGTCAAGGATTACTT |
| | | CCCTGAGCCTGTGACAGTGTCTTGGAACTCAGGGGCTCTGACC |
| | | TCTGGGGTGCACACATTTCCAGCTGTGCTGCAGTCCTCTGGCC |
| | | TGTACTCTCTGTCCTCTGTGGTCACAGTGCCTAGCTCTAGCCT |
| | | GGGCACCCAGACCTACATCTGCAATGTGAACCACAAGCCTAGC |
| | | AACACCAAGGTGGACAAGAAGGTGGAACCCAAGAGCTGTGACA |
| | | AGACCCACAGAAAGAGAAGAGGCTCTGGAGAAGGCAGAGGCTC |
| | | CCTGCTGACATGTGGGGATGTTGAAGAGAATCCTGGGCCTATG |
| | | TATAGGATGCAACTGCTCCTCCTGATTGCTCTGAGCCTGGCTC |
| | | TTGTGACCAACTCTGACATCCAGATGACACAGAGCCCTAGCAG |
| | | CCTGTCTGCTTCTGTGGGAGACAGAGTGACCATCACATGCAGA |
| | | GCCAGCCAGGGCATCAGAAACTACCTGGCCTGGTATCAGCAGA |
| | | AGCCAGGCAAGGCCCCTAAGCTGCTGATCTATGCAGCCAGCAC |
| | | ACTGCAGAGTGGGGTGCCAAGCAGATTTTCAGGCTCTGGCTCT |
| | | GGCACAGACTTCACCCTGACCATTTCTAGCCTGCAGCCTGAGG |
| | | ATGTGGCCACCTACTACTGCCAGAGATACAACAGAGCCCCATA |
| | | CACCTTTGGACAGGGCACAAAGGTGGAAATCAAGAGAACAgtt |
| | | gcagcaccctcagttttcatcttccccccctcagatgaacagC |
| | | TGAAGTCTGGCACTGCCTCTGTTGTGTGCCTGCTGAACAACTT |
| | | CTACCCCAGAGAAGCCAAGGTGCAGTGGAAGGTTGACAATGCC |
| | | CTGCAGTCAGGCAACAGCCAAGAATCTGTGACTGAACAGGATT |
| | | CCAAGGATAGCACCTACAGCCTGAGCAGCACCCTGACACTGAG |
| | | CAAGGCTGACTATGAGAAGCACAAAGTGTATGCCTGTGAAGTG |
| | | ACCCACCAGGGACTGAGCAGCCCAGTGACCAAGAGCTTCAACA |
| | | GGGGAGAGTGCTGATAAaagcttgatctttttccctctgccaa |
| | | aaattatggggacatcatgaagccccttgagcatctgacttct |
| | | ggctaataaaggaaatttattttcattgcaatagtgtgttgga |
| | | attttttgtgtctctcactcggctagcgaagcaattctagcag |
| | | gcatgctggggagagatcgatctgaggaacccctagtgatgga |
| | | gttggccactccctctctgcgcgctcgctcgctcactgaggcc |
| | | gcccgggcaaagcccgggcgtcgggcgacctttggtcgcccgg |
| | | cctcagtgagcgagcgagcgcgcagagagggagtggccaa |
| pAAV.sc.mU1a. | SEQ ID | ATGGAGGCGGTACTATGTAGATGAGAATTCAGGAGCAAACTGG |
| Vh4i.Adalimumab. | NO: 225 | GAAAAGCAACTGCTTCCAAATATTTGTGATTTTTACAGTGTAG |
| Fab | | TTTTGGAAAAACTCTTAGCCTACCAATTCTTCTAAGTGTTTTA |
| (promoter to | | AAATGTGGGAGCCAGTACACATGAAGTTATAGAGTGTTTTAAT |
| polyA) | | GAGGCTTAAATATTTACCGTAACTATGAAATGCTACGCATATC |
| | | ATGCTGTTCAGGCTCCGTGGCCACGCAACTCATACTCAGGTGA |
| | | GTATCTCAGGGATCCAGACATGGGGATATGGGAGGTGCCTCTG |
| | | ATCCCAGGGCTCACTGTGGGTCTCTCTGTTCACAGGTTaccgg |
| | | tGCCACCATGTACAGAATGCAGCTGCTGCTGCTCATTGCCCTG |
| | | TCTCTGGCCCTGGTCACCAATTCTGAAGTGCAGCTGGTGGAAA |
| | | GTGGTGGTGGACTGGTGCAGCCTGGCAGAAGCCTGAGACTGTC |
| | | TTGTGCTGCCTCTGGCTTCACCTTTGATGACTATGCCATGCAC |
| | | TGGGTCAGACAGGCCCCTGGCAAAGGACTGGAATGGGTGTCAG |
| | | CCATCACCTGGAACTCTGGCCACATTGACTATGCTGACTCTGT |
| | | GGAAGGCAGATTCACCATCAGCAGAGACAATGCCAAGAACAGC |
| | | CTGTACCTGCAGATGAACTCCCTGAGAGCTGAGGACACAGCAG |
| | | TGTACTACTGTGCCAAGGTGTCCTACCTGAGCACAGCCAGCAG |
| | | CCTGGATTATTGGGGCCAGGGCACACTGGTTACAGTGTCCTCT |
| | | GCCAGCACAAAGGGCCCCTCTGTTTTTCCACTGGCTCCCAGCA |
| | | GCAAGAGCACCAGTGGTGGAACAGCTGCCCTGGGCTGTCTGGT |
| | | CAAGGATTACTTCCCTGAGCCTGTGACAGTGTCTTGGAACTCA |
| | | GGGGCTCTGACCTCTGGGGTGCACACATTTCCAGCTGTGCTGC |
| | | AGTCCTCTGGCCTGTACTCTCTGTCCTCTGTGGTCACAGTGCC |
| | | TAGCTCTAGCCTGGGCACCCAGACCTACATCTGCAATGTGAAC |
| | | CACAAGCCTAGCAACACCAAGGTGGACAAGAAGGTGGAACCCA |
| | | AGAGCTGTGACAAGACCCACAGAAAGAGAAGAGGCTCTGGAGA |
| | | AGGCAGAGGCTCCCTGCTGACATGTGGGGATGTTGAAGAGAAT |
| | | CCTGGGCCTATGTATAGGATGCAACTGCTCCTCCTGATTGCTC |
| | | TGAGCCTGGCTCTTGTGACCAACTCTGACATCCAGATGACACA |
| | | GAGCCCTAGCAGCCTGTCTGCTTCTGTGGGAGACAGAGTGACC |
| | | ATCACATGCAGAGCCAGCCAGGGCATCAGAAACTACCTGGCCT |
| | | GGTATCAGCAGAAGCCAGGCAAGGCCCCTAAGCTGCTGATCTA |
| | | TGCAGCCAGCACACTGCAGAGTGGGGTGCCAAGCAGATTTTCA |
| | | GGCTCTGGCTCTGGCACAGACTTCACCCTGACCATTICTAGCC |
| | | TGCAGCCTGAGGATGTGGCCACCTACTACTGCCAGAGATACAA |
| | | CAGAGCCCCATACACCTTTGGACAGGGCACAAAGGTGGAAATC |
| | | AAGAGAACAgttgcagcaccctcagttttcatcttccccccct |
| | | cagatgaacagCTGAAGTCTGGCACTGCCTCTGTTGTGTGCCT |
| | | GCTGAACAACTTCTACCCCAGAGAAGCCAAGGTGCAGTGGAAG |
| | | GTTGACAATGCCCTGCAGTCAGGCAACAGCCAAGAATCTGTGA |
| | | CTGAACAGGATTCCAAGGATAGCACCTACAGCCTGAGCAGCAC |
| | | CCTGACACTGAGCAAGGCTGACTATGAGAAGCACAAAGTGTAT |
| | | GCCTGTGAAGTGACCCACCAGGGACTGAGCAGCCCAGTGACCA |
| | | AGAGCTTCAACAGGGGAGAGTGCTGATAAaagcttgatctttt |
| | | tccctctgccaaaaattatggggacatcatgaagccccttgag |
| | | catctgacttctggctaataaaggaaatttattttcattgcaa |
| | | tagtgtgttggaattttttgtgtctctcactcg |
| pAAV.CB.VH4. | SEQ ID | gacgacattgattattgactagttattaatagtaatcaattac |
| Adalimumab | NO: 276 | ggggtcattagttcatagcccatatatggagttccgcgttaca |
| (promoter to | | taacttacggtaaatggcccgcctggctgaccgcccaacgacc |
| polyA) | | cccgcccattgacgtcaataatgacgtatgttcccatagtaac |
| | | gccaatagggactttccattgacgtcaatgggtggagtattta |
| | | cggtaaactgcccacttggcagtacatcaagtgtatcatatgc |
| | | caagtacgccccctattgacgtcaatgacggtaaatggcccgc |
| | | ctggcattatgcccagtacatgaccttatgggactttcctact |
| | | tggcagtacatctacgtattagtcatcgctattaccatggtcg |
| | | aggtgagccccacgttctgcttcactctccccatctccccccc |
| | | ctccccacccccaattttgtatttatttattttttaattattt |
| | | tgtgcagcgatgggggcggggggggggggggggcgcgcgccag |
| | | gcggggcggggcggggcgaggggcggggcggggcgaggcggag |
| | | aggtgcggcggcagccaatcagagcggcgcgctccgaaagttt |
| | | ccttttatggcgaggcggcggcggcggcggccctataaaaagc |
| | | gaagcgcgcggcgggcggacgcgtCAGGTGAGTATCTCAGGGA |
| | | TCCAGACATGGGGATATGGGAGGTGCCTCTGATCCCAGGGCTC |
| | | ACTGTGGGTCTCTCTGTTCACAGGTTgAccgagtgAATTCGCC |
| | | ACCATGTACAGAATGCAGCTGCTGCTGCTCATTGCCCTGTCTC |
| | | TGGCCCTGGTCACCAATTCTGAAGTGCAGCTGGTGGAAAGTGG |
| | | TGGTGGACTGGTGCAGCCTGGCAGAAGCCTGAGACTGTCTTGT |
| | | GCTGCCTCTGGCTTCACCTTTGATGACTATGCCATGCACTGGG |
| | | TCAGACAGGCCCCTGGCAAAGGACTGGAATGGGTGTCAGCCAT |
| | | CACCTGGAACTCTGGCCACATTGACTATGCTGACTCTGTGGAA |
| | | GGCAGATTCACCATCAGCAGAGACAATGCCAAGAACAGCCTGT |
| | | ACCTGCAGATGAACTCCCTGAGAGCTGAGGACACAGCAGTGTA |
| | | CTACTGTGCCAAGGTGTCCTACCTGAGCACAGCCAGCAGCCTG |
| | | GATTATTGGGGCCAGGGCACACTGGTTACAGTGTCCTCTGCCA |
| | | GCACAAAGGGCCCCTCTGTTTTTCCACTGGCTCCCAGCAGCAA |
| | | GAGCACCAGTGGTGGAACAGCTGCCCTGGGCTGTCTGGTCAAG |
| | | GATTACTTCCCTGAGCCTGTGACAGTGTCTTGGAACTCAGGGG |
| | | CTCTGACCTCTGGGGTGCACACATTTCCAGCTGTGCTGCAGTC |
| | | CTCTGGCCTGTACTCTCTGTCCTCTGTGGTCACAGTGCCTAGC |
| | | TCTAGCCTGGGCACCCAGACCTACATCTGCAATGTGAACCACA |
| | | AGCCTAGCAACACCAAGGTGGACAAGAAGGTGGAACCCAAGAG |
| | | CTGTGACAAGACCCACACCTGTCCTCCATGTCCTGCTCCAGAA |
| | | CTGCTTGGAGGCCCTTCTGTGTTCCTGTTTCCTCCAAAGCCTA |
| | | AGGACACCCTGATGATCAGCAGAACCCCTGAAGTGACCTGTGT |
| | | GGTGGTTGATGTGTCCCATGAGGACCCAGAAGTGAAGTTCAAT |
| | | TGGTATGTGGATGGGGTTGAAGTGCACAATGCTAAGACCAAGC |
| | | CTAGAGAGGAACAGTACAACAGCACCTACAGAGTGGTGTCTGT |
| | | GCTGACAGTGCTGCATCAGGACTGGCTGAATGGCAAAGAGTAC |
| | | AAGTGCAAAGTGTCCAACAAGGCCCTGCCTGCTCCTATTGAGA |
| | | AAACCATCTCCAAGGCCAAGGGCCAGCCAAGAGAACCCCAGGT |
| | | TTACACACTGCCACCTAGCAGAGATGAGCTGACCAAGAACCAG |
| | | GTGTCCCTGACCTGCCTGGTTAAGGGCTTCTACCCCTCTGACA |
| | | TTGCTGTGGAATGGGAGAGCAATGGCCAGCCTGAGAACAACTA |
| | | CAAGACAACCCCTCCTGTGCTGGACTCTGATGGCTCATTCTTC |
| | | CTGTACAGCAAGCTGACTGTGGACAAGTCCAGATGGCAGCAGG |
| | | GGAATGTGTTCAGCTGCTCTGTGATGCATGAGGCCCTGCACAA |
| | | CCACTACACCCAGAAAAGTCTGAGTCTGAGCCCTGGCAAGAGA |
| | | AAGAGAAGAGGCTCTGGAGAAGGCAGAGGCTCCCTGCTGACAT |
| | | GTGGGGATGTTGAAGAGAATCCTGGGCCTATGTATAGGATGCA |
| | | ACTGCTCCTCCTGATTGCTCTGAGCCTGGCTCTTGTGACCAAC |
| | | TCTGACATCCAGATGACACAGAGCCCTAGCAGCCTGTCTGCTT |
| | | CTGTGGGAGACAGAGTGACCATCACATGCAGAGCCAGCCAGGG |
| | | AATCAGAAACTACCTGGCCTGGTATCAGCAAAAGCCTGGCAAG |
| | | GCCCCTAAGCTGCTGATCTATGCAGCCAGCACACTGCAGTCAG |
| | | GGGTGCCAAGCAGATTTTCAGGCTCTGGCTCTGGCACAGACTT |
| | | CACCCTGACCATTTCTAGCCTGCAGCCTGAGGATGTGGCCACC |
| | | TACTACTGCCAGAGATACAACAGAGCCCCATACACCTTTGGAC |
| | | AGGGCACAAAGGTGGAAATCAAGAGAACAGTGGCTGCCCCATC |
| | | TGTGTTCATCTTCCCACCATCTGATGAACAGCTGAAGTCTGGC |
| | | ACTGCCTCTGTTGTGTGCCTGCTGAACAACTTCTACCCTAGAG |
| | | AAGCCAAGGTGCAGTGGAAGGTTGACAATGCCCTGCAGTCTGG |
| | | CAATAGCCAAGAATCTGTGACAGAGCAGGACTCCAAGGATTCC |
| | | ACCTACAGCCTGAGCAGCACCCTGACACTGAGCAAGGCTGACT |
| | | ATGAGAAGCACAAAGTGTATGCCTGTGAAGTGACACACCAGGG |
| | | ACTGAGCAGCCCAGTGACCAAGAGCTTCAACAGGGGAGAGTGC |
| | | TGATAACtcgaggacggggtgaactacgcctgaggatccgatc |
| | | tttttccctctgccaaaaattatggggacatcatgaagcccct |
| | | tgagcatctgacttctggctaataaaggaaatttattttcatt |
| | | gcaatagtgtgttggaattttttgtgtctctcactcg |
| pAAV.CB.VH4. | SEQ ID | ctgcgcgctcgctcgctcactgaggccgcccgggcaaagcccg |
| Adalimumab | NO: 277 | ggcgtcgggcgacctttggtcgcccggcctcagtgagcgagcg |
| (ITR to ITR) | | agcgcgcagagagggagtggccaactccatcactaggggttcc |
| | | ttgtagttaatgattaacccgccatgctacttatctaccaggg |
| | | taatggggatcctctagaactatagctagtcgacgacattgat |
| | | tattgactagttattaatagtaatcaattacggggtcattagt |
| | | tcatagcccatatatggagttccgcgttacataacttacggta |
| | | aatggcccgcctggctgaccgcccaacgacccccgcccattga |
| | | cgtcaataatgacgtatgttcccatagtaacgccaatagggac |
| | | tttccattgacgtcaatgggtggagtatttacggtaaactgcc |
| | | cacttggcagtacatcaagtgtatcatatgccaagtacgcccc |
| | | ctattgacgtcaatgacggtaaatggcccgcctggcattatgc |
| | | ccagtacatgaccttatgggactttcctacttggcagtacatc |
| | | tacgtattagtcatcgctattaccatggtcgaggtgagcccca |
| | | cgttctgcttcactctccccatctcccccccctccccaccccc |
| | | aattttgtatttatttattttttaattattttgtgcagcgatg |
| | | ggggcggggggggggggggggcgcgcgccaggcggggcggggc |
| | | ggggcgaggggcggggcggggcgaggcggagaggtgcggcggc |
| | | agccaatcagagcggcgcgctccgaaagtttccttttatggcg |
| | | aggcggcggcggcggcggccctataaaaagcgaagcgcgcggc |
| | | gggcggacgcgtCAGGTGAGTATCTCAGGGATCCAGACATGGG |
| | | GATATGGGAGGTGCCTCTGATCCCAGGGCTCACTGTGGGTCTC |
| | | TCTGTTCACAGGTTgAccgagtgAATTCGCCACCATGTACAGA |
| | | ATGCAGCTGCTGCTGCTCATTGCCCTGTCTCTGGCCCTGGTCA |
| | | CCAATTCTGAAGTGCAGCTGGTGGAAAGTGGTGGTGGACTGGT |
| | | GCAGCCTGGCAGAAGCCTGAGACTGTCTTGTGCTGCCTCTGGC |
| | | TTCACCTTTGATGACTATGCCATGCACTGGGTCAGACAGGCCC |
| | | CTGGCAAAGGACTGGAATGGGTGTCAGCCATCACCTGGAACTC |
| | | TGGCCACATTGACTATGCTGACTCTGTGGAAGGCAGATTCACC |
| | | ATCAGCAGAGACAATGCCAAGAACAGCCTGTACCTGCAGATGA |
| | | ACTCCCTGAGAGCTGAGGACACAGCAGTGTACTACTGTGCCAA |
| | | GGTGTCCTACCTGAGCACAGCCAGCAGCCTGGATTATTGGGGC |
| | | CAGGGCACACTGGTTACAGTGTCCTCTGCCAGCACAAAGGGCC |
| | | CCTCTGTTTTTCCACTGGCTCCCAGCAGCAAGAGCACCAGTGG |
| | | TGGAACAGCTGCCCTGGGCTGTCTGGTCAAGGATTACTTCCCT |
| | | GAGCCTGTGACAGTGTCTTGGAACTCAGGGGCTCTGACCTCTG |
| | | GGGTGCACACATTTCCAGCTGTGCTGCAGTCCTCTGGCCTGTA |
| | | CTCTCTGTCCTCTGTGGTCACAGTGCCTAGCTCTAGCCTGGGC |
| | | ACCCAGACCTACATCTGCAATGTGAACCACAAGCCTAGCAACA |
| | | CCAAGGTGGACAAGAAGGTGGAACCCAAGAGCTGTGACAAGAC |
| | | CCACACCTGTCCTCCATGTCCTGCTCCAGAACTGCTTGGAGGC |
| | | CCTTCTGTGTTCCTGTTTCCTCCAAAGCCTAAGGACACCCTGA |
| | | TGATCAGCAGAACCCCTGAAGTGACCTGTGTGGTGGTTGATGT |
| | | GTCCCATGAGGACCCAGAAGTGAAGTTCAATTGGTATGTGGAT |
| | | GGGGTTGAAGTGCACAATGCTAAGACCAAGCCTAGAGAGGAAC |
| | | AGTACAACAGCACCTACAGAGTGGTGTCTGTGCTGACAGTGCT |
| | | GCATCAGGACTGGCTGAATGGCAAAGAGTACAAGTGCAAAGTG |
| | | TCCAACAAGGCCCTGCCTGCTCCTATTGAGAAAACCATCTCCA |
| | | AGGCCAAGGGCCAGCCAAGAGAACCCCAGGTTTACACACTGCC |
| | | ACCTAGCAGAGATGAGCTGACCAAGAACCAGGTGTCCCTGACC |
| | | TGCCTGGTTAAGGGCTTCTACCCCTCTGACATTGCTGTGGAAT |
| | | GGGAGAGCAATGGCCAGCCTGAGAACAACTACAAGACAACCCC |
| | | TCCTGTGCTGGACTCTGATGGCTCATTCTTCCTGTACAGCAAG |
| | | CTGACTGTGGACAAGTCCAGATGGCAGCAGGGGAATGTGTTCA |
| | | GCTGCTCTGTGATGCATGAGGCCCTGCACAACCACTACACCCA |
| | | GAAAAGTCTGAGTCTGAGCCCTGGCAAGAGAAAGAGAAGAGGC |
| | | TCTGGAGAAGGCAGAGGCTCCCTGCTGACATGTGGGGATGTTG |
| | | AAGAGAATCCTGGGCCTATGTATAGGATGCAACTGCTCCTCCT |
| | | GATTGCTCTGAGCCTGGCTCTTGTGACCAACTCTGACATCCAG |
| | | ATGACACAGAGCCCTAGCAGCCTGTCTGCTTCTGTGGGAGACA |
| | | GAGTGACCATCACATGCAGAGCCAGCCAGGGAATCAGAAACTA |
| | | CCTGGCCTGGTATCAGCAAAAGCCTGGCAAGGCCCCTAAGCTG |
| | | CTGATCTATGCAGCCAGCACACTGCAGTCAGGGGTGCCAAGCA |
| | | GATTTTCAGGCTCTGGCTCTGGCACAGACTTCACCCTGACCAT |
| | | TTCTAGCCTGCAGCCTGAGGATGTGGCCACCTACTACTGCCAG |
| | | AGATACAACAGAGCCCCATACACCTTTGGACAGGGCACAAAGG |
| | | TGGAAATCAAGAGAACAGTGGCTGCCCCATCTGTGTTCATCTT |
| | | CCCACCATCTGATGAACAGCTGAAGTCTGGCACTGCCTCTGTT |
| | | GTGTGCCTGCTGAACAACTTCTACCCTAGAGAAGCCAAGGTGC |
| | | AGTGGAAGGTTGACAATGCCCTGCAGTCTGGCAATAGCCAAGA |
| | | ATCTGTGACAGAGCAGGACTCCAAGGATTCCACCTACAGCCTG |
| | | AGCAGCACCCTGACACTGAGCAAGGCTGACTATGAGAAGCACA |
| | | AAGTGTATGCCTGTGAAGTGACACACCAGGGACTGAGCAGCCC |
| | | AGTGACCAAGAGCTTCAACAGGGGAGAGTGCTGATAACtcgag |
| | | gacggggtgaactacgcctgaggatccgatctttttccctctg |
| | | ccaaaaattatggggacatcatgaagccccttgagcatctgac |
| | | ttctggctaataaaggaaatttattttcattgcaatagtgtgt |
| | | tggaattttttgtgtctctcactcggaagcaattcgttgatct |
| | | gaatttcgaccacccataatacccattaccctggtagataagt |
| | | agcatggcgggttaatcattaactacaaggaacccctagtgat |
| | | ggagttggccactccctctctgcgcgctcgctcgctcactgag |
| | | gccgggcgaccaaaggtcgcccgacgcccgggctttgcccggg |
| | | cggcctcagtgagcgagcgagcgcgcag |
| Adalimumab | SEQ ID | gaagtgcagctggtggaaagtggtggtggactggtgcagcctg |
| Heavy Chain | NO: 54 | gcagaagcctgagactgtcttgtgctgcctctggcttcacctt |
| (HC) IgG1 | | tgatgactatgccatgcactgggtcagacaggcccctggcaaa |
| | | ggactggaatgggtgtcagccatcacctggaactctggccaca |
| | | ttgactatgctgactctgtggaaggcagattcaccatcagcag |
| | | agacaatgccaagaacagcctgtacctgcagatgaactccctg |
| | | agagctgaggacacagcagtgtactactgtgccaaggtgtcct |
| | | acctgagcacagccagcagcctggattattggggccagggcac |
| | | actggttacagtgtcctctgccagcacaaagggcccctctgtt |
| | | tttccactggctcccagcagcaagagcaccagtggtggaacag |
| | | ctgccctgggctgtctggtcaaggattacttccctgagcctgt |
| | | gacagtgtcttggaactcaggggctctgacctctggggtgcac |
| | | acatttccagctgtgctgcagtcctctggcctgtactctctgt |
| | | cctctgtggtcacagtgcctagctctagcctgggcacccagac |
| | | ctacatctgcaatgtgaaccacaagcctagcaacaccaaggtg |
| | | gacaagaaggtggaacccaagagctgtgacaagacccacacct |
| | | gtcctccatgtcctgctccagaactgcttggaggcccttctgt |
| | | gttcctgtttcctccaaagcctaaggacaccctgatgatcagc |
| | | agaacccctgaagtgacctgtgtggtggttgatgtgtcccatg |
| | | aggacccagaagtgaagttcaattggtatgtggatggggttga |
| | | agtgcacaatgctaagaccaagcctagagaggaacagtacaac |
| | | agcacctacagagtggtgtctgtgctgacagtgctgcatcagg |
| | | actggctgaatggcaaagagtacaagtgcaaagtgtccaacaa |
| | | ggccctgcctgctcctattgagaaaaccatctccaaggccaag |
| | | ggccagccaagagaaccccaggtttacacactgccacctagca |
| | | gagatgagctgaccaagaaccaggtgtccctgacctgcctggt |
| | | taagggcttctacccctctgacattgctgtggaatgggagagc |
| | | aatggccagcctgagaacaactacaagacaacccctcctgtgc |
| | | tggactctgatggctcattcttcctgtacagcaagctgactgt |
| | | ggacaagtccagatggcagcaggggaatgtgttcagctgctct |
| | | gtgatgcatgaggccctgcacaaccactacacccagaaaagtc |
| | | tgagtctgagccctggcaag |
| | | IgG1 CH1 and CH2 are shown bold; hinge |
| | | region is shown in italic. |
| Adalimumab | SEQ ID | Gaagtgcagctggtggaaagtggtggtggactggtgcagcctg |
| VH | NO: 55 | gcagaagcctgagactgtcttgtgctgcctctggcttcacctt |
| | | tgatgactatgccatgcactgggtcagacaggcccctggcaaa |
| | | ggactggaatgggtgtcagccatcacctggaactctggccaca |
| | | ttgactatgctgactctgtggaaggcagattcaccatcagcag |
| | | agacaatgccaagaacagcctgtacctgcagatgaactccctg |
| | | agagctgaggacacagcagtgtactactgtgccaaggtgtcct |
| | | acctgagcacagccagcagcctggattattggggccagggcac |
| | | actggttacagtgtcctct |
| | | CDRs are underlined. |
| IgG1 CH1 | SEQ ID | gccagcacaaagggcccctctgtttttccactggctcccagca |
| | NO: 56 | gcaagagcaccagtggtggaacagctgccctgggctgtctggt |
| | | caaggattacttccctgagcctgtgacagtgtcttggaactca |
| | | ggggctctgacctctggggtgcacacatttccagctgtgctgc |
| | | agtcctctggcctgtactctctgtcctctgtggtcacagtgcc |
| | | tagctctagcctgggcacccagacctacatctgcaatgtgaac |
| | | cacaagcctagcaacaccaaggtggacaagaaggtg |
| IgG1 CH2 | SEQ ID | gctccagaactgcttggaggcccttctgtgttcctgtttcctc |
| | NO: 57 | caaagcctaaggacaccctgatgatcagcagaacccctgaagt |
| | | gacctgtgtggtggttgatgtgtcccatgaggacccagaagtg |
| | | aagttcaattggtatgtggatggggttgaagtgcacaatgcta |
| | | agaccaagcctagagaggaacagtacaacagcacctacagagt |
| | | ggtgtctgtgctgacagtgctgcatcaggactggctgaatggc |
| | | aaagagtacaagtgcaaagtgtccaacaaggccctgcctgctc |
| | | ctattgagaaaaccatctccaaggccaagggccagccaagaga |
| | | accccaggtttacacactgccacctagcagagatgagctgacc |
| | | aagaaccaggtgtccctgacctgcctggttaagggcttctacc |
| | | cctctgacattgctgtggaatgggagagcaatggccagcctga |
| | | gaacaactacaagacaacccctcctgtgctggactctgatggc |
| | | tcattcttcctgtacagcaagctgactgtggacaagtccagat |
| | | ggcagcaggggaatgtgttcagctgctctgtgatgcatgaggc |
| | | cctgcacaaccactacacccagaaaagtctgagtctgagccct |
| | | ggcaag |
| Adalimumab | SEQ ID | gctccagaactgcttggaggcccttctgtgttcctgtttcctc |
| Light Chain | NO: 58 | caaagcctaaggacaccctgatgatcagcagaacccctgaagt |
| (kappa) | | gacctgtgtggtggttgatgtgtcccatgaggacccagaagtg |
| | | aagttcaattggtatgtggatggggttgaagtgcacaatgcta |
| | | agaccaagcctagagaggaacagtacaacagcacctacagagt |
| | | ggtgtctgtgctgacagtgctgcatcaggactggctgaatggc |
| | | aaagagtacaagtgcaaagtgtccaacaaggccctgcctgctc |
| | | ctattgagaaaaccatctccaaggccaagggccagccaagaga |
| | | accccaggtttacacactgccacctagcagagatgagctgacc |
| | | aagaaccaggtgtccctgacctgcctggttaagggcttctacc |
| | | cctctgacattgctgtggaatgggagagcaatggccagcctga |
| | | gaacaactacaagacaacccctcctgtgctggactctgatggc |
| | | tcattcttcctgtacagcaagctgactgtggacaagtccagat |
| | | ggcagcaggggaatgtgttcagctgctctgtgatgcatgaggc |
| | | cctgcacaaccactacacccagaaaagtctgagtctgagccct |
| | | ggcaag |
| Adalimumab VL | SEQ ID | Gacatccagatgacacagagccctagcagcctgtctgcttctg |
| | NO: 59 | tgggagacagagtgaccatcacatgcagagccagccagggaat |
| | | cagaaactacctggcctggtatcagcaaaagcctggcaaggcc |
| | | cctaagctgctgatctatgcagccagcacactgcagtcagggg |
| | | tgccaagcagattttcaggctctggctctggcacagacttcac |
| | | cctgaccatttctagcctgcagcctgaggatgtggccacctac |
| | | tactgccagagatacaacagagccccatacacctttggacagg |
| | | gcacaaaggtggaaatcaag |
| | | CDRs are underlined. |
| Adalimumab | SEQ ID | agaacagttgcagcaccctcagttttcatcttccccccctcag |
| Light chain C- | NO: 60 | atgaacagctgaagtctggcactgcctctgttgtgtgcctgct |
| Domain (CL) | | gaacaacttctaccccagagaagccaaggtgcagtggaaggtt |
| | | gacaatgccctgcagtcaggcaacagccaagaatctgtgactg |
| | | aacaggattccaaggatagcacctacagcctgagcagcaccct |
| | | gacactgagcaaggctgactatgagaagcacaaagtgtatgcc |
| | | tgtgaagtgacccaccagggactgagcagcccagtgaccaaga |
| | | gcttcaacaggggagagtgctga |

Gene Therapy Methods

Provided are methods of treating human subjects for non-infectious uveitis by administration of a viral vector containing a transgene encoding an anti-TNFα antibody, or antigen binding fragment thereof. The antibody may be adalimumab, infliximab, or golimumab and is, e.g., a full length or substantially full length antibody or Fab fragment thereof, or other antigen-binding fragment thereof.

In embodiments, the patient has been diagnosed with and/or has symptoms associated with non-infectious uveitis. Recombinant vectors used for delivering the transgene are described in Section 5.1 and exemplary transgenes are provided above. Such vectors should have a tropism for human ocular tissue cells and can include non-replicating rAAV, particularly those bearing an AAV8, AAV3B or AAVrh73 capsid. The recombinant vectors, such as shown in FIGS. 2A-2C, can be administered in any manner such that the recombinant vector enters one or more ocular tissue cells. In particular embodiments, the transgene is CAG.Adalimumab.T2A.IgG (SEQ ID NO: 48); CAG.Adalimumab.Fab (SEQ ID NO: 51); GRK1.Vh4i.Adalimumab.IgG (SEQ ID NO: 53), mU1a.Vh4i.Adalimumab.Fab (SEQ ID NO:224), EF1a.Vh4i.Adalimumab.Fab (SEQ ID NO:222), CB.VH4.adalimumab (SEQ ID NO: 276), CBlong.VH4.adalimumab or Best1.GRK1.VH4i.adalimumab in an AAV8 vector.

Subjects to whom such gene therapy is administered can be those responsive to anti-TNFα therapy. In certain embodiments, the methods encompass treating patients who have been diagnosed with non-infectious uveitis, or have one or more symptoms associated therewith, and identified as responsive to treatment with an anti-TNFα antibody, anti-TNFα Fc fusion protein, or considered a good candidate for therapy with an anti-TNFα antibody or anti-TNFα Fc fusion protein. In specific embodiments, the patients have previously been treated with etanercept, adalimumab, infliximab, or golimumab, and have been found to be responsive to etanercept, adalimumab, infliximab, or golimumab. In other embodiments, the patients have been previously treated with an anti-TNF-alpha antibody or fusion protein such as etanercept, certolizumab, or other anti-TNF-alpha agent. To determine responsiveness, the anti-TNFα transgene product (e.g., produced in cell culture, bioreactors, etc.) may be administered directly to the subject.

Human Post Translationally Modified Antibodies

The production of the anti-TNFα HuPTM mAb or HuPTM Fab, should result in a “biobetter” molecule for the treatment of angioedema accomplished via gene therapy—e.g., by administering a viral vector or other DNA expression construct encoding the anti-TNFα HuPTM Fab, subretinally, intravitreally, intracamerally, suprachoroidally, or intravenously to human subjects (patients) diagnosed with or having one or more symptoms of non-infectious uveitis, to create a permanent depot in the eye (and/or liver and/or muscle) that continuously supplies the fully-human post-translationally modified, e.g., human-glycosylated, sulfated transgene product produced by transduced ocular tissue cells.

In specific embodiments, the anti-TNFα HuPTM mAb or antigen-binding fragment thereof has heavy and light chains with the amino acid sequences of the heavy and light chain Fab portions of adalimumab as set forth in FIG. 2A (with glutamine (Q) glycosylation sites; asparaginal (N) glycosylation sites, non-consensus asparaginal (N) glycosylation sites; and tyrosine-O-sulfation sites (Y) are as indicated in the legend) has a glycosylation, particularly a 2,6-sialylation, at one or more of the amino acid positions N54, Q113, and/or N163 of the heavy chain (SEQ ID NO: 1) or Q100, N158, and/or N210 of the light chain (SEQ ID NO: 2). Alternatively or in addition to, the HuPTM mAb or antigen binding-fragment thereof with the heavy and light chain variable domain sequences of adalimumab has a sulfation group at Y32, Y94 and/or Y95 of the heavy chain (SEQ ID NO: 1) and/or Y86 and/or Y87 of the light chain (SEQ ID NO: 2). In other embodiments, the anti-TNFα HuPTM mAb or antigen-binding fragment thereof does not contain any detectable NeuGc moieties and/or does not contain any detectable alpha-Gal moieties. In certain embodiments, the HuPTM mAb is a full length or substantially full length mAb with an Fc region.

In specific embodiments, the anti-TNFα HuPTM mAb or antigen-binding fragment thereof has heavy and light chains with the amino acid sequences of the heavy and light chain Fab portions of infliximab as set forth in FIG. 2B (with glutamine (Q) glycosylation sites; asparaginal (N) glycosylation sites, non-consensus asparaginal (N) glycosylation sites; and tyrosine-O-sulfation sites (Y) are as indicated in the legend) has a glycosylation, particularly a 2,6-sialylation, at one or more of the amino acid positions N57, N101, Q112 and/or N162 of the heavy chain (SEQ ID NO: 3) or N41, N76, N158 and/or N210 of the light chain (SEQ ID NO: 4). Alternatively or in addition to, the HuPTM mAb or antigen binding-fragment thereof with the heavy and light chain variable domain sequences of infliximab has a sulfation group at Y96 and/or Y97 of the heavy chain (SEQ ID NO: 3) and/or Y86 and/or Y87 of the light chain (SEQ ID NO: 4). In other embodiments, the anti-TNFα HuPTM mAb or antigen-binding fragment thereof does not contain any detectable NeuGc moieties and/or does not contain any detectable alpha-Gal moieties. In certain embodiments, the HuPTM mAb is a full length or substantially full length mAb with an Fc region.

In specific embodiments, the anti-TNFα HuPTM mAb or antigen-binding fragment thereof has heavy and light chains with the amino acid sequences of the heavy and light chain Fab portions of golimumab as set forth in FIG. 2C (with glutamine (Q) glycosylation sites; asparaginal (N) glycosylation sites, non-consensus asparaginal (N) glycosylation sites; and tyrosine-O-sulfation sites (Y) are as indicated in the legend) has a glycosylation, particularly a 2,6-sialylation, at one or more of the amino acid positions N80, Q121, and/or N171 of the heavy chain (SEQ ID NO: 5) or N162 and/or N214 of the light chain (SEQ ID NO: 6). Alternatively or in addition to, the HuPTM mAb or antigen binding-fragment thereof with the heavy and light chain variable domain sequences of golimumab has a sulfation group at Y112, Y113 and/or Y114 of the heavy chain (SEQ ID NO: 5) and/or Y89 and/or Y90 of the light chain (SEQ ID NO: 6). In other embodiments, the anti-TNFα HuPTM mAb or antigen-binding fragment thereof does not contain any detectable NeuGc moieties and/or does not contain any detectable alpha-Gal moieties. In certain embodiments, the HuPTM mAb is a full length or substantially full length mAb with an Fc region.

In certain embodiments, the HuPTM mAb or Fab is therapeutically effective and is at least 0.5%, 1% or 2% glycosylated and/or sulfated and may be at least 5%, 10% or even 50% or 100% glycosylated and/or sulfated. The goal of gene therapy treatment provided herein is to slow or arrest the progression of or relieve one or more symptoms of non-infectious uveitis, such as to reduce the levels of pain, redness of the eye, sensitivity to light, and/or other discomfort for the patient. Efficacy may be monitored by measuring a reduction in pain, redness of the eye, and/or photophobia and/or an improvement in vision.

Combinations of delivery of the anti-TNFα HuPTM mAb or antigen-binding fragment thereof, to the eye, liver and/or muscles accompanied by delivery of other available treatments are encompassed by the methods provided herein. The additional treatments may be administered before, concurrently, or subsequent to the gene therapy treatment. Available treatments for a subject with non-infectious uveitis that could be combined with the gene therapy provided herein include but are not limited to, azathioprine, methotrexate, mycophenolate mofetil, cyclosporine, cyclophosphamide, corticosteroids (local and/or systemic), and others and administration with anti-TNFα agents, including but not limited to adalimumab, infliximab, or golimumab.

5.4.2. Dose Administration of Anti-TNFα Antibodies

Section 5.2. and 5.4.1 describe recombinant vectors that contain a transgene encoding a HuPTM mAb or HuPTM Fab (or other antigen binding fragment of the HuPTM mAb) that binds to TNFα. Therapeutically effective doses of any such recombinant vector should be administered in any manner such that the recombinant vector enters ocular tissue cells (e.g., retinal cells), e.g. by introducing the recombinant vector into the bloodstream. Alternatively, the vector may be administered directly to the eye, e.g., via subretinal, intravitreal, intracameral, suprachoroidal injection. In specific, embodiments, the vector is administered subretinally, intravitreally, intracamerally, suprachoroidally, subcutaneously, intramuscularly or intravenously. Subretinal, intravitreal, intracameral, suprachoroidal administration should result in expression of the soluble transgene product in cells of the eye. The expression of the transgene encoding an anti-TNFα antibody creates a permanent depot in one or more ocular tissue cells of the patient that continuously supplies the anti-TNFα HuPTM mAb, or antigen binding fragment of the anti-TNFα mAb to ocular tissues of the subject.

In specific embodiments, doses that maintain a plasma concentration of the anti-TNFα antibody transgene product at a Cₘᵢₙ of at least 0.5 μg/mL or at least 1 μg/mL (e.g., Cₘᵢₙ of 1 to 10 μg/ml, 3 to 30 μg/ml or 5 to 15 μg/mL or 5 to 30 μg/mL) are provided.

In specific embodiments, doses that maintain a plasma concentration of the adalimumab antibody, or antigen-binding fragment thereof, at a Cₘᵢₙ of at least 5 μg/mL (e.g., Cₘᵢₙ of 5 to 10 μg/ml or 10 to 20 μg/ml), preferably a Cₘᵢₙ of about 8 μg/mL to 9 μg/mL are provided.

In specific embodiments, doses that maintain a plasma concentration of the infliximab antibody, or antigen-binding fragment thereof, at a Cₘᵢₙ of at least 2 μg/mL (e.g., Cₘᵢₙ of 2 to 10 μg/ml or 10 to 20 μg/ml), preferably at a Cₘᵢₙ of about 5 μg/mL to 6 μg/mL, are provided.

However, in all cases because the transgene product is continuously produced, maintenance of lower concentrations can be effective. Notwithstanding, because the transgene product is continuously produced, maintenance of lower concentrations can be effective. The concentration of the transgene product can be measured in patient blood serum samples.

Pharmaceutical compositions suitable for intravenous, intramuscular, subcutaneous or hepatic administration comprise a suspension of the recombinant vector comprising the transgene encoding the anti-TNFα antibody, or antigen-binding fragment thereof, in a formulation buffer comprising a physiologically compatible aqueous buffer. The formulation buffer can comprise one or more of a polysaccharide, a surfactant, polymer, or oil.

5.5 Anti-IL6 and Anti-IL6R HuPTM Constructs and Formulations for Non-Infectious Uveitis

Compositions and methods are described for the delivery of HuPTM mAbs and antigen-binding fragments thereof, such as HuPTM Fabs, that bind to interleukin-6 receptor (IL6R) or interleukin-6 (IL6) derived from an anti-IL6R or anti-IL6 antibody, such as satralizumab, sarilumab, tocilizumab, siltuximab, clazakizumab, sirukumab, olokizumab, or gerilizumab (FIGS. 3A-31I), indicated for treating non-infectious uveitis. In certain embodiments, the HuPTM mAb has the amino acid sequence of satralizumab, sarilumab, tocilizumab, siltuximab, clazakizumab, sirukumab, olokizumab, or gerilizumab, or an antigen binding fragment thereof. Amino acid sequences of Fab fragments of the antibody are provided in FIGS. 3A-31I. Delivery may be accomplished via gene therapy—e.g., by administering a viral vector or other DNA expression construct encoding an IL6R-binding or IL6-binding HuPTM mAb (or an antigen binding fragment and/or a hyperglycosylated derivative or other derivative, thereof) to patients (human subjects) diagnosed with one or more symptoms of non-infectious uveitis, or alternatively in need of treating, inhibiting or ameliorating a detrimental immune response to create a permanent depot that continuously supplies the human PTM, e.g., human-glycosylated, transgene product.

Transgenes

Provided are recombinant vectors containing a transgene encoding a HuPTM mAb or HuPTM Fab (or other antigen binding fragment of the HuPTM mAb) that binds to IL6R or IL6 that can be administered to deliver the HuPTM mAb or antigen binding fragment in a patient. The transgene is a nucleic acid comprising the nucleotide sequences encoding an antigen binding fragment of an antibody that binds to IL6R or IL6, such as satralizumab, sarilumab, tocilizumab, siltuximab, clazakizumab, sirukumab, olokizumab, or gerilizumab, or variants thereof as detailed herein. The transgene may also encode an anti-IL6R or IL6 antigen binding fragment that contains additional glycosylation sites (e.g., see Courtois et al.).

In certain embodiments, the anti-IL6R antigen-binding fragment transgene comprises the nucleotide sequences encoding the heavy and light chains of the Fab portion of satralizumab (having amino acid sequences of SEQ ID NOs. 7 and 8, respectively, see Table 7 and FIG. 3A). The nucleotide sequences may be codon optimized for expression in human cells. Nucleotide sequences may, for example, comprise the nucleotide sequences of SEQ ID NO: 32 (encoding the satralizumab heavy chain Fab portion) and SEQ ID NO: 33 (encoding the satralizumab light chain Fab portion) as set forth in Table 8. The heavy and light chain sequences both have a signal or leader sequence at the N-terminus appropriate for expression and secretion in human cells, in particular, human ocular tissue cells (e.g., retinal cells) or liver and/or muscle cells. The signal sequence may have the amino acid sequence of MYRMQLLLLIALSLALVTNS (SEQ ID NO:85). Alternatively, the signal sequence may have an amino acid sequence selected from any one of the signal sequences set forth in Table 2 that correspond to the proteins secreted by ocular tissue cell types. Alternatively, the signal sequence may be appropriate for expression in muscle or liver cells, such as those listed in Tables 3 and 4 infra.

In addition to the heavy and light chain variable domain and CH1 and CL domain sequences, the transgenes may comprise, at the C-terminus of the heavy chain CH1 domain sequence, all or a portion of the hinge region. In specific embodiments, the anti-IL6R-antigen binding domain has a heavy chain Fab domain of SEQ ID NO: 7 with additional hinge region sequence starting after the C-terminal valine (V), contains all or a portion of the amino acid sequence ERKSCVECPPCPAPPVAG (SEQ ID NO:163) or ERKSCVECPPCPA (SEQ ID NO:164) as set forth in FIG. 3A. These hinge regions may be encoded by nucleotide sequences at the 3′ end of SEQ ID NO: 32 by the hinge region encoding sequences set forth in Table 6 (SEQ ID NO: 32). In another embodiment, the transgenes comprise the amino acid sequences encoding the full length (or substantially full length) heavy and light chains of the antibody, comprising the Fc domain at the C terminus of the heavy chain, e.g., having an amino acid sequence of SEQ ID NO: 67 (Table 6) or an IgG2 Fc domain, such as SEQ ID No. 62 or as depicted in FIG. 6, or a mutant or variant thereof. The Fc domain may be engineered for altered binding to one or more Fc receptors and/or effector function as disclosed in Section 5.1.9, infra.

In certain embodiments, the anti-IL6R antigen-binding fragment transgene encodes an IL6R antigen-binding fragment comprising a light chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 8. In certain embodiments, the anti-IL6R antigen-binding fragment transgene encodes an IL6R antigen-binding fragment comprising a heavy chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 7. In certain embodiments, the anti-IL6R antigen-binding fragment transgene encodes an antigen-binding fragment comprising a light chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 8 and a heavy chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 7. In specific embodiments, the IL6R antigen binding fragment comprises a heavy chain comprising an amino acid sequence of SEQ ID NO: 7 with 1, 2, 3, 4, 5, 6, 7, 8, 9, 10, 11, 12, 13, 14, or more amino acid substitutions, insertions or deletions, and the substitutions, insertions or deletions are made, e.g., in the framework regions (e.g., those regions outside of the CDRs, which CDRs are underlined in FIG. 3A) or are substitutions with an amino acid present at that position in the heavy chain of one or more of the other therapeutic antibodies, for example, as identified by the alignment in FIG. 9A. In specific embodiments, the IL6R antigen binding fragment comprises a light chain comprising an amino acid sequence of SEQ ID NO: 8 with 1, 2, 3, 4, 5, 6, 7, 8, 9, 10, 11, 12, 13, 14, 15 or more amino acid substitutions, insertions or deletions, and the substitutions, insertions or deletions are made, e.g., in the framework regions (e.g., those regions outside of the CDRs, which CDRs are underlined in FIG. 3A) or are substitutions with an amino acid present at that position in the light chain of one or more of the other therapeutic antibodies, for example, as identified by the alignment in FIG. 9B.

In certain embodiments, the anti-IL6R antigen-binding fragment transgene encodes a hyperglycosylated satralizumab Fab, comprising a heavy chain and a light chain of SEQ ID NOs: 7 and 8, respectively, with one or more of the following mutations: L114N (heavy chain), Q160N or Q160S (light chain), and/or E195N (light chain) (see FIGS. 9A (heavy chain) and 9B (light chain)).

In certain embodiments, the anti-IL6R antigen-binding fragment transgene encodes an antigen-binding fragment and comprises the nucleotide sequences encoding the six satralizumab CDRs which are underlined in the heavy and light chain variable domain sequences of FIG. 3A which are spaced between framework regions, generally human framework regions, and associated with constant domains depending upon the form of the antigen-binding molecule, as is known in the art to form the heavy and/or light chain variable domain of an anti-IL6R antibody or antigen-binding fragment thereof.

In certain embodiments, the anti-IL6R antigen-binding fragment transgene comprises the nucleotide sequences encoding the heavy and light chains of the Fab portion of sarilumab (having amino acid sequences of SEQ ID NOs. 9 and 10, respectively, see Table 7 and FIG. 3B). The nucleotide sequences may be codon optimized for expression in human cells. Nucleotide sequences may, for example, comprise the nucleotide sequences of SEQ ID NO: 34 (encoding the sarilumab heavy chain Fab portion) and SEQ ID NO: 35 (encoding the sarilumab light chain Fab portion) as set forth in Table 8. The heavy and light chain sequences both have a signal or leader sequence at the N-terminus appropriate for expression and secretion in human cells, in particular, human ocular tissue cells (e.g., retinal cells) or liver and/or muscle cells. The signal sequence may have the amino acid sequence of MYRMQLLLLIALSLALVTNS (SEQ ID NO:85). Alternatively, the signal sequence may have an amino acid sequence selected from any one of the signal sequences set forth in Table 2 that correspond to the proteins secreted by ocular tissue cell types. Alternatively, the signal sequence may be appropriate for expression in muscle or liver cells, such as those listed in Tables 3 and 4 infra.

In addition to the heavy and light chain variable domain and CH1 and CL domain sequences, the transgenes may comprise, at the C-terminus of the heavy chain CH1 domain sequence, all or a portion of the hinge region. In specific embodiments, the anti-IL6R-antigen binding domain has a heavy chain Fab domain of SEQ ID NO: 9 with additional hinge region sequence starting after the C-terminal valine (V), contains all or a portion of the amino acid sequence EPKSCDKTHTCPPCPAPELLGG (SEQ ID NO:153), and specifically, EPKSCDKTHL (SEQ ID NO:155), EPKSCDKTHT (SEQ ID NO:156), EPKSCDKTHTCPPCPA (SEQ ID NO:157), EPKSCDKTHLCPPCPA (SEQ ID NO:158), EPKSCDKTHTCPPCPAPELLGGPSVFL (SEQ ID NO:159) or EPKSCDKTHLCPPCPAPELLGGPSVFL (SEQ ID NO:160) as set forth in FIG. 3B. These hinge regions may be encoded by nucleotide sequences at the 3′ end of SEQ ID NO: 34 by the hinge region encoding sequences set forth in Table 8 (SEQ ID NO: 34). In another embodiment, the transgenes comprise the amino acid sequences encoding the full length (or substantially full length) heavy and light chains of the antibody, comprising the Fc domain at the C terminus of the heavy chain, e.g. having an amino acid sequence of SEQ ID NO:185 (Table 6) or an IgG1 Fc domain, such as SEQ ID No. 61 or as depicted in FIG. 6, or a mutant or variant thereof. The Fc domain may be engineered for altered binding to one or more Fc receptors and/or effector function as disclosed in Section 5.1.9, infra.

In certain embodiments, the anti-IL6R antigen-binding fragment transgene encodes an IL6R antigen-binding fragment comprising a light chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 10. In certain embodiments, the anti-IL6R antigen-binding fragment transgene encodes an IL6R antigen-binding fragment comprising a heavy chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 9. In certain embodiments, the anti-IL6R antigen-binding fragment transgene encodes an antigen-binding fragment comprising a light chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 10 and a heavy chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 9. In specific embodiments, the IL6R antigen binding fragment comprises a heavy chain comprising an amino acid sequence of SEQ ID NO: 9 with 1, 2, 3, 4, 5, 6, 7, 8, 9, 10, 11, 12, 13, 14, or more amino acid substitutions, insertions or deletions, and the substitutions, insertions or deletions are made, e.g., in the framework regions (e.g., those regions outside of the CDRs, which CDRs are underlined in FIG. 3B) or are substitutions with an amino acid present at that position in the heavy chain of one or more of the other therapeutic antibodies, for example, as identified by the alignment in FIG. 9A. In specific embodiments, the IL6R antigen binding fragment comprises a light chain comprising an amino acid sequence of SEQ ID NO: 10 with 1, 2, 3, 4, 5, 6, 7, 8, 9, 10, 11, 12, 13, 14, 15 or more amino acid substitutions, insertions or deletions, and the substitutions, insertions or deletions are made, e.g., in the framework regions (e.g., those regions outside of the CDRs, which CDRs are underlined in FIG. 3B) or are substitutions with an amino acid present at that position in the light chain of one or more of the other therapeutic antibodies, for example, as identified by the alignment in FIG. 9B.

In certain embodiments, the anti-IL6R antigen-binding fragment transgene encodes a hyperglycosylated sarilumab Fab, comprising a heavy chain and a light chain of SEQ ID NOs: 9 and respectively, with one or more of the following mutations: M111N (heavy chain), Q160N or Q160S (light chain), and/or E195N (light chain) (see FIGS. 9A (heavy chain) and 9B (light chain)).

In certain embodiments, the anti-IL6R antigen-binding fragment transgene encodes an antigen-binding fragment and comprises the nucleotide sequences encoding the six sarilumab CDRs which are underlined in the heavy and light chain variable domain sequences of FIG. 3B which are spaced between framework regions, generally human framework regions, and associated with constant domains depending upon the form of the antigen-binding molecule, as is known in the art to form the heavy and/or light chain variable domain of an anti-IL6R antibody or antigen-binding fragment thereof.

In certain embodiments, the anti-IL6R antigen-binding fragment transgene comprises the nucleotide sequences encoding the heavy and light chains of the Fab portion of tocilizumab (having amino acid sequences of SEQ ID NOs. 21 and 22, respectively, see Table 7 and FIG. 3H). The nucleotide sequences may be codon optimized for expression in human cells. Nucleotide sequences may, for example, comprise the nucleotide sequences of SEQ ID NO:183 (encoding the tocilizumab heavy chain Fab portion) and SEQ ID NO:184 (encoding the tocilizumab light chain Fab portion) as set forth in Table 8. The heavy and light chain sequences both have a signal or leader sequence at the N-terminus appropriate for expression and secretion in human cells, in particular, human ocular tissue cells (e.g., retinal cells) or liver and/or muscle cells. The signal sequence may have the amino acid sequence of MYRMQLLLLIALSLALVTNS (SEQ ID NO:85). Alternatively, the signal sequence may have an amino acid sequence selected from any one of the signal sequences set forth in Table 2 that correspond to the proteins secreted by ocular tissue cell types. Alternatively, the signal sequence may be appropriate for expression in muscle or liver cells, such as those listed in Tables 3 and 4 infra.

In addition to the heavy and light chain variable domain and CH1 and CL domain sequences, the transgenes may comprise, at the C-terminus of the heavy chain CH1 domain sequence, all or a portion of the hinge region. In specific embodiments, the anti-IL6R-antigen binding domain has a heavy chain Fab domain of SEQ ID NO: 21 with additional hinge region sequence starting after the C-terminal valine (V), contains all or a portion of the amino acid sequence EPKSCDKTHTCPPCPAPELLGG (SEQ ID NO:153), and specifically, EPKSCDKTHL (SEQ ID NO:155), EPKSCDKTHT (SEQ ID NO:156), EPKSCDKTHTCPPCPA (SEQ ID NO:157), EPKSCDKTHLCPPCPA (SEQ ID NO:158), EPKSCDKTHTCPPCPAPELLGGPSVFL (SEQ ID NO:159) or EPKSCDKTHLCPPCPAPELLGGPSVFL (SEQ ID NO:160) as set forth in FIG. 3H. These hinge regions may be encoded by nucleotide sequences at the 3′ end of SEQ ID NO:183 by the hinge region encoding sequences set forth in Table 8 (SEQ ID NO:183). In another embodiment, the transgenes comprise the amino acid sequences encoding the full length (or substantially full length) heavy and light chains of the antibody, comprising the Fc domain at the C terminus of the heavy chain, e.g., having an amino acid sequence of SEQ ID NO: 72 (Table 6) or an IgG1 Fc domain, such as SEQ ID No. 61 or as depicted in FIG. 6, or a mutant or variant thereof. The Fc domain may be engineered for altered binding to one or more Fc receptors and/or effector function as disclosed in Section 5.1.9, infra.

In certain embodiments, the anti-IL6R antigen-binding fragment transgene encodes an IL6R antigen-binding fragment comprising a light chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 22. In certain embodiments, the anti-IL6R antigen-binding fragment transgene encodes an IL6R antigen-binding fragment comprising a heavy chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 21. In certain embodiments, the anti-IL6R antigen-binding fragment transgene encodes an antigen-binding fragment comprising a light chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 22 and a heavy chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 21. In specific embodiments, the IL6R antigen binding fragment comprises a heavy chain comprising an amino acid sequence of SEQ ID NO: 21 with 1, 2, 3, 4, 5, 6, 7, 8, 9, 10, 11, 12, 13, 14, or more amino acid substitutions, insertions or deletions, and the substitutions, insertions or deletions are made, e.g., in the framework regions (e.g., those regions outside of the CDRs, which CDRs are underlined in FIG. 3H) or are substitutions with an amino acid present at that position in the heavy chain of one or more of the other therapeutic antibodies, for example, as identified by the alignment in FIG. 9A. In specific embodiments, the IL6R antigen binding fragment comprises a light chain comprising an amino acid sequence of SEQ ID NO: 22 with 1, 2, 3, 4, 5, 6, 7, 8, 9, 10, 11, 12, 13, 14, 15 or more amino acid substitutions, insertions or deletions, and the substitutions, insertions or deletions are made, e.g., in the framework regions (e.g., those regions outside of the CDRs, which CDRs are underlined in FIG. 3H) or are substitutions with an amino acid present at that position in the light chain of one or more of the other therapeutic antibodies, for example, as identified by the alignment in FIG. 9B.

In certain embodiments, the anti-IL6R antigen-binding fragment transgene encodes a hyperglycosylated tocilizumab Fab, comprising a heavy chain and a light chain of SEQ ID NOs: 21 and 22, respectively, with one or more of the following mutations: L115N (heavy chain), Q160N or Q160S (light chain), and/or E195N (light chain) (see FIGS. 9A (heavy chain) and 9B (light chain)).

In certain embodiments, the anti-IL6R antigen-binding fragment transgene encodes an antigen-binding fragment and comprises the nucleotide sequences encoding the six tocilizumab CDRs which are underlined in the heavy and light chain variable domain sequences of FIG. 3H which are spaced between framework regions, generally human framework regions, and associated with constant domains depending upon the form of the antigen-binding molecule, as is known in the art to form the heavy and/or light chain variable domain of an anti-IL6R antibody or antigen-binding fragment thereof.

In certain embodiments, the anti-IL6 antigen-binding fragment transgene comprises the nucleotide sequences encoding the heavy and light chains of the Fab portion of siltuximab (having amino acid sequences of SEQ ID NOs. 11 and 12, respectively, see Table 7 and FIG. 3C). The nucleotide sequences may be codon optimized for expression in human cells. Nucleotide sequences may, for example, comprise the nucleotide sequences of SEQ ID NO: 36 (encoding the siltuximab heavy chain Fab portion) and SEQ ID NO: 37 (encoding the siltuximab light chain Fab portion) as set forth in Table 8. The heavy and light chain sequences both have a signal or leader sequence at the N-terminus appropriate for expression and secretion in human cells, in particular, human ocular tissue cells (e.g., retinal cells) or liver and/or muscle cells. The signal sequence may have the amino acid sequence of MYRMQLLLLIALSLALVTNS (SEQ ID NO:85). Alternatively, the signal sequence may have an amino acid sequence selected from any one of the signal sequences set forth in Table 2 that correspond to the proteins secreted by ocular tissue cell types. Alternatively, the signal sequence may be appropriate for expression in muscle or liver cells, such as those listed in Tables 3 and 4 infra.

In addition to the heavy and light chain variable domain and CH1 and CL domain sequences, the transgenes may comprise, at the C-terminus of the heavy chain CH1 domain sequence, all or a portion of the hinge region. In specific embodiments, the anti-IL6-antigen binding domain has a heavy chain Fab domain of SEQ ID NO: 11 with additional hinge region sequence starting after the C-terminal valine (V), contains all or a portion of the amino acid sequence EPKSCDKTHTCPPCPAPELLGG (SEQ ID NO:153), and specifically, EPKSCDKTHL (SEQ ID NO:155), EPKSCDKTHT (SEQ ID NO:156), EPKSCDKTHTCPPCPA (SEQ ID NO:157), EPKSCDKTHLCPPCPA (SEQ ID NO:158), EPKSCDKTHTCPPCPAPELLGGPSVFL (SEQ ID NO:159) or EPKSCDKTHLCPPCPAPELLGGPSVFL (SEQ ID NO:160) as set forth in FIG. 3C. These hinge regions may be encoded by nucleotide sequences at the 3′ end of SEQ ID NO: 36 by the hinge region encoding sequences set forth in Table 8 (SEQ ID NO: 36). In another embodiment, the transgenes comprise the amino acid sequences encoding the full length (or substantially full length) heavy and light chains of the antibody, comprising the Fc domain at the C terminus of the heavy chain, e.g. having an amino acid sequence of SEQ ID NO: 68 (Table 6) or an IgG1 Fc domain, such as SEQ ID No. 61 or as depicted in FIG. 6, or a mutant or variant thereof. The Fc domain may be engineered for altered binding to one or more Fc receptors and/or effector function as disclosed in Section 5.1.9, infra.

In certain embodiments, the anti-IL6 antigen-binding fragment transgene encodes an IL6 antigen-binding fragment comprising a light chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 12. In certain embodiments, the anti-IL6 antigen-binding fragment transgene encodes an IL6 antigen-binding fragment comprising a heavy chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 11. In certain embodiments, the anti-IL6 antigen-binding fragment transgene encodes an antigen-binding fragment comprising a light chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 12 and a heavy chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 11. In specific embodiments, the IL6 antigen binding fragment comprises a heavy chain comprising an amino acid sequence of SEQ ID NO: 11 with 1, 2, 3, 4, 5, 6, 7, 8, 9, 10, 11, 12, 13, 14, or more amino acid substitutions, insertions or deletions, and the substitutions, insertions or deletions are made, e.g., in the framework regions (e.g., those regions outside of the CDRs, which CDRs are underlined in FIG. 3C) or are substitutions with an amino acid present at that position in the heavy chain of one or more of the other therapeutic antibodies, for example, as identified by the alignment in FIG. 9A. In specific embodiments, the IL6 antigen binding fragment comprises a light chain comprising an amino acid sequence of SEQ ID NO: 12 with 1, 2, 3, 4, 5, 6, 7, 8, 9, 10, 11, 12, 13, 14, 15 or more amino acid substitutions, insertions or deletions, and the substitutions, insertions or deletions are made, e.g., in the framework regions (e.g., those regions outside of the CDRs, which CDRs are underlined in FIG. 3C) or are substitutions with an amino acid present at that position in the light chain of one or more of the other therapeutic antibodies, for example, as identified by the alignment in FIG. 9B.

In certain embodiments, the anti-IL6 antigen-binding fragment transgene encodes a hyperglycosylated siltuximab Fab, comprising a heavy chain and a light chain of SEQ ID NOs: 11 and 12, respectively, with one or more of the following mutations: S114N (heavy chain), Q159N or Q159S (light chain), and/or E194N (light chain) (see FIGS. 9A (heavy chain) and 9B (light chain)).

In certain embodiments, the anti-IL6 antigen-binding fragment transgene encodes an antigen-binding fragment and comprises the nucleotide sequences encoding the six siltuximab CDRs which are underlined in the heavy and light chain variable domain sequences of FIG. 3C which are spaced between framework regions, generally human framework regions, and associated with constant domains depending upon the form of the antigen-binding molecule, as is known in the art to form the heavy and/or light chain variable domain of an anti-IL6 antibody or antigen-binding fragment thereof.

In certain embodiments, the anti-IL6 antigen-binding fragment transgene comprises the nucleotide sequences encoding the heavy and light chains of the Fab portion of clazakizumab (having amino acid sequences of SEQ ID NOs. 13 and 14, respectively, see Table 7 and FIG. 3D). The nucleotide sequences may be codon optimized for expression in human cells. Nucleotide sequences may, for example, comprise the nucleotide sequences of SEQ ID NO: 38 (encoding the clazakizumab heavy chain Fab portion) and SEQ ID NO: 39 (encoding the clazakizumab light chain Fab portion) as set forth in Table 8. The heavy and light chain sequences both have a signal or leader sequence at the N-terminus appropriate for expression and secretion in human cells, in particular, human ocular tissue cells (e.g., retinal cells) or liver and/or muscle cells. The signal sequence may have the amino acid sequence of MYRMQLLLLIALSLALVTNS (SEQ ID NO:85). Alternatively, the signal sequence may have an amino acid sequence selected from any one of the signal sequences set forth in Table 2 that correspond to the proteins secreted by ocular tissue cell types. Alternatively, the signal sequence may be appropriate for expression in muscle or liver cells, such as those listed in Tables 3 and 4 infra.

In addition to the heavy and light chain variable domain and CH1 and CL domain sequences, the transgenes may comprise, at the C-terminus of the heavy chain CH1 domain sequence, all or a portion of the hinge region. In specific embodiments, the anti-IL6-antigen binding domain has a heavy chain Fab domain of SEQ ID NO: 13 with additional hinge region sequence starting after the C-terminal valine (V), contains all or a portion of the amino acid sequence EPKSCDKTHTCPPCPAPELLGG (SEQ ID NO:153), and specifically, EPKSCDKTHL (SEQ ID NO:155), EPKSCDKTHT (SEQ ID NO:156), EPKSCDKTHTCPPCPA (SEQ ID NO:157), EPKSCDKTHLCPPCPA (SEQ ID NO:158), EPKSCDKTHTCPPCPAPELLGGPSVFL (SEQ ID NO:159) or EPKSCDKTHLCPPCPAPELLGGPSVFL (SEQ ID NO:160) as set forth in FIG. 3D. These hinge regions may be encoded by nucleotide sequences at the 3′ end of SEQ ID NO: 38 by the hinge region encoding sequences set forth in Table 8 (SEQ ID NO: 38). In another embodiment, the transgenes comprise the amino acid sequences encoding the full length (or substantially full length) heavy and light chains of the antibody, comprising the Fc domain at the C terminus of the heavy chain, e.g. having an amino acid sequence of SEQ ID NO: 69 (Table 6) or an IgG1 Fc domain, such as SEQ ID No. 61 or as depicted in FIG. 6, or a mutant or variant thereof. The Fc domain may be engineered for altered binding to one or more Fc receptors and/or effector function as disclosed in Section 5.1.9, infra.

In certain embodiments, the anti-IL6 antigen-binding fragment transgene encodes an IL6 antigen-binding fragment comprising a light chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 14. In certain embodiments, the anti-IL6 antigen-binding fragment transgene encodes an IL6 antigen-binding fragment comprising a heavy chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 13. In certain embodiments, the anti-IL6 antigen-binding fragment transgene encodes an antigen-binding fragment comprising a light chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 14 and a heavy chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 13. In specific embodiments, the IL6 antigen binding fragment comprises a heavy chain comprising an amino acid sequence of SEQ ID NO: 13 with 1, 2, 3, 4, 5, 6, 7, 8, 9, 10, 11, 12, 13, 14, or more amino acid substitutions, insertions or deletions, and the substitutions, insertions or deletions are made, e.g., in the framework regions (e.g., those regions outside of the CDRs, which CDRs are underlined in FIG. 3D) or are substitutions with an amino acid present at that position in the heavy chain of one or more of the other therapeutic antibodies, for example, as identified by the alignment in FIG. 9A. In specific embodiments, the IL6 antigen binding fragment comprises a light chain comprising an amino acid sequence of SEQ ID NO: 14 with 1, 2, 3, 4, 5, 6, 7, 8, 9, 10, 11, 12, 13, 14, 15 or more amino acid substitutions, insertions or deletions, and the substitutions, insertions or deletions are made, e.g., in the framework regions (e.g., those regions outside of the CDRs, which CDRs are underlined in FIG. 3D) or are substitutions with an amino acid present at that position in the light chain of one or more of the other therapeutic antibodies, for example, as identified by the alignment in FIG. 9B.

In certain embodiments, the anti-IL6 antigen-binding fragment transgene encodes a hyperglycosylated clazakizumab Fab, comprising a heavy chain and a light chain of SEQ ID NOs: 13 and 14, respectively, with one or more of the following mutations: L115N (heavy chain), Q163N or Q163 S (light chain), and/or E198N (light chain) (see FIGS. 9A (heavy chain) and 9B (light chain)).

In certain embodiments, the anti-IL6 antigen-binding fragment transgene encodes an antigen-binding fragment and comprises the nucleotide sequences encoding the six clazakizumab CDRs which are underlined in the heavy and light chain variable domain sequences of FIG. 3D which are spaced between framework regions, generally human framework regions, and associated with constant domains depending upon the form of the antigen-binding molecule, as is known in the art to form the heavy and/or light chain variable domain of an anti-IL6 antibody or antigen-binding fragment thereof.

In certain embodiments, the anti-IL6 antigen-binding fragment transgene comprises the nucleotide sequences encoding the heavy and light chains of the Fab portion of sirukumab (having amino acid sequences of SEQ ID NOs. 15 and 16, respectively, see Table 7 and FIG. 3E). The nucleotide sequences may be codon optimized for expression in human cells. Nucleotide sequences may, for example, comprise the nucleotide sequences of SEQ ID NO: 40 (encoding the sirukumab heavy chain Fab portion) and SEQ ID NO: 41 (encoding the sirukumab light chain Fab portion) as set forth in Table 8. The heavy and light chain sequences both have a signal or leader sequence at the N-terminus appropriate for expression and secretion in human cells, in particular, human ocular tissue cells (e.g., retinal cells) or liver and/or muscle cells. The signal sequence may have the amino acid sequence of MYRMQLLLLIALSLALVTNS (SEQ ID NO:85). Alternatively, the signal sequence may have an amino acid sequence selected from any one of the signal sequences set forth in Table 2 that correspond to the proteins secreted by ocular tissue cell types. Alternatively, the signal sequence may be appropriate for expression in muscle or liver cells, such as those listed in Tables 3 and 4 infra.

In addition to the heavy and light chain variable domain and CH1 and CL domain sequences, the transgenes may comprise, at the C-terminus of the heavy chain CH1 domain sequence, all or a portion of the hinge region. In specific embodiments, the anti-IL6-antigen binding domain has a heavy chain Fab domain of SEQ ID NO: 15 with additional hinge region sequence starting after the C-terminal valine (V), contains all or a portion of the amino acid sequence EPKSCDKTHTCPPCPAPELLGG (SEQ ID NO:153), and specifically, EPKSCDKTHL (SEQ ID NO:155), EPKSCDKTHT (SEQ ID NO:156), EPKSCDKTHTCPPCPA (SEQ ID NO:157), EPKSCDKTHLCPPCPA (SEQ ID NO:158), EPKSCDKTHTCPPCPAPELLGGPSVFL (SEQ ID NO:159) or EPKSCDKTHLCPPCPAPELLGGPSVFL (SEQ ID NO:160) as set forth in FIG. 3E. These hinge regions may be encoded by nucleotide sequences at the 3′ end of SEQ ID NO: 40 by the hinge region encoding sequences set forth in Table 8 (SEQ ID NO: 40). In another embodiment, the transgenes comprise the amino acid sequences encoding the full length (or substantially full length) heavy and light chains of the antibody, comprising the Fc domain at the C terminus of the heavy chain, e.g. having an amino acid sequence of SEQ ID NO: 70 (Table 6) or an IgG1 Fc domain, such as SEQ ID No. 61 or as depicted in FIG. 6, or a mutant or variant thereof. The Fc domain may be engineered for altered binding to one or more Fc receptors and/or effector function as disclosed in Section 5.1.9, infra.

In certain embodiments, the anti-IL6 antigen-binding fragment transgene encodes an IL6 antigen-binding fragment comprising a light chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 16. In certain embodiments, the anti-IL6 antigen-binding fragment transgene encodes an IL6 antigen-binding fragment comprising a heavy chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 15. In certain embodiments, the anti-IL6 antigen-binding fragment transgene encodes an antigen-binding fragment comprising a light chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 16 and a heavy chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 15. In specific embodiments, the IL6 antigen binding fragment comprises a heavy chain comprising an amino acid sequence of SEQ ID NO: 15 with 1, 2, 3, 4, 5, 6, 7, 8, 9, 10, 11, 12, 13, 14, or more amino acid substitutions, insertions or deletions, and the substitutions, insertions or deletions are made, e.g., in the framework regions (e.g., those regions outside of the CDRs, which CDRs are underlined in FIG. 3E) or are substitutions with an amino acid present at that position in the heavy chain of one or more of the other therapeutic antibodies, for example, as identified by the alignment in FIG. 9A. In specific embodiments, the IL6 antigen binding fragment comprises a light chain comprising an amino acid sequence of SEQ ID NO: 16 with 1, 2, 3, 4, 5, 6, 7, 8, 9, 10, 11, 12, 13, 14, 15 or more amino acid substitutions, insertions or deletions, and the substitutions, insertions or deletions are made, e.g., in the framework regions (e.g., those regions outside of the CDRs, which CDRs are underlined in FIG. 3E) or are substitutions with an amino acid present at that position in the light chain of one or more of the other therapeutic antibodies, for example, as identified by the alignment in FIG. 9B.

In certain embodiments, the anti-IL6 antigen-binding fragment transgene encodes a hyperglycosylated sirukumab Fab, comprising a heavy chain and a light chain of SEQ ID NOs: 15 and 16, respectively, with one or more of the following mutations: T114N (heavy chain), Q159N or Q159S (light chain), and/or E194N (light chain) (see FIGS. 9A (heavy chain) and 9B (light chain)).

In certain embodiments, the anti-IL6 antigen-binding fragment transgene encodes an antigen-binding fragment and comprises the nucleotide sequences encoding the six sirukumab CDRs which are underlined in the heavy and light chain variable domain sequences of FIG. 3E which are spaced between framework regions, generally human framework regions, and associated with constant domains depending upon the form of the antigen-binding molecule, as is known in the art to form the heavy and/or light chain variable domain of an anti-IL6 antibody or antigen-binding fragment thereof.

In certain embodiments, the anti-IL6 antigen-binding fragment transgene comprises the nucleotide sequences encoding the heavy and light chains of the Fab portion of olokizumab (having amino acid sequences of SEQ ID NOs. 17 and 18, respectively, see Table 7 and FIG. 3F). The nucleotide sequences may be codon optimized for expression in human cells. Nucleotide sequences may, for example, comprise the nucleotide sequences of SEQ ID NO: 42 (encoding the olokizumab heavy chain Fab portion) and SEQ ID NO: 43 (encoding the olokizumab light chain Fab portion) as set forth in Table 8. The heavy and light chain sequences both have a signal or leader sequence at the N-terminus appropriate for expression and secretion in human cells, in particular, human ocular tissue cells (e.g., retinal cells) or liver and/or muscle cells. The signal sequence may have the amino acid sequence of MYRMQLLLLIALSLALVTNS (SEQ ID NO:85). Alternatively, the signal sequence may have an amino acid sequence selected from any one of the signal sequences set forth in Table 2 that correspond to the proteins secreted by ocular tissue cell types. Alternatively, the signal sequence may be appropriate for expression in muscle or liver cells, such as those listed in Tables 3 and 4 infra.

In addition to the heavy and light chain variable domain and CH1 and CL domain sequences, the transgenes may comprise, at the C-terminus of the heavy chain CH1 domain sequence, all or a portion of the hinge region. In specific embodiments, the anti-IL6-antigen binding domain has a heavy chain Fab domain of SEQ ID NO: 17 with additional hinge region sequence starting after the C-terminal valine (V), contains all or a portion of the amino acid sequence ESKYGPPCPPCPAPEFLGG (SEQ ID NO:165), and specifically, ESKYGPPCPPCPA (SEQ ID NO:166), ESKYGPPCPSCPA (SEQ ID NO:167), ESKYGPPCPSCPAPEFLGGPSVFL (SEQ ID NO:168), or ESKYGPPCPPCPAPEFLGGPSVFL (SEQ ID NO:169) as set forth in FIG. 4F. These hinge regions may be encoded by nucleotide sequences at the 3′ end of SEQ ID NO: 42 by the hinge region encoding sequences set forth in Table 8 (SEQ ID NO: 42). In another embodiment, the transgenes comprise the amino acid sequences encoding the full length (or substantially full length) heavy and light chains of the antibody, comprising the Fc domain at the C terminus of the heavy chain, e.g. having an amino acid sequence of SEQ ID NO: 71 (Table 6) or an IgG4 Fc domain, such as SEQ ID No. 63 or as depicted in FIG. 6, or a mutant or variant thereof. The Fc domain may be engineered for altered binding to one or more Fc receptors and/or effector function as disclosed in Section 5.1.9, infra.

In certain embodiments, the anti-IL6 antigen-binding fragment transgene encodes an IL6 antigen-binding fragment comprising a light chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 18. In certain embodiments, the anti-IL6 antigen-binding fragment transgene encodes an IL6 antigen-binding fragment comprising a heavy chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 17. In certain embodiments, the anti-IL6 antigen-binding fragment transgene encodes an antigen-binding fragment comprising a light chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 18 and a heavy chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 17. In specific embodiments, the IL6 antigen binding fragment comprises a heavy chain comprising an amino acid sequence of SEQ ID NO: 17 with 1, 2, 3, 4, 5, 6, 7, 8, 9, 10, 11, 12, 13, 14, or more amino acid substitutions, insertions or deletions, and the substitutions, insertions or deletions are made, e.g., in the framework regions (e.g., those regions outside of the CDRs, which CDRs are underlined in FIG. 3F) or are substitutions with an amino acid present at that position in the heavy chain of one or more of the other therapeutic antibodies, for example, as identified by the alignment in FIG. 9A. In specific embodiments, the IL6 antigen binding fragment comprises a light chain comprising an amino acid sequence of SEQ ID NO: 18 with 1, 2, 3, 4, 5, 6, 7, 8, 9, 10, 11, 12, 13, 14, 15 or more amino acid substitutions, insertions or deletions, and the substitutions, insertions or deletions are made, e.g., in the framework regions (e.g., those regions outside of the CDRs, which CDRs are underlined in FIG. 3F) or are substitutions with an amino acid present at that position in the light chain of one or more of the other therapeutic antibodies, for example, as identified by the alignment in FIG. 9B.

In certain embodiments, the anti-IL6 antigen-binding fragment transgene encodes a hyperglycosylated olokizumab Fab, comprising a heavy chain and a light chain of SEQ ID NOs: 17 and 18, respectively, with one or more of the following mutations: L115N (heavy chain), Q160N or Q160S (light chain), and/or E195N (light chain) (see FIGS. 9A (heavy chain) and 9B (light chain)).

In certain embodiments, the anti-IL6 antigen-binding fragment transgene encodes an antigen-binding fragment and comprises the nucleotide sequences encoding the six olokizumab CDRs which are underlined in the heavy and light chain variable domain sequences of FIG. 3F which are spaced between framework regions, generally human framework regions, and associated with constant domains depending upon the form of the antigen-binding molecule, as is known in the art to form the heavy and/or light chain variable domain of an anti-IL6 antibody or antigen-binding fragment thereof.

In certain embodiments, the anti-IL6 antigen-binding fragment transgene comprises the nucleotide sequences encoding the heavy and light chains of the Fab portion of gerilizumab (having amino acid sequences of SEQ ID NOs. 19 and 20, respectively, see Table 7 and FIG. 2G). The nucleotide sequences may be codon optimized for expression in human cells. Nucleotide sequences may, for example, comprise the nucleotide sequences of SEQ ID NO: 44 (encoding the gerilizumab heavy chain Fab portion) and SEQ ID NO: 45 (encoding the gerilizumab light chain Fab portion) as set forth in Table 8. The heavy and light chain sequences both have a signal or leader sequence at the N-terminus appropriate for expression and secretion in human cells, in particular, human ocular tissue cells (e.g., retinal cells) or liver and/or muscle cells. The signal sequence may have the amino acid sequence of MYRMQLLLLIALSLALVTNS (SEQ ID NO:85). Alternatively, the signal sequence may have an amino acid sequence selected from any one of the signal sequences set forth in Table 2 that correspond to the proteins secreted by ocular tissue cell types. Alternatively, the signal sequence may be appropriate for expression in muscle or liver cells, such as those listed in Tables 3 and 4 infra.

In addition to the heavy and light chain variable domain and CH1 and CL domain sequences, the transgenes may comprise, at the C-terminus of the heavy chain CH1 domain sequence, all or a portion of the hinge region. In specific embodiments, the anti-IL6-antigen binding domain has a heavy chain Fab domain of SEQ ID NO: 19 with additional hinge region sequence starting after the C-terminal valine (V), contains all or a portion of the amino acid sequence EPKSCDKTHTCPPCPAPELLGG (SEQ ID NO:153), and specifically, EPKSCDKTHL (SEQ ID NO: 160), EPKSCDKTHT (SEQ ID NO:156), EPKSCDKTHTCPPCPA (SEQ ID NO:157), EPKSCDKTHLCPPCPA (SEQ ID NO:158), EPKSCDKTHTCPPCPAPELLGGPSVFL (SEQ ID NO:159) or EPKSCDKTHLCPPCPAPELLGGPSVFL (SEQ ID NO:160) as set forth in FIG. 3G. These hinge regions may be encoded by nucleotide sequences at the 3′ end of SEQ ID NO: 44 by the hinge region encoding sequences set forth in Table 8 (SEQ ID NO: 44). In another embodiment, the transgenes comprise the amino acid sequences encoding the full length (or substantially full length) heavy and light chains of the antibody, comprising the Fc domain at the C terminus of the heavy chain, e.g. having an IgG1 Fc domain, such as SEQ ID No. 72 (Table 6) or 61 (depicted in FIG. 6), or a mutant or variant thereof. The Fc domain may be engineered for altered binding to one or more Fc receptors and/or effector function as disclosed in Section 5.1.9, infra.

In certain embodiments, the anti-IL6 antigen-binding fragment transgene encodes an IL6 antigen-binding fragment comprising a light chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 20. In certain embodiments, the anti-IL6 antigen-binding fragment transgene encodes an IL6 antigen-binding fragment comprising a heavy chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 19. In certain embodiments, the anti-IL6 antigen-binding fragment transgene encodes an antigen-binding fragment comprising a light chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 20 and a heavy chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 19. In specific embodiments, the IL6 antigen binding fragment comprises a heavy chain comprising an amino acid sequence of SEQ ID NO: 19 with 1, 2, 3, 4, 5, 6, 7, 8, 9, 10, 11, 12, 13, 14, or more amino acid substitutions, insertions or deletions, and the substitutions, insertions or deletions are made, e.g., in the framework regions (e.g., those regions outside of the CDRs, which CDRs are underlined in FIG. 3G) or are substitutions with an amino acid present at that position in the heavy chain of one or more of the other therapeutic antibodies, for example, as identified by the alignment in FIG. 9. In specific embodiments, the IL6 antigen binding fragment comprises a light chain comprising an amino acid sequence of SEQ ID NO: 20 with 1, 2, 3, 4, 5, 6, 7, 8, 9, 10, 11, 12, 13, 14, 15 or more amino acid substitutions, insertions or deletions, and the substitutions, insertions or deletions are made, e.g., in the framework regions (e.g., those regions outside of the CDRs, which CDRs are underlined in FIG. 3G) or are substitutions with an amino acid present at that position in the light chain of one or more of the other therapeutic antibodies, for example, as identified by the alignment in FIG. 9B.

In certain embodiments, the anti-IL6 antigen-binding fragment transgene encodes a hyperglycosylated gerilizumab Fab, comprising a heavy chain and a light chain of SEQ ID NOs: 19 and 20, respectively, with one or more of the following mutations: M117N (heavy chain), and/or Q198N (light chain) (see FIGS. 9A (heavy chain) and 9B (light chain)).

In certain embodiments, the anti-IL6 antigen-binding fragment transgene encodes an antigen-binding fragment and comprises the nucleotide sequences encoding the six gerilizumab CDRs which are underlined in the heavy and light chain variable domain sequences of FIG. 3G which are spaced between framework regions, generally human framework regions, and associated with constant domains depending upon the form of the antigen-binding molecule, as is known in the art to form the heavy and/or light chain variable domain of an anti-IL6 antibody or antigen-binding fragment thereof.

Gene Therapy Methods

Provided are methods of treating human subjects for non-infectious uveitis by administration of a viral vector containing a transgene encoding an anti-IL6R or anti-IL6 antibody, or antigen binding fragment thereof. The antibody may be satralizumab, sarilumab, tocilizumab, siltuximab, clazakizumab, sirukumab, olokizumab, or gerilizumab, and is, e.g., a full length, substantially full length or Fab fragment thereof, or other antigen-binding fragment thereof.

In embodiments, the patient has been diagnosed with and/or has symptoms associated with non-infectious uveitis. Recombinant vectors used for delivering the transgene are described in Section 5.1 and exemplary transgenes are provided above. Such vectors should have a tropism for human ocular tissue cells and can include non-replicating rAAV, particularly those bearing an AAV8, AAV3B or AAVrh73 capsid. The recombinant vectors, such as shown in FIGS. 3A-3H, can be administered in any manner such that the recombinant vector enters one or more ocular tissue cells, e.g., by introducing the recombinant vector into the eye, for example by subretinal, intravitreal, intracameral, or suprachoroidal administration, or into the bloodstream, for example by intravenous or intramuscular administration. See below for details regarding the methods of treatment.

Subjects to whom such gene therapy is administered can be those responsive to anti-IL6R or anti-IL6 therapy. In certain embodiments, the methods encompass treating patients who have been diagnosed with one or more ocular disorders, or have one or more symptoms associated therewith, and identified as responsive to treatment with an anti-IL6R or anti-IL6 antibody or considered a good candidate for therapy with an anti-IL6 or anti-IL6 antibody. In specific embodiments, the patients have previously been treated with satralizumab, sarilumab, tocilizumab, siltuximab, clazakizumab, sirukumab, olokizumab, or gerilizumab, and have been found to be responsive to satralizumab, sarilumab, tocilizumab, siltuximab, clazakizumab, sirukumab, olokizumab, or gerilizumab. In other embodiments, the patients have been previously treated with an anti-IL6R or anti-IL6 antibody. To determine responsiveness, the anti-IL6R or anti-IL6 antibody or antigen-binding fragment transgene product (e.g., produced in cell culture, bioreactors, etc.) may be administered directly to the subject.

### Human Post Translationally Modified Antibodies

The production of anti-IL6R or anti-IL6 HuPTM mAb or HuPTM Fab, should result in a “biobetter” molecule for the treatment of angioedema accomplished via gene therapy—e.g., by administering a viral vector or other DNA expression construct encoding the anti-IL6 or anti-IL6R HuPTM Fab, subretinally, intravitreally, intracamerally, suprachoroidally, or intravenously to human subjects (patients) diagnosed with or having one or more symptoms of non-infectious uveitis, to create a permanent depot in the eye (and/or liver and/or muscle) that continuously supplies the fully-human post-translationally modified, e.g., human-glycosylated, sulfated transgene product produced by transduced ocular tissue cells.

In specific embodiments, the anti-IL6R HuPTM mAb or antigen-binding fragment thereof has heavy and light chains with the amino acid sequences of the heavy and light chain Fab portions of satralizumab as set forth in FIG. 3A (with glutamine (Q) glycosylation sites; asparaginal (N) glycosylation sites, non-consensus asparaginal (N) glycosylation sites; and tyrosine-O-sulfation sites (Y) are as indicated in the legend) has a glycosylation, particularly a 2,6-sialylation, at one or more of the amino acid positions N77, N161, N194, and/or N203 of the heavy chain (SEQ ID NO: 7) or Q100, N158, and/or N210 of the light chain (SEQ ID NO: 8). Alternatively or in addition to, the HuPTM mAb or antigen binding-fragment thereof with the heavy and light chain variable domain sequences of satralizumab has a sulfation group at Y94, Y95, and/or Y200 of the heavy chain (SEQ ID NO: 7) and/or Y49, Y50, Y86, and/or Y87 of the light chain (SEQ ID NO: 8). In other embodiments, the anti-IL6R HuPTM mAb or antigen-binding fragment thereof does not contain any detectable NeuGc moieties and/or does not contain any detectable alpha-Gal moieties. In certain embodiments, the HuPTM mAb is a full length or substantially full length mAb with an Fc region.

In specific embodiments, the anti-IL6R HuPTM mAb or antigen-binding fragment thereof has heavy and light chains with the amino acid sequences of the heavy and light chain Fab portions of sarilumab as set forth in FIG. 3B (with glutamine (Q) glycosylation sites; asparaginal (N) glycosylation sites, non-consensus asparaginal (N) glycosylation sites; and tyrosine-O-sulfation sites (Y) are as indicated in the legend) has a glycosylation, particularly a 2,6-sialylation, at one or more of the amino acid positions N54, Q108, and/or N158 of the heavy chain (SEQ ID NO: 9) or Q100, N158, and/or N210 of the light chain (SEQ ID NO: 10). Alternatively or in addition to, the HuPTM mAb or antigen binding-fragment thereof with the heavy and light chain variable domain sequences of sarilumab has a sulfation group at Y32, Y94, and/or Y95 of the heavy chain (SEQ ID NO: 9) and/or Y86, Y87, and/or Y192 of the light chain (SEQ ID NO: 10). In other embodiments, the anti-IL6R HuPTM mAb or antigen-binding fragment thereof does not contain any detectable NeuGc moieties and/or does not contain any detectable alpha-Gal moieties. In certain embodiments, the HuPTM mAb is a full length or substantially full length mAb with an Fc region.

In specific embodiments, the anti-IL6R HuPTM mAb or antigen-binding fragment thereof has heavy and light chains with the amino acid sequences of the heavy and light chain Fab portions of tocilizumab as set forth in FIG. 3H (with glutamine (Q) glycosylation sites; asparaginal (N) glycosylation sites, non-consensus asparaginal (N) glycosylation sites; and tyrosine-O-sulfation sites (Y) are as indicated in the legend) has a glycosylation, particularly a 2,6-sialylation, at one or more of the amino acid positions N61, N77, and/or N161 of the heavy chain (SEQ ID NO: 21) or Q100, N158, and/or N210 of the light chain (SEQ ID NO: 22). Alternatively or in addition to, the HuPTM mAb or antigen binding-fragment thereof with the heavy and light chain variable domain sequences of satralizumab has a sulfation group at Y94 and/or Y95 of the heavy chain (SEQ ID NO: 21) and/or Y86 and/or Y87 of the light chain (SEQ ID NO: 22). In other embodiments, the anti-IL6R HuPTM mAb or antigen-binding fragment thereof does not contain any detectable NeuGc moieties and/or does not contain any detectable alpha-Gal moieties. In certain embodiments, the HuPTM mAb is a full length or substantially full length mAb with an Fc region.

In specific embodiments, the anti-IL6 HuPTM mAb or antigen-binding fragment thereof has heavy and light chains with the amino acid sequences of the heavy and light chain Fab portions of siltuximab as set forth in FIG. 3C (with glutamine (Q) glycosylation sites; asparaginal (N) glycosylation sites, non-consensus asparaginal (N) glycosylation sites; and tyrosine-O-sulfation sites (Y) are as indicated in the legend) has a glycosylation, particularly a 2,6-sialylation, at one or more of the amino acid positions Q111 and/or N161 of the heavy chain (SEQ ID NO: 11) or N60, N157, and/or N209 of the light chain (SEQ ID NO: 12). Alternatively or in addition to, the HuPTM mAb or antigen binding-fragment thereof with the heavy and light chain variable domain sequences of siltuximab has a sulfation group at Y94 and/or Y95 of the heavy chain (SEQ ID NO: 11) and/or Y85 and/or Y86 of the light chain (SEQ ID NO: 12). In other embodiments, the anti-IL6 HuPTM mAb or antigen-binding fragment thereof does not contain any detectable NeuGc moieties and/or does not contain any detectable alpha-Gal moieties. In certain embodiments, the HuPTM mAb is a full length or substantially full length mAb with an Fc region.

In specific embodiments, the anti-IL6 HuPTM mAb or antigen-binding fragment thereof has heavy and light chains with the amino acid sequences of the heavy and light chain Fab portions of clazakizumab as set forth in FIG. 3D (with glutamine (Q) glycosylation sites; asparaginal (N) glycosylation sites, non-consensus asparaginal (N) glycosylation sites; and tyrosine-O-sulfation sites (Y) are as indicated in the legend) has a glycosylation, particularly a 2,6-sialylation, at one or more of the amino acid positions N76, Q112, and/or N162 of the heavy chain (SEQ ID NO: 13) or N30, N161, and/or N213 of the light chain (SEQ ID NO: 14). Alternatively or in addition to, the HuPTM mAb or antigen binding-fragment thereof with the heavy and light chain variable domain sequences of clazakizumab has a sulfation group at Y93 and/or Y94 of the heavy chain (SEQ ID NO: 13) and/or Y86 and/or Y87 of the light chain (SEQ ID NO: 14). In other embodiments, the anti-IL6 HuPTM mAb or antigen-binding fragment thereof does not contain any detectable NeuGc moieties and/or does not contain any detectable alpha-Gal moieties. In certain embodiments, the HuPTM mAb is a full length or substantially full length mAb with an Fc region.

In specific embodiments, the anti-IL6 HuPTM mAb or antigen-binding fragment thereof has heavy and light chains with the amino acid sequences of the heavy and light chain Fab portions of sirukumab as set forth in FIG. 3E (with glutamine (Q) glycosylation sites; asparaginal (N) glycosylation sites, non-consensus asparaginal (N) glycosylation sites; and tyrosine-O-sulfation sites (Y) are as indicated in the legend) has a glycosylation, particularly a 2,6-sialylation, at one or more of the amino acid positions Q111 and/or N161 of the heavy chain (SEQ ID NO: 15) or N157 and/or N209 of the light chain (SEQ ID NO: 16). Alternatively or in addition to, the HuPTM mAb or antigen binding-fragment thereof with the heavy and light chain variable domain sequences of sirukumab has a sulfation group at Y94 and/or Y95 of the heavy chain (SEQ ID NO: 15) and/or Y85 and/or Y86 of the light chain (SEQ ID NO: 16). In other embodiments, the anti-IL6 HuPTM mAb or antigen-binding fragment thereof does not contain any detectable NeuGc moieties and/or does not contain any detectable alpha-Gal moieties. In certain embodiments, the HuPTM mAb is a full length or substantially full length mAb with an Fc region.

In specific embodiments, the anti-IL6 HuPTM mAb or antigen-binding fragment thereof has heavy and light chains with the amino acid sequences of the heavy and light chain Fab portions of olokizumab as set forth in FIG. 3F (with glutamine (Q) glycosylation sites; asparaginal (N) glycosylation sites, non-consensus asparaginal (N) glycosylation sites; and tyrosine-O-sulfation sites (Y) are as indicated in the legend) has a glycosylation, particularly a 2,6-sialylation, at one or more of the amino acid positions N79, Q112, N162, and/or N204 of the heavy chain (SEQ ID NO: 17) or Q100, N158 and/or N210 of the light chain (SEQ ID NO: 18). Alternatively or in addition to, the HuPTM mAb or antigen binding-fragment thereof with the heavy and light chain variable domain sequences of olokizumab has a sulfation group at Y96 and/or Y97 of the heavy chain (SEQ ID NO: 17) and/or Y86 and/or Y87 of the light chain (SEQ ID NO: 18). In other embodiments, the anti-IL6 HuPTM mAb or antigen-binding fragment thereof does not contain any detectable NeuGc moieties and/or does not contain any detectable alpha-Gal moieties. In certain embodiments, the HuPTM mAb is a full length or substantially full length mAb with an Fc region.

In specific embodiments, the anti-IL6 HuPTM mAb or antigen-binding fragment thereof has heavy and light chains with the amino acid sequences of the heavy and light chain Fab portions of gerilizumab as set forth in FIG. 3G (with glutamine (Q) glycosylation sites; asparaginal (N) glycosylation sites, non-consensus asparaginal (N) glycosylation sites; and tyrosine-O-sulfation sites (Y) are as indicated in the legend) has a glycosylation, particularly a 2,6-sialylation, at one or more of the amino acid positions N78, Q114, N164 of the heavy chain (SEQ ID NO: 19) or N71 and/or N174 of the light chain (SEQ ID NO: 20). Alternatively or in addition to, the HuPTM mAb or antigen binding-fragment thereof with the heavy and light chain variable domain sequences of gerilizumab has a sulfation group at Y95 and/or Y96 of the heavy chain (SEQ ID NO: 19) and/or Y88 and/or Y89 of the light chain (SEQ ID NO: 20). In other embodiments, the anti-IL6 HuPTM mAb or antigen-binding fragment thereof does not contain any detectable NeuGc moieties and/or does not contain any detectable alpha-Gal moieties. In certain embodiments, the HuPTM mAb is a full length or substantially full length mAb with an Fc region.

In certain embodiments, the HuPTM mAb or Fab is therapeutically effective and is at least 0.5%, 1% or 2% glycosylated and/or sulfated and may be at least 5%, 10% or even 50% or 100% glycosylated and/or sulfated. The goal of gene therapy treatment provided herein is to slow or arrest the progression of or relieve one or more symptoms of non-infectious uveitis. Efficacy may be monitored by measuring a reduction in pain, redness, and/or photophobia and/or an improvement in vision from baseline.

Combinations of delivery of the anti-IL6R or anti-IL6 HuPTM mAb or antigen-binding fragment thereof, to one or more ocular tissues accompanied by delivery of other available treatments are encompassed by the methods provided herein. The additional treatments may be administered before, concurrently, or subsequent to the gene therapy treatment. Available treatments for a subject with non-infectious uveitis that could be combined with the gene therapy provided herein include but are not limited to, azathioprine, methotrexate, mycophenolate mofetil, cyclosporine, cyclophosphamide, corticosteroids (local and/or systemic), and others and administration with anti-IL6R or anti-IL6, including but not limited to sarilumab, satralizumab, tocilizumab, siltuximab, clazakizumab, sirukumab, olokizumab, or gerilizumab.

5.6. Monitoring of Efficacy

The compositions and methods described herein may be assessed for efficacy using any method for assessing efficacy in treating, preventing, or ameliorating NIU. The assessment may be determined in animal models or in human subjects. The efficacy on visual deficits may be measured by best corrected visual acuity (BCVA), for example, assessing the increase in numbers of letters or lines and where efficacy may be assessed as an increase in greater than or equal to 2 ETDRS lines or an increase in logMAR, reduced inflammatory activity of the anterior and posterior chamber according to the SUN classification, and/or reduction in grade of vitreous haze. Physical changes to the eye may be measured by Optical Coherence Tomography, using methods known in the aft.

The compositions and methods described herein may be assessed for efficacy using any method for assessing efficacy in treating, preventing, or ameliorating NIU. The assessment may be determined in animal models or in human subjects. The efficacy on visual deficits may be measured by best corrected visual acuity (BCVA), for example, assessing the increase in numbers of letters or lines and where efficacy may be assessed as an increase in greater than or equal to 2 ETDRS lines or an increase in logMAR, reduced inflammatory activity of the anterior and posterior chamber according to the SUN classification, and/or reduction in grade of vitreous haze. Physical changes to the eye may be measured by Optical Coherence Tomography, using methods known in the aft. Efficacy may further be monitored by determining flare and/or relapse rates, anterior chamber cell, vitreous cell, and vitreous haze grades (e.g. grade of ≤0.5+), and/or number of active retinal or choroidal (inflammatory) lesions (e.g. see Kim J. S. et al, Int Ophthalmol Clin. 2015 Summer; 55(3): 79-110 or Rosenbaum J. T. et al Volume 49, Issue 3, December 2019, Pages 438-445; which are incorporated by reference herein in its entirety).

Endpoints may include, but are not limited to, mean change in vitreous haze grade in the study eye from baseline to 12, 16, 20, 24, or 28 weeks or at time of rescue, if earlier, proportion of responders with no recurrence of active intermediate, posterior, or panuveitis in the study eye at 12, 16, 20, 24, or 28 weeks, mean change in best corrected visual acuity from baseline to 12, 16, 20, 24, or 28 weeks, change from baseline in quality of life/patient reported outcome assessments, mean change in vitreous haze grade and anterior chamber cell grade from baseline to 12, 16, 20, 24, or 28 weeks, or change in immunosuppressive medication score from baseline to 12, 16, 20, 24, or 28 weeks.

### 6 EXAMPLES

### 6.1 Example 1: Adalimumab Fab cDNA-Based Vector

An adalimumab Fab cDNA-based vector was constructed comprising a transgene comprising nucleotide sequences encoding the Fab portion of the heavy and light chain sequences of adalimumab (amino acid sequences being SEQ ID NOs. 1 and 2, respectively). The nucleotide sequence coding for the Fab portion of the heavy and light chain is the nucleotide sequence of SEQ ID NOs. 26 and 27, respectively. Alternatively, the nucleotide sequence of representative adalimumab Fab transgene cassettes are exemplified in the nucleotide sequence of SEQ ID NOs. 49-51 or 222-225. The transgene also comprises nucleotide sequences that encodes a signal peptide, e.g., MYRMQLLLLIALSLALVTNS (SEQ ID NO:85). The nucleotide sequences encoding the light chain and heavy chain are separated by IRES elements or 2A cleavage sites (See Table 4, particularly, SEQ ID NO:142 or 144) to create a bicistronic vector. The vector additionally includes a constitutive promoter, such as CAG, mU1a, EF1a, CB7, a CB or CB long promoter, a tissue-specific promoter, such as a ocular tissue-specific promoter, particularly GRK1 promoter (SEQ ID NO:77), or a BEST1/GRK1 tandem promoter (SEQ ID NO: 275), or an inducible promoter, such as a hypoxia-inducible promoter.

### 6.2. Example 2: Infliximab Fab cDNA-Based Vector

An infliximab Fab cDNA-based vector is constructed comprising a transgene comprising nucleotide sequences encoding the Fab portion of the heavy and light chain sequences of infliximab (amino acid sequences being SEQ ID NOs. 3 and 4, respectively). The nucleotide sequence coding for the Fab portion of the heavy and light chain may be the nucleotide sequence of SEQ ID NOs. 28 and 29, respectively. The transgene also comprises nucleotide sequences that encodes a signal peptide, e.g., MYRMQLLLLIALSLALVTNS (SEQ ID NO:85). The nucleotide sequences encoding the light chain and heavy chain are separated by IRES elements or 2A cleavage sites (See Table 4, particularly, SEQ ID NO:142 or 144) to create a bicistronic vector. The vector additionally includes a constitutive promoter, such as CAG, mU1a, EF1a, CB7, a CB or CB long promoter, a tissue-specific promoter, such as a ocular tissue-specific promoter, particularly GRK1 promoter (SEQ ID NO:77), or a BEST1/GRK1 tandem promoter (SEQ ID NO: 275), or an inducible promoter, such as a hypoxia-inducible promoter.

### 6.3. Example 3: Golimumab Fab cDNA-Based Vector

A golimumab Fab cDNA-based vector is constructed comprising a transgene comprising nucleotide sequences encoding the Fab portion of the heavy and light chain sequences of golimumab (amino acid sequences being SEQ ID NOs. 5 and 6, respectively). The nucleotide sequence coding for the Fab portion of the heavy and light chain may be the nucleotide sequence of SEQ ID NOs. 30 and 31, respectively. The transgene also comprises nucleotide sequences that encodes a signal peptide, e.g., MYRMQLLLLIALSLALVTNS (SEQ ID NO:85). The nucleotide sequences encoding the light chain and heavy chain are separated by IRES elements or 2A cleavage sites (See Table 4, particularly, SEQ ID NO:142 or 144) to create a bicistronic vector. The vector additionally includes a constitutive promoter, such as CAG, mU1a, EF1a, CB7, a CB or CB long promoter, a tissue-specific promoter, such as a ocular tissue-specific promoter, particularly GRK1 promoter (SEQ ID NO:77), or a BEST1/GRK1 tandem promoter (SEQ ID NO: 275), or an inducible promoter, such as a hypoxia-inducible promoter.

### 6.4. Example 4: Satralizumab Fab cDNA-Based Vector

An satralizumab Fab cDNA-based vector is constructed comprising a transgene comprising nucleotide sequences encoding the Fab portion of the heavy and light chain sequences of satralizumab (amino acid sequences being SEQ ID NOs. 7 and 8, respectively). The nucleotide sequence coding for the Fab portion of the heavy and light chain may be the nucleotide sequence of SEQ ID NOs. 32 and 33, respectively. The transgene also comprises nucleotide sequences that encodes a signal peptide, e.g., MYRMQLLLLIALSLALVTNS (SEQ ID NO:85). The nucleotide sequences encoding the light chain and heavy chain are separated by IRES elements or 2A cleavage sites (See Table 4, particularly, SEQ ID NO:142 or 144) to create a bicistronic vector. The vector additionally includes a constitutive promoter, such as CAG, mU1a, EF1a, CB7, a CB or CB long promoter, a tissue-specific promoter, such as a ocular tissue-specific promoter, particularly GRK1 promoter (SEQ ID NO:77), or a BEST1/GRK1 tandem promoter (SEQ ID NO: 275), or an inducible promoter, such as a hypoxia-inducible promoter.

### 6.5. Example 5: Sarilumab Fab cDNA-Based Vector

A sarilumab Fab cDNA-based vector is constructed comprising a transgene comprising nucleotide sequences encoding the Fab portion of the heavy and light chain sequences of sarilumab (amino acid sequences being SEQ ID NOs. 9 and 10, respectively). The nucleotide sequence coding for the Fab portion of the heavy and light chain may be the nucleotide sequence of SEQ ID NOs. 34 and 35, respectively. The transgene also comprises nucleotide sequences that encodes a signal peptide, e.g., MYRMQLLLLIALSLALVTNS (SEQ ID NO:85). The nucleotide sequences encoding the light chain and heavy chain are separated by IRES elements or 2A cleavage sites (See Table 4, particularly, SEQ ID NO:142 or 144) to create a bicistronic vector. The vector additionally includes a constitutive promoter, such as CAG, mU1a, EF1a, CB7, a CB or CB long promoter, such as a ocular tissue-specific promoter, particularly GRK1 promoter (SEQ ID NO:77), or a BEST1/GRK1 tandem promoter (SEQ ID NO: 275), or an inducible promoter, such as a hypoxia-inducible promoter.

### 6.6. Example 6: Siltuximab Fab cDNA-Based Vector

A siltuximab Fab cDNA-based vector is constructed comprising a transgene comprising nucleotide sequences encoding the Fab portion of the heavy and light chain sequences of siltuximab (amino acid sequences being SEQ ID NOs. 11 and 12, respectively). The nucleotide sequence coding for the Fab portion of the heavy and light chain may be the nucleotide sequence of SEQ ID NOs. 36 and 37, respectively. The transgene also comprises nucleotide sequences that encodes a signal peptide, e.g., MYRMQLLLLIALSLALVTNS (SEQ ID NO:85). The nucleotide sequences encoding the light chain and heavy chain are separated by IRES elements or 2A cleavage sites (See Table 4, particularly, SEQ ID NO:142 or 144) to create a bicistronic vector. The vector additionally includes a constitutive promoter, such as CAG, mU1a, EF1a, CB7, a CB or CB long promoter, a tissue-specific promoter, such as a ocular tissue-specific promoter, particularly GRK1 promoter (SEQ ID NO:77), or a BEST1/GRK1 tandem promoter (SEQ ID NO: 275), or an inducible promoter, such as a hypoxia-inducible promoter.

### 6.6. Example 6: Clazakizumab Fab cDNA-Based Vector

A clazakizumab Fab cDNA-based vector is constructed comprising a transgene comprising nucleotide sequences encoding the Fab portion of the heavy and light chain sequences of clazakizumab (amino acid sequences being SEQ ID NOs. 13 and 14, respectively). The nucleotide sequence coding for the Fab portion of the heavy and light chain may be the nucleotide sequence of SEQ ID NOs. 38 and 39, respectively. The transgene also comprises nucleotide sequences that encodes a signal peptide, e.g., MYRMQLLLLIALSLALVTNS (SEQ ID NO:85). The nucleotide sequences encoding the light chain and heavy chain are separated by IRES elements or 2A cleavage sites (See Table 4, particularly, SEQ ID NO:142 or 144) to create a bicistronic vector. The vector additionally includes a constitutive promoter, such as CAG, mU1a, EF1a, CB7, a CB or CB long promoter, a tissue-specific promoter, such as a ocular tissue-specific promoter, particularly GRK1 promoter (SEQ ID NO:77), or a BEST1/GRK1 tandem promoter (SEQ ID NO: 275), or an inducible promoter, such as a hypoxia-inducible promoter.

### 6.7. Example 7: Sirukumab Fab cDNA-Based Vector

An sirukumab Fab cDNA-based vector is constructed comprising a transgene comprising nucleotide sequences encoding the Fab portion of the heavy and light chain sequences of sirukumab (amino acid sequences being SEQ ID NOs. 15 and 16, respectively). The nucleotide sequence coding for the Fab portion of the heavy and light chain may be the nucleotide sequence of SEQ ID NOs. 40 and 41, respectively. The transgene also comprises nucleotide sequences that encodes a signal peptide, e.g., MYRMQLLLLIALSLALVTNS (SEQ ID NO:85). The nucleotide sequences encoding the light chain and heavy chain are separated by IRES elements or 2A cleavage sites (See Table 4, particularly, SEQ ID NO:142 or 144) to create a bicistronic vector. The vector additionally includes a constitutive promoter, such as CAG, mU1a, EF1a, CB7, a CB or CB long promoter, a tissue-specific promoter, such as a ocular tissue-specific promoter, particularly GRK1 promoter (SEQ ID NO:77), or a BEST1/GRK1 tandem promoter (SEQ ID NO: 275), or an inducible promoter, such as a hypoxia-inducible promoter.

### 6.8. Example 8: Olokizumab Fab cDNA-Based Vector

An olokizumab Fab cDNA-based vector is constructed comprising a transgene comprising nucleotide sequences encoding the Fab portion of the heavy and light chain sequences of olokizumab (amino acid sequences being SEQ ID NOs. 17 and 18, respectively). The nucleotide sequence coding for the Fab portion of the heavy and light chain may be the nucleotide sequence of SEQ ID NOs. 42 and 43, respectively. The transgene also comprises nucleotide sequences that encodes a signal peptide, e.g., MYRMQLLLLIALSLALVTNS (SEQ ID NO:85). The nucleotide sequences encoding the light chain and heavy chain are separated by IRES elements or 2A cleavage sites (See Table 4, particularly, SEQ ID NO:142 or 144) to create a bicistronic vector. The vector additionally includes a constitutive promoter, such as CAG, mU1a, EF1a, CB7, a CB or CB long promoter, a tissue-specific promoter, such as a ocular tissue-specific promoter, particularly GRK1 promoter (SEQ ID NO:77), or a BEST1/GRK1 tandem promoter (SEQ ID NO: 275), or an inducible promoter, such as a hypoxia-inducible promoter.

### 6.9. Example 9: Gerilizumab Fab cDNA-Based Vector

A gerilizumab Fab cDNA-based vector is constructed comprising a transgene comprising nucleotide sequences encoding the Fab portion of the heavy and light chain sequences of gerilizumab (amino acid sequences being SEQ ID NOs. 19 and 20, respectively). The nucleotide sequence coding for the Fab portion of the heavy and light chain may be the nucleotide sequence of SEQ ID NOs. 44 and 45, respectively. The transgene also comprises nucleotide sequences that encodes a signal peptide, e.g., MYRMQLLLLIALSLALVTNS (SEQ ID NO:85). The nucleotide sequences encoding the light chain and heavy chain are separated by IRES elements or 2A cleavage sites (See Table 4, particularly, SEQ ID NO:142 or 144) to create a bicistronic vector. The vector additionally includes a constitutive promoter, such as CAG, mU1a, EF1a, CB7, a CB or CB long promoter, a tissue-specific promoter, such as a ocular tissue-specific promoter, particularly GRK1 promoter (SEQ ID NO:77), or a BEST1/GRK1 tandem promoter (SEQ ID NO: 275), or an inducible promoter, such as a hypoxia-inducible promoter.

### 6.10. Example 10: Tocilizumab Fab cDNA-Based Vector

A tocilizumab Fab cDNA-based vector is constructed comprising a transgene comprising nucleotide sequences encoding the Fab portion of the heavy and light chain sequences of tocilizumab (amino acid sequences being SEQ ID NOs. 21 and 22, respectively). The nucleotide sequence coding for the Fab portion of the heavy and light chain may be the nucleotide sequence of SEQ ID NOs:183 and 184, respectively. The transgene also comprises nucleotide sequences that encodes a signal peptide, e.g., MYRMQLLLLIALSLALVTNS (SEQ ID NO:85). The nucleotide sequences encoding the light chain and heavy chain are separated by IRES elements or 2A cleavage sites (See Table 4, particularly, SEQ ID NO:142 or 144) to create a bicistronic vector. The vector additionally includes a constitutive promoter, such as CAG, mU1a, EF1a, CB7, a CB or CB long promoter, a tissue-specific promoter, such as a ocular tissue-specific promoter, particularly GRK1 promoter (SEQ ID NO:77), or a BEST1/GRK1 tandem promoter (SEQ ID NO: 275), or an inducible promoter, such as a hypoxia-inducible promoter.

### 6.11 Example 11: Vectorized Adalimumab IgG and Fab Cassettes: Design and Characterization

An AAV transgene cassette was constructed (SEQ ID NOs: 46 and 47) that drives ubiquitous expression of vectorized adalimumab IgG (SEQ ID NO: 48). The protein coding sequence is composed of the heavy and light chains of adalimumab separated by a Furin cleavage site (SEQ ID NO:146), Gly-Ser-Gly (GSG) linker (SEQ ID NO:148), and T2A self-processing peptide sequence (SEQ ID NO:149). The specific sequence configuration yields expression of separate heavy and light chain peptides. The entire reading frame is codon-optimized and depleted of CpG dinucleotides. Expression is driven by the CAG promoter (SEQ ID NO: 74). Alternatively, an AAV transgene cassette was constructed (SEQ ID NOs: 52 and 53) that drives tissue-specific expression of vectorized adalimumab IgG (SEQ ID NO: 48) driven by the GRK1 promoter (SEQ ID NO:77). In addition, constructs are provided where the CB promoter (SEQ ID NO: 273) or the tandem Best1/GRK promoter (SEQ ID NO: 275) drives expression, and, optionally, the construct includes the VH4 intron (SEQ ID NO: 80), including constructs pAAVCB.VH4.adalimumab (SEQ ID Nos: 276 and 277) or pAAV.CBlong.VH4.adalimumab, pAAV.Best1.GRK1.VH4.adalimumab. Similarly, an additional cassette was developed (SEQ ID NOs: 49 and 50) that drives expression of a Fab containing the adalimumab variable regions (SEQ ID NO: 51). Constructs are outlined in FIGS. 1A and 1B, and sequences are provided in Table 8.

Plasmid expression of adalimumab IgG and Fab fragment from pAAV.CAG.adalimumab.IgG (SEQ ID NO: 46) or pAAV.CAG.adalimumab.Fab (SEQ ID NO: 49) in the supernatant of transfected 293T cells was characterized via western blot and ELISA with recombinant human TNFα. Western blot analysis confirmed expression of heavy and light chains of the full length adalimumab and the light chain of the Fab fragment using goat anti-human Fc domain (1:3000) to detect the full length heavy chain and goat anti-human kappa light chain (1:3000) to detect the light chain, as compared to a control antibody. Both vectors produced adalimumab that bound human TNFα in an ELISA assay. In addition, the pAAV.CAG.adalimumab.IgG (SEQ ID NO: 46) and pAAV.CAG.adalimumab.Fab (SEQ ID NO: 49) plasmids were used to produce recombinant AAV8 vectors. Expression and TNFα binding activity of adalimumab produced from these recombinant AAV8 were confirmed by western blot and ELISA at 1E4 and 1E5 multiplicity of infection (MOI) with the aforementioned assays.

### 6.12 Example 12: Self-Complementary Adalimumab Fab Transgene Cassettes: Design and Characterization

Two self-complementary AAV (scAAV) transgene cassettes encoding vectorized adalimumab Fab were generated (SEQ ID NOS:222, 223, 224, and 225). The transgenes are driven by the ubiquitous mU1a (SEQ ID NO: 75) or EF-1α (SEQ ID NO: 76) core promoters. These plasmids were compared for Fab expression via transfection into 293T cells. The mU1a-driven vector displayed a higher absorbance value suggesting higher Fab concentration within the cell supernatant.

### 6.13 Example 13: TNFα Binding Across Model Species with Vectorized Adalimumab IgG and Fab

Vectorized adalimumab candidates were assessed for binding to TNFα isolated from model species including human, mouse, and rat. Vectorized antibodies were expressed and secreted into cell supernatant following cis plasmid transfection into 293T cells. The cell supernatant was tested in an ELISA where the plates were coated with recombinant TNFα derived human, mouse and rat. Adalimumab IgG effectively bound human and mouse derived TNFα. The Fab demonstrates a similar binding profile to human TNFα as the IgG. However, the Fab displays poor binding to mouse TNFα compared to adalimumab IgG. Both IgG and Fab display reduced binding to rat TNFα as compared to mouse or human.

### 6.14 Example 14: In Vivo Study 1

In this study, full length adalimumab AAV8.CAG.adalimumab.IgG was evaluated for AAV-mediated antibody expression in vivo in mouse ocular tissues via local administration (subretinal, SR). AAV8.GFP and vehicle served as a controls.

TABLE 9
Study Layout

| | | | Inj. | | |
|---|---|---|---|---|---|
| | | | Volume | Dose | N |
| Group | Vector | ROA | (μL) | (vg/eye) | (animals) |
| 1 | AAV8.CAG.Adalim- | SR | 1 | 1.00E+07 | 3 |
| 2 | umab.IgG | SR | 1 | 1.00E+08 | 3 |
| 3 | | SR | 1 | 1.00E+09 | 3 |
| 4 | AAV8.GFP | SR | 1 | 1.00E+08 | 3 |
| 5 | Vehicle | SR | 1 | — | 3 |

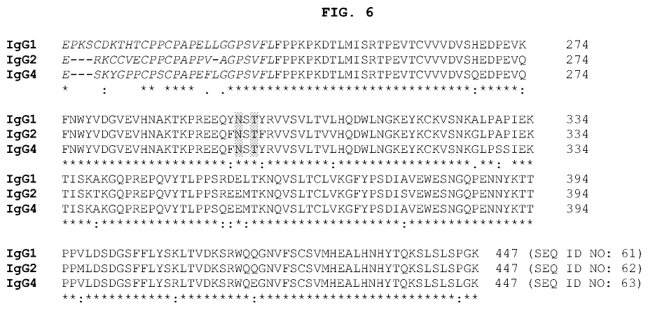

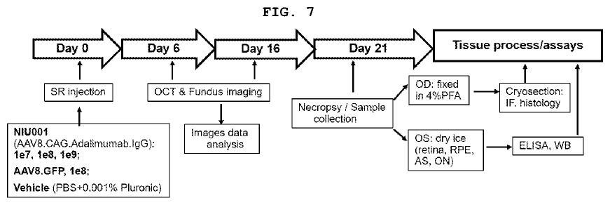

Young adult C57BL/6 (8-10 weeks old) were used for this study. The AAV8.CAG.adalimumab.IgG and AAV8.CAG.GFP vectors were delivered in mouse eyes via subretinal (SR) injection at different doses (1×10⁷, 1×10⁸ and 1×10⁹ vg/eye) in 1 μl of formulation buffer (Table 9). Fundus and OCT imaging were performed at days 6 and 16 after SR injection. Ocular samples were collected at 21 days post administration. Levels of antibody protein expression in ocular tissues (RPE, Retina and Anterior Segment) were quantified by ELISA (FIG. 7). Cell type specificity was determined by immunofluorescent staining with various retinal cell markers. Retina structure changes and neuron survival were evaluated by histology and immunofluorescent staining at 6 and 16 days post administration.

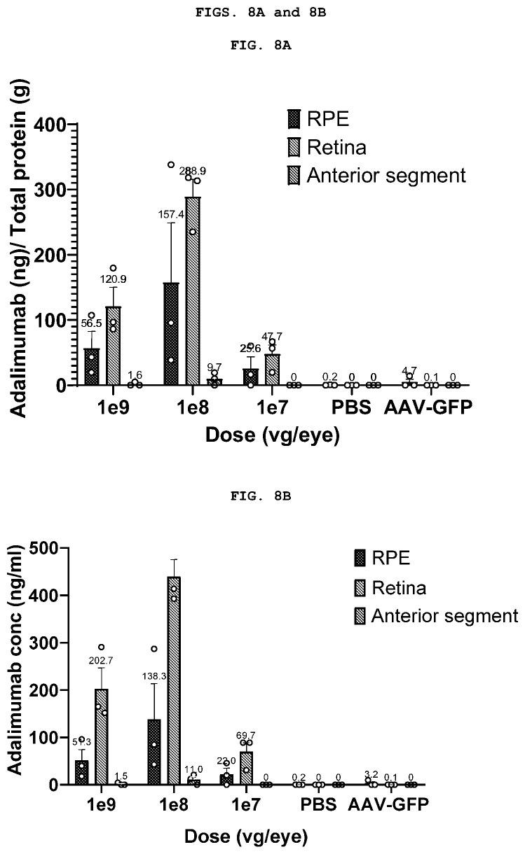

Subretinal injection of at different doses (1×10⁷, 1×10⁸ and 1×10⁹ vg/eye) of vectorized full-length adalimumab (AAV8.CAG.Adalimumab.IgG) resulted in dose-dependent transgene expression (FIGS. 7 and 8) and retinal inflammation (Table 10). At each dose, expression levels were found to be highest in the retina followed by the RPE and anterior segment. Retinal inflammation was detected in 5 out of 6 mice injected with a dose of 1×10⁹ vg/eye at 16 days post administration. No signs of inflammation were detected in mice receiving lower doses. Retinal inflammation/toxicity may be the cause for the lower expression levels detected in mice receiving 1×10⁹ vg/eye (120.9 ng adalimumab/g protein, or adalimumab concentration of 202.7 ng/ml in the retina) compared to 1×10⁸ vg/eye (288.9 ng adalimumab/g protein in the retina, which is equivalent to an adalimumab concentration of 439.3 mg/ml). Adalimumab expression levels are depicted as adalimumab levels (ng) per total protein (g) (FIG. 7) or adalimumab concentration ng per mL (FIG. 8).

Immunofluorescence double staining confirmed expression of adalimumab (as determined by using an antibody against human IgG) in the RPE.

TABLE 10

| Time points/ | Ocular/retinal inflammation | |
|---|---|---|
| treatment | Vehicle | AAV8.CAG.Adalimumab.IgG |

| | | | | |
|---|---|---|---|---|
| Day 6 | N | N | N | N |
| Day 16 | N | N | N | Yes *5/6) |

### 6.15 Example 15: In Vivo Study 2

In this study, full length and Fab adalimumab antibody in an adeno-associated virus (AAV) vector (AAV8.CAG.adalimumab.IgG and AAV8.CAG.adalimumab.Fab), as well as etanercept Fc fusion protein (AAV8.CAG.etanercept), were evaluated for AAV-mediated antibody expression in vivo in mouse ocular tissues via local administration (subretinal (SR), Table 11).

TABLE 11
Study Layout

| | | | Inj. Volume | Dose | N | N |
|---|---|---|---|---|---|---|
| Group | Vector | ROA | (μL) | (vg/eye) | (animals) | (eyes) |
| 1 | AAV8.CAG.Adalimumab.IgG | SR | 1 | 1.00E+09 | 2 | 4 |
| 2 | | SR | 1 | 1.00E+08 | 2 | 4 |
| 3 | AAV8.CAG.Adalimumab.Fab | SR | 1 | 1.00E+09 | 3 | 6 |
| 4 | | SR | 1 | 1.00E+08 | 3 | 6 |
| 5 | AAV8.CAG.Etanercept | SR | 1 | 1.00E+09 | 3 | 6 |
| 6 | | SR | 1 | 1.00E+08 | 3 | 6 |
| 7 | Vehicle | SR | 1 | 1.00E+09 | 2 | 4 |

Vectorized adalimumab and etanercept sequences have been constructed and tested in vitro. Young adult B10.RIII mice (6-8 weeks old) were used for this study. Vectors including AAV8.CAG.adalimumab.IgG (SEQ ID NO: 46), AAV8.CAG.adalimumab.Fab (SEQ ID NO: 49), AAV8.CAG.etanercept, and vehicle were delivered in mouse eyes via subretinal (SR) injection at two different doses (1×10⁸ and 1×10⁹ vg/eye) in 1 μl of formulation buffer (Table 11).

Fundus and OCT imaging was performed at 2 and 4 weeks after SR injection. Ocular samples were collected at 4 weeks post administration. Levels of antibody or fusion protein expression in ocular tissues were quantified by ELISA. Cell type specificity was determined by immunofluorescent staining with various retinal cell markers. Retina structure changes and neuron survival were evaluated by histology and immunofluorescent staining at 2 and 4 weeks post administration.

AAV8.CAG.adalimumab.IgG was well up to the 1×10⁹ dose level (data not shown).

### 6.16 Example 16: In Vivo Study 3

In this study, full length and Fab adalimumab antibody in an adeno-associated virus (AAV) vector (AAV8.CAG.adalimumab.IgG and AAV8.GRK1.adalimumab.Fab, as well as control AAV8.CAG.GFP and AAV9.CAG.GFP were evaluated for AAV-mediated antibody expression in vivo in mouse ocular tissues via local administration (Table 12).

TABLE 12
Study Layout

| | | | Inj. Volume | Dose | N | N |
|---|---|---|---|---|---|---|
| Group | Vector | ROA | (μL) | (vg/eye) | (animals) | (eyes) |
| 1 | AAV8.CAG.Adalimumab.IgG | SR | 1 | 1.00E+09 | 2 | 4 |
| 2 | | SR | 1 | 1.00E+08 | 2 | 4 |
| 3 | AAV8.GRK1.Adalimumab.Fab | SR | 1 | 1.00E+09 | 3 | 6 |
| 4 | | SR | 1 | 1.00E+08 | 3 | 6 |
| 5 | AAV8.GFP | SR | 1 | 1.00E+09 | 2 | 4 |
| 6 | | SR | 1 | 1.00E+08 | 2 | 4 |
| 7 | AAV9.GFP | SR | 1 | 1.00E+09 | 2 | 4 |
| 8 | | SR | 1 | 1.00E+08 | 2 | 4 |
| 9 | Vehicle | SR | 1 | — | 2 | 4 |

Vectorized adalimumab sequences have been constructed and tested in vitro. Young adult B10.RIII mice (6-8 weeks old) were used for this study. Vectors including AAV8.CAG.adalimumab.IgG (SEQ ID NO: 46), AAV8.GRK1.adalimumab.Fab (SEQ ID NO: 49), AAV8.GFP, and AAV9.GFP were delivered in mouse eyes via subretinal (SR) injection at two different doses (1×10⁸ and 1×10⁹ vg/eye) in 111.1 of formulation buffer (Table 12).

Fundus and OCT imaging was performed at 2 and 4 weeks after SR injection. Ocular samples were collected at 4 weeks post administration. Levels of antibody or GFP expression in ocular tissues were quantified by ELISA. Cell type specificity was determined by immunofluorescent staining with various retinal cell markers. Retina structure changes and neuron survival were evaluated by histology and immunofluorescent staining at 2 and 4 weeks post administration.

### 6.17 Example 17: Evaluation of Vector-Expressed Adalimumab Binding Kinetics

Expression and purification of vectorized Adalimumab from AAV produced in mouse eyes was assessed. The purified vectorized adalimumab kinetics of binding to various species of TNFα protein was compared to commercially produced adalimumab in various ligand binding assays.

Binding affinity using Biacore™ (surface plasmon resonance (SPR)) assays: A study was performed to measure the binding affinity of different TNF-alpha (TNFα) molecules to purified antibodies using BiacoreT200. First, binding affinity of TNFα to pAAV.CAG.Adalimumab-produced antibody and was compared to binding of TNFα to commercial adalimumab antibody. Second, binding affinity of TNFα from different species were tested in order to determine the suitability of various species TNFα proteins for later animal model studies. The Biacore assay was performed at 25° C. using HBS-EP+ as the running buffer. Diluted antibodies were captured on the sensor chip through Fc capture method (15-20 minutes capture time). Different species TNFα proteins (human, macaque, porcine, mouse, canine, rabbit and rat) were tested individually as the analyte, followed by injecting running buffer in the dissociation phase. Dissociation rates were calculated [K_{off}=K_{d}=antibody dissociation rate; Kₒₙ=Kₐ=antibody association rate; K_{D}=K_{off}/Kₒₙ], and smaller (lower) K_{D} values indicated the greater the affinity of the antibody for its target.

TABLE 13

| Ligand | Analyte | kₐ (1/Ms) | k_{d} (1/s) | K_{D} (M) |
|---|---|---|---|---|
| Adalimumab | Human TNFα | 4.08E+05 | 1.87E−04 | 4.58E−10 |
| pAAV.CAG.Adalimumab | Human TNFα | 3.98E+05 | 1.70E−04 | 4.28E−10 |
| Adalimumab | Macaque TNFα | 2.60E+05 | 2.01E−04 | 7.72E−10 |
| pAAV.CAG. Adalimumab | Macaque TNFα | 2.46E+05 | 1.77E−04 | 7.20E−10 |
| Adalimumab | Porcine TNFα | 3.03E+05 | 6.59E−04 | 2.17E−09 |
| pAAV.CAG. Adalimumab | Porcine TNFα | 2.69E+05 | 5.52E−04 | 2.05E−09 |
| Adalimumab | Mouse TNFα | 1.39E+05 | 3.16E−04 | 2.27E−09 |
| pAAV.CAG. Adalimumab | Mouse TNFα | 1.51E+05 | 3.23E−04 | 2.13E−09 |
| Adalimumab | Canine TNFα | 1.42E+05 | 2.11E−04 | 1.49E−09 |
| pAAV.CAG. Adalimumab | Canine TNFα | 1.47E+05 | 2.27E−04 | 1.54E−09 |
| Adalimumab | Rabbit TNFα | ND | ND | 5.28E−07 |
| pAAV.CAG. Adalimumab | Rabbit TNFα | ND | ND | 6.14E−07 |
| Adalimumab | Rat TNFα | ND | ND | 6.81E−07 |
| pAAV.CAG. Adalimumab | Rat TNFα | ND | ND | 7.26E−07 |

ND: not detectable

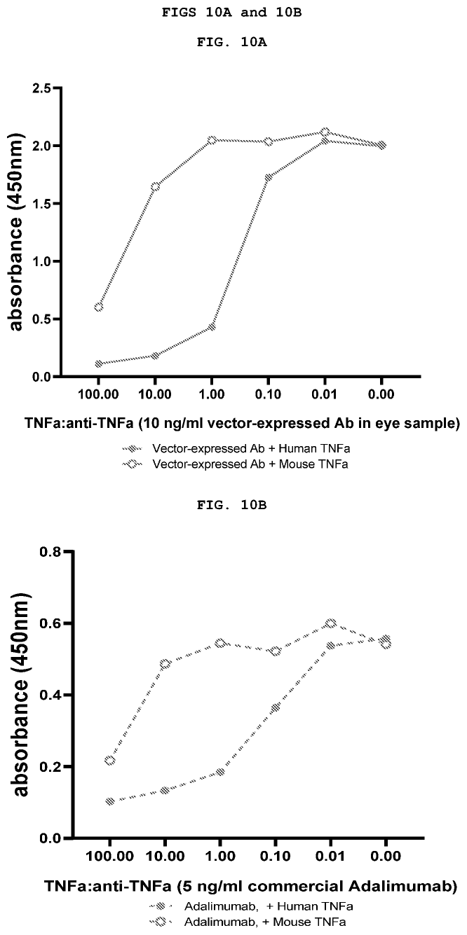

Binding Kinetics by Competitive ELISA: Binding to various concentrations of mouse or human TNFα was compared in a competitive ELISA assay for both vector-expressed adalimumab extracted from mouse eye (following SR administration) and commercial adalimumab (FIGS. 10A and 10B, respectively).

In the Biacore assays, each species TNF-alpha bound to adalimumab produced by CHO cells transfected with cis plasmids expressing adalimumab and commercial adalimumab at essentially the same level. Binding affinity (KD) of different species TNFα to vectorized adalimumab/adalimumab was ranked as follows: Human≥Macaque>Porcine=Mouse=Canine>Rabbit>Rat. Rat TNFα is not expected to compete with human TNFα in a rat model of uveitis (where IVT injection of human TNFα is introduced to induce uveitis).

According to the competitive ELISA assay data, the human TNFα displayed >100×higher affinity to adalimumab compared to mouse TNFα. In the Biacore studies, the human TNFα displayed 5×higher affinity to adalimumab compared to mouse TNFα. Adalimumab binding affinity to rat TNFα was negligible, as reported in the literature for HUMIRA.

### 6.18 Example 18: Measurement of Antibody Effector Function

Antibody effector functions, antibody-dependent cell-mediated cytotoxicity (ADCC) and complement-dependent cytotoxicity (CDC), of the vector-produced adalimumab were evaluated by in vitro assays and compared to commercially produced adalimumab (HUMIRA).

### Materials and Methods

Target cells (CHO/DG44-tm TNFα; GenScript Cat. #RD00746) were maintained with corresponding complete culture medium at 37° C. with 5% CO2. Effector cells (peripheral blood mononuclear cells, PBMCs; Saily Bio Cat. #XFB-HP100B) were thawed at 37° C. and maintained with 1640 complete culture medium at 37° C. with 5% CO2.

For the ADCC dose-response assay, CHO/DG44-tm TNFα and PBMCs were target and effector cells, respectively. With the E/T (effector cell to target cell) ratio at 25:1, adalimumab (commercial) and human IgG1 against CHO/DG44-tm TNFα were used as positive and negative control, respectively. Briefly, the method steps were:

Effector cells (PBMCs) were thawed and resuspended with assay buffer (CellTiter-Glo®Detection Kit (Promega, Cat. #G7573). Target cells were also thawed and resuspended with ADCC assay buffer, then transferred in suspension to an assay plate following a plate map. Controls and test samples in solution were transferred to the assay plate as well, and the assay plate incubated at RT for 30 minutes. The effector cell density was adjusted according to the E/T ratio, then the effector cell suspension was transferred to the assay plate. The assay plate was then incubated in a cell incubator (37° C./5% CO2) for 6 hours, removed, then the supernatant of corresponding wells of the assay plate were transferred to another 96-well assay plate. LDH Mixture (LDH Cytotoxicity Detection Kit, Roche Cat #11644793001) was transferred to the corresponding wells of the second 96-well assay plate and luminescence/absorbance was read with a PHERAStar® (BMG LABTECH) plate reader.

For the CDC dose-response study, CHO/DG44-tm TNFα was used as the target cell. With 5% NHSC (normal human serum complement), adalimumab and human IgG1 against CHO/DG44-tm TNFα were used as positive and negative control, respectively. Briefly, the CDC assay method steps were:

Target cells were harvested by centrifugation and resuspended with assay buffer (CellTiter-Glo®Detection Kit (Promega, Cat. #G7573). Samples and controls were prepared in solution with CDC assay buffer. Target cell density was adjusted and then cell suspension transferred to the assay plate. Controls and test samples in working solution were also transferred to the assay plate, and then assay plate was incubated at RT for 30 minutes, before the Normal Human Serum Complement (NHSC) working solution (Quidel, Cat. #A113) was added to the assay plate. The assay plate was incubated in the cell incubator (37° C./5% CO2) for 4 hours, removed, and the Cell Titer-Glo® working solution was added to the corresponding wells and the plate incubated for about 10-30 minutes at RT. Luminescence data was read on a PHERAStar® FSX (BMG LABTECH) plate reader to determine the number of viable cells. Raw data of ADCC and CDC study were exported from the PHERAStar® FSX system and analyzed using Microsoft Office Excel 2016 and GraphPad Prism 6 software. The formula of ADCC % Target cell lysis=100*(ODSamples−ODTumor cells plus effector cells)/(ODMaximum release−ODMinimum release). The formula of CDC % Target cell lysis=100*(1-(RLUSamples−RLUNHSC)/(RLUCell+NHSC−RLUNHSC)). Relative EC50 values were obtained using four-parameter function as follows, characterizing sigmoid curve where % target cell lysis is against the concentration of the test samples: Y=Bottom+(Top−Bottom)/(1+10\hat{ }((LogEC50−X)*Hill Slope))=Percentage of target cell lysis; and X=Concentration.

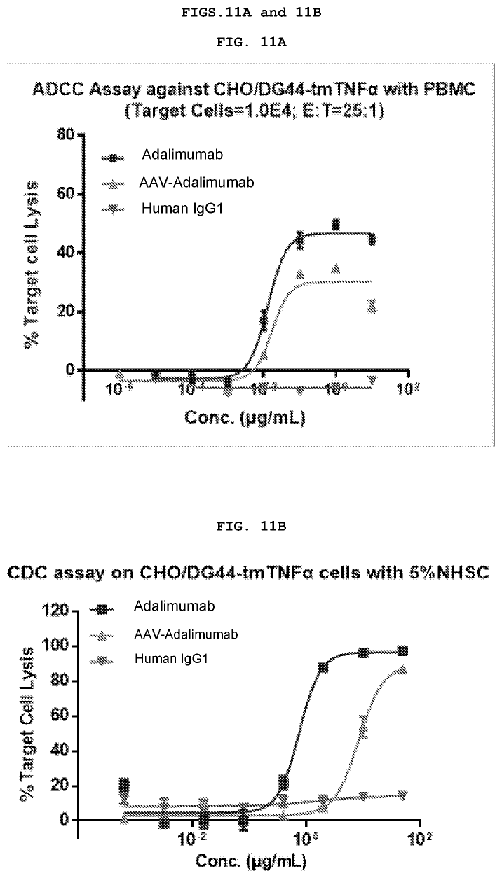

With E/T ratio at 25:1, CHO/DG44-tm TNFα cells were used as the target cells in ADCC dose-response study. Dose-responses and Best-fit values of positive control (Adalimumab), samples and negative control (Human IgG1) are provided in Table 14 and shown in FIG. 11A. EC50 value of Adalimumab was 0.01288 μm/mL.

TABLE 14
ADCC assay.
log(agonist) vs. response - Variable slope (four parameters)
Best-fit values

| ID | Adalimumab | Human IgG1 | AAV-Adalimumab |
|---|---|---|---|
| Bottom | −2.723 | N/A | −3.469 |
| Top | 46.74 | | 30.26 |
| LogEC50 | −1.89 | | −1.775 |
| HillSlope | 1.681 | | ~1.800 |
| EC50 | 0.01288 | | 0.01678 |
| Span | 49.46 | | 33.73 |

With 5% NHSC, CHO/DG44-tm TNFα cells were used as the target cells in CDC dose-response study. Dose-responses and Best-fit values of positive control (Adalimumab), samples and negative control (Human IgG1) are provided in Table 15 and shown in FIG. 11B. EC₅₀ value of Adalimumab was 0.4402 μg/mL.

TABLE 15
CDC Assay.
log(agonist) vs. response - Variable slope (four parameters)
Best-fit values

| ID | Adalimumab | Human IgG1 | AAV-Adalimumab |
|---|---|---|---|
| Bottom | 4.576 | N/A | 3.099 |
| Top | 96.68 | | 89.35 |
| LogEC50 | −0.1203 | | 0.9249 |
| HillSlope | 2.343 | | 2.033 |
| EC50 | 0.7580 | | 8.413 |
| Span | 92.11 | | 86.25 |

### Results and Conclusions:

EC50 value of the positive control (adalimumab) in the ADCC assay was 0.01288 μg/mL and EC50 value of positive control in the CDC assay was 0.758 μg/mL. Under the experimental conditions, both test samples effectively mediated ADCC and CDC activity, and the negative control (human IgG1) was not observed to induce ADCC and CDC activity against CHO/DG44-tm TNFα cells. AAV-adalimumab displayed lower ADCC and CDC activity compared to Adalimumab (HUMIRA®). Without being bound to any one theory, the difference may be due to the post-translational modification such as glycosylation which is expected to differ in manufacturing cell culture. Lower ADCC/CDC-mediated cell lysis with adalimumab from pAAV.CAG.adalimumab transfected cells compared to HUMIRA® at the same dose may be beneficial in terms of immunogenicity for an ocular administered AAV-adalimumab.

### 6.19 Example 19: Human TNF-Alpha Target Engagement Model Characterization (TNF Target Engagement Animal Model)

Binding affinity evaluations confirmed (Example 17, Table 13) that mouse TNFα binds considerably weaker than human TNFα, and adalimumab does not bind rat TNFα. Therefore, target (TNFα) enrichment in this model can be accomplished by injecting human TNFα into a rat eye where endogenous TNFα if stimulated will not be blocked (neutralized) or engaged by exogenous adalimumab, thus allowing normal endogenous receptor activation. In this model, the excess human TNFα target injected into the eye induces local inflammation and can be measured before and after engagement with exogenous antibody (adalimumab or AAV-adalimumab) by ophthalmic exams. The effect of adalimumab or AAV-adalimumab on uveitis caused by the TNF-induced inflammation will also be observed and measured by ophthalmic examination and tissue analysis.

To characterize a dose response and time-course of IVT-administered human TNFα, three (3) doses of human TNFα were given to the eyes of female Lewis rats: low dose/50 ng/eye, middle dose/100 ng/eye and high dose/170 ng/eye. Ocular samples were collected at each time point: 4 hours, 24 hours, 72 hours (Day 3), and 168 hours (Day 7), and human TNFα was measured in each sample.

TABLE 16
Details of the hTNFα target engagement model characterization study

| | | | Route and | |
|---|---|---|---|---|
| | # of | Induction Agent | Volume | Experimental Endpoints |
| Group | Animals | (does, per eye) | (OU) | (to Day 21) |
| 1 | 8 | Vehicle | Intravitreal | Body weights: prior to dose and at necropsy |
| 2 | 8 | hTNFα (50 ng) | (5 uL) | Ophthalmic exams: Baseline, 4 hrs, 7 hrs, |
| 3 | 8 | hTNFα (100 ng) | | Day 3 and Day 7 |
| 4 | 8 | hTNFα (170 ng) | | Ocular tissues (whole eyeballs): collected |
| | | | | from two animals at each time point after |
| | | | | injection (4 hrs, 7 hrs, Day 3 and Day 7). |
| | | | | Both eyes stored frozen at −80° C. until |
| | | | | analysis. |
| 5 | 2 | Naïve | N/A | Ocular tissues (whole eyeballs) were |
| | | | | collected, and served as baseline samples |

Female Lewis rats (6-8 weeks) were used; eyeballs were collected fresh, weighed and snap frozen (−80° C.).

The ability of human TNFα to induce inflammation in the rat eye was measured upon examination of the eye per the Clinical Grading of EAU guidelines of Agarwal, R J et al. (“Rodent Models of Experimental Autoimmune Uveitis.” Methods in Molecular Medicine. 2004: Vol. 102, pp 395-419), as described in Table 17.

TABLE 17
Clinical Grading of EAU in the Rat

| Grade^{a} | Criteria |
|---|---|
| 0 | No disease; eye is translucent and reflects light (red reflex) |
| 0.5 (trace) | Dilated blood vessels in the tris |
| 1 | Engorged blood vessels in the iris; abnormal pupil contraction |
| 2 | Hazy anterior chamber; decreased red reflux |
| 3 | Moderately opaque anterior chamber, but pupil still visible; |
| | dull red reflex |
| 4 | Opaque anterior chamber and obscured pupil; red reflex |
| | absent; proptosis |

^{a}Each higher grade includes the criteria of the preceding one.
Agarwal, R J, et al. Rodent Models of Experimental Autoimmune Uveitis. Methods in Molecular Medicine. 2004: Vol 102, pp 395-419

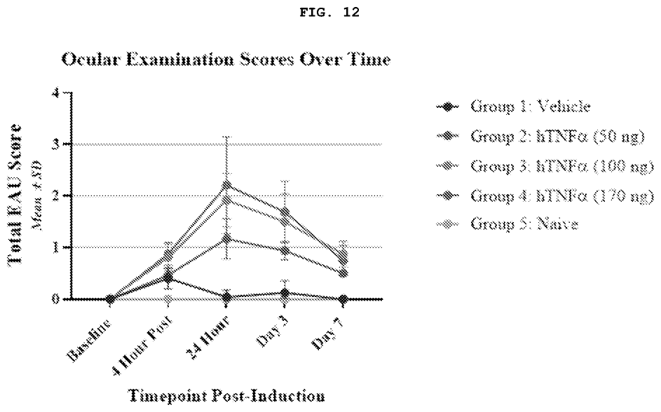

The study shows total EAU scores over time for 3 (rat) groups administered with varying doses of hTNFα. The highest EAU score was approximately 2 for a dose of 170 ng hTNFα administered IVT. After the 24 hour mark, the grade decreases over time to an EAU score of a 1 by 168 hours. See FIG. 12.

### 6.20. Example 20: Human TNF-Alpha Induced Uveitis (Target Engagement Model)

TNFα is an inflammatory cytokine produced by T cells and macrophages/monocytes during acute inflammation. TNF-α is thought to play a key role in uveitic inflammation, such as mediating reactive oxygen species, promotion of angiogenesis, breakdown of the blood-retinal barrier—Retinal cell death—T cell activation and migration. hTNFα is elevated in the aqueous humor and serum in patients with non-infectious uveitis, and is considered a “master regulator” of the inflammatory (immune) response in many organ systems (Tracey D. et al., Pharmacology & Therapeutics 2008, 117, 244-27, Forrester J V et al., American J Ophthalmology, 2018, 189: 77-85; Lee R W et al., Semin Immunopathol, 2014 36:581-59)

Tolerability and Dose Response in normal rats: Three dose cohorts of Lewis rats (low dose/1.0E+7 GC/eye, mid dose/3.0E+8 GC/eye and high dose/1.0E+9 GC/eye) were administered AAV8.CAG.adalimumab subretinally (2.5 μL volume injections). Ophthalmic examinations were performed at day 7, 14 and 21 post-administration. For each rat, one eye was dissected and evaluated at the end of study (21 days) for measurement of adalimumab (e.g. ELISA), and one eye for histology.

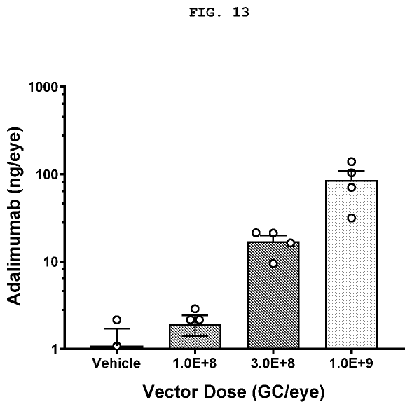

Adalimumab was measured by ELISA with wells coated with recombinant human TNF (as in previous Example). Subretinal injection with AAV8.CAG.Adalimumab at 1.0E+9 GC/eye and 3.0E+8 GC/eye have 86.0 ng/eye and 17.1 ng/eye of adalimumab/eye, respectively, at 21 days post-administration (Lewis rats). See FIG. 13.

The averaged clinical score of dose-dependent hTNFα-induced inflammation was measured at several timepoints in the TNFα model characterization study (see Example 19 above). In order to further demonstrate by different routes of administration (e.g. subretinal or suprachoroidal injection) that AAV-delivered vectorized adalimumab can attenuate the intravitreally-injected hTNFα in a rat eye, further dose characterization was performed in order to select appropriate doses for the TNF model.

Time course evaluation of peak ocular hTNFα levels and adalimumab levels: To further evaluate hTNFα levels at 24 hours following IVT administration in the rat (see Example 19: Human TNFα engagement characterization study), a minimal dilution (matrix) effect was examined.

A solid phase ELISA designed to measure human TNFα in cell culture supernatants (Quantikine Human TNF-alpha Immunoassay, R&D Systems, Cat. #DTA00D) was used to measure hTNFα in spiked samples vs. serial dilutions from 1:2 through 1:256 of hTNFα (170 ng) samples taken from the 24 hour eye sample in the previous characterization study (Example 19).

hTNFα-induced Uveitis models at 170 ng hTNFα/eye quickly decreased to −2.8 ng/eye hTNF-α at 24 hours post IVT injection. It has been reported that Adalimumab—TNF complexes are most likely formed in a 3:1 ratio (Bloemendaal et al. J. Crohns and Colitis, Volume 12, Issue 9, September 2018, pp. 1122-1130; Hu et al. J. Biol. Chem. 288, 27059-27067 (2013); Berkhout et al., Sci Transl Med. 11(477), 2019). Adalimumab has a molecular weight (MW) of 148 KDa, and hTNFα has a subunit molecular mass of 17.3 KDa (with homotrimer MW=51.9 KDa).

Based on adalimumab expression at the highest dose, 1.0E+9 GC/eye of vectorized adalimumab, 50 ng hTNF was selected for model induction.

Efficacy of vectorized AAV-adalimumab in TNFα model: This study is designed to determine potential efficacy and distribution of AAV.adalimumab in a hTNFα-induced engagement model in the rat. The number of animals, data collection time points and parameters for measurement were chosen based on the minimum required to meet the objectives of the study.

Briefly, to evaluate the efficacy of vectorized adalimumab treatment: (i) vector (AAV8.CAG.adalimumab) is administered subretinally (SR) in both eyes (OU) at a dose of 1.0E+9 GC/eye at day −21 (21 days before TNF-α administration), or (ii) 100, 150, 200 or 500 ng/eye commercial adalimumab (in 5 μL) administered IVT at day −1 (1 day prior to TNF-α administration), followed by 50 ng hTNF alpha (induction) administered to Lewis rats by intravitreal (IVT) injection at day 0. Body Weights are measured prior to dose and at necropsy; Ophthalmic Exams are done at baseline, 4, 24 hours and Day 3, and Day 7. Necropsy will be performed at Day 7, whereas one eye per animal/group is analyzed for transgene/TNFα levels, and one eye per animal/group is analyzed for histopathology. The study is summarized in Table 18.

TABLE 18
Rat TNF-α Target Enrichment Experimental Design:

| | | Treatment | Induction | | |
|---|---|---|---|---|---|
| | # of | Route, (OU) | Agent | | |
| Group | Animals | 5 μL/eye | Route, (OU) | Experimental Endpoints | Necropsy |
| 1 | 10 | Formulation Buffer, IVT | hTNFα, IVT | Body Weights: Prior to | Day 7 |
| | | (5 μL/eye) | (50 ng, | dose and at necropsy | One eye per animal/group |
| | | Day −1 | 5 μL/eye) | Ophthalmic Exams: | for transgene/TNFα levels |
| 2 | 10 | Formulation Buffer, SR | | Baseline, 4, 24 hours and Days | One eye per animal/group |
| | | (2.5 μL/eye) | | 3, and 7 | for histopathology |
| | | Day −21 | | | |
| 3 | 10 | Adalimumab, IVT | | | |
| | | (5 μL/eye, 500 ng) | | | |
| | | Day −1 | | | |
| 4 | 10 | AAV.Adalimumab, SR | | | |
| | | (2.5 μL/eye, 1.00E09) | | | |
| | | Day −21 | | | |
| 5* | 4 | Saline, IVT | NA | Body Weights: Prior to | 24 & 48 Hours |
| | | Day −14 | | dose and at necropsy | Post-Dose |
| 6* | 4 | Adalimumab, IVT | | Ophthalmic Exams: | Both eyes from n = 2 |
| | | (100 ng/eye) | | baseline, Day −13, Day −12 | animals/group for |
| | | Day −14 | | Ocular tissues (whole | adalimumab tissue levels |
| 7* | 4 | Adalimumab, IVT | | eyes): | Serum from terminal |
| | | (150 ng/eye) | | 24 and 48 hours (n = 2 animals | animals (n = 2 |
| | | Day −14 | | OU per timepoint) post IVT | animals/group/time point) |
| 8* | 4 | Adalimumab, IVT | | injection (Day −13 and Day −12) | for systemic adalimumab |
| | | (200 ng/eye) | | Serum: 24 and 48 hours | levels |
| | | Day −14 | | (terminal animals) post IVT | |
| 9 | 4 | Adalimumab, IVT | | injection (Day −13 and Day −12) | |
| | | (500 ng/eye) | | | |
| | | Day −14 | | | |

*Note, animals in Group 5-9 will be dosed prior to Groups 1 & 3 to determine dose level needed for Group 3.

Tissue Collections Groups 1˜4 (One eye), Groups 5-8 (All eyes): At the timepoints specified in the experimental design table, animals will be euthanized (protocols will be approved by IACUC). Post euthanasia, aqueous humor (AH) will be collected from both (OU) eyes using a 31-gauge insulin syringe. The AH (10-15 μL) will be dispensed into a polypropylene tube, briefly centrifuged to collect the fluid into the bottom of the tube, and then 10 μL will be transferred to a pre-labelled, 2 mL screw-cap, polypropylene tube. Tubes will then be snap-frozen and stored at −80° C. until analysis. After AH collection, eyes will be enucleated and snap frozen in individual tubes and subsequently stored at −80° C.

### 6.21 Example 21: Evaluation of Regulatory Elements (Promoters) in Retinal Cells

Several AAV constructs were made with GFP or adalimumab under the control of different promoters and optionally a VH4 intron, as such:
AAV8.CAG.GFP or adalimumab
AAV8.U1a.VH4.GFP or adalimumab
AAV8.CB.VH4.GFP or adalimumab
AAV8.CBlong.VH4.GFP or adalimumab
AAV8.GRK1.VH4.GFP or adalimumab
AAV8.Best1.VH4.GFP or adalimumab
AAV8.Best1.GRK1.VH4. GFP or adalimumab

The sequence of each promoter is provided in Table 1 (supra). CAG is considered a strong ubiquitous promoter, while U1a or CB drive expression at a medium level and are ubiquitous with respect to cell type. CB long (CB promoter extended+100 nucleotides of 5′UTR from the chicken beta-actin promoter) will also be tested for promoter strength under the test conditions. BEST1 is considered an RPE specific promoter, whereas GRK1 displays specificity for transcriptional control in photoreceptor cells. A BEST1/GRK1 tandem promoter was also made. The tandem promoter contains a modified GRK1 sequence, such that any start codons (ATG) are modified (T removed) to prevent unintended or aberrant transcripts. An intron is optionally placed proximal to the promoter, upstream of the coding sequence. Sequences of the adalimumab IgG constructs are provided in Table 8.

AAV8.CAG.adalimumab and AAV8.GRK1.adalimumab were tested in a mouse model following subretinal vector administration at two different doses (1.0E=8 or 1.0E+09), and total adalimumab was extracted and measured. Ophthalmic tests (fundus and OCT imaging) were performed at various time points. Animals were euthanized and necropsied at week 4-5 after injection, and eyeballs were collected. Ocular tissues (retinas, RPE & Choroid, and anterior segments) were collected into separate tubes and snap frozen in liquid nitrogen. Tubes were stored at −80° C. until analysis. The right eyes were fixed in 4% paraformaldehyde (PFA) for 1-2 hours, then transferred to 1×PBS. Under the current conditions, Adalimumab concentrations were highest in RPE when driven by CAG promoter at the 1.0E+8 dose.

Additionally, ARPE-19 retinal cells were transfected with AAV receptor (AAVR; Pillay et al. Curr Opin Viral. 2017 June; 24: 124-131. doi:10.1016/j.coviro.2017.06.003). ARPE-AAVR cells were then transfected with AAV cis plasmids expressing GFP under the control of different promoters, and examined for GFP expression. Strong CB promoter-driven expression of GFP is observed in ARPE cells, whereas BEST1, GRK1 and BEST1/GRK promoter-driven genes were comparable, in the tested conditions.

### EQUIVALENTS

Although the invention is described in detail with reference to specific embodiments thereof, it will be understood that variations which are functionally equivalent are within the scope of this invention. Indeed, various modifications of the invention in addition to those shown and described herein will become apparent to those skilled in the art from the foregoing description and accompanying drawings. Such modifications are intended to fall within the scope of the appended claims. Those skilled in the art will recognize or be able to ascertain using no more than routine experimentation, many equivalents to the specific embodiments of the invention described herein. Such equivalents are intended to be encompassed by the following claims.

All publications, patents and patent applications mentioned in this specification are herein incorporated by reference into the specification to the same extent as if each individual publication, patent or patent application was specifically and individually indicated to be incorporated herein by reference in their entireties.

What is claimed is:

1. A pharmaceutical composition for treating non-infectious uveitis in a human subject in need thereof, comprising an adeno-associated virus (AAV) vector having:
(c) a viral capsid that has a tropism for ocular tissue cells; and
(d) an artificial genome comprising an expression cassette flanked by AAV inverted terminal repeats (ITRs), wherein the expression cassette comprises a transgene encoding a heavy chain and a light chain of a substantially full-length or full-length anti-TNFα mAb, anti-IL6 mAb, or anti-IL6R mAb, or an antigen-binding fragment thereof, operably linked to one or more regulatory sequences that promote expression of the transgene in human ocular tissue cells;
wherein said AAV vector is formulated for subretinal, intravitreal, intranasal, intracameral, suprachoroidal, or systemic administration to said human subject.

2. The pharmaceutical composition of claim 1, wherein the viral capsid is at least 95% identical to the amino acid sequence of AAV serotype 1 (AAV1), serotype 2 (AAV2), serotype AAV2.7m8 (AAV2.7m8), serotype 3 (AAV3), serotype 3B (AAV3B), serotype 4 (AAV4), serotype 5 (AAV5), serotype 6 (AAV6), serotype 7 (AAV7), serotype 8 (AAV8), serotype rh8 (AAVrh8), serotype 9 (AAV9), serotype 9e (AAV9e), serotype rh10 (AAVrh10), serotype rh20 (AAVrh20), serotype rh39 (AAVrh39), serotype hu.37 (AAVhu.37), serotype rh73 (AAVrh73), or serotype rh74 (AAVrh74), serotype hu51 (AAV.hu51), serotype hu21 (AAV.hu21), serotype hu12 (AAV.hu12), or serotype hu26 (AAV.hu26).

3. The pharmaceutical composition of claim 1 or 2, wherein the AAV capsid is AAV8, AAV3B, AAV2.7m8, or AAVrh73.

4. The pharmaceutical composition of claims 1 to 3, wherein the ocular tissue cell is a cornea cell, an iris cell, a ciliary body cell, a schlemm's canal cell, a trabecular meshwork cell, a retinal cell, a RPE-choroid tissue cell, or an optic nerve cell.

5. The pharmaceutical composition of claims 1 to 4, wherein the regulatory sequence includes a regulatory sequence from Table 1.

6. The pharmaceutical composition of claim 5, wherein the regulatory sequence is a CAG promoter (SEQ ID NO: 74), a CB promoter (SEQ ID NO: 273 or 274), a human rhodopsin kinase (GRK1) promoter (SEQ ID NOS:77 or 217), a mouse cone arresting (CAR) promoter (SEQ ID NOS: 214-216), a human red opsin (RedO) promoter (SEQ ID NO: 212) or a Best1/GRK1 tandem promoter (SEQ ID NO: 275).

7. The pharmaceutical composition of any of claims 1 to 6, wherein the transgene comprises a Furin/2A linker between the nucleotide sequences coding for the heavy and light chains of said mAb.

8. The pharmaceutical composition of claim 7, wherein said Furin 2A linker is a Furin/T2A linker having the amino acid sequence RKRR(GSG) APVKQTLNFDLLKLAGDVESNPGP (SEQ ID NOS:143 or 144).

9. The pharmaceutical composition of any of claims 1 to 8, wherein the transgene encodes a signal sequence at the N-terminus of the heavy chain and the light chain of said antigen-binding fragment that directs secretion and post translational modification in said human ocular tissue cells.

10. The pharmaceutical composition of claim 9, wherein said signal sequence is MYRMQLLLLIALSLALVTNS (SEQ ID NO:85) or a signal sequence from Table 2.

11. The pharmaceutical composition of any of claims 1 to 10, wherein transgene has the structure:
signal sequence-Heavy chain-Furin site-2A site-signal sequence-Light chain-PolyA.

12. The pharmaceutical composition of any of claims 1 to 11, wherein the anti-TNFα antibody is adalimumab, infliximab, or golimumab, or an antigen binding fragment thereof.

13. The pharmaceutical composition of any of claims 1 to 12, wherein the full-length mAb or the antigen-binding fragment comprises a heavy chain with an amino acid sequence of SEQ ID NO: 1 and optionally an Fc polypeptide with an amino acid sequence of SEQ ID NO: 64 and a light chain with an amino acid sequence of SEQ ID NO: 2; a heavy chain with an amino acid sequence of SEQ ID NO: 3 and optionally an Fc polypeptide with an amino acid sequence of SEQ ID NO: 65 and a light chain with an amino acid sequence of SEQ ID NO: 4; or a heavy chain with an amino acid sequence of SEQ ID NO: 5 and optionally an Fc polypeptide with an amino acid sequence of SEQ ID NO: 65 and a light chain with an amino acid sequence of SEQ ID NO: 6.

14. The pharmaceutical composition of any of claims 1 to 13, wherein the transgene comprises a nucleotide sequence of SEQ ID NO: 26 encoding the heavy chain and a nucleotide sequence of SEQ ID NO: 27 encoding the light chain; a nucleotide sequence of SEQ ID NO: 28 encoding the heavy chain and a nucleotide sequence of SEQ ID NO: 29 encoding the light chain; or a nucleotide sequence of SEQ ID NO: 30 encoding the heavy chain and a nucleotide sequence of SEQ ID NO: 31 encoding the light chain.

15. The pharmaceutical composition of any of claims 1 to 11, wherein the antiIL6 or anti-IL6R antibody is satralizumab, sarilumab, siltuximab, clazakizumab, sirukumab, olokizumab, gerilizumab, or tocilizumab, or an antigen binding fragment thereof.

16. The pharmaceutical composition of any of claim 1 to 11 or 15, wherein the full-length mAb or the antigen-binding fragment comprises a heavy chain with an amino acid sequence of SEQ ID NO: 7 and optionally an Fc polypeptide with an amino acid sequence of SEQ ID NO: 67 and a light chain with an amino acid sequence of SEQ ID NO: 8; a heavy chain with an amino acid sequence of SEQ ID NO: 9 and optionally an Fc polypeptide with an amino acid sequence of SEQ ID NO:185 and a light chain with an amino acid sequence of SEQ ID NO: 10; a heavy chain with an amino acid sequence of SEQ ID NO: 11 and optionally an Fc polypeptide with an amino acid sequence of SEQ ID NO: 68 and a light chain with an amino acid sequence of SEQ ID NO: 12; comprises a heavy chain with an amino acid sequence of SEQ ID NO: 13 and optionally an Fc polypeptide with an amino acid sequence of SEQ ID NO: 69 and a light chain with an amino acid sequence of SEQ ID NO: 14; a heavy chain with an amino acid sequence of SEQ ID NO: 15 and optionally an Fc polypeptide with an amino acid sequence of SEQ ID NO: 70 and a light chain with an amino acid sequence of SEQ ID NO: 16; a heavy chain with an amino acid sequence of SEQ ID NO: 17 and optionally an Fc polypeptide with an amino acid sequence of SEQ ID NO: 71 and a light chain with an amino acid sequence of SEQ ID NO: 18; a heavy chain with an amino acid sequence of SEQ ID NO: 19 and a light chain with an amino acid sequence of SEQ ID NO: 20; or a heavy chain with an amino acid sequence of SEQ ID NO: 21 and optionally an Fc polypeptide with an amino acid sequence of SEQ ID NO: 72 and a light chain with an amino acid sequence of SEQ ID NO: 22.

17. The pharmaceutical composition of any of claim 1, 11 or 15-16, wherein the transgene comprises a nucleotide sequence of SEQ ID NO: 32 encoding the heavy chain and a nucleotide sequence of SEQ ID NO: 33 encoding the light chain; a nucleotide sequence of SEQ ID NO: 34 encoding the heavy chain and a nucleotide sequence of SEQ ID NO: 35 encoding the light chain; a nucleotide sequence of SEQ ID NO: 36 encoding the heavy chain and a nucleotide sequence of SEQ ID NO: 37 encoding the light chain; wherein the transgene comprises a nucleotide sequence of SEQ ID NO: 38 encoding the heavy chain and a nucleotide sequence of SEQ ID NO: 39 encoding the light chain; a nucleotide sequence of SEQ ID NO: 40 encoding the heavy chain and a nucleotide sequence of SEQ ID NO: 41 encoding the light chain; a nucleotide sequence of SEQ ID NO: 42 encoding the heavy chain and a nucleotide sequence of SEQ ID NO: 43 encoding the light chain; wherein the transgene comprises a nucleotide sequence of SEQ ID NO: 44 encoding the heavy chain and a nucleotide sequence of SEQ ID NO: 45 encoding the light chain; or a nucleotide sequence of SEQ ID NO:183 encoding the heavy chain and a nucleotide sequence of SEQ ID NO:184 encoding the light chain.

18. The pharmaceutical composition of claims 1 to 17, wherein the antigen-binding fragment is a Fab, a F(ab′)₂, or an scFv.

19. The pharmaceutical composition of any of claims 1 to 18, wherein the mAb or the antigen-binding fragment thereof is a hyperglycosylated mutant or wherein the Fc polypeptide of the mAb is glycosylated or aglycosylated.

20. The pharmaceutical composition of any of claims 1 to 19, wherein the artificial genome is self complementary.

21. The pharmaceutical composition of any of claims 1 to 20 wherein the artificial genome is the construct EF1ac.Vh4i.Adalimumab.Fab scAAV, mU1a.Vh4i.Adalimumab.Fab scAAV, CAG.Adalimumab.IgG, CAG.Adalimumab.Fab, GRK1.Vh4i.Adalimumab.IgG, CB.VH4i.adalimumab.IgG, or Best1/GRK1.VH4i.adalimumab.IgG.

22. A composition comprising an adeno-associated virus (AAV) vector having:
a. a viral AAV capsid, that is optionally at least 95% identical to the amino acid sequence of AAV serotype 1 (AAV1), serotype 2 (AAV2), serotype 3 (AAV3), serotype 3B (AAV3B), serotype 4 (AAV4), serotype 5 (AAV5), serotype 6 (AAV6), serotype 7 (AAV7), serotype 8 (AAV8), serotype rh8 (AAVrh8), serotype 9 (AAV9), serotype 9e (AAV9e), serotype rh10 (AAVrh10), serotype rh20 (AAVrh20), serotype rh39 (AAVrh39), serotype hu.37 (AAVhu.37), serotype rh73 (AAVrh73), or serotype rh74 (AAVrh74), serotype hu51 (AAV.hu51), serotype hu21 (AAV.hu21), serotype hu12 (AAV.hu12), or serotype hu26 (AAV.hu26); and
b. an artificial genome comprising an expression cassette flanked by AAV inverted terminal repeats (ITRs), wherein the expression cassette comprises a transgene encoding a heavy and a light chain of a substantially full-length or full-length anti-TNFα mAb anti-IL6 antibody or anti-IL6R antibody, or an antigen-binding fragment thereof, operably linked to one or more regulatory sequences that promote expression of the transgene in human ocular tissue cells;
c. wherein the transgene encodes a signal sequence at the N-terminus of the heavy chain and the light chain of said mAb that directs secretion and post translational modification of said mAb in ocular tissue cells.

23. The composition of claim 22, wherein the ocular tissue cell is a cornea cell, an iris cell, a ciliary body cell, a schlemm's canal cell, a trabecular meshwork cell, a retinal cell, a RPE-choroid tissue cell, or an optic nerve cell.

24. The composition of claim 22 or 23, wherein the AAV capsid is AAV2.7m8, AAV8, AAV3B, or AAVrh73.

25. The composition of claims 22 to 24, wherein the anti-TNFα antibody is adalimumab, infliximab, or golimumab, or an antigen binding fragment thereof.

26. The pharmaceutical composition of any of claims 22 to 25, wherein the full-length mAb or the antigen-binding fragment comprises a heavy chain with an amino acid sequence of SEQ ID NO: 1 and optionally an Fc polypeptide with an amino acid sequence of SEQ ID NO: 64 and a light chain with an amino acid sequence of SEQ ID NO: 2; a heavy chain with an amino acid sequence of SEQ ID NO: 3 and optionally an Fc polypeptide with an amino acid sequence of SEQ ID NO: 65 and a light chain with an amino acid sequence of SEQ ID NO: 4; or a heavy chain with an amino acid sequence of SEQ ID NO: 5 and optionally an Fc polypeptide with an amino acid sequence of SEQ ID NO: 65 and a light chain with an amino acid sequence of SEQ ID NO: 6.

27. The pharmaceutical composition of claims 22 to 26, wherein the transgene comprises a nucleotide sequence of SEQ ID NO: 26 encoding the heavy chain and a nucleotide sequence of SEQ ID NO: 27 encoding the light chain; a nucleotide sequence of SEQ ID NO: 28 encoding the heavy chain and a nucleotide sequence of SEQ ID NO: 29 encoding the light chain; or a nucleotide sequence of SEQ ID NO: 30 encoding the heavy chain and a nucleotide sequence of SEQ ID NO: 31 encoding the light chain.

28. The pharmaceutical composition of any of claims 22 to 27, wherein the antiIL6 or anti-IL6R antibody is satralizumab, sarilumab, siltuximab, clazakizumab, sirukumab, olokizumab, gerilizumab, or tocilizumab, or an antigen binding fragment thereof.

29. The pharmaceutical composition of any of claim 22 to 24 or 27, wherein the full-length mAb or the antigen-binding fragment comprises a heavy chain with an amino acid sequence of SEQ ID NO: 7 and optionally an Fc polypeptide with an amino acid sequence of SEQ ID NO: 67 and a light chain with an amino acid sequence of SEQ ID NO: 8; a heavy chain with an amino acid sequence of SEQ ID NO: 9 and optionally an Fc polypeptide with an amino acid sequence of SEQ ID NO:185 and a light chain with an amino acid sequence of SEQ ID NO: 10; a heavy chain with an amino acid sequence of SEQ ID NO: 11 and optionally an Fc polypeptide with an amino acid sequence of SEQ ID NO: 68 and a light chain with an amino acid sequence of SEQ ID NO: 12; comprises a heavy chain with an amino acid sequence of SEQ ID NO: 13 and optionally an Fc polypeptide with an amino acid sequence of SEQ ID NO: 69 and a light chain with an amino acid sequence of SEQ ID NO: 14; a heavy chain with an amino acid sequence of SEQ ID NO: 15 and optionally an Fc polypeptide with an amino acid sequence of SEQ ID NO: 70 and a light chain with an amino acid sequence of SEQ ID NO: 16; a heavy chain with an amino acid sequence of SEQ ID NO: 17 and optionally an Fc polypeptide with an amino acid sequence of SEQ ID NO: 71 and a light chain with an amino acid sequence of SEQ ID NO: 18; a heavy chain with an amino acid sequence of SEQ ID NO: 19 and a light chain with an amino acid sequence of SEQ ID NO: 20; or a heavy chain with an amino acid sequence of SEQ ID NO: 21 and optionally an Fc polypeptide with an amino acid sequence of SEQ ID NO: 72 and a light chain with an amino acid sequence of SEQ ID NO: 22.

30. The pharmaceutical composition of any of claim 22-24 or 28-29, wherein the transgene comprises a nucleotide sequence of SEQ ID NO: 32 encoding the heavy chain and a nucleotide sequence of SEQ ID NO: 33 encoding the light chain; a nucleotide sequence of SEQ ID NO: 34 encoding the heavy chain and a nucleotide sequence of SEQ ID NO: 35 encoding the light chain; a nucleotide sequence of SEQ ID NO: 36 encoding the heavy chain and a nucleotide sequence of SEQ ID NO: 37 encoding the light chain; wherein the transgene comprises a nucleotide sequence of SEQ ID NO: 38 encoding the heavy chain and a nucleotide sequence of SEQ ID NO: 39 encoding the light chain; a nucleotide sequence of SEQ ID NO: 40 encoding the heavy chain and a nucleotide sequence of SEQ ID NO: 41 encoding the light chain; a nucleotide sequence of SEQ ID NO: 42 encoding the heavy chain and a nucleotide sequence of SEQ ID NO: 43 encoding the light chain; wherein the transgene comprises a nucleotide sequence of SEQ ID NO: 44 encoding the heavy chain and a nucleotide sequence of SEQ ID NO: 45 encoding the light chain; or a nucleotide sequence of SEQ ID NO:183 encoding the heavy chain and a nucleotide sequence of SEQ ID NO:184 encoding the light chain.

31. The composition of any of claims 22 to 30, wherein the transgene comprises a Furin/2A linker between the nucleotide sequences coding for the heavy and light chains of said mAb.

32. The composition of any of claims 22 to 31, wherein the nucleic acid encoding a Furin 2A linker is incorporated into the expression cassette in between the nucleotide sequences encoding the heavy and light chain sequences, resulting in a construct with the structure: Signal sequence-Heavy chain-Furin site-2A site-Signal sequence-Light chain-PolyA.

33. The composition of claims 22 to 32, wherein said Furin 2A linker is a Furin/T2A linker having the amino acid sequence RKRR(GSG) APVKQTLNFDLLKLAGDVESNPGP (SEQ ID NOS:143 or 144).

34. The composition of any of claims 22 to 33, wherein said signal sequence is MYRMQLLLLIALSLALVTNS (SEQ ID NO:85) or a signal sequence from Table 2.

35. The composition of any of claims 22 to 34, wherein the artificial genome is self complementary.

36. The composition of any of claims 22 to 27 or 31 to 35, wherein the artificial genome is the construct EF1ac.Vh4i.Adalimumab.Fab scAAV, mU1a.Vh4i.Adalimumab.Fab scAAV, CAG.Adalimumab.IgG, CAG.Adalimumab.Fab, GRK1.Vh4i.Adalimumab.IgG, CB.VH4i.adalimumab.IgG, or Best1/GRK1.VH4i.adalimumab.IgG.

37. A method of treating non-infectious uveitis in a human subject in need thereof, comprising subretinally, intravitreally, intranasally, intracamerally, suprachoroidally, or systemically administering to the subject a therapeutically effective amount of a composition comprising a recombinant AAV comprising a transgene encoding an anti-TNFα mAb, anti-IL6 mAb, or anti-IL6R mAb, or antigen-binding fragment thereof, operably linked to one or more regulatory sequences that control expression of the transgene in ocular tissue cells.

38. A method of treating non-infectious uveitis in a human subject in need thereof, comprising: subretinally, intravitreally, intranasally, intracamerally, suprachoroidally, or systemically administering to said subject a therapeutically effective amount of a recombinant nucleotide expression vector comprising a transgene encoding an anti-TNFα mAb, anti-IL6 mAb, or anti-IL6R mAb, or antigen-binding fragment thereof, operably linked to one or more regulatory sequences that control expression of the transgene in human ocular tissue cells, so that a depot is formed that releases a human post-translationally modified (HuPTM) form of anti-TNFα mAb, anti-IL6 mAb, or anti-IL6R mAb or antigen-binding fragment thereof.

39. The method of claim 37 or 38 wherein the anti-TNFα mAb is adalimumab, infliximab or golimumab.

40. The method of claims 37 to 39, wherein the full-length anti-TNFα mAb or the antigen-binding fragment comprises a heavy chain with an amino acid sequence of SEQ ID NO: 1 and optionally an Fc polypeptide with an amino acid sequence of SEQ ID NO: 64 and a light chain with an amino acid sequence of SEQ ID NO: 2; or a heavy chain with an amino acid sequence of SEQ ID NO: 3 and optionally an Fc polypeptide with an amino acid sequence of SEQ ID NO: 65 and a light chain with an amino acid sequence of SEQ ID NO: 4; a heavy chain with an amino acid sequence of SEQ ID NO: 5 and optionally an Fc polypeptide with an amino acid sequence of SEQ ID NO: 65 and a light chain with an amino acid sequence of SEQ ID NO: 6.

41. The method of claims 37 to 40, wherein the transgene comprises a nucleotide sequence of SEQ ID NO: 26 encoding the heavy chain and a nucleotide sequence of SEQ ID NO: 27 encoding the light chain; a nucleotide sequence of SEQ ID NO: 28 encoding the heavy chain and a nucleotide sequence of SEQ ID NO: 29 encoding the light chain; or a nucleotide sequence of SEQ ID NO: 30 encoding the heavy chain and a nucleotide sequence of SEQ ID NO: 31 encoding the light chain.

42. The method of any of claims 37 to 38, wherein the antiIL6 or anti-IL6R antibody is satralizumab, sarilumab, siltuximab, clazakizumab, sirukumab, olokizumab, gerilizumab, or tocilizumab, or an antigen binding fragment thereof.

43. The method of any of claim 37 to 38 or 42, wherein the full-length mAb or the antigen-binding fragment comprises a heavy chain with an amino acid sequence of SEQ ID NO: 7 and optionally an Fc polypeptide with an amino acid sequence of SEQ ID NO: 67 and a light chain with an amino acid sequence of SEQ ID NO: 8; a heavy chain with an amino acid sequence of SEQ ID NO: 9 and optionally an Fc polypeptide with an amino acid sequence of SEQ ID NO:185 and a light chain with an amino acid sequence of SEQ ID NO: 10; a heavy chain with an amino acid sequence of SEQ ID NO: 11 and optionally an Fc polypeptide with an amino acid sequence of SEQ ID NO: 68 and a light chain with an amino acid sequence of SEQ ID NO: 12; comprises a heavy chain with an amino acid sequence of SEQ ID NO: 13 and optionally an Fc polypeptide with an amino acid sequence of SEQ ID NO: 69 and a light chain with an amino acid sequence of SEQ ID NO: 14; a heavy chain with an amino acid sequence of SEQ ID NO: 15 and optionally an Fc polypeptide with an amino acid sequence of SEQ ID NO: 70 and a light chain with an amino acid sequence of SEQ ID NO: 16; a heavy chain with an amino acid sequence of SEQ ID NO: 17 and optionally an Fc polypeptide with an amino acid sequence of SEQ ID NO: 71 and a light chain with an amino acid sequence of SEQ ID NO: 18; a heavy chain with an amino acid sequence of SEQ ID NO: 19 and a light chain with an amino acid sequence of SEQ ID NO: 20; or a heavy chain with an amino acid sequence of SEQ ID NO: 21 and optionally an Fc polypeptide with an amino acid sequence of SEQ ID NO: 72 and a light chain with an amino acid sequence of SEQ ID NO: 22.

44. The method of any of claims 37 to 38 or 42 to 43, wherein the transgene comprises a nucleotide sequence of SEQ ID NO: 32 encoding the heavy chain and a nucleotide sequence of SEQ ID NO: 33 encoding the light chain; a nucleotide sequence of SEQ ID NO: 34 encoding the heavy chain and a nucleotide sequence of SEQ ID NO: 35 encoding the light chain; a nucleotide sequence of SEQ ID NO: 36 encoding the heavy chain and a nucleotide sequence of SEQ ID NO: 37 encoding the light chain; wherein the transgene comprises a nucleotide sequence of SEQ ID NO: 38 encoding the heavy chain and a nucleotide sequence of SEQ ID NO: 39 encoding the light chain; a nucleotide sequence of SEQ ID NO: 40 encoding the heavy chain and a nucleotide sequence of SEQ ID NO: 41 encoding the light chain; a nucleotide sequence of SEQ ID NO: 42 encoding the heavy chain and a nucleotide sequence of SEQ ID NO: 43 encoding the light chain; wherein the transgene comprises a nucleotide sequence of SEQ ID NO: 44 encoding the heavy chain and a nucleotide sequence of SEQ ID NO: 45 encoding the light chain; or a nucleotide sequence of SEQ ID NO:183 encoding the heavy chain and a nucleotide sequence of SEQ ID NO:184 encoding the light chain.

45. The method of claims 37 to 44, wherein the ocular tissue cell is a cornea cell, an iris cell, a ciliary body cell, a schlemm's canal cell, a trabecular meshwork cell, a retinal cell, a RPE-choroid tissue cell, or an optic nerve cell.

46. The method of any of claims 37 to 44 wherein the viral capsid is at least 95% identical to the amino acid sequence of an AAV serotype 1 (AAV1), serotype 2 (AAV2), serotype AAV2.7m8 (AAV2.7m8), serotype 3 (AAV3), serotype 3B (AAV3B), serotype 4 (AAV4), serotype 5 (AAV5), serotype 6 (AAV6), serotype 7 (AAV7), serotype 8 (AAV8), serotype rh8 (AAVrh8), serotype 9 (AAV9), serotype 9e (AAV9e), serotype rh10 (AAVrh10), serotype rh20 (AAVrh20), serotype rh39 (AAVrh39), serotype hu.37 (AAVhu.37), serotype rh73 (AAVrh73), or serotype rh74 (AAVrh74), serotype hu51 (AAV.hu51), serotype hu21 (AAV.hu21), serotype hu12 (AAV.hu12), or serotype hu26 (AAV.hu26).

47. The method of any of claims 37 to 46, wherein the AAV capsid is AAV2.7m8, AAV8, AAV3B, or AAVrh73.

48. The method of any of claims 37 to 46, wherein the regulatory sequence includes a regulatory sequence from Table 1 or Table 1a.

49. The method of claim 48, wherein the regulator sequence is a CAG promoter (SEQ ID NO: 74), a CB promoter (SEQ ID NO: 273 or 274), human rhodopsin kinase (GRK1) promoter (SEQ ID NOS:77 or 217), a mouse cone arresting (CAR) promoter (SEQ ID NOS: 214-216), a human red opsin (RedO) promoter (SEQ ID NO: 212) or a Best1/GRK1 tandem promoter (SEQ ID NO: 275).

50. The method of any of claims 37 to 49, wherein the transgene comprises a Furin/2A linker between the nucleotide sequences coding for the heavy and light chains of said mAb.

51. The method of claim 50, wherein said Furin 2A linker is a Furin/T2A linker having the amino acid sequence RKRR(GSG) APVKQTLNFDLLKLAGDVESNPGP (SEQ ID NOS:143 or 144).

52. The method of any of claims 37 to 51, wherein the transgene encodes a signal sequence at the N-terminus of the heavy chain and the light chain of said antigen-binding fragment that directs secretion and post translational modification in said human ocular tissue cells.

53. The method of claim 52, wherein said signal sequence is MYRMQLLLLIALSLALVTNS (SEQ ID NO:85) or a signal sequence from Table 2.

54. The method of any of claims 37 to 53, wherein transgene has the structure: Signal sequence-Heavy chain-Furin site-2A site-Signal sequence-Light chain-PolyA.

55. The method of any of claims 37 to 54, wherein the mAb is a hyperglycosylated mutant or wherein the Fc polypeptide of the mAb is glycosylated or aglycosylated.

56. The method of any of claims 37 to 55 wherein the mAb contains an alpha 2,6-sialylated glycan.

57. The method of any of claims 37 to 56 wherein the mAb is glycosylated but does not contain detectable NeuGc and/or α-Gal.

58. The method of any of claims 37 to 57 wherein the mAb contains a tyrosine sulfation.

59. The method of any of claims 37 to 58 in which production of said HuPTM form of said mAb or antigen-binding fragment thereof is confirmed by transducing human ocular tissue cells in culture with said recombinant nucleotide expression vector and expressing said mAb or antigen-binding fragment thereof.

60. The method of claim 37 or 59, wherein the therapeutically effective amount is determined to be sufficient to maintain a concentration of at least 10 ng/ml in aqueous humor, vitreous humor, RPE, retina, and/or anterior segment/chamber.

61. The method of claim 37 or 60, wherein the therapeutically effective amount is determined to be sufficient to improve best corrected visual acuity (BCVA) by >=2 ETDRS lines or increase in logMAR, reduced inflammatory activity of the anterior and posterior chamber according to the SUN classification, and/or reduction in grade of vitreous haze.

62. The method of any of claims 37 to 61, wherein the rAAV is self complementary.

63. The method of any of claims 37 to 62 wherein the transgene within the construct EF1ac.Vh4i.Adalimumab.Fab scAAV, mU1a.Vh4i.Adalimumab.Fab scAAV, CAG.Adalimumab.IgG, CAG.Adalimumab.Fab, GRK1.Vh4i.Adalimumab.IgG, CB.VH4i.adalimumab.IgG, or Best1/GRK1.VH4i.adalimumab.IgG.

64. A method of producing recombinant AAVs comprising:
(c) culturing a host cell containing:
(i) an artificial genome comprising a cis expression cassette flanked by AAV ITRs, wherein the cis expression cassette comprises comprising a transgene encoding a substantially full-length or full-length anti-TNFα mAb, anti-IL6, or anti-IL6R or antigen-binding fragment thereof, operably linked to one or more regulatory sequences that promote expression of the transgene in human ocular tissue cells;
(ii) a trans expression cassette lacking AAV ITRs, wherein the trans expression cassette encodes an AAV rep and an AAV capsid protein operably linked to expression control elements that drive expression of the AAV rep and the AAV capsid protein in the host cell in culture and supply the AAV rep and the AAV capsid protein in trans, wherein the capsid has ocular tissue cell tropism;
(iii) sufficient adenovirus helper functions to permit replication and packaging of the artificial genome by the AAV capsid protein; and
(d) recovering recombinant AAV encapsidating the artificial genome from the cell culture.

65. The method of claim 64, wherein the transgene encodes a substantially full-length or full-length mAb or antigen binding fragment that comprises the heavy and light chain variable domains of adalimumab, infliximab, golimumab, satralizumab, sarilumab, siltuximab, clazakizumab, sirukumab, olokizumab, gerilizumab, or tocilizumab wherein the AAV capsid protein is an AAV2.7m8, AAV8, AAV3B, or AAVrh73, capsid protein.

66. The method of claim 64 or 65, wherein the ocular tissue cell is a cornea cell, an iris cell, a ciliary body cell, a schlemm's canal cell, a trabecular meshwork cell, a retinal cell, a RPE-choroid tissue cell, or an optic nerve cell.

67. A host cell containing:
d. an artificial genome comprising a cis expression cassette flanked by AAV ITRs, wherein the cis expression cassette comprises comprising a transgene encoding a substantially full-length or full-length anti-TNFα mAb, anti-IL6 mAb or anti-IL6R mAb, or antigen-binding fragment thereof, operably linked to one or more regulatory sequences that promote expression of the transgene in human ocular tissue cells;
e. a trans expression cassette lacking AAV ITRs, wherein the trans expression cassette encodes an AAV rep and an AAV capsid protein operably linked to expression control elements that drive expression of the AAV rep and the AAV capsid protein in the host cell in culture and supply the AAV rep and the AAV capsid protein in trans, wherein the capsid has ocular tissue cell tropism;
f. sufficient adenovirus helper functions to permit replication and packaging of the artificial genome by the AAV capsid protein.

68. The host cell of claim 67, wherein the transgene encodes a substantially full-length or full-length mAb or antigen binding fragment that comprises the heavy and light chain variable domains of adalimumab, infliximab, golimumab, satralizumab, sarilumab, siltuximab, clazakizumab, sirukumab, olokizumab, gerilizumab, or tocilizumab.

69. The host cell of claim 67 or 68, wherein the AAV capsid protein is an AAV2.7m8, AAV8, AAV3B, or AAVrh73 capsid protein.

70. The host cell of claims 67 to 69, wherein the ocular tissue cell is a cornea cell, an iris cell, a ciliary body cell, a schlemm's canal cell, a trabecular meshwork cell, a retinal cell, a RPE-choroid tissue cell, or an optic nerve cell.
